(19) United States
(12) Patent Application Publication
(10) Pub. No.: US 2026/0327610 A1
(43) Pub. Date: Oct. 22, 2026

(54) ENGINEERED CONSTRUCTS WITH CASSETTE ARRANGEMENTS FOR INCREASED TRANSCRIPTION OF RNA PAYLOADS

(71) Applicant: Shape Therapeutics Inc., Seattle, WA (US)

(72) Inventors: Stephen BURLEIGH, Shoreline, WA (US); Yiannis SAVVA, Seattle, WA (US)

(21) Appl. No.: 19/483,196

(22) PCT Filed: May 14, 2024
(86) PCT No.: PCT/US2024/029340

§ 371 Date: Nov. 10, 2025

Related U.S. Application Data
(60) Provisional application No. 63/466,627, filed on May 15, 2023.
(60) Provisional application No. 63/613,541, filed on Dec. 21, 2023.
(60) Provisional application No. 63/559,087, filed on Feb. 28, 2024.
(60) Provisional application No. 63/569,019, filed on Mar. 22, 2024.

(51) Int. Cl. C12N 15/11; C12N 15/10; C12N 15/86

(52) U.S. Cl. CPC C12N 15/111; C12N 15/102; C12N 15/86; C12N 2750/14143

(57) ABSTRACT

Described herein are viral vectors comprising a plurality of expression cassettes encoding small RNA payloads, such as engineered guide RNAs. The viral vectors comprising a plurality of expression cassettes may be engineered to increase expression of the small RNA payload encoded by one or more expression cassette. The viral vectors comprising a plurality of expression cassettes include various sequence elements that may enhance expression of the small RNA payload, such as transcription factor binding sequences, transcriptional termination sequences, and core promoter sequences. Sequence elements may be combined or interchanged to tune small RNA payload expression levels. The expression cassettes may be arranged in an orientation to enhance small RNA payload expression levels. Also described herein are methods of editing a target gene using a small RNA payload encoded by a viral vector comprising a plurality of expression cassettes.

### CROSS-REFERENCE

This application claims the benefit of U.S. Provisional Application No. 63/466,627, filed May 15, 2023, U.S. Provisional Application No. 63/613,541, filed Dec. 21, 2023, U.S. Provisional Application No. 63/559,087, filed Feb. 28, 2024, and U.S. Provisional Application No. 63/569,019, filed Mar. 22, 2024, which applications are incorporated herein by reference in their entireties.

### SEQUENCE LISTING

The instant application contains a Sequence Listing which has been submitted electronically in extensible Markup Language (XML) format and is hereby incorporated by reference in its entirety. Said XML copy, created on May 6, 2024, is named “421688-722021_SL.xml” and is 117,230 bytes in size.

### BACKGROUND

A wide variety of diseases and disorders are caused by mutations, deletions, altered expression, or altered splicing of genes. RNAs can serve as a mechanism for gene therapy, such as by editing a mutated RNA sequence associated with a disease. There is a need for engineered vectors to increase or modulate expression of RNA payloads.

### SUMMARY

In various aspects, the present disclosure provides a polynucleotide comprising a plurality of expression cassette sequences comprising a first expression cassette sequence and a second expression cassette sequence, wherein: the first expression cassette sequence and the second expression cassette sequence each independently comprise: a DNA sequence encoding a promoter sequence, a DNA sequence encoding an engineered guide RNA (gRNA) sequence under transcriptional control of the promoter sequence, a DNA sequence encoding a SmOPT and a small nuclear RNA (snRNA) processing hairpin sequence, and a DNA sequence encoding a transcription termination sequence; at least one of the DNA sequences encoding a promoter sequence comprises at least 80% sequence identity to SEQ ID NO: 5; the first expression cassette sequence and the second expression cassette sequence are each independently arranged in a 5′ to 3′ orientation to have a read directionality of forward; the DNA sequence encoding an engineered guide RNA (gRNA) sequence of the first expression cassette sequence and the second expression cassette sequence encodes the same engineered guide RNA (gRNA) sequence, and the first and second expression cassette sequences are different sequences.

In some aspects, the first expression cassette sequence and the second expression cassette sequence are orientated in a tandem read orientation. In some aspects, the DNA sequence encoding a promoter sequence comprises at least 80%, at least 85%, at least 90%, at least 95%, or 100% sequence identity to SEQ ID NO: 5 or SEQ ID NO: 67. In some aspects, the DNA sequence encoding a transcription termination comprises at least 80%, at least 85%, at least 90%, at least 95%, or 100% sequence identity to SEQ ID NO: 78, SEQ ID NO: 79, or SEQ ID NO: 80. In some aspects, the DNA sequence encoding a SmOPT and a small nuclear RNA (snRNA) processing hairpin sequence comprises at least 80%, at least 85%, at least 90%, at least 95%, or 100% sequence identity to SEQ ID NO: 92.

In some aspects, the DNA sequence encoding a promoter sequence of the first expression cassette sequence comprises at least 80%, at least 85%, at least 90%, at least 95%, or 100% sequence identity to SEQ ID NO: 5, and the DNA sequence encoding a transcription termination sequence of the first expression cassette sequence comprises at least 80%, at least 85%, at least 90%, at least 95%, or 100% sequence identity to SEQ ID NO: 78 or SEQ ID NO: 79.

In some aspects, the DNA sequence encoding a promoter sequence of the second expression cassette sequence comprises at least 80%, at least 85%, at least 90%, at least 95%, or 100% sequence identity to SEQ ID NO: 67, and the DNA sequence encoding a transcription termination sequence of the second expression cassette sequence comprises at least 80%, at least 85%, at least 90%, at least 95%, or 100% sequence identity to SEQ ID NO: 79 or SEQ ID NO: 80.

In some aspects, the DNA sequence encoding a promoter sequence of the first expression cassette sequence comprises at least 80%, at least 85%, at least 90%, at least 95%, or 100% sequence identity to SEQ ID NO: 5, and the DNA sequence encoding a promoter sequence of the second expression cassette sequence comprises at least 80%, at least 85%, at least 90%, at least 95%, or 100% sequence identity to SEQ ID NO: 67.

In some aspects, the DNA sequence encoding a transcription termination sequence of the first expression cassette sequence comprises at least 80%, at least 85%, at least 90%, at least 95%, or 100% sequence identity to SEQ ID NO: 78 or SEQ ID NO: 79, and the DNA sequence encoding a transcription termination sequence of the second expression cassette sequence comprises at least 80%, at least 85%, at least 90%, at least 95%, or 100% sequence identity to SEQ ID NO: 79 or SEQ ID NO: 80.

In some aspects, the DNA sequence encoding a promoter sequence of the first expression cassette sequence comprises at least 80%, at least 85%, at least 90%, at least 95%, or 100% sequence identity to SEQ ID NO: 5, the DNA sequence encoding a SmOPT and a small nuclear RNA (snRNA) processing hairpin sequence of the first expression cassette sequence comprises at least 80%, at least 85%, at least 90%, at least 95%, or 100% sequence identity to SEQ ID NO: 92, and the DNA sequence encoding a transcription termination sequence of the first expression cassette sequence comprises at least 80%, at least 85%, at least 90%, at least 95%, or 100% sequence identity to SEQ ID NO: 78 or SEQ ID NO: 79.

In some aspects, the DNA sequence encoding a promoter sequence of the second expression cassette sequence comprises at least 80%, at least 85%, at least 90%, at least 95%, or 100% sequence identity to SEQ ID NO: 67, the DNA sequence encoding a SmOPT and a small nuclear RNA (snRNA) processing hairpin sequence of the second expression cassette sequence comprises at least 80%, at least 85%, at least 90%, at least 95%, or 100% sequence identity to SEQ ID NO: 92, and the DNA sequence encoding a transcription termination sequence of the second expression cassette sequence comprises at least 80%, at least 85%, at least 90%, at least 95%, or 100% sequence identity to SEQ ID NO: 79 or SEQ ID NO: 80.

In some aspects, the DNA sequence encoding a promoter sequence of the first expression cassette sequence comprises at least 80%, at least 85%, at least 90%, at least 95%, or 100% sequence identity to SEQ ID NO: 5, the DNA sequence encoding a SmOPT and a small nuclear RNA (snRNA) processing hairpin sequence of the first expression cassette sequence comprises at least 80%, at least 85%, at least 90%, at least 95%, or 100% sequence identity to SEQ ID NO: 92, the DNA sequence encoding a transcription termination sequence of the first expression cassette sequence comprises at least 80%, at least 85%, at least 90%, at least 95%, or 100% sequence identity to SEQ ID NO: 78, the DNA sequence encoding a promoter sequence of the second expression cassette sequence comprises at least 80%, at least 85%, at least 90%, at least 95%, or 100% sequence identity to SEQ ID NO: 67, the DNA sequence encoding a SmOPT and a small nuclear RNA (snRNA) processing hairpin sequence of the second expression cassette sequence comprises at least 80%, at least 85%, at least 90%, at least 95%, or 100% sequence identity to SEQ ID NO: 92, and the DNA sequence encoding a transcription termination sequence of the second expression cassette sequence comprises at least 80%, at least 85%, at least 90%, at least 95%, or 100% sequence identity to SEQ ID NO: 79.

In some aspects, the DNA sequence encoding a promoter sequence of the first expression cassette sequence comprises at least 80%, at least 85%, at least 90%, at least 95%, or 100% sequence identity to SEQ ID NO: 5, the DNA sequence encoding a SmOPT and a small nuclear RNA (snRNA) processing hairpin sequence of the first expression cassette sequence comprises at least 80%, at least 85%, at least 90%, at least 95%, or 100% sequence identity to SEQ ID NO: 92, the DNA sequence encoding a transcription termination sequence of the first expression cassette sequence comprises at least 80%, at least 85%, at least 90%, at least 95%, or 100% sequence identity to SEQ ID NO: 79, the DNA sequence encoding a promoter sequence of the second expression cassette sequence comprises at least 80%, at least 85%, at least 90%, at least 95%, or 100% sequence identity to SEQ ID NO: 67, the DNA sequence encoding a SmOPT and a small nuclear RNA (snRNA) processing hairpin sequence of the second expression cassette sequence comprises at least 80%, at least 85%, at least 90%, at least 95%, or 100% sequence identity to SEQ ID NO: 92, and the DNA sequence encoding a transcription termination sequence of the second expression cassette sequence comprises at least 80%, at least 85%, at least 90%, at least 95%, or 100% sequence identity to SEQ ID NO: 80.

In various aspects, the present disclosure provides a polynucleotide comprising a plurality of expression cassette sequences comprising a first expression cassette sequence and a second expression cassette sequence, wherein: the first, second, and plurality of expression cassette sequences each independently comprise: a promoter sequence, a payload sequence under transcriptional control of the promoter sequence, the payload sequence comprising a small RNA payload, and a transcription termination sequence; the first, second, and plurality of expression cassette sequences are each independently arranged in a 5′ to 3′ orientation to have a read directionality of forward or reverse; and the first and second expression cassette sequences are different sequences.

In some aspects, the first expression cassette sequence has a read directionality of forward and the second expression cassette sequence has a read directionality of reverse. In some aspects, the first expression cassette sequence has a read directionality of reverse and the second expression cassette sequence has a read directionality of forward. In some aspects, the first expression cassette sequence has a read directionality of forward and the second expression cassette sequence has a read directionality of forward.

In some aspects, the plurality of expression cassette sequences comprises two expression cassette sequences, three expression cassette sequences, four expression cassette sequences, five expression cassette sequences, six expression cassette sequences, seven expression cassette sequences, eight expression cassette sequences, nine expression cassette sequences, or ten expression cassette sequences.

In some aspects, the small RNA payload comprises an engineered guide RNA sequence. In some aspects, the engineered guide RNA sequence is capable of hybridizing to a target sequence. In some aspects, the engineered guide RNA sequence is at least 70%, at least 75%, at least 80%, at least 85%, at least 90%, at least 95%, or 100% reverse complementary to the target sequence.

In some aspects, the engineered guide RNA sequence is capable of forming a guide-target RNA scaffold comprising one or more structural features upon hybridization to a target sequence. In some aspects, the one or more structural features comprise a bulge, a mismatch, an internal loop, a hairpin, or combinations thereof. In some aspects, the one or more structural features comprises the bulge, and wherein the bulge is a symmetric bulge. In some aspects, the one or more structural features comprises the bulge, and wherein the bulge is an asymmetric bulge. In some aspects, the one or more structural features comprises the internal loop, and wherein the internal loop is a symmetric internal loop. In some aspects, the one or more structural features comprises the internal loop, and wherein the internal loop is an asymmetric internal loop. In some aspects, the one or more structural features comprises the hairpin, and wherein the hairpin is a recruitment hairpin or a non-recruitment hairpin. In some aspects, the guide-target RNA scaffold comprises one or more wobble base pairs. In some aspects, the one or more of the wobble base pairs are GU wobble base pairs.

In some aspects, the engineered guide RNA sequence comprises at least one base pair mismatch relative to the target sequence. In some aspects, the target sequence comprises an adenosine residue. In some aspects, the target sequence is an RNA sequence. In some aspects, the RNA sequence is a mRNA or a pre-mRNA. In some aspects, the target sequence comprises a G to A mutation relative to a wild type sequence. In some aspects, the target sequence comprises a missense mutation or a nonsense mutation relative to a wild type sequence.

In some aspects, the target sequence encodes α-synuclein (SNCA). In some aspects, the target sequence encodes peripheral myelin protein 22 (PMP22). In some aspects, the target sequence encodes double homeobox 4 (DUX4). In some aspects, the target sequence encodes leucine rich repeat kinase 2 (LRRK2). In some aspects, the target sequence encodes Tau (MAPT). In some aspects, the target sequence encodes ATP-binding cassette sub-family A member 4 (ABCA4). In some aspects, the target sequence encodes alpha-1 antitrypsin (SERPINA1). In some aspects, the target sequence encodes methyl CpG binding protein 2 (MECP2).

In some aspects, the engineered guide RNA sequence is not less than 20 nucleotide residues and not more than 500 nucleotide residues long. In some aspects, the engineered guide RNA sequence is not less than 60 and not more than 100 residues long. In some aspects, the engineered guide RNA sequence is not less than 80 and not more than 120 residues long. In some aspects, the engineered guide RNA sequence is not less than 100 and not more than 140 residues long. In some aspects, the engineered guide RNA sequence is not less than 130 and not more than 170 residues long.

In some aspects, the promoter sequence comprises at least 80%, at least 85%, at least 90%, at least 95%, or 100% sequence identity to any one of SEQ ID NO: 1-SEQ ID NO: 9, SEQ ID NO: 66-SEQ ID NO: 76, SEQ ID NO: 90, or SEQ ID NO: 91. In some aspects, the transcription termination sequence comprises at least 80%, at least 85%, at least 90%, at least 95%, or 100% sequence identity to any one of SEQ ID NO: 10-SEQ ID NO: 16, SEQ ID NO: 34, SEQ ID NO: 35, SEQ ID NO: 77-SEQ ID NO: 82, or SEQ ID NO: 85-SEQ ID NO: 89.

In some aspects, the payload sequence further comprises an Sm binding sequence or a hairpin sequence. In some aspects, the hairpin sequence comprises a U7 hairpin.

In some aspects, the first expression cassette sequence, the second expression cassette sequence, or each expression cassette of the plurality of expression cassette sequences independently has a length of not less than 1300 nucleotide residues and not more than 2160 nucleotide residues. In some aspects, the first expression cassette sequence, the second expression cassette sequence, or each expression cassette of the plurality of expression cassette sequences independently comprises at least 80% sequence identity to a U1 sequence or a U7 sequence. In some aspects, the U1 sequence is a mouse U1 sequence or a human U1 sequence. In some aspects, the U7 sequence is a mouse U7 sequence or a human U7 sequence.

In various aspects, the present disclosure provides a viral vector encoding the polynucleotide of the present disclosure.

In some aspects, the viral vector is an adeno-associated viral vector. In some aspects, the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, chimeras thereof, variants or derivatives thereof, and combinations thereof.

In various aspects, the present disclosure provides a pharmaceutical composition comprising the polynucleotide of the present disclosure or the viral vector of the present disclosure and a pharmaceutically acceptable excipient, carrier, diluent, or combination thereof.

In various aspects, the present disclosure provides a method of expressing an engineered guide RNA in a cell, the method comprising: (i) delivering a composition comprising a polynucleotide to a cell, wherein the polynucleotide comprises a plurality of expression cassette sequences comprising a first expression cassette sequence and a second expression cassette sequence, wherein: the first expression cassette sequence and the second expression cassette sequence each independently comprise: a DNA sequence encoding a promoter sequence, a DNA sequence encoding an engineered guide RNA (gRNA) sequence under transcriptional control of the promoter sequence, a DNA sequence encoding a SmOPT and a small nuclear RNA (snRNA) processing hairpin sequence, and a DNA sequence encoding a transcription termination sequence; at least one of the DNA sequences encoding a promoter sequence comprises at least 80% sequence identity to SEQ ID NO: 5; the first expression cassette sequence and the second expression cassette sequence are each independently arranged in a 5′ to 3′ orientation to have a read directionality of forward; the DNA sequence encoding an engineered guide RNA (gRNA) sequence of the first expression cassette sequence and the second expression cassette sequence encodes the same engineered guide RNA (gRNA) sequence; and the first and second expression cassette sequences are different sequences; and (ii) expressing the engineered guide RNA (gRNA) sequence in the cell.

In various aspects, the present disclosure provides a method of editing a target sequence, the method comprising: (i) delivering a composition comprising a polynucleotide to a cell encoding a target sequence, wherein the polynucleotide comprises a plurality of expression cassette sequences comprising a first expression cassette sequence and a second expression cassette sequence, wherein: the first expression cassette sequence and the second expression cassette sequence each independently comprise: a DNA sequence encoding a promoter sequence, a DNA sequence encoding an engineered guide RNA (gRNA) sequence under transcriptional control of the promoter sequence, a DNA sequence encoding a SmOPT and a small nuclear RNA (snRNA) processing hairpin sequence, and a DNA sequence encoding a transcription termination sequence; at least one of the DNA sequences encoding a promoter sequence comprises at least 80% sequence identity to SEQ ID NO: 5; the first expression cassette sequence and the second expression cassette sequence are each independently arranged in a 5′ to 3′ orientation to have a read directionality of forward; the DNA sequence encoding an engineered guide RNA (gRNA) sequence of the first expression cassette sequence and the second expression cassette sequence encodes the same engineered guide RNA (gRNA) sequence; and the first and second expression cassette sequences are different sequences; (ii) expressing the engineered guide RNA (gRNA) sequence in the cell; (iii) forming a guide-target RNA scaffold upon hybridization of the engineered guide RNA (gRNA) sequence to the target sequence; (iv) recruiting an editing enzyme to the target sequence; and (v) editing the target sequence with the editing enzyme.

In various aspects, the present disclosure provides a method of expressing an engineered guide RNA sequence in a cell, the method comprising: (i) delivering a composition comprising a polynucleotide to a cell, wherein the polynucleotide comprises a plurality of expression cassette sequences comprising a first expression cassette sequence and a second expression cassette sequence, wherein: the first, second, and plurality of expression cassette sequences each independently comprise: a promoter sequence, a payload sequence under transcriptional control of the promoter sequence, the payload sequence comprising an engineered guide RNA sequence, and a transcription termination sequence; the first, second, and plurality of expression cassette sequences are each independently arranged in a 5′ to 3′ orientation to have a read directionality of forward or reverse; and the first and second expression cassette sequences are different sequences; and (ii) expressing the engineered guide RNA sequence in the cell.

In various aspects, the present disclosure provides a method of editing a target sequence, the method comprising: (i) delivering a composition comprising a polynucleotide to a cell encoding a target sequence, wherein the polynucleotide comprises a plurality of expression cassette sequences comprising a first expression cassette sequence and a second expression cassette sequence, wherein: the first, second, and plurality of expression cassette sequences each independently comprise: a promoter sequence, a payload sequence under transcriptional control of the promoter sequence, the payload sequence comprising an engineered guide RNA sequence, and a transcription termination sequence; the first, second, and plurality of expression cassette sequences are each independently arranged in a 5′ to 3′ orientation to have a read directionality of forward or reverse; and the first and second expression cassette sequences are different sequences; (ii) expressing the engineered guide RNA sequence in the cell; (iii) forming a guide-target RNA scaffold upon hybridization of the engineered guide RNA sequence to the target sequence; (iv) recruiting an editing enzyme to the target sequence; and (v) editing the target sequence with the editing enzyme.

In various aspects, the present disclosure provides a method of administering a polynucleotide to a subject with a disease, the method comprising: (i) administering to the subject a composition comprising the polynucleotide, wherein the polynucleotide comprises a plurality of expression cassette sequences comprising a first expression cassette sequence and a second expression cassette sequence, wherein: the first expression cassette sequence and the second expression cassette sequence each independently comprise: a DNA sequence encoding a promoter sequence, a DNA sequence encoding an engineered guide RNA (gRNA) sequence under transcriptional control of the promoter sequence, a DNA sequence encoding a SmOPT and a small nuclear RNA (snRNA) processing hairpin sequence, and a DNA sequence encoding a transcription termination sequence; at least one of the DNA sequences encoding a promoter sequence comprises at least 80% sequence identity to SEQ ID NO: 5; the first expression cassette sequence and the second expression cassette sequence are each independently arranged in a 5′ to 3′ orientation to have a read directionality of forward; the DNA sequence encoding an engineered guide RNA (gRNA) sequence of the first expression cassette sequence and the second expression cassette sequence encodes the same engineered guide RNA (gRNA) sequence; and the first and second expression cassette sequences are different sequence; (ii) delivering the polynucleotide to a cell of the subject; and (iii) expressing the engineered guide RNA (gRNA) sequence in the cell.

In various aspects, the present disclosure provides a method of treating a disease in a subject, the method comprising: (i) administering to the subject a composition comprising a polynucleotide, wherein the polynucleotide comprises a plurality of expression cassette sequences comprising a first expression cassette sequence and a second expression cassette sequence, wherein: the first expression cassette sequence and the second expression cassette sequence each independently comprise: a DNA sequence encoding a promoter sequence, a DNA sequence encoding an engineered guide RNA (gRNA) sequence under transcriptional control of the promoter sequence, a DNA sequence encoding a SmOPT and a small nuclear RNA (snRNA) processing hairpin sequence, and a DNA sequence encoding a transcription termination sequence; at least one of the DNA sequences encoding a promoter sequence comprises at least 80% sequence identity to SEQ ID NO: 5; the first expression cassette sequence and the second expression cassette sequence are each independently arranged in a 5′ to 3′ orientation to have a read directionality of forward; the DNA sequence encoding an engineered guide RNA (gRNA) sequence of the first expression cassette sequence and the second expression cassette sequence encodes the same engineered guide RNA (gRNA) sequence; and the first and second expression cassette sequences are different sequence; (ii) delivering the polynucleotide to a cell of the subject; and (iii) expressing the engineered guide RNA (gRNA) sequence in the cell, thereby treating the disease.

In various aspects, the present disclosure provides a method of administering a polynucleotide to a subject with a disease, the method comprising: (i) administering to the subject a composition comprising the polynucleotide, wherein the polynucleotide comprises a plurality of expression cassette sequences comprising a first expression cassette sequence and a second expression cassette sequence, wherein: the first, second, and plurality of expression cassette sequences each independently comprise: a promoter sequence, a payload sequence under transcriptional control of the promoter sequence, the payload sequence comprising an engineered guide RNA sequence, and a transcription termination sequence; the first, second, and plurality of expression cassette sequences are each independently arranged in a 5′ to 3′ orientation to have a read directionality of forward or reverse; and the first and second expression cassette sequences are different sequences; (ii) delivering the therapeutic polynucleotide to a cell of the subject; and (iii) expressing the engineered guide RNA sequence in the cell.

In various aspects, the present disclosure provides a method of treating a disease in a subject, the method comprising: (i) administering to the subject a composition comprising a polynucleotide, wherein the polynucleotide comprises a plurality of expression cassette sequences comprising a first expression cassette sequence and a second expression cassette sequence, wherein: the first, second, and plurality of expression cassette sequences each independently comprise: a promoter sequence, a payload sequence under transcriptional control of the promoter sequence, the payload sequence comprising an engineered guide RNA sequence, and a transcription termination sequence; the first, second, and plurality of expression cassette sequences are each independently arranged in a 5′ to 3′ orientation to have a read directionality of forward or reverse; and the first and second expression cassette sequences are different sequences; (ii) delivering the therapeutic polynucleotide to a cell of the subject; and (iii) expressing the engineered guide RNA sequence in the cell, thereby treating the disease.

In some aspects, the cell is in a central nervous system tissue. In some aspects, the cell is in a liver tissue, muscle tissue, ocular tissue, retinal tissue, heart tissue, skeletal muscle tissue, or kidney tissue. In some aspects, the composition is the pharmaceutical composition of the present disclosure. In some aspects, the composition comprises the polynucleotide of the present disclosure or the viral vector of the present disclosure. In some aspects, the disease is a synucleinopathy, Parkinson's disease, Lewy body dementia, multiple system atrophy, Charcot-Marie-Tooth disease, hereditary neuropathy with liability to pressure palsies, Yuan-Harel-Lupski syndrome, a tauopathy, Alzheimer's disease, frontotemporal dementia, progressive supranuclear palsy, corticobasal degeneration, chronic traumatic encephalopathy, autism, traumatic brain injury, Dravet syndrome, Crohn's disease, muscular dystrophy, B-cell leukemia, Dejerine-Sottas disease, Stargardt disease, alpha-1 antitrypsin deficiency, Tay-Sachs disease, cystic fibrosis, liposomal acid lipase deficiency, or Gaucher disease.

In some aspects, the engineered guide RNA sequence hybridizes to a target sequence, and wherein the cell encodes the target sequence. In some aspects, the method further comprises forming a guide-target RNA scaffold upon hybridization of the engineered guide RNA to the target sequence, recruiting an editing enzyme to the target sequence, and editing the target sequence with the editing enzyme.

In some aspects, the target sequence encodes α-synuclein (SNCA). In some aspects, the target sequence encodes peripheral myelin protein 22 (PMP22). In some aspects, the target sequence encodes double homeobox 4 (DUX4). In some aspects, the target sequence encodes leucine rich repeat kinase 2 (LRRK2). In some aspects, the target sequence encodes Tau (MAPT). In some aspects, the target sequence encodes ATP-binding cassette sub-family A member 4 (ABCA4). In some aspects, the target sequence encodes alpha-1 antitrypsin (SERPINA1). In some aspects, the target sequence encodes methyl CpG binding protein 2 (MECP2).

In some aspects, the target sequence comprises a mutation relative to a wild type sequence. In some aspects, editing the target sequence corrects the mutation in the target sequence. In some aspects, the mutation is a missense mutation. In some aspects, the mutation is a nonsense mutation. In some aspects, the mutation is a G to A mutation. In some aspects, the mutation is associated with the disease.

In some aspects, editing the target sequence comprises editing an untranslated region of the target sequence. In some aspects, the untranslated region is a 5′ untranslated region or a 3′ untranslated region. In some aspects, the 3′ untranslated region is a polyadenylation sequence. In some aspects, editing the target sequence comprises editing a translation initiation site.

In some aspects, editing the target sequence alters expression of the target sequence. In some aspects, editing the target sequence increases expression of the target sequence. In some aspects, editing the target sequence decreases expression of the target sequence. In some aspects, the editing enzyme comprises an ADAR, an APOBEC, or a Cas nuclease. In some aspects, the ADAR comprises ADAR1, ADAR2, or a combination thereof. In some aspects, the target sequence comprises RNA or DNA. In some aspects, the target sequence is a mRNA or a pre-mRNA. In some aspects, editing the target sequence comprises deamidating a nucleotide of the target sequence. In some aspects, the target sequence is edited with an efficiency of at least 10%, at least 20%, or at least 25%.

In various aspects, the present disclosure provides a method of increasing a vector genome integrity of a multi-expression cassette vector comprising a first expression cassette and a second expression cassette, the method comprising: a) generating a sequence of the second expression cassette by altering a sequence of the first expression cassette, wherein the first expression cassette and the second expression cassette each independently comprise: a DNA sequence encoding a promoter sequence, a DNA sequence encoding an engineered guide RNA (gRNA) sequence under transcriptional control of the promoter sequence, a DNA sequence encoding a SmOPT and a small nuclear RNA (snRNA) processing hairpin sequence, and a DNA sequence encoding a transcription termination sequence; b) generating the multi-expression cassette vector by combining the sequence of the first expression cassette and the sequence of the second expression cassette; and c) increasing the vector genome integrity as compared to a vector comprising a first expression cassette and a second expression cassette that each have the sequence of the first expression cassette.

In some aspects, the sequence of the first expression cassette and the sequence of the second expression cassette have different DNA sequences encoding a promoter sequence. In some aspects, the sequence of the first expression cassette and the sequence of the second expression cassette have different DNA sequences encoding a transcription termination sequence. In some aspects, the sequence of the first expression cassette and the sequence of the second expression cassette have different DNA sequences encoding a SmOPT and a small nuclear RNA (snRNA) processing hairpin sequence. In some aspects, the DNA sequence encoding a promoter sequence comprises at least 80%, at least 85%, at least 90%, at least 95%, or 100% sequence identity to any one of SEQ ID NO: 1-SEQ ID NO: 9, SEQ ID NO: 66-SEQ ID NO: 76, SEQ ID NO: 90, or SEQ ID NO: 91. In some aspects, the DNA sequence encoding a transcription termination sequence comprises at least 80%, at least 85%, at least 90%, at least 95%, or 100% sequence identity to any one of SEQ ID NO: 10-SEQ ID NO: 16, SEQ ID NO: 34, SEQ ID NO: 35, SEQ ID NO: 77-SEQ ID NO: 82, or SEQ ID NO: 85-SEQ ID NO: 89.

In various aspects, the present disclosure provides a polynucleotide comprising a plurality of expression cassette sequences comprising a first expression cassette sequence and a second expression cassette sequence, wherein: the first, second, and plurality of expression cassette sequences each independently comprise: a promoter sequence, a payload sequence under transcriptional control of the promoter sequence, the payload sequence comprising a small RNA payload, and a transcription termination sequence; the first, second, and plurality of expression cassette sequences are each independently arranged in a 5′ to 3′ orientation to have a read directionality of forward or reverse; and the first and second expression cassette sequences are different sequences.

In some aspects, the first expression cassette sequence has a read directionality of forward and the second expression cassette sequence has a read directionality of reverse. In some aspects, the first expression cassette sequence has a read directionality of reverse and the second expression cassette sequence has a read directionality of forward. In some aspects, the first expression cassette sequence has a read directionality of forward and the second expression cassette sequence has a read directionality of forward. In some aspects, the plurality of expression cassette sequences comprises two expression cassette sequences, three expression cassette sequences, four expression cassette sequences, five expression cassette sequences, six expression cassette sequences, seven expression cassette sequences, eight expression cassette sequences, nine expression cassette sequences, or ten expression cassette sequences.

In some aspects, the small RNA payload comprises an engineered guide RNA sequence capable of hybridizing to a target sequence. In some aspects, the first expression cassette sequence comprises a first engineered guide RNA sequence capable of hybridizing to a target sequence and the second expression cassette comprises a second engineered guide RNA sequence capable of hybridizing to the target sequence. In some aspects, the second engineered guide RNA sequence has at least one and no more than 30 nucleotide alterations from the first engineered guide RNA sequence. In some aspects, the nucleotide alterations from the first engineered guide RNA sequence are dispersed at a frequency of at least one and no more than 4 nucleotide alterations per every 10 nucleotides in the second engineered guide RNA sequence. In some aspects, the nucleotide alterations from the first engineered guide RNA sequence are dispersed at a frequency of 3 nucleotide alterations per every 10 nucleotides in the second engineered guide RNA sequence.

In some aspects, the engineered guide RNA sequence, the first engineered guide RNA sequence, or the second engineered guide RNA sequence are each independently at least 70%, at least 75%, at least 80%, at least 85%, at least 90%, at least 95%, or 100% reverse complementary to the target sequence. In some aspects, the engineered guide RNA sequence, the first engineered guide RNA sequence, or the second engineered guide RNA sequence are each independently capable of forming a guide-target RNA scaffold comprising one or more structural features upon hybridization of the small RNA payload to a target sequence.

In some aspects, the guide-target RNA scaffold of the first engineered guide RNA sequence and the guide-target RNA scaffold of the second engineered guide RNA sequence comprise the same one or more structural features. In some aspects, the one or more structural features comprise a bulge, a mismatch, an internal loop, a hairpin, or combinations thereof. In some aspects, the one or more structural features comprises the bulge, and wherein the bulge is a symmetric bulge. In some aspects, the one or more structural features comprises the bulge, and wherein the bulge is an asymmetric bulge. In some aspects, the one or more structural features comprises the internal loop, and wherein the internal loop is a symmetric internal loop. In some aspects, the one or more structural features comprises the internal loop, and wherein the internal loop is an asymmetric internal loop. In some aspects, the one or more structural features comprises the hairpin, and wherein the hairpin is a recruitment hairpin or a non-recruitment hairpin.

In some aspects, the guide-target RNA scaffold comprises one or more wobble base pairs. In some aspects, the one or more of the wobble base pairs are GU wobble base pairs. In some aspects, the guide-target RNA scaffold of the second engineered guide RNA sequence has between at least one and no more than 15 additional wobble base pairs than the guide-target RNA scaffold of the first engineered guide RNA sequence. In some aspects, the engineered guide RNA sequence, the first engineered guide RNA sequence, or the second engineered guide RNA sequence comprise at least one base pair mismatch relative to the target sequence.

In various aspects, the present disclosure provides a method of editing a target sequence in a cell with an increased specificity, the method comprising: delivering the polynucleotide as described herein, the viral vector as described herein, or the pharmaceutical composition as described herein to a cell encoding the target sequence; expressing the small RNA payload in the cell, wherein, the small RNA payload comprises an engineered guide RNA sequence capable of hybridizing to the target sequence; forming a guide-target RNA scaffold upon hybridization of the engineered guide RNA sequence to the target sequence, wherein the guide-target RNA scaffold comprises at least one and no more than 15 wobble base pairs; recruiting an editing enzyme to the target sequence; and editing the target sequence with the editing enzyme with the increased specificity as compared to a specificity of a guide-target RNA scaffold comprising 0 wobble base pairs.

In some aspects, the wobble base pairs comprise one or more GU wobble base pairs. In some aspects, the method further comprises over twisting a helical structure of the guide-target RNA scaffold.

In various aspects, the present disclosure provides a method of increasing a vector genome integrity of a vector with a first expression cassette and a second expression cassette, the method comprising: a) generating a sequence of the second expression cassette by altering a sequence of the first expression cassette, wherein the first expression cassette and the second expression cassette each independently comprise: a promoter sequence, a payload sequence under transcriptional control of the promoter sequence, the payload sequence comprising a small RNA payload, and a transcription termination sequence; b) generating the multiple payload vector by combining the sequence of the first expression cassette sequence and the sequence of the second expression cassette; and c) increasing the vector genome integrity as compared to a vector comprising a first expression cassette and a second expression cassette that each have the sequence of the first expression cassette.

In some aspects, the sequence of the first expression cassette and the sequence of the second expression cassette have different promoter sequences. In some aspects, the sequence of the first expression cassette and the sequence of the second expression cassette have different transcription termination sequences. In some aspects, the sequence of the first expression cassette and the sequence of the second expression cassette have different payload sequences. In some aspects, the promoter sequence comprises at least 80%, at least 85%, at least 90%, at least 95%, or 100% sequence identity to any one of SEQ ID NO: 1-SEQ ID NO: 9, SEQ ID NO: 66
SEQ ID NO: 76, SEQ ID NO: 90, or SEQ ID NO: 91. In some aspects, the transcription termination sequence comprises at least 80%, at least 85%, at least 90%, at least 95%, or 100% sequence identity to any one of SEQ ID NO: 10-SEQ ID NO: 16, SEQ ID NO: 34, SEQ ID NO: 35, SEQ ID NO: 77-SEQ ID NO: 82, or SEQ ID NO: 85-SEQ ID NO: 89.

In some aspects, the method further comprises a) altering a sequence of a first payload comprising a first engineered guide RNA sequence to generate a second engineered guide RNA sequence to be comprised by a second payload by: i) hybridizing the first engineered guide RNA sequence to a target sequence; ii) forming a first guide-target RNA scaffold comprising one or more structural features; iv) altering at least 20 and no more than 40 nucleotides in the first engineered guide RNA sequence to a different nucleotide resulting in the second engineered guide RNA sequence, wherein hybridizing the second engineered guide RNA sequence to the target sequence forms a second guide-target RNA scaffold comprising the same one or more features at the first guide-target RNA scaffold; b) encoding the first engineered guide RNA sequence in the first payload and the second engineered guide RNA sequence in the second payload in the multiple payload vector; and c) increasing the vector genome integrity of the multiple payload vector as compared to a multiple payload vector comprising a first and second payload each comprising the first engineered guide RNA sequence.

In some aspects, the one or more structural features comprise a bulge, a mismatch, an internal loop, a hairpin, or combinations thereof. In some aspects, the one or more structural features comprises the bulge, and wherein the bulge is a symmetric bulge. In some aspects, the one or more structural features comprises the bulge, and wherein the bulge is an asymmetric bulge. In some aspects, the one or more structural features comprises the internal loop, and wherein the internal loop is a symmetric internal loop. In some aspects, the one or more structural features comprises the internal loop, and wherein the internal loop is an asymmetric internal loop. In some aspects, the one or more structural features comprises the hairpin, and wherein the hairpin is a recruitment hairpin or a non-recruitment hairpin.

In some aspects, the second guide-target RNA scaffold comprises at least one and no more than 15 additional wobble base pairs as compared to the first guide-target RNA scaffold. In some aspects, the one or more of the additional wobble base pairs is a GU wobble base pair. In some aspects, the structure of the first guide-target RNA scaffold and the structure of the second guide-target RNA scaffold comprise a helical structure. In some aspects, the helical structure is over twisted in the structure of the second guide-target RNA scaffold compared to the structure of the first guide-target RNA scaffold.

In various aspects, the present disclosure provides a viral vector comprising: a plurality of expression cassettes, wherein each expression cassette independently comprises: a promoter sequence; a payload sequence under transcriptional control of the promoter sequence, the payload sequence comprising a small RNA payload; and a transcription termination sequence, wherein each expression cassette of the plurality of expression cassettes is arranged in a 5′ to 3′ orientation to have a read directionality of forward or reverse.

In some aspects, the plurality of expression cassettes comprises two expression cassettes, three expression cassettes, four expression cassettes, five expression cassettes, six expression cassettes, seven expression cassettes, eight expression cassettes, nine expression cassettes, or ten expression cassettes.

In some aspects, the plurality of expression cassettes comprises a first expression cassette and a second expression cassette, wherein: a) the first expression cassette has the read directionality of forward and the second expression cassette has the read directionality of reverse; b) the first expression cassette has the read directionality of reverse and the second expression cassette has the read directionality of forward; c) the first expression cassette has the read directionality of forward and the second expression cassette has the read directionality of forward; or d) the first expression cassette has the read directionality of reverse and the second expression cassette has the read directionality of reverse.

In some aspects, the plurality of expression cassettes comprises a first expression cassette, a second expression cassette and a third expression cassette, wherein: a) the first expression cassette has the read directionality of forward, the second expression cassette has the read directionality of forward, and the third expression cassette has the read directionality of reverse; b) the first expression cassette has the read directionality of forward, the second expression cassette has the read directionality of reverse, and the third expression cassette has the read directionality of forward; c) the first expression cassette has the read directionality of forward, the second expression cassette has the read directionality of reverse, and the third expression cassette has the read directionality of reverse; d) the first expression cassette has the read directionality of reverse, the second expression cassette has the read directionality of forward, and the third expression cassette has the read directionality of forward; e) the first expression cassette has the read directionality of reverse, the second expression cassette has the read directionality of forward, and the third expression cassette has the read directionality of reverse; f) the first expression cassette has the read directionality of reverse, the second expression cassette has the read directionality of reverse, and the third expression cassette has the read directionality of forward; g) the first expression cassette has the read directionality of forward, the second expression cassette has the read directionality of forward, and the third expression cassette has the read directionality of forward; or h) the first expression cassette has the read directionality of reverse, the second expression cassette has the read directionality of reverse, and the third expression cassette has the read directionality of reverse.

In some aspects, the plurality of expression cassettes comprises a first expression cassette, a second expression cassette, a third expression cassette, and a fourth expression cassette, wherein: a) the first expression cassette has the read directionality of reverse, the second expression cassette has the read directionality of reverse, the third expression cassette has the read directionality of forward, and the fourth expression cassette has the read directionality of forward; b) the first expression cassette has the read directionality of forward, the second expression cassette has the read directionality of forward, the third expression cassette has the read directionality of forward, and the fourth expression cassette has the read directionality of forward; c) the first expression cassette has the read directionality of reverse, the second expression cassette has the read directionality of reverse, the third expression cassette has the read directionality of reverse, and the fourth expression cassette has the read directionality of reverse; d) the first expression cassette has the read directionality of reverse, the second expression cassette has the read directionality of forward, the third expression cassette has the read directionality of forward, and the fourth expression cassette has the read directionality of forward; e) the first expression cassette has the read directionality of reverse, the second expression cassette has the read directionality of reverse, the third expression cassette has the read directionality of reverse, and the fourth expression cassette has the read directionality of forward; f) the first expression cassette has the read directionality of forward, the second expression cassette has the read directionality of forward, the third expression cassette has the read directionality of forward, and the fourth expression cassette has the read directionality of reverse; g) the first expression cassette has the read directionality of forward, the second expression cassette has the read directionality of forward, the third expression cassette has the read directionality of reverse, and the fourth expression cassette has the read directionality of forward; h) the first expression cassette has the read directionality of forward, the second expression cassette has the read directionality of forward, the third expression cassette has the read directionality of reverse, and the fourth expression cassette has the read directionality of reverse; i) the first expression cassette has the read directionality of forward, the second expression cassette has the read directionality of reverse, the third expression cassette has the read directionality of forward, and the fourth expression cassette has the read directionality of forward; j) the first expression cassette has the read directionality of forward, the second expression cassette has the read directionality of reverse, the third expression cassette has the read directionality of forward, and the fourth expression cassette has the read directionality of reverse; k) the first expression cassette has the read directionality of forward, the second expression cassette has the read directionality of reverse, the third expression cassette has the read directionality of reverse, and the fourth expression cassette has the read directionality of forward; 1) the first expression cassette has the read directionality of forward, the second expression cassette has the read directionality of reverse, the third expression cassette has the read directionality of reverse, and the fourth expression cassette has the read directionality of reverse; m) the first expression cassette has the read directionality of reverse, the second expression cassette has the read directionality of forward, the third expression cassette has the read directionality of forward, and the fourth expression cassette has the read directionality of reverse; n) the first expression cassette has the read directionality of reverse, the second expression cassette has the read directionality of forward, the third expression cassette has the read directionality of reverse, and the fourth expression cassette has the read directionality of forward; the first expression cassette has the read directionality of reverse, the second expression cassette has the read directionality of forward, the third expression cassette has the read directionality of reverse, and the fourth expression cassette has the read directionality of reverse; or p) the first expression cassette has the read directionality of reverse, the second expression cassette has the read directionality of reverse, the third expression cassette has the read directionality of forward, and the fourth expression cassette has the read directionality of reverse.

In some aspects, the plurality of expression cassettes comprises a first expression cassette, a second expression cassette, a third expression cassette, and a fourth expression cassette, wherein: a) the first expression cassette has the read directionality of reverse, the second expression cassette has the read directionality of reverse, the third expression cassette has the read directionality of forward, and the fourth expression cassette has the read directionality of forward; b) the first expression cassette has the read directionality of forward, the second expression cassette has the read directionality of forward, the third expression cassette has the read directionality of forward, and the fourth expression cassette has the read directionality of forward; c) the first expression cassette has the read directionality of reverse, the second expression cassette has the read directionality of reverse, the third expression cassette has the read directionality of reverse, and the fourth expression cassette has the read directionality of reverse; d) the first expression cassette has the read directionality of reverse, the second expression cassette has the read directionality of forward, the third expression cassette has the read directionality of forward, and the fourth expression cassette has the read directionality of forward; or e) the first expression cassette has the read directionality of reverse, the second expression cassette has the read directionality of reverse, the third expression cassette has the read directionality of reverse, and the fourth expression cassette has the read directionality of forward.

In some aspects, the first expression cassette and the second expression cassette comprise a different promoter sequence. In some aspects, the first expression cassette and the second expression cassette comprise same promoter sequence. In some aspects, the first expression cassette, the second expression cassette and the third expression cassette each comprise a different promoter sequence. In some aspects, at least two expression cassettes comprise same promoter sequence. In some aspects, the first expression cassette, the second expression cassette, the third expression cassette and fourth expression cassette each comprise a different promoter sequence. In some aspects, at least two of the four expression cassettes comprise same promoter sequence.

In various aspects, the present disclosure provides a viral vector encoding the polynucleotide as described herein.

In some aspects, the viral vector is an adeno-associated viral vector. In some aspects, the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, chimeras thereof, variants or derivatives thereof, and combinations thereof.

In some aspects, the promoter sequence comprises at least 80%, at least 85%, at least 90%, at least 95%, or 100% sequence identity to any one of SEQ ID NO: 1-SEQ ID NO: 9, SEQ ID NO: 66-SEQ ID NO: 76, SEQ ID NO: 90, or SEQ ID NO: 91. In some aspects, the transcription termination sequence comprises at least 80%, at least 85%, at least 90%, at least 95%, or 100% sequence identity to any one of SEQ ID NO: 10-SEQ ID NO: 16. In some aspects, the transcription termination sequence comprises at least 80%, at least 85%, at least 90%, at least 95%, or 100% sequence identity to any one of SEQ ID NO: 10-SEQ ID NO: 16, SEQ ID NO: 34, SEQ ID NO: 35, SEQ ID NO: 77-SEQ ID NO: 82, or SEQ ID NO: 85-SEQ ID NO: 89.

In some aspects, the small RNA payload comprises an engineered guide RNA capable of hybridizing to a target sequence. In some aspects, the engineered guide RNA is at least 70%, at least 75%, at least 80%, at least 85%, at least 90%, at least 95%, or 100% reverse complementary to the target sequence. In some aspects, the engineered guide RNA comprises at least one base pair mismatch relative to the target sequence. In some aspects, the target sequence comprises an adenosine residue. In some aspects, the target sequence is an RNA sequence. In some aspects, the RNA sequence is a mRNA or a pre-mRNA. In some aspects, the target sequence comprises a G to A mutation relative to a wild type sequence. In some aspects, the target sequence comprises a missense mutation or a nonsense mutation relative to a wild type sequence.

In some aspects, the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2).

In some aspects, the small RNA payload comprises an antisense oligonucleotide, an siRNA, an shRNA, a miRNA, or a tracrRNA. In some aspects, the small RNA payload is not less than 20 nucleotide residues and not more than 500 nucleotide residues long. In some aspects, the small RNA payload is not less than 60 and not more than 100 residues long. In some aspects, the small RNA payload is not less than 80 and not more than 120 residues long. In some aspects, the small RNA payload is not less than 100 and not more than 140 residues long. In some aspects, the small RNA payload is not less than 130 and not more than 170 residues long.

In some aspects, the payload sequence further comprises an Sm binding sequence or a hairpin sequence. In some aspects, the hairpin sequence comprises a U7 hairpin. In some aspects, each expression cassette of the plurality of expression cassettes independently has a length of not less than 1300 nucleotide residues and not more than 2160 nucleotide residues. In some aspects, each expression cassette of the plurality of expression cassettes independently comprises at least 80% sequence identity to a U1 sequence or a U7 sequence. In some aspects, the U1 sequence is a mouse U1 sequence or a human U1 sequence. In some aspects, the U7 sequence is a mouse U7 sequence or a human U7 sequence.

In some aspects, the engineered guide RNA is capable of forming a guide-target RNA scaffold comprising a structural feature upon hybridization of the small RNA payload to a target sequence. In some aspects, the structural feature is a bulge, a mismatch, an internal loop, a hairpin, or combinations thereof. In some aspects, the structural feature comprises the bulge, and wherein the bulge is a symmetric bulge. In some aspects, the structural feature comprises the bulge, and wherein the bulge is an asymmetric bulge. In some aspects, the structural feature comprises the internal loop, and wherein the internal loop is a symmetric internal loop. In some aspects, the structural feature comprises the internal loop, and wherein the internal loop is an asymmetric internal loop. In some aspects, the structural feature comprises the hairpin, and wherein the hairpin is a recruitment hairpin or a non-recruitment hairpin. In some aspects, the guide-target RNA scaffold comprises a wobble base pair.

In various aspects, the present disclosure provides a pharmaceutical composition comprising the viral vector as described herein and a pharmaceutically acceptable excipient, carrier, diluent, or combination thereof.

In various aspects, the present disclosure provides a pharmaceutical composition comprising the polynucleotide as described herein or the viral vector as described herein and a pharmaceutically acceptable excipient, carrier, diluent, or combination thereof.

In various aspects, the present disclosure provides a method of expressing a small RNA payload in a cell, the method comprising delivering the viral vector as described herein or the pharmaceutical composition as described herein to a cell and expressing the small RNA payload encoded by the expression cassette in the cell. In various aspects, the method of expressing a small RNA payload in a cell is conducted in vitro or ex vivo.

In various aspects, the present disclosure provides a method of expressing a small RNA payload in a cell, the method comprising delivering the polynucleotide as described herein, the viral vector as described herein, or the pharmaceutical composition as described herein to a cell and expressing the small RNA payload encoded by the expression cassette in the cell. In various aspects, the method of expressing a small RNA payload in a cell is conducted in vitro or ex vivo.

In various aspects, the present disclosure provides a method of editing a target sequence, the method comprising: delivering the viral vector as described herein or the pharmaceutical composition as described herein to a cell encoding the target sequence, expressing the small RNA payload in the cell; forming a guide-target RNA scaffold upon hybridization of the small RNA payload to the target sequence; recruiting an editing enzyme to the target sequence; and editing the target sequence with the editing enzyme. In various aspects, the method of editing a target sequence is conducted in vitro or ex vivo.

In various aspects, the present disclosure provides a method of editing a target sequence, the method comprising: delivering the polynucleotide as described herein, the viral vector as described herein, or the pharmaceutical composition as described herein to a cell encoding the target sequence, expressing the small RNA payload in the cell; forming a guide-target RNA scaffold upon hybridization of the small RNA payload to the target sequence; recruiting an editing enzyme to the target sequence; and editing the target sequence with the editing enzyme. In various aspects, the method of editing a target sequence is conducted in vitro or ex vivo.

In various aspects, the present disclosure provides a method of administering a viral vector to a subject with a disease, the method comprising: administering to the subject a composition comprising the viral vector as described herein or the pharmaceutical composition as described herein; delivering the expression cassette to a cell of the subject; and expressing a small RNA payload in the cell.

In various aspects, the present disclosure provides a method of administering a therapeutic polynucleotide to a subject with a disease, the method comprising: administering to the subject a composition comprising the polynucleotide as described herein, the viral vector as described herein, or the pharmaceutical composition as described herein; delivering the therapeutic polynucleotide to a cell of the subject; and expressing a small RNA payload in the cell.

In various aspects, the present disclosure provides a method of treating a disease in a subject, the method comprising: administering to the subject a composition comprising the viral vector as described herein or the pharmaceutical composition of claim 46 as described herein; delivering the expression cassette to a cell of the subject; and expressing a small RNA payload in the cell, thereby treating the disease.

In various aspects, the present disclosure provides a method of treating a disease in a subject, the method comprising: administering to the subject a composition comprising the polynucleotide as described herein, the viral vector as described herein, or the pharmaceutical composition as described herein; delivering a therapeutic polynucleotide to a cell of the subject; and expressing a small RNA payload in the cell, thereby treating the disease.

In some aspects, the disease is a synucleinopathy, Parkinson's disease, Lewy body dementia, multiple system atrophy, Charcot-Marie-Tooth disease, hereditary neuropathy with liability to pressure palsies, Yuan-Harel-Lupski syndrome, a tauopathy, Alzheimer's disease, frontotemporal dementia, progressive supranuclear palsy, corticobasal degeneration, chronic traumatic encephalopathy, autism, traumatic brain injury, Dravet syndrome, Crohn's disease, muscular dystrophy, B-cell leukemia, Dejerine-Sottas disease, Stargardt disease, alpha-1 antitrypsin deficiency, Tay-Sachs disease, cystic fibrosis, liposomal acid lipase deficiency, or Gaucher disease.

In some aspects, the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2).

In some aspects, the small RNA payload comprises an engineered guide RNA that hybridizes to a target sequence, and wherein the cell encodes the target sequence. In some aspects, the method further comprises forming a guide-target RNA scaffold upon hybridization of the engineered guide RNA to the target sequence, recruiting an editing enzyme to the target sequence, and editing the target sequence with the editing enzyme. In some aspects, the target sequence comprises a mutation relative to a wild type sequence. In some aspects, editing the target sequence corrects the mutation in the target sequence. In some aspects, the mutation is a missense mutation. In some aspects, the mutation is a nonsense mutation. In some aspects, the mutation is a G to A mutation. In some aspects, the mutation is associated with the disease. In some aspects, editing the target sequence comprises editing an untranslated region of the target.

In some aspects, the untranslated region is a 5′ untranslated region or a 3′ untranslated region. In some aspects, the 3′ untranslated region is a polyadenylation sequence. In some aspects, editing the target sequence comprises editing a translation initiation site. In some aspects, editing the target sequence alters expression of the target sequence. In some aspects, editing the target sequence increases expression of the target sequence. In some aspects, editing the target sequence decreases expression of the target sequence.

In some aspects, the editing enzyme comprises an ADAR, an APOBEC, or a Cas nuclease. In some aspects, the ADAR comprises ADAR1, ADAR2, ADAR3, or combinations thereof. In some aspects, the target sequence comprises RNA or DNA. In some aspects, the target sequence is a mRNA or a pre-mRNA. In some aspects, editing the target sequence comprises deamidating a nucleotide of the target sequence. In some aspects, the target sequence is edited with an efficiency of at least 10%, at least 20%, or at least 25%.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A schematically illustrates an example configuration that includes an engineered guide RNA expression cassette based on a human U1 cassette in the forward read direction.

FIG. 1B schematically illustrates an example configuration that includes a human U1 expression cassette having a forward read directionality and a mouse U7 expression cassette having a forward read directionality.

FIG. 1C, schematically illustrates an example configuration that includes a mouse U7 expression cassette having a reverse read directionality and a human U1 expression cassette having a forward read directionality.

FIG. 1D schematically illustrates an example configuration that includes a human U1 cassette having a forward read directionality and a mouse U7 cassette having a reverse read directionality.

FIG. 2A is a bar graph showing expression of guide RNA luciferase reporter, Reporter 1, when configurations described in FIG. 1A-FIG. 1D are tested using plasmid delivery of vectors including: a viral vector with a human U1 cassette in the forward read direction with a PMP22 guide RNA payload (SEQ ID NO: 26); a viral vector with a human U1 cassette having a forward read directionality and a mouse U7 cassette having a forward read directionality each having a PMP22 guide RNA payload (SEQ ID NO: 27); a viral vector with mouse U7 cassette having a reverse read directionality and a human U1 cassette having a forward read directionality each having a PMP22 guide RNA payload (SEQ ID NO: 28); and a viral vector with human U1 cassette having a forward read directionality and a mouse U7 cassette having a reverse read directionality each having a PMP22 guide RNA payload (SEQ ID NO: 29) compared to the expression of no plasmid delivery (“no plasmid”).

FIG. 2B is a bar graph showing expression of guide RNA luciferase reporter, Reporter 2, when configurations described in FIG. 1A-FIG. 1D are tested using plasmid delivery of vectors including: a viral vector with a human U1 cassette in the forward read direction with an SNCA guide RNA payload (SEQ ID NO: 30); a viral vector with a human U1 cassette having a forward read directionality and a mouse U7 cassette having a forward read directionality each having an SNCA guide RNA payload (SEQ ID NO: 31); a viral vector with mouse U7 cassette having a reverse read directionality and a human U1 cassette having a forward read directionality each having an SNCA guide RNA payload (SEQ ID NO: 32); and a viral vector with human U1 cassette having a forward read directionality and a mouse U7 cassette having a reverse read directionality each having an SNCA guide RNA payload (SEQ ID NO: 33) compared to the expression of no plasmid delivery (“no plasmid”).

FIG. 2C is a bar graph showing expression of guide RNA luciferase reporter, Reporter 1, when configurations described in FIG. 1A-FIG. 1D are tested using viral (AAV) delivery of vectors including: a viral vector with a human U1 cassette in the forward read direction with a PMP22 guide RNA payload (SEQ ID NO: 26); a viral vector with a human U1 cassette having a forward read directionality and a mouse U7 cassette having a forward read directionality each having a PMP22 guide RNA payload (SEQ ID NO: 27); a viral vector with mouse U7 cassette having a reverse read directionality and a human U1 cassette having a forward read directionality each having a PMP22 guide RNA payload (SEQ ID NO: 28); and a viral vector with human U1 cassette having a forward read directionality and a mouse U7 cassette having a reverse read directionality each having a PMP22 guide RNA payload (SEQ ID NO: 29) compared to the expression of no plasmid delivery (“no plasmid”).

FIG. 2D is a bar graph showing expression of guide RNA luciferase reporter, Reporter 2, when configurations described in FIG. 1A-FIG. 1D are tested using viral (AAV) delivery of vectors including: a viral vector with a human U1 cassette in the forward read direction with an SNCA guide RNA payload (SEQ ID NO: 30); a viral vector with a human U1 cassette having a forward read directionality and a mouse U7 cassette having a forward read directionality each having an SNCA guide RNA payload (SEQ ID NO: 31); a viral vector with mouse U7 cassette having a reverse read directionality and a human U1 cassette having a forward read directionality each having an SNCA guide RNA payload (SEQ ID NO: 32); and a viral vector with human U1 cassette having a forward read directionality and a mouse U7 cassette having a reverse read directionality each having an SNCA guide RNA payload (SEQ ID NO: 33). compared to the expression of no plasmid delivery (“no plasmid”).

FIG. 3A is a line graph showing expression of the guide RNA luciferase reporter over the GAPDH control (Guide/GAPDH) over increasing multiplicity of infection (MOI) of the wild type single copy vector, as shown in FIG. 3C, and the engineered two copy vector as shown in FIG. 3C.

FIG. 3B shows a line graph showing Sanger editing of an ATG sequence to GTG to evaluate expression and editing activity over increasing multiplicity of infection (MOI) of the wild type single copy vector, as shown in FIG. 3C, and the engineered two copy vector as shown in FIG. 3C.

FIG. 3C shows a schematic of the wild type single copy vector (top) and the engineered two copy vector (bottom).

FIG. 4A schematically illustrates the 16 permutations of 4 expression cassettes with each cassette having a forward (F) or a reverse (R) read directionality.

FIG. 4B schematically illustrates the 5 permutations of 4 expression cassettes where the expression cassettes do not read into each other.

FIG. 5A shows a line graph showing Sanger editing of an ATG sequence to GTG to evaluate editing activity of vector constructs in mouse primary neurons. Editing activity was tested for a control AAV transduction without a vector (“Control”), an AAV transduction of a vector with one expression cassette (“WT single copy”), an AAV transduction of a vector with two expression cassettes with the first cassette having a reverse read orientation and the second cassette having a forward read orientation (“Eng two copy”), and a vector with two expression cassettes with the first cassette having a reverse read orientation and the second cassette having a forward read orientation, and with additional engineered sequence elements, including an hnRNP motif, an OPT sequence, and a hairpin sequence (“Eng two copy+gRNA opt”).

FIG. 5B shows a line graph showing Sanger editing of an ATG sequence to GTG to evaluate editing activity of vector constructs in SH-SY5Y cells. Editing activity was tested for a control AAV transduction without a vector (“Control”), an AAV transduction of a vector with one expression cassette (“WT single copy”), an AAV transduction of a vector with two expression cassettes with the first cassette having a reverse read orientation and the second cassette having a forward read orientation (“Eng two copy”), and a vector with two expression cassettes with the first cassette having a reverse read orientation and the second cassette having a forward read orientation, and with additional engineered sequence elements, including an hnRNP motif, an OPT sequence, and a hairpin sequence (“Eng two copy+gRNA opt”).

FIG. 6 shows a schematic of the two-copy vectors tested in two orientations including a tandem orientation wherein the guide RNAs have the same read directionality of forward and a bidirectional orientation wherein the guide RNAs have different read directionalities. Specifically, the bidirectional orientation has a first guide RNA with a read directionality of reverse, and a second guide RNA with a read directionality of forward and the tandem orientation has a first guide RNA with a read directionality of forward, and a second guide RNA with a read directionality of forward.

FIG. 7A shows a bar graph of a guide RNA (gRNA1) expression relative to a GAPDH control (gRNA/GAPDH) of a bidirectional orientation (2×-bidirectional) two-copy vector, a tandem orientation (2×-tandem) two-copy vector, and a vector with a single guide RNA (Single) that were delivered via plasmid transfection.

FIG. 7B shows a bar graph of a guide RNA (gRNA1) expression relative to a GAPDH control (gRNA/GAPDH) of a bidirectional orientation (2×-bidirectional) two-copy vector, a tandem orientation (2×-tandem) two-copy vector, and a vector with a single guide RNA (Single) that were delivered via AAV transfection.

FIG. 7C shows a bar graph of a guide RNA (gRNA1) expression relative to a GAPDH control (gRNA/GAPDH) of a bidirectional orientation (Bidirectional) two-copy vector, a tandem orientation (Tandem) two-copy vector, and a vector with a single guide RNA (Single) that were delivered via AAV transfection at different multiplicities of infection (MOI) of 10 k and 100 k.

FIG. 8A shows a bar graph of a guide RNA (gRNA2) expression relative to a GAPDH control (gRNA/GAPDH) of a bidirectional orientation (2×-bidirectional) two-copy vector, a tandem orientation (2×-tandem) two-copy vector, and a vector with a single guide RNA (Single) that were delivered via plasmid transfection.

FIG. 8B shows a bar graph of a guide RNA (gRNA2) expression relative to a GAPDH control (gRNA/GAPDH) of a bidirectional orientation (2×-bidirectional) two-copy vector, a tandem orientation (2×-tandem) two-copy vector, and a vector with a single guide RNA (Single) that were delivered via AAV transfection.

FIG. 8C shows a bar graph of a guide RNA (gRNA2) expression relative to a GAPDH control (gRNA/GAPDH) of a tandem orientation (Tandem) two-copy vector and a vector with a single guide RNA (Single) that were delivered via AAV transfection at different multiplicities of infection (MOI) of 10 k and 100 k.

FIG. 9A shows a bar graph of percent A to G editing of a CNS target in mouse primary neurons of a bidirectional orientation (2× bidirectional v2.0) two-copy vector and a single-copy guide RNA (single WT) vector.

FIG. 9B shows a plot of guide expression relative to a GAPDH control (gRNA/GAPDH) of a bidirectional orientation (2× bidirectional v2.0) two-copy vector and a single-copy guide RNA (single WT) vector across various multiplicities of infection (MOIs) including 10, 100, 1,000, and 10,000.

FIG. 10 shows an alkaline gel evaluation of vector genome integrity of two-copy vectors comprising two expression cassettes each with the same guide RNA, hairpin structure, and terminator sequence (about 250 nucleotides of sequence homology) also compared to single-copy vectors.

FIG. 11 shows an alkaline gel evaluation of vector genome integrity of two-copy vectors comprising two guide RNAs with different sequences (distinct sequences), vectors with two guide RNAs with similar sequences (diverged guide RNAs), and vectors with two guide RNAs with the same sequence (identical guide RNAs). The two-copy vectors were in a bidirectional orientation, as shown in the schematic in FIG. 11, or were in a tandem orientation.

FIG. 12 shows an alkaline gel evaluation of vector genome integrity of two-copy vectors comprising two guide RNAs with 10 bp of divergence with each expression cassette and with distinct hairpin and terminator sequences. The two-copy vectors were tested in both bidirectional and tandem orientations.

FIG. 13 shows a schematic of sequence diverged guide RNA designs. 100mer gRNAs were designed and tested that had 10 regions of 1 nucleotide alteration (10×1), 10 regions of 2 nucleotide alterations (10×2), 10 regions of 3 nucleotide alterations (10×3), 2 regions of 5 nucleotide alterations (2×5), 4 regions of 5 nucleotide alterations (4×5), 6 regions of 5 nucleotide alterations (6×5), 1 region of 10 nucleotide alterations (1×10), 2 regions of 10 nucleotide alterations (2×10), 3 regions of 10 nucleotide alterations (3×10), 2 regions of 15 nucleotide alterations (2×15), 1 region of 20 nucleotide alterations (1×20), or 1 region of 30 nucleotide alterations (1×20).

FIG. 14 shows an alkaline gel evaluation of vector genome integrity of two-copy vectors in a bidirectional orientation comprising sequence divergent guide RNAs designed as provided in FIG. 13.

FIG. 15 shows a bar graph of the percent intact for each of the vector genomes evaluated in the alkaline gel of FIG. 14. The percent intact values for each of the 100mer gRNAs were calculated using the intensities of each gel band as measured by ImageJ analysis software and the following equation: Percent Intact=(Intensity (Full length vector)/(Intensity (Full length vector)+Intensity (Vector truncations)))*100%; wherein the Intensity (Full length vector) is the intensity of the highest molecular weight band in the gel and the Intensity (Vector truncations) is the intensity of the lower molecular weight band.

FIG. 16 shows the alkaline gel evaluation of vector genome integrity of FIG. 14 with the contrast increased to show the presence of additional lower bands around 1.3 kb, as indicated by the brackets.

FIG. 17 shows an alkaline gel evaluation of vector genome integrity of two-copy vectors in a tandem orientation comprising sequence divergent guide RNAs designed as provided in FIG. 13.

FIG. 18 shows a bar graph of the percent intact for each of the vector genomes evaluated in the alkaline gel of FIG. 17. The percent intact values for each of the 100mer gRNAs were calculated using the intensities of each gel band as measured by ImageJ analysis software and the following equation: Percent Intact=(Intensity (Full length vector)/(Intensity (Full length vector)+Intensity (Vector truncations)))*100%; wherein the Intensity (Full length vector) is the intensity of the highest molecular weight band in the gel and the Intensity (Vector truncations) is the intensity of the lower molecular weight band.

FIG. 19 shows the alkaline gel evaluation of vector genome integrity of FIG. 17 with the contrast increased to show the presence of additional lower bands.

FIG. 20 shows a schematic for developing guide RNAs with sequence divergence. Nucleotide alterations were introduced at nucleotide positions in the guide RNA sequence that would either be an alternative mismatch position in the guide-target RNA scaffold or would introduce a GU wobble base pair in the guide-target RNA scaffold. Alternative mismatches were introduced in regions of internal structure in the guide-target RNA scaffold (e.g., internal loops) and GU wobble base pairs were introduced at A and C nucleotides by replacement with G and T nucleotides, respectively.

FIG. 21 shows a schematic of sequence divergent guide RNA designs. Sequence divergent guide RNAs were designed with i) only alternative mismatches, ii) only GU wobble base pairs and iii) combinatorial design with both alternative mismatches and GU wobble base pairs.

FIG. 22A shows a schematic of a sequence divergent guide RNA design. The sequence divergent guide RNA had both GU wobble base pairs introduced into the guide-target RNA scaffold (dark blue circles) and alternative mismatches in the internal loops of the guide-target RNA scaffold (light blue circles).

FIG. 22B shows a bar graph of RNA editing (% RNA editing) from the sequence divergent guide RNAs with alternative mismatches (gRNA 1-6), GU wobble base pairs, and the combinatorial designs of both alternative mismatches and GU wobble base pairs compared to the RNA editing to the original guide RNA sequence (gRNA, “primary design”).

FIG. 23A shows a schematic of sequence divergent guide RNAs that were developed by introducing alternative mismatches and GU wobble base pairs. The nucleotide alterations were confirmed to not change the guide-target RNA scaffold as shown for 12 (“P12”) and 30 (“P30”) nucleotide alterations.

FIG. 23B shows a bar graph of RNA editing (% RNA editing) of sequence divergent guide RNAs that were developed by introducing alternative mismatches and GU wobble base pairs for a total of 12 (“P12”), 16 (“P16”), 18 (“P18”), 20 (“P20”), 21 (“P21”), 22 (“P22”), 23 (“P23”), 24 (“P24”), 25 (“P25”), 26 (“P26”), 27 (“P27”), 28 (“P28”), 29 (“P29”), and 30 (“P30”) total nucleotide alterations from the original guide RNA sequence (“P0”). The RNA editing profiles for the original guide RNA sequence (“P0”) and the sequence divergent guide RNA with 20 nucleotide alterations (“P20”) are also provided.

FIG. 24 shows a graph of the mean percent editing for each position of a target sequence by sequence divergent guide RNAs that were developed by introducing alternative mismatches and GU wobble base pairs for a total of 12 (“P12”), 16 (“P16”), 18 (“P18”), 20 (“P20”), 21 (“P21”), 22 (“P22”), 23 (“P23”), 24 (“P24”), 25 (“P25”), 26 (“P26”), 27 (“P27”), 28 (“P28”), 29 (“P29”), and 30 (“P30”) total nucleotide alterations from the original guide RNA sequence (“P0”). The RNA editing profiles for the original guide RNA sequence (“P0”) and the sequence divergent guide RNA with 24 nucleotide alterations (“P24”) are also provided.

FIG. 25 shows a bar graph of RNA editing of a CNS target sequence by a sequence diverged guide RNA with 20 total nucleotide alterations (“P20”) compared to the original guide RNA sequence (“P0”). The total editing by a sequence diverged guide RNA with 20 total nucleotide alterations (“P20”) was 58% and the original guide RNA sequence (“P0”) was 49%. The RNA editing is also shown for each target position of the CNS target sequence and shows that the sequence diverged guide RNA with 20 total nucleotide alterations (“P20”) had less RNA editing at off-target positions and increased editing at the target position (0 target position) compared to the original guide RNA sequence (“P0”).

FIG. 26 shows a schematic of the effect of GU wobble base pairing on the structure of the double stranded RNA (dsRNA) helix. As shown, GU wobble base pairs have a different base pairing structure than the canonical GC base pair introducing a different angle in the dsRNA helix that is offset by 14.0° and a decrease in the radius of the dsRNA helix by −0.05 Å. This may cause the dsRNA with GU wobble base pairs to over-twist as compared to the canonical dsRNA helix without GU wobble base pairs.

FIG. 27 shows a schematic of the two-copy vector designs evaluated for transduction marker-free vector designs and includes the bidirectional vector designs (e.g., top of FIG. 27) and tandem vector designs (e.g., middle of FIG. 27) that were compared to single copy vector designs (e.g., bottom of FIG. 27).

FIG. 28A shows an alkaline gel evaluation of the vector genome integrity for two-copy vector designs evaluated for transduction marker-free vector designs. The two-copy vector designs evaluated for transduction marker-free vector designs included bidirectional vector designs (Bidirectional) and tandem vector designs (Tandem) that were compared to the genome integrity of single copy vector designs (Single).

FIG. 28B shows a bar graph of the percent intact for each of the vector genomes evaluated in the alkaline gel of FIG. 28A. The percent intact values for each of the transduction marker-free vectors were calculated using the intensities of each gel band as measured by ImageJ analysis software and the following equation: Percent Intact=(Intensity (Full length vector)/(Intensity (Full length vector)+Intensity (Vector truncations)))*100%; wherein the Intensity (Full length vector) is the intensity of the highest molecular weight band in the gel and the Intensity (Vector truncations) is the intensity of the lower molecular weight band.

FIG. 29A shows a bar graph of the RNA editing of a target sequence (On Target Editing (%)) for each of the transduction marker-free vector designs. The two-copy vector designs evaluated for transduction marker-free vector designs included bidirectional vector designs and tandem vector designs that were compared to the RNA editing of a two-copy vector design with a transduction marker. Each of the vector designs were tested at 5 k, 50 k, and 500 k multiplicities of infection (MOI).

FIG. 29B shows a bar graph of the fold change RNA editing of the transduction marker-free vector designs including the bidirectional vector designs and the tandem vector designs relative to the RNA editing of a two-copy vector design with a transduction marker. Each of the fold change values are provided at 5 k, 50 k, and 500 k multiplicities of infection (MOI).

FIG. 30A shows a bar graph of the expression of a guide RNA (“05450”) relative to a GAPDH control (gRNA/GAPDH) for the transduction marker-free vector designs including the bidirectional vector designs and the tandem vector designs and compared to the gRNA expression of a two-copy vector design with a transduction marker. Each of the gRNA expression values are provided at 5 k, 50 k, and 500 k multiplicities of infection (MOI).

FIG. 30B shows a bar graph of the expression of a guide RNA (“38764”) relative to a GAPDH control (gRNA/GAPDH) for the transduction marker-free vector designs including the bidirectional vector designs and the tandem vector designs and compared to the gRNA expression of a two-copy vector design with a transduction marker. Each of the gRNA expression values are provided at 5 k, 50 k, and 500 k multiplicities of infection (MOI).

FIG. 31A and FIG. 31B provide schematics of vector with synthetic filler additions in three different positions, addition on the 5′ end of the expression cassette (5′), addition on both the 5′ and 3′ end of the expression cassette (Mid), and addition on the 3′ end of the expression cassette (3′).

FIG. 32A, FIG. 32B, FIG. 32C, and FIG. 32D provide bar graphs of SNCA-TIS editing by vectors with synthetic filler additions in three different positions, addition on the 5′ end of the expression cassette (5′), addition on both the 5′ and 3′ end of the expression cassette (Mid), and addition on the 3′ end of the expression cassette (3′) at four different multiplicities of infection (MOIs) of the AAV dosage including 5 MOI (FIG. 32A), 500 MOI (FIG. 32B), 5000 MOI (FIG. 32C), and 50000 MOI (FIG. 32D).

FIG. 33A provides a bar graph of percent SNCA-TIS editing by vector constructs with extended termination sequences as compared to vector constructs with the non-extended termination sequences equivalent of each termination sequence tested.

FIG. 33B provides a bar graph of GFP fluorescence by vector constructs with extended termination sequences as compared to vector constructs with the non-extended termination sequences equivalent of each termination sequence tested.

FIG. 34A provides a bar graph of percent SNCA-TIS editing by vector constructs with extended promoter sequences as compared to vector constructs with the non-extended promoter sequences equivalent of each promoter sequence tested.

FIG. 34B provides a bar graph of GFP fluorescence by vector constructs with extended promoter sequences as compared to vector constructs with the non-extended promoter sequences equivalent of each promoter sequence tested and a vehicle-only control to show the baseline fluorescence (“Baseline”).

FIG. 35 provides a graph of percent A→G SNCA TIS editing as a function of AAV dosage MOI for tandem, bidirectional, and single expression cassette vector constructs.

FIG. 36A provides a bar graph of SNCA TIS editing at an AAV dosage of 5 MOI of the vector constructs tested.

FIG. 36B provides a bar graph of SNCA TIS editing at an AAV dosage of 50 MOI of the vector constructs tested.

FIG. 37A provides a schematic of the tandem orientation of the developmental vector constructs tested.

FIG. 37B shows an alkaline gel evaluation of the tandem developmental vector genome integrity for the vector designs provided in TABLE 9.

FIG. 37C shows a bar graph of the percent intact for each of the vector genomes evaluated in the alkaline gel of FIG. 37A. The percent intact values for each of the tandem developmental vector genome were calculated using the intensities of each gel band as measured by ImageJ analysis software and the following equation: Percent Intact=(Intensity (Full length vector)/(Intensity (Full length vector)+Intensity (Vector truncations)))*100%; wherein the Intensity (Full length vector) is the intensity of the highest molecular weight band in the gel and the Intensity (Vector truncations) is the intensity of the lower molecular weight band.

FIG. 38 provides a schematic of moving to the developmental vector design from the research vector design by removing the transduction marker sequence and extending the cassette element sequences to retain vector size.

FIG. 39 provides a graph of percent SNCA TIS RNA editing in HEK293 cells compared between the developmental vector design (“dual cassette”), a single expression cassette vector (“single cassette”), and the research vector design with a transduction marker sequence (“research vector”).

FIG. 40 provides a bar graph of percent SNCA TIS RNA editing of vector constructs with elongated expression cassette elements (e.g., promoter or termination sequences) as present in the developmental vector design as compared to the non-elongated expression cassette elements as present in the research vector design.

FIG. 41A provides a graph of guide RNA expression in mouse primary neuron cells of two developmental vector candidate designs (Candidate 1 and 2), compared to the guide RNA expression from the research vector design and a vector design with a single expression cassette at various AAV doses (MOI).

FIG. 41B provides a bar graph of the guide RNA expression in mouse primary neuron cells from each of the expression cassettes in a developmental vector design (Candidate 1). Each of the expression cassette guide RNA expressions (Cassette 1 and Cassette 2) was measured at low (500), middle (5000), and high (50000) AAV doses (MOI).

FIG. 41C provides a graph of guide RNA expression in human iPSC-derived neuron cells of two developmental vector candidate designs (Candidate 1 and 2) at various AAV doses (MOI).

FIG. 41D provides a bar graph of the guide RNA expression in human iPSC-derived neuron cells from each of the expression cassettes in a developmental vector design (Candidate 1). Each of the expression cassette guide RNA expressions (Cassette 1 and Cassette 2) was measured at low (10000), middle (50000), and high (100000) AAV doses (MOI).

### DETAILED DESCRIPTION

Vectors with multiple expression cassettes may be beneficial for increasing RNA payload expression by providing additional copies of a polynucleotide encoding an RNA payload per each vector delivered. Variation in the read orientation (i.e., forward or reverse) of each expression cassette in a vector with a plurality of expression cassettes may influence the expression of an RNA payload and frequency of unwanted events, such as recombination. For example, by orienting expression cassettes with opposing read orientations, expression of an RNA payload may be increased, and the frequency of recombination events may be decreased. Provided herein are various polynucleotides (e.g., viral vectors) comprising a plurality of expression cassettes encoding small RNA payloads that are designed to reduce the frequency of unwanted events as well as methods of making and using those viral vectors.

The present disclosure provides viral vectors with a plurality of expression cassettes for expressing RNA payloads. The expression cassettes described herein may be engineered for increased expression of the encoded RNA payload sequence. In some embodiments, certain elements of the expression cassette, such as enhancer sequences, core promoter sequences, or transcriptional termination sequences, may be engineered for enhanced payload expression. These sequence elements may be engineered from various endogenous promoters, such as U1, U6, or U7 promoters, for increased payload expression. The individual sequence elements of the expression cassette may be engineered to enhance expression of the encoded RNA payload.

### Expression Cassette Multitude

The present disclosure provides polynucleotides (e.g., viral vectors) with a plurality of expression cassettes for expressing RNA payloads e.g., small RNA payloads. A plurality of expression cassettes can include two or more expression cassettes, three or more expression cassettes, four or more expression cassettes, five or more expression cassettes, six or more expression cassettes, seven or more expression cassettes, eight or more expression cassettes, nine or more expression cassettes, or ten or more expression cassettes. In some embodiments, the plurality of expression cassette sequences comprises two expression cassette sequences, three expression cassette sequences, four expression cassette sequences, five expression cassette sequences, six expression cassette sequences, seven expression cassette sequences, eight expression cassette sequences, nine expression cassette sequences, or ten expression cassette sequences. For example, a polynucleotide (e.g., a viral vector) with a plurality of expression cassettes may be a polynucleotide with two expression cassettes. In another example, a polynucleotide with a plurality of expression cassettes may be a polynucleotide with three expression cassettes. In another example, a polynucleotide with a plurality of expression cassettes may be a polynucleotide with four expression cassettes. In another example, a polynucleotide with a plurality of expression cassettes may be a polynucleotide with five expression cassettes. In another example, a polynucleotide with a plurality of expression cassettes may be a polynucleotide with six expression cassettes. In another example, a polynucleotide with a plurality of expression cassettes may be a polynucleotide with seven expression cassettes. In another example, a polynucleotide with a plurality of expression cassettes may be a polynucleotide with eight expression cassettes. In another example, a polynucleotide with a plurality of expression cassettes may be a polynucleotide with nine expression cassettes. In another example, a polynucleotide with a plurality of expression cassettes may be a polynucleotide with ten expression cassettes. Each expression cassette of the plurality of expression cassettes can each independently include a promoter sequence, a payload sequence, and a transcription termination sequence. The payload sequence can be under the transcriptional control of the promoter sequence.

When a plurality of expression cassettes is included in a polynucleotide, the arrangement of the plurality of expression cassettes can be designed for enhanced expression. For example, the arrangement of the plurality of expression cassettes can be designed to increase the expression of the small RNA payload. The plurality of expression cassettes of a polynucleotide may be arranged in the polynucleotide relative to the 5′ and 3′ ends of the polynucleotide. For example, a first expression cassette may be included in the polynucleotide with a second expression cassette included on the 3′ end of the first expression cassette. In another embodiment, a third expression cassette could be included on the 3′ end of the second expression cassette. In another embodiment, a fourth expression cassette could be included on the 3′ end of the third expression cassette.

Each expression cassette within the polynucleotide can have a read directionality relative to the 5′ to 3′ orientation of the sequence of the polynucleotide. As used herein, the term “read directionality” refers to the direction of transcription for a given expression cassette. Forward read directionality can be used to describe that the direction of transcription is from 5′ to 3′ whereas the reverse read directionality can be used to describe that the direction of transcription is 3 to 5′. In some embodiments, an expression cassette having a reverse read directionality can include the antisense strand of the expression cassette.

In some embodiments, all of the expression cassettes in the plurality of expression cassettes in a polynucleotide can have the same read directionality. In some embodiments, more than one expression cassette in a polynucleotide can have the same read directionality. In some embodiments, at least one expression cassette in a polynucleotide can have a different read directionality compared to other expression cassettes in the polynucleotide.

In some embodiments, a polynucleotide can include two expression cassettes with a first expression cassette and a second expression cassette. In one embodiment, the first expression cassette has the read directionality of forward and the second expression cassette has the read directionality of reverse. In one embodiment, the first expression cassette has the read directionality of reverse and the second expression cassette has the read directionality of forward. In one embodiment, the first expression cassette has the read directionality of forward and the second expression cassette has the read directionality of forward. In one embodiment, the first expression cassette has the read directionality of reverse and the second expression cassette has the read directionality of reverse.

A “bidirectional” vector orientation as used herein is used to describe a vector orientation where a first expression cassette and a second expression cassette have read directions that are not the same. For example, a bidirectional vector orientation may include a vector orientation wherein the first expression cassette has a read directionality of forward and the second expression cassette has the read directionality of reverse. In another example, a bidirectional vector orientation may include a vector orientation wherein the first expression cassette has a read directionality of reverse and the second expression cassette has the read directionality of forward.

A “tandem” vector orientation as used herein is used to describe a vector orientation where a first expression cassette and a second expression cassette have read directions that are the same. For example, a tandem vector orientation may include a vector orientation wherein the first expression cassette has a read directionality of forward and the second expression cassette has a read directionality of forward. In another example, a tandem vector orientation may include a vector orientation wherein the first expression cassette has a read directionality of reverse and the second expression cassette has a read directionality of reverse. A tandem vector orientation may refer to a vector orientation wherein the first expression cassette sequence and the second expression cassette sequence are each independently arranged in a 5′ to 3′ orientation to have a read directionality of forward.

In some embodiments, the polynucleotides can include three expression cassettes with a first expression cassette, a second expression cassette and a third expression cassette. In one embodiment, the first expression cassette has the read directionality of forward, the second expression cassette has the read directionality of forward, and the third expression cassette has the read directionality of reverse. In one embodiment, the first expression cassette has the read directionality of forward, the second expression cassette has the read directionality of reverse, and the third expression cassette has the read directionality of forward. In one embodiment, the first expression cassette has the read directionality of forward, the second expression cassette has the read directionality of reverse, and the third expression cassette has the read directionality of reverse. In one embodiment, the first expression cassette has the read directionality of reverse, the second expression cassette has the read directionality of forward, and the third expression cassette has the read directionality of forward. In one embodiment, the first expression cassette has the read directionality of reverse, the second expression cassette has the read directionality of forward, and the third expression cassette has the read directionality of reverse. In one embodiment, the first expression cassette has the read directionality of reverse, the second expression cassette has the read directionality of reverse, and the third expression cassette has the read directionality of forward. In one embodiment, the first expression cassette has the read directionality of forward, the second expression cassette has the read directionality of forward, and the third expression cassette has the read directionality of forward. In one embodiment, the first expression cassette has the read directionality of reverse, the second expression cassette has the read directionality of reverse, and the third expression cassette has the read directionality of reverse.

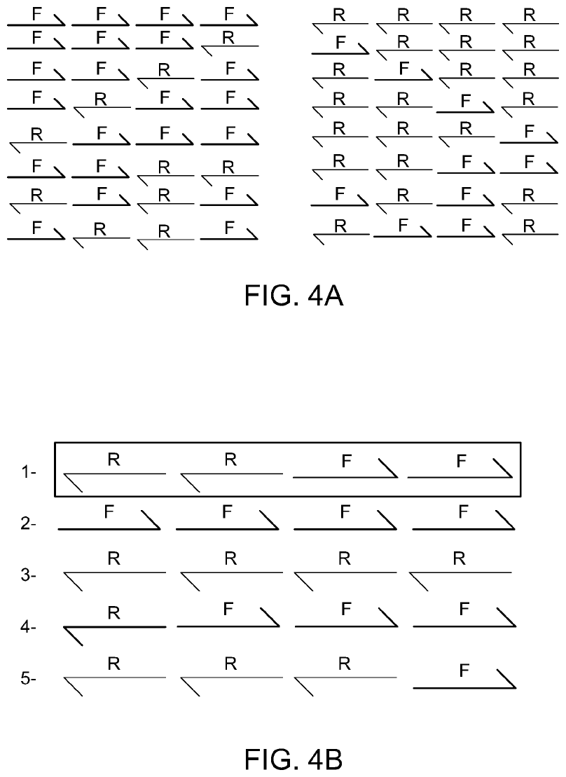

In some embodiments, the polynucleotide can include four expression cassettes with a first expression cassette, a second expression cassette, a third expression cassette, and a fourth expression cassette. FIG. 4A provides exemplary arrangements of four expression cassettes. In one embodiment, the first expression cassette has the read directionality of reverse, the second expression cassette has the read directionality of reverse, the third expression cassette has the read directionality of forward, and the fourth expression cassette has the read directionality of forward. In one embodiment, the first expression cassette has the read directionality of forward, the second expression cassette has the read directionality of forward, the third expression cassette has the read directionality of forward, and the fourth expression cassette has the read directionality of forward. In one embodiment, the first expression cassette has the read directionality of reverse, the second expression cassette has the read directionality of reverse, the third expression cassette has the read directionality of reverse, and the fourth expression cassette has the read directionality of reverse. In one embodiment, the first expression cassette has the read directionality of reverse, the second expression cassette has the read directionality of forward, the third expression cassette has the read directionality of forward, and the fourth expression cassette has the read directionality of forward. In one embodiment, the first expression cassette has the read directionality of reverse, the second expression cassette has the read directionality of reverse, the third expression cassette has the read directionality of reverse, and the fourth expression cassette has the read directionality of forward. In one embodiment, the first expression cassette has the read directionality of forward, the second expression cassette has the read directionality of forward, the third expression cassette has the read directionality of forward, and the fourth expression cassette has the read directionality of reverse. In one embodiment, the first expression cassette has the read directionality of forward, the second expression cassette has the read directionality of forward, the third expression cassette has the read directionality of reverse, and the fourth expression cassette has the read directionality of forward. In one embodiment, the first expression cassette has the read directionality of forward, the second expression cassette has the read directionality of forward, the third expression cassette has the read directionality of reverse, and the fourth expression cassette has the read directionality of reverse. In one embodiment, the first expression cassette has the read directionality of forward, the second expression cassette has the read directionality of reverse, the third expression cassette has the read directionality of forward, and the fourth expression cassette has the read directionality of forward. In one embodiment, the first expression cassette has the read directionality of forward, the second expression cassette has the read directionality of reverse, the third expression cassette has the read directionality of forward, and the fourth expression cassette has the read directionality of reverse. In one embodiment, the first expression cassette has the read directionality of forward, the second expression cassette has the read directionality of reverse, the third expression cassette has the read directionality of reverse, and the fourth expression cassette has the read directionality of forward. In one embodiment, the first expression cassette has the read directionality of forward, the second expression cassette has the read directionality of reverse, the third expression cassette has the read directionality of reverse, and the fourth expression cassette has the read directionality of reverse. In one embodiment, the first expression cassette has the read directionality of reverse, the second expression cassette has the read directionality of forward, the third expression cassette has the read directionality of forward, and the fourth expression cassette has the read directionality of reverse. In one embodiment, the first expression cassette has the read directionality of reverse, the second expression cassette has the read directionality of forward, the third expression cassette has the read directionality of reverse, and the fourth expression cassette has the read directionality of forward. In one embodiment, the first expression cassette has the read directionality of reverse, the second expression cassette has the read directionality of forward, the third expression cassette has the read directionality of reverse, and the fourth expression cassette has the read directionality of reverse. In one embodiment, the first expression cassette has the read directionality of reverse, the second expression cassette has the read directionality of reverse, the third expression cassette has the read directionality of forward, and the fourth expression cassette has the read directionality of reverse.

In some embodiments, the read directionality of each of the two or more expression cassettes is selected such that the expression cassette does not read into each other. For example, an expression cassette with a forward read directionality may not be placed 5′ of an expression cassette with a reverse read directionality within the polynucleotide. As another example, an expression cassette with a reverse read directionality may not be placed 3′ of an expression cassette with a forward read directionality within the polynucleotide. Non-limiting examples of configurations with four expression cassettes that do not read into each other are provided in FIG. 4B.

In some embodiments, each of the expression cassettes can have a different promoter sequence. In some embodiments, two or more of the expression cassettes can have the same promoter sequences. In some embodiments, the first expression cassette and the second expression cassette in a polynucleotide with two expression cassettes can have a different promoter sequence. In one embodiment, the first expression cassette and the second expression cassette in a polynucleotide with two expression cassettes can have the same promoter sequence. In some embodiments, the first expression cassette, the second expression cassette and the third expression cassette in a polynucleotide with three expression cassettes each comprise a different promoter sequence. In some embodiments, at least two of the three expression cassettes in a polynucleotide with three expression cassettes can have the same promoter sequence. In some embodiments, the first expression cassette, the second expression cassette, the third expression cassette and fourth expression cassette in a vector with four expression cassettes can each have a different promoter sequence. In some embodiments, at least two of the four expression cassettes in a vector with four expression cassettes can have the same promoter sequence.

### Promoter Sequences, Termination Sequences, and Hairpin Sequences

A vector comprising a plurality of expression cassettes of the present disclosure may include an expression cassette that has a promoter sequence, an RNA payload coding sequence, a termination sequence, and a hairpin sequence (e.g., a non-recruitment hairpin). The promoter may recruit transcription factors, polymerases (e.g., RNA polymerase II or RNA polymerase III), or other transcriptional machinery to promote transcription of the RNA payload. For example, the vector comprising a plurality of expression cassettes may promote transcription of a guide RNA for RNA editing, a guide RNA for DNA editing, a tracrRNA, an siRNA, an shRNA, or a miRNA, or an antisense oligonucleotide). In some embodiments, the promoter may be engineered for increased expression of the RNA payload under transcriptional control of the promoter. The termination sequence may enhance termination of transcription and promote transcriptional turnover, increasing transcription of the payload. In some embodiments, the termination sequence may be engineered for enhanced expression of the RNA payload. Sequence elements within the promoter or termination sequence (e.g., transcription factor binding sequences, transcription initiation sequences, termination sequences, or combinations thereof) may be engineered for enhanced payload expression. The sequence elements may be interchangeable with sequence elements from endogenous RNA promoters, such as U1, U6, or U7 promoters.

An expression cassette may be engineered from an endogenous sequence. For example, an expression cassette may be engineered from an endogenous U1, U2, U3, U4, U5, U6, or U7 sequence. The endogenous sequence may be from any organism, including human, mouse, or other mammals. In some embodiments, an expression cassette may comprise a promoter engineered from an endogenous promoter, such as an endogenous U1, U2, U3, U4, U5, U6, or U7 promoter. In some embodiments, an expression cassette may comprise a transcriptional termination sequence engineered from an endogenous transcriptional termination sequence, such as an endogenous U1, U2, U3, U4, U5, U6, or U7 transcriptional termination sequence. Examples of sequence elements that may be inserted or substituted into an expression cassette are provided in TABLE 1. In some embodiments, the present disclosure provides for a vector comprising a plurality of expression cassettes and where each expression cassette has a distinct promoter. Further, said plurality of expression cassettes can have distinct transcriptional terminators. For example, a vector provided herein may have two expression cassettes. The first expression cassette may have a first promoter and a first transcriptional terminator. The second expression cassette may have a second promoter and a second transcriptional terminator. The first promoter and the second promoter may be different. The first transcriptional terminator and second transcriptional terminator may be different. In some cases, it is advantageous for the promoter and terminator sequences to differ from expression cassette to expression cassette in vectors comprising a plurality of expression cassettes. This may be because having repetitive sequences within a single vector genome can lead to unwanted effects such as recombination, which can result in improper, diminished, or abolished expression of the therapeutic payload.

TABLE 1
Exemplary Sequence Elements

| | |
|---|---|
| SEQ ID NO | Sequence |

Promoter Sequences

| | |
|---|---|
| SEQ ID NO: 1 | TAAGGACCAGCTTCTTTGGGAGAGAACAGACGCAGGGGCGG |
| | GAGGGAAAAAGGGAGAGGCAGACGTCACTTCCTCTTGGCGA |
| | CTCTGGCAGCAGATTGGTCGGTTGAGTGGCAGAAAGGCAGAC |
| | GGGGACTGGGCAAGGCACTGTCGGTGACATCACGGACAGGG |
| | CGACTTCTATGTAGATGAGGCAGCGCAGAGGCTGCTGCTTCG |
| | CCACTTGCTGCTTCGCCACGAAGGGAGTTCCCGTGCCCTGGG |
| | AGCGGGTTCAGGACCGCTGATCGGAAGTGAGAATCCCAGCTG |
| | TGTGTCAGGGCTGGAAAGGGCTCGGGAGTGCGCGGGGCAAG |
| | TGACCGTGTGTGTAAAGAGTGAGGCGTATGAGGCTGTGTCGG |
| | GGCAGAGCCCGAAGATCTC |
| SEQ ID NO: 2 | TTAACAACAACGAAGGGGCTGTGACTGGCTGCTTTCTCAACC |
| | AATCAGCACCGAACTCATTTGCATGGGCTGAGAACAAATGTT |
| | CGCGAACTCTAGAAATGAATGACTTAAGTAAGTTCCTTAGAA |
| | TATTATTTTTCCTACTGAAAGTTACCACATGCGTCGTTGTTTA |
| | TACAGTAATAGGAACAAGAAAAAAGTCACCTAAGCTCACCCT |
| | CATCAATTGTGGAGTTCCTTTATATCCCATCTTCTCTCCAAAC |
| | ACATACGCA |
| SEQ ID NO: 3 | TAAGGACCAGCTTCTTTGGGAGAGAACAGACGCAGGGGCGG |
| | GAGGGAAAAAGGGAGAGGCAGACGTCACTTCCTCTTGGCGA |
| | CTCTGGCAGCAGATTGGTCGGTTGAGTGGCAGAAAGGCAGAC |
| | GGGGACTGGGCAAGGCACTGTCGGTGACATCACGGACAGGG |
| | CGACTTCTATGCAAATCAAGAGAAATGCAAATGAGGCAGCGC |
| | AGAGGCTGCTGCTTCGCCACTTGCTGCTTCGCCACGAAGGGA |
| | GTTCCCGTGCCCTGGGAGCGGGTTCAGGACCGCTGATCGGAA |
| | GTGAGAATCCCAGCTGTGTGTCAGGGCTGGAAAGGGCTCGGG |
| | AGTGCGCGGGGCAAAAGTCACCATGAGTGTAAAGGGTGAGG |
| | CGTATGAGGCTGTGTCGGGGCAGAGCCCGAAGATCTC |
| SEQ ID NO: 4 | TAACAACATAGGAGCTGTGATTGGCTGTTTTCAGCCAATCAG |
| | CACTGACTCATTTGCATAGCCTTTACAAGCGGTCACAAACTC |
| | AAGAAACGAGCGGTTTTAATAGTCTTTTAGAATATTGTTTATC |
| | GAACCGAATAAGGAACTGTGCTTTGTGATTCACATATCAGTG |
| | GAGGGGTGTGGAAATGGCACCTTGATCTCACCCTCATCGAAA |
| | GTGGAGTTGATGTCCTTCCCTGGCTCGCTACAGACGCACTTCC |
| | GC |
| SEQ ID NO: 5 | TAACAACATAGGAGCTGTGATTGGCTGTTTTCAGCCAATCAG |
| | CACTGACTCATGCAAATCAAGAGAAATGCAAATAGCCTTTAC |
| | AAGCGGTCACAAACTCAAGAAACGAGCGGTTTTAATAGTCTT |
| | TTAGAATATTGTTTATCGAACCGAATAAGGAACTGTGCTTTGT |
| | GATTCACATATCAGTGGAGGGGTGTGGAAATGGCACCTTGAT |
| | AAGTCACCATGAGTGTAAAGGGAGTTGATGTCCTTCCCTGGC |
| | TCGCTACAGACGCACTTCCGC |
| SEQ ID NO: 6 | GCCAGCCAGTCTTCGGCTTCGCCCCCTAACGGTGACATAAGG |
| | CACTCTGTGAAATGCTCTGTTCCGGAATCAAAAGATTGATCC |
| | GATTATTTGCATACCCATAATGCACTGCTCACAGTACAAATTT |
| | AAAAAGGCAAAATCAAACATTTTTATTCTAAGCATATTCTGT |
| | GAAAGTTAGACTTTTGTTTAAACAATACTCTTAAAATTTTTTT |
| | CTAGGTATAGAACCTTGGCATTCACTAGTCACCATCACTATAC |
| | TAGGAGTTTCTGTTACCCGAGAAACGAGTTATGAAATTAACA |
| | AGC |
| SEQ ID NO: 7 | TGGTGGCCTCAGGCGCAGCGCTAAAACGCATGAACCATTTAA |
| | GGTATTTCCTGAAACTGGAGCGTGATTGGTGAGACTTTATTTG |
| | CATACCCACAATGCATTGCGCACTAAATAATGTTCGTCTTTAA |
| | AATTATTTCCCCTTTTTCCTTCAACATATCTTTCTCGGAACCGA |
| | GATTGCTGTCCCAGAATTGTCTGAAGAAAAAGGCTGAAGTCA |
| | ATAGCTCTTTTGGGCCGAAGGAAAGTTACCATTACCCGTTTA |
| | GGAGTAGCCGTTACCTGAGAACTGTAGTGTCGACGACTGATG |
| | TT |
| SEQ ID NO: 8 | GTGGCCTCAGGCGCAGCGCTAAAACGCATGAACCATTTAAGG |
| | TATTTCCTGAAACTGGAGCGTGATTGGTGAGACTTTATGCAA |
| | ATCAAGAGAAATGCAAATACCCACAATGCATTGCGCACTAAA |
| | TAATGTTCGTCTTTAAAATTATTTCCCCTTTTTCCTTCAACATA |
| | TCTTTCTCGGAACCGAGATTGCTGTCCCAGAATTGTCTGAAGA |
| | AAAAGGCTGAAGTCAATAGCTCTTTTGGGCCGAAGGAAAGTC |
| | ACCATGAGTGTAAAGGGAGTAGCCGTTACCTGAGAACTGTAG |
| | TGTCGACGACTGATGTTAT |
| SEQ ID NO: 9 | GTCAAGTGCCTCTCTCCATTTACTGGTAAGAGAGAGAGGGTT |
| | TAGAGGAACTCTTGTTCCGGCGCTCAGCTCATGCAAATCAAG |
| | AGAAATGCAAATCCCAGAATGCATTGTAGATACGAGAATTAT |
| | TACCAGGGTTATCTGTTTGAATAATAATATTTAAACTTTTTTT |
| | CTTTGTCAGGAGATTTTACCCAGTGAGAACATGTTTAGGACA |
| | CTTTTCTACAGTGGAAGAAAAGCTTCTGTCTGCAGGTCCATTC |
| | TCGCCATCAGTTAAAAGTTACCAGTCAATAGCTGGGAAGCCA |
| | GGCAAAAGGCTAACAGGCAG |
| SEQ ID NO: 66 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCACTAGTCACCATCACTATACTAGGAGTTTCTGTTACCCG |
| | AGAAACGAGTTATGAAATTAACAAGC |
| SEQ ID NO: 67 | ATTTAATAGCAGTCTTTATTTAAAAGAAATCAAACTCAGACG |
| | TACAAATACACAAAACAGATAAAACCCGAGTCTCTGACCAGG |
| | AAAGCGTTATTTTCCAGCCAGCCAGTCTTCGGCTTCGCCCCCT |
| | AACGGTGACATAAGGCACTCTGTGAAATGCTCTGTTCCGGAA |
| | TCAAAAGATTGATCCGATTATTTGCATACCCATAATGCACTGC |
| | TCACAGTACAAATTTAAAAAGGCAAAATCAAACATTTTTATT |
| | CTAAGCATATTCTGTGAAAGTTAGACTTTTGTTTAAACAATAC |
| | TCTTAAAATTTTTTTCTAGGTATAGAACCTTGGCATTCACTAG |
| | TCACCATCACTATACTAGGAGTTTCTGTTACCCGAGAAACGA |
| | GTTATGAAATTAACAAGC |
| SEQ ID NO: 68 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCACAAGTCACCATGAGTGTAAAGGGAGTTTCTGTTACCC |
| | GAGAAACGAGTTATGAAATTAACAAGC |
| SEQ ID NO: 69 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCAGATCTCACCATGAGTGTAAAGGGAGTTTCTGTTACCC |
| | GAGAAACGAGTTATGAAATTAACAAGC |
| SEQ ID NO: 70 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCACAAATCACCATAAACGTGAAATGAGTTTCTGTTACCC |
| | GAGAAACGAGTTATGAAATTAACAAGC |
| SEQ ID NO: 71 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCAGATCTCACCATAAACGTGAAATGAGTTTCTGTTACCCG |
| | AGAAACGAGTTATGAAATTAACAAGC |
| SEQ ID NO: 72 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCAGATCTCACCATCACTATACTAGGAGTTTCTGTTACCCG |
| | AGAAACGAGTTATGAAATTAACAAGC |
| SEQ ID NO: 73 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCACAATTTGCCATGAGTATGTTGTGAGTTTCTGTTACCCG |
| | AGAAACGAGTTATGAAATTAACAAGC |
| SEQ ID NO: 74 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCAGATCTTGCCATGAGTATGTTGTGAGTTTCTGTTACCCG |
| | AGAAACGAGTTATGAAATTAACAAGC |
| SEQ ID NO: 75 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCACTAGACACCATCAGTGTACTAGGAGTTTCTGTTACCCG |
| | AGAAACGAGTTATGAAATTAACAAGC |
| SEQ ID NO: 76 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCAGATCACACCATCAGTGTACTAGGAGTTTCTGTTACCCG |
| | AGAAACGAGTTATGAAATTAACAAGC |
| SEQ ID NO: 90 | CAATTACTTTTGCACCGATCTAATAGCTCGCCCACTGTAAGTA |
| | AAGCAGGTAAAGTCAGCCTTTTTCTTCTGGGACCAGACTCTG |
| | CTCTGCCCCGCGGTGGTGGCCTCAGGCGCAGCGCTAAAACGC |
| | ATGAACCATTTAAGGTATTTCCTGAAACTGGAGCGTGATTGG |
| | TGAGACTTTATGCAAATCAAGAGAAATGCAAATACCCACAAT |
| | GCATTGCGCACTAAATAATGTTCGTCTTTAAAATTATTTCCCC |
| | TTTTTCCTTCAACATATCTTTCTCGGAACCGAGATTGCTGTCC |
| | CAGAATTGTCTGAAGAAAAAGGCTGAAGTCAATAGCTCTTTT |
| | GGGCCGAAGGAAAGTCACCATGAGTGTAAAGGGAGTAGCCG |
| | TTACCTGAGAACTGTAGTGTCGACGACTGATGTTAT |
| SEQ ID NO: 91 | GATTACAGGTGTGAGTCACTGTGCACCGGGCACTGCTACTCT |
| | GAACACATGTGGCTACATGGAAGACAGGACTTGGCCATGTGC |
| | TCTCCCCAGCTCAGTAGTCAAGTGCCTCTCTCCATTTACTGGT |
| | AAGAGAGAGAGGGTTTAGAGGAACTCTTGTTCCGGCGCTCAG |
| | CTCATGCAAATCAAGAGAAATGCAAATCCCAGAATGCATTGT |
| | AGATACGAGAATTATTACCAGGGTTATCTGTTTGAATAATAA |
| | TATTTAAACTTTTTTTCTTTGTCAGGAGATTTTACCCAGTGAG |
| | AACATGTTTAGGACACTTTTCTACAGTGGAAGAAAAGCTTCT |
| | GTCTGCAGGTCCATTCTCGCCATCAGTTAAAAGTTACCAGTCA |
| | ATAGCTGGGAAGCCAGGCAAAAGGCTAACAGGCAG |

Termination Sequences

| | |
|---|---|
| SEQ ID NO: 10 | CCCAATTTCACTGGTCTACAATGAAAGCAAAACAGTTCTCTTC |
| | CCCGCTCCCCGGTGTGTGAGAGGGGCTTTGATCCTTCTCTGGT |
| | TTCCTAGGAAACGCGTATGTG |
| SEQ ID NO: 11 | CCCAATTTCACTGGTTTCAAAAACAGAAAAACAGTTCTCTTCC |
| | CCGCTCCCCGGTGTGTGAGAGGGGCTTTGATCCTTCTCTGGTT |
| | TCCTAGGAAACGCGTATGTG |
| SEQ ID NO: 12 | CCCAATTTCACTGGTTTCAAAAACAGAAAAACAGTTCTCTTCC |
| | CCG |
| SEQ ID NO: 13 | ATATAAAGCTGTTAAAAAATCAGATTGACTTCATTTAGGGTG |
| | TTTCTTACAGATATCGTTTAAGTTTTCGGTTCTGCTTGTAAAC |
| | GCTTCAATCGCTCAT |
| SEQ ID NO: 14 | TATTTTAAGTGTTTTAAAAACAGATGCGATTCCGTTAAATCGC |
| | GTGTGGAGCTATGTAAAGTGTATTATAGAACAAATGCGAGTT |
| | ACGGTTTTTCAGCTT |
| SEQ ID NO: 15 | ATTTTAAGTGTTTCAAAAACAGATGCGATTCCGTTAAATCGC |
| | GTGTGGAGCTATGTAAAGTGTATTATAGAACAAATGCGAGTT |
| | ACGGTTTTTCAGCTTT |
| SEQ ID NO: 16 | GAATGTTCTGTTGCCAATGATAGACGTGTGGGTGGGGTGTTT |
| | CATGCTTTGGGAGGTTGGGGTAGCTCCACAAATGTCACCCAG |
| | GTTGTAGCGGAGTGGT |
| SEQ ID NO: 34 | AATTTTTGTAATGAAAAAATAGACGGCAAGGGTTATTCTTAA |
| | AACTGCAGTTTTGTAGCTTGGGTGGCATGTTAAGTGTTCTCCT |
| | TACAGTCGCAACGAT |
| SEQ ID NO: 35 | ATCATGTTTTATAAAAAAAGACTTAAAGAGGAAAACATTATG |
| | GTGCAACTTTAGGCTTAAGTGATTCATTGTCACTGTTTGTTTA |
| | AACATTGTGTAACAG |
| SEQ ID NO: 77 | TATAAAGCTGTTAAAAAATCAGATTGACTTCATTTAGGGTGTT |
| | TCTTACAGATATCGTTTAAGTTTTCGGTTCTGCTTGTAAACGC |
| | TTCAATCGC |
| SEQ ID NO: 78 | AATTTTTGTAATGAAAAAATAGACGGCAAGGGTTATTCTTAA |
| | AACTGCAGTTTTGTAGCTTGGGTGGCATGTTAAGTGTTCTCCT |
| | TACAGTCGCAACGATGGGAAACAGAAAGTAACGTGTTATCCT |
| | CTCCGCCGCCGTGAGCTCTTTTAACACTAGCTAAGTGGCCGC |
| | AGGGCTCTTCTCTTTCCTTTCCACTTGGGGC |
| SEQ ID NO: 79 | ATCATGTTTTATAAAAAAAGACTTAAAGAGGAAAACATTATG |
| | GTGCAACTTTAGGCTTAAGTGATTCATTGTCACTGTTTGTTTA |
| | AACATTGTGTAACAGAACTTGCAAAGACAGTTAACTCTTGTT |
| | TTCCATGTCAAAGGTCTGAATACTTGCATGATAAAAGTCTGT |
| | GTAACTTTCCCTGGTGACATCTGACTTGCTA |
| SEQ ID NO: 80 | CTTAGTAAGTTTAAAAACAGAAAAAAAACCGTGTTGCTACAG |
| | CTATAAACTTCAAACATGCAGTTTATAGCAGTGGGCAACACG |
| | TCTCATCTCAAAAATTCAGCACTCAAACATTACACAGGAAGA |
| | GGATGTAATTTTTTAAAATGAAAGCTCTGAAGTAATTTGAGT |
| | ATTCTCTGTCCTTTTTGTAAAAAAAATTACGA |
| SEQ ID NO: 81 | CCCAATTTCACTGGTTTCAAAAACAGAAAAACAGTTCTCTTCC |
| | CCGCTCCCCGGTGTGTGAGAGGGGCTTTGATCCTTCTCTGGTT |
| | TCCTAGGAAACGCGTATGTGCTAGAGCCACGCTCTGAGACTT |
| | CCGCCTCGTGCGGTCCCGCTTCCTTTCTGCCTCCTCTGGCCTG |
| | CATCCGTGGGGGAGGTGGCTGGCTGCAG |
| SEQ ID NO: 82 | CTTAGTAAGTTTAAAAACAGAAAAAAAACCGTGTTGCTACAG |
| | CTATAAACTTCAAACATGCAGTTTATAGCAGTGGGCAACACG |
| | TCTCATCTCAAAAATT |
| SEQ ID NO: 85 | TATAAAGCTGTTAAAAAATCAGATTGACTTCATTTAGGGTGTT |
| | TCTTACAGATATCGTTTAAGTTTTCGGTTCTGCTTGTAAACGC |
| | TTCAATCGCTCATGCGTTTAAACTTTGTGTAAGTGCAAGCGTT |
| | CTTTTTCTAGTGAAGCTTCTTCCAACAAGTTAGAAGTATTTAC |
| | TTCAGTGTCATTAATGTGAAAGATTCTT |
| SEQ ID NO: 87 | GTCACTCTTGTCCAATGAGAGATCATAACTTGAAGTCGGTGG |
| | TCTTTATTGTATAATTTATTTATTATAAAAATGCATACAACAT |
| | AAAAGCATCTTCAGCGTTTTGGAGTGTACACTGAGTGGTATT |
| | ACATACATTCACATTGTCGTGCACCCATCACCCCCATCCATCT |
| | CCAGAGCTCATTTCATCTTGCAGAACTGAA |
| SEQ ID NO: 89 | AATTTTTGTAATGAAAAAATAGACTCCCCTATAAGGGTTATTC |
| | TTAAAACTGCAGTTTTGTGGCTTGGGTGGCATGTTAAGTGTTC |
| | TCCTTACAGTCGCAATGATGGGAAACAGAAAGTAACGTGTTA |
| | TCCTCTCCGCCGCCGTGAGCTCTTTTAACACTAAGTGGCCGCA |
| | GGGCTCTTCTCTTCCCTTTCTACTTGGGG |

snRNA Hairpin Sequences

| | |
|---|---|
| SEQ ID NO: 83 | AACAGGGTTTTCTGACTTCGGTCGGAAAACCCCCT |
| SEQ ID NO: 84 | TAGTGGGGGACTGCGTTCGCGCTTTCCCCTG |

SmOPT and snRNA Hairpin Sequences

| | |
|---|---|
| SEQ ID NO: 92 | AATTTTTGGAACAGGGTTTTCTGCCTTCGGGCGGAAAACCCC |
| | CT |

In some embodiments, a vector comprising a plurality of expression cassettes can have a promoter (e.g., a DNA sequence encoding a promoter sequence) for enhanced expression of an RNA payload that may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or 100% sequence identity to any of SEQ ID NO: 1-SEQ ID NO: 9, SEQ ID NO: 66-SEQ ID NO: 76, SEQ ID NO: 90, or SEQ ID NO: 91. In some embodiments, a promoter sequence may enhance transcription of an RNA payload. The promoter sequence may be positioned upstream of the payload sequence. In some embodiments, the DNA sequence encoding a promoter sequence comprises at least 80%, at least 85%, at least 90%, at least 95%, or 100% sequence identity to SEQ ID NO: 5 or SEQ ID NO: 67. In some embodiments, at least one of the DNA sequences encoding a promoter sequence of the plurality of expression cassettes in a multi-expression cassette vector comprises at least 80% sequence identity to SEQ ID NO: 5.

In some embodiments, a vector comprising a plurality of expression cassettes can additionally have a transcriptional termination sequence (e.g., a DNA sequence encoding a transcriptional termination sequence), including an engineered termination sequence. The termination sequence may enhance expression of a payload (e.g., a small RNA payload) encoded by the expression cassette. In some embodiments, the termination sequence may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 10-SEQ ID NO: 16, SEQ ID NO: 34, SEQ ID NO: 35, SEQ ID NO: 77-SEQ ID NO: 82, or SEQ ID NO: 85-SEQ ID NO: 89. In some embodiments, a termination sequence, also referred to as a terminator, may enhance transcription of an RNA payload. The termination sequence may be positioned downstream of the payload sequence. In some embodiments, the DNA sequence encoding a transcription termination comprises at least 80%, at least 85%, at least 90%, at least 95%, or 100% sequence identity to SEQ ID NO: 78, SEQ ID NO: 79, or SEQ ID NO: 80.

The small nuclear RNA (snRNA) hairpin sequence (e.g., a non-recruitment hairpin sequence) may enhance expression of a payload (e.g., a small RNA payload) encoded by the expression cassette. In some embodiments, the snRNA hairpin sequence (e.g., a non-recruitment hairpin sequence) may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 83 or SEQ ID NO: 84. In some embodiments, a snRNA hairpin sequence (e.g., a non-recruitment hairpin sequence) may enhance transcription of an RNA payload. The snRNA hairpin sequence (e.g., a non-recruitment hairpin sequence) may be positioned downstream of the payload sequence. In some embodiments, a snRNA hairpin sequence is paired with an sm-binding sequence (e.g., an SmOPT sequence). In some embodiments, an SmOPT and snRNA hairpin sequence may be encoded by a DNA sequence comprising at least 80%, at least 85%, at least 90%, at least 95%, or 100% sequence identity to SEQ ID NO: 92.

### Multi-Expression Cassette Vector Architecture

In some embodiments, the present disclosure provides a vector comprising a plurality of expression cassettes. A vector comprising a plurality of expression cassettes may comprise a first pair of a promoter sequence and a terminator sequence (also referred to as a promoter-terminator pair) and a second promoter-terminator pair. The first promoter-terminator pair may be different than the second promoter-terminator pair. The first promoter-terminator pair and second promoter-terminator pair may be arranged in the same or different read orientations. The first promoter-terminator pair, the second promoter-terminator pair, or both may comprise a promoter sequence of SEQ ID NO: 5 paired with a terminator sequence of SEQ ID NO: 11; a promoter sequence of SEQ ID NO: 6 paired with a terminator sequence of SEQ ID NO: 13; a promoter sequence of SEQ ID NO: 8 paired with a terminator sequence of SEQ ID NO: 15; or a promoter sequence of SEQ ID NO: 6 paired with a terminator sequence of SEQ ID NO: 13. A vector comprising a plurality of expression cassettes may comprise a first pair of a promoter sequence and a terminator sequence, a second pair of a promoter sequence and a terminator sequence, and a third pair of a promoter sequence and a terminator sequence. The first promoter-terminator pair, the second promoter-terminator pair, and the third promoter-terminator pair may be different. The first promoter-terminator pair, second promoter-terminator pair, third promoter-terminator pair, or a combination thereof may be arranged in the same or different read orientations. The first promoter-terminator pair, second promoter-terminator pair, third promoter-terminator pair, or a combination thereof, may comprise a promoter sequence of SEQ ID NO: 5 paired with a terminator sequence of SEQ ID NO: 11; a promoter sequence of SEQ ID NO: 6 paired with a terminator sequence of SEQ ID NO: 13; a promoter sequence of SEQ ID NO: 8 paired with a terminator sequence of SEQ ID NO: 15; or a promoter sequence of SEQ ID NO: 6 paired with a terminator sequence of SEQ ID NO: 13. A vector comprising a plurality of expression cassettes may comprise a first promoter-terminator pair, a second promoter-terminator pair, a third promoter-terminator pair, and a fourth promoter-terminator pair. The first promoter-terminator pair, the second promoter-terminator pair, the third promoter-terminator pair, and the fourth promoter-terminator pair may be different. The first promoter-terminator pair, second promoter-terminator pair, third promoter-terminator pair, fourth promoter-terminator pair, or a combination thereof may be arranged in the same or different read orientations. The first promoter-terminator pair, second promoter-terminator pair, third promoter-terminator pair, fourth promoter-terminator pair, or a combination thereof may comprise a promoter sequence of SEQ ID NO: 5 paired with a terminator sequence of SEQ ID NO: 11; a promoter sequence of SEQ ID NO: 6 paired with a terminator sequence of SEQ ID NO: 13; a promoter sequence of SEQ ID NO: 8 paired with a terminator sequence of SEQ ID NO: 15; or a promoter sequence of SEQ ID NO: 6 paired with a terminator sequence of SEQ ID NO: 13.

In some embodiments, the present disclosure provides a vector comprising two expression cassettes. In such a vector, the first expression cassette has a first promoter selected from TABLE 1 and a first terminator selected from TABLE 1 and the second expression cassette has a second promoter selected from TABLE 1, which is different from the first promoter, and a second terminator selected from TABLE 1, which is different from the first terminator. For example, a vector comprising two expression cassettes may have a first expression cassette with a first promoter comprising SEQ ID NO: 5 and a first terminator comprising SEQ ID NO: 11 and a second expression cassette with a second promoter comprising SEQ ID NO: 6 and a second terminator comprising SEQ ID NO: 13. In another example, a vector comprising two expression cassettes may have a first expression cassette with a first promoter comprising SEQ ID NO: 5 and a first terminator comprising SEQ ID NO: 34 and a second expression cassette with a second promoter comprising SEQ ID NO: 6 and a second terminator comprising SEQ ID NO: 35.

In some embodiments, a multi-expression cassette vector may comprise a DNA sequence encoding a promoter sequence of the first expression cassette comprising at least 80%, at least 85%, at least 90%, at least 95%, or 100% sequence identity to SEQ ID NO: 5 and a DNA sequence encoding a transcription termination sequence of the first expression cassette comprising at least 80%, at least 85%, at least 90%, at least 95%, or 100% sequence identity to SEQ ID NO: 78 or SEQ ID NO: 79. In some embodiments, a multi-expression cassette vector may comprise a DNA sequence encoding a promoter sequence of the second expression cassette comprising at least 80%, at least 85%, at least 90%, at least 95%, or 100% sequence identity to SEQ ID NO: 67 and a DNA sequence encoding a transcription termination sequence of the second expression cassette comprising at least 80%, at least 85%, at least 90%, at least 95%, or 100% sequence identity to SEQ ID NO: 79 or SEQ ID NO: 80.

In some embodiments, a multi-expression cassette vector may comprise a DNA sequence encoding a promoter sequence of the first expression cassette comprising at least 80%, at least 85%, at least 90%, at least 95%, or 100% sequence identity to SEQ ID NO: 5 and a DNA sequence encoding a promoter sequence of the second expression cassette comprising at least 80%, at least 85%, at least 90%, at least 95%, or 100% sequence identity to SEQ ID NO: 67. In some embodiments, a multi-expression cassette vector may comprise a DNA sequence encoding a transcription termination sequence of the first expression cassette comprising at least 80%, at least 85%, at least 90%, at least 95%, or 100% sequence identity to SEQ ID NO: 78 or SEQ ID NO: 79 and a DNA sequence encoding a transcription termination sequence of the second expression cassette comprising at least 80%, at least 85%, at least 90%, at least 95%, or 100% sequence identity to SEQ ID NO: 79 or SEQ ID NO: 80.

In some embodiments, a multi-expression cassette vector may comprise an expression cassette that comprises a DNA sequence encoding a promoter sequence of the first expression cassette comprising at least 80%, at least 85%, at least 90%, at least 95%, or 100% sequence identity to SEQ ID NO: 5, a DNA sequence encoding a SmOPT and a small nuclear RNA (snRNA) processing hairpin sequence of the first expression cassette comprising at least 80%, at least 85%, at least 90%, at least 95%, or 100% sequence identity to SEQ ID NO: 92, and a DNA sequence encoding a transcription termination sequence of the first expression cassette comprising at least 80%, at least 85%, at least 90%, at least 95%, or 100% sequence identity to SEQ ID NO: 78 or SEQ ID NO: 79.

In some embodiments, a multi-expression cassette vector may comprise an expression cassette that comprises a DNA sequence encoding a promoter sequence of the second expression cassette comprising at least 80%, at least 85%, at least 90%, at least 95%, or 100% sequence identity to SEQ ID NO: 67, a DNA sequence encoding a SmOPT and a small nuclear RNA (snRNA) processing hairpin sequence of the second expression cassette comprising at least 80%, at least 85%, at least 90%, at least 95%, or 100% sequence identity to SEQ ID NO: 92, and DNA sequence encoding a transcription termination sequence of the second expression cassette comprising at least 80%, at least 85%, at least 90%, at least 95%, or 100% sequence identity to SEQ ID NO: 79 or SEQ ID NO: 80.

In some embodiments, a multi-expression cassette vector may comprise a first expression cassette that comprises: a DNA sequence encoding a promoter sequence of the first expression cassette comprising at least 80%, at least 85%, at least 90%, at least 95%, or 100% sequence identity to SEQ ID NO: 5, a DNA sequence encoding a SmOPT and a small nuclear RNA (snRNA) processing hairpin sequence of the first expression cassette comprising at least 80%, at least 85%, at least 90%, at least 95%, or 100% sequence identity to SEQ ID NO: 92, and a DNA sequence encoding a transcription termination sequence of the first expression cassette comprising at least 80%, at least 85%, at least 90%, at least 95%, or 100% sequence identity to SEQ ID NO: 78; and a second expression cassette comprising: DNA sequence encoding a promoter sequence of the second expression cassette comprising at least 80%, at least 85%, at least 90%, at least 95%, or 100% sequence identity to SEQ ID NO: 67, a DNA sequence encoding a SmOPT and a small nuclear RNA (snRNA) processing hairpin sequence of the second expression cassette comprising at least 80%, at least 85%, at least 90%, at least 95%, or 100% sequence identity to SEQ ID NO: 92, and a DNA sequence encoding a transcription termination sequence of the second expression cassette comprising at least 80%, at least 85%, at least 90%, at least 95%, or 100% sequence identity to SEQ ID NO: 79. In some embodiments, the first expression cassette and the second expression cassette are oriented in a tandem read orientation.

In some embodiments, a multi-expression cassette vector may comprise a first expression cassette that comprises: a DNA sequence encoding a promoter sequence of the first expression cassette comprising at least 80%, at least 85%, at least 90%, at least 95%, or 100% sequence identity to SEQ ID NO: 5, a DNA sequence encoding a SmOPT and a small nuclear RNA (snRNA) processing hairpin sequence of the first expression cassette comprising at least 80%, at least 85%, at least 90%, at least 95%, or 100% sequence identity to SEQ ID NO: 92, and a DNA sequence encoding a transcription termination sequence of the first expression cassette comprising at least 80%, at least 85%, at least 90%, at least 95%, or 100% sequence identity to SEQ ID NO: 79; and a second expression cassette comprising: a DNA sequence encoding a promoter sequence of the second expression cassette comprising at least 80%, at least 85%, at least 90%, at least 95%, or 100% sequence identity to SEQ ID NO: 67, a DNA sequence encoding a SmOPT and a small nuclear RNA (snRNA) processing hairpin sequence of the second expression cassette comprising at least 80%, at least 85%, at least 90%, at least 95%, or 100% sequence identity to SEQ ID NO: 92, and a DNA sequence encoding a transcription termination sequence of the second expression cassette comprising at least 80%, at least 85%, at least 90%, at least 95%, or 100% sequence identity to SEQ ID NO: 80. In some embodiments, the first expression cassette and the second expression cassette are oriented in a tandem read orientation.

TABLE 2
Particular Vector Constructs Comprising a First Expression
Cassette and a Second Expression Cassette

| | First Expression Cassette | | | Second Expression Cassette | | |
|---|---|---|---|---|---|---|
| | | SmOPT | | | SmOPT | |
| | | and | | | and | |
| | | snRNA | | | snRNA | |
| | | Processing | Transcription | | Processing | Transcription |
| | Promoter | Hairpin | Termination | Promoter | Hairpin | Termination |
| | Sequence | Sequence | Sequence | Sequence | Sequence | Sequence |
| 1 | SEQ ID NO: | SEQ ID NO: | SEQ ID NO: | SEQ ID NO: | SEQ ID NO: | SEQ ID NO: |
| | 5 | 92 | 78 | 67 | 92 | 79 |
| 2 | SEQ ID NO: | SEQ ID NO: | SEQ ID NO: | SEQ ID NO: | SEQ ID NO: | SEQ ID NO: |
| | 5 | 92 | 79 | 67 | 92 | 80 |
| 3 | SEQ ID NO: | SEQ ID NO: | SEQ ID NO: | SEQ ID NO: | SEQ ID NO: | SEQ ID NO: |
| | 5 | 92 | 78 | 5 | 92 | 78 |
| 4 | SEQ ID NO: | SEQ ID NO: | SEQ ID NO: | SEQ ID NO: | SEQ ID NO: | SEQ ID NO: |
| | 5 | 92 | 79 | 5 | 92 | 78 |
| 5 | SEQ ID NO: | SEQ ID NO: | SEQ ID NO: | SEQ ID NO: | SEQ ID NO: | SEQ ID NO: |
| | 5 | 92 | 80 | 5 | 92 | 78 |
| 6 | SEQ ID NO: | SEQ ID NO: | SEQ ID NO: | SEQ ID NO: | SEQ ID NO: | SEQ ID NO: |
| | 67 | 92 | 78 | 5 | 92 | 78 |
| 7 | SEQ ID NO: | SEQ ID NO: | SEQ ID NO: | SEQ ID NO: | SEQ ID NO: | SEQ ID NO: |
| | 67 | 92 | 79 | 5 | 92 | 78 |
| 8 | SEQ ID NO: | SEQ ID NO: | SEQ ID NO: | SEQ ID NO: | SEQ ID NO: | SEQ ID NO: |
| | 67 | 92 | 80 | 5 | 92 | 78 |
| 9 | SEQ ID NO: | SEQ ID NO: | SEQ ID NO: | SEQ ID NO: | SEQ ID NO: | SEQ ID NO: |
| | 5 | 92 | 78 | 5 | 92 | 79 |
| 10 | SEQ ID NO: | SEQ ID NO: | SEQ ID NO: | SEQ ID NO: | SEQ ID NO: | SEQ ID NO: |
| | 5 | 92 | 79 | 5 | 92 | 79 |
| 11 | SEQ ID NO: | SEQ ID NO: | SEQ ID NO: | SEQ ID NO: | SEQ ID NO: | SEQ ID NO: |
| | 5 | 92 | 80 | 5 | 92 | 79 |
| 12 | SEQ ID NO: | SEQ ID NO: | SEQ ID NO: | SEQ ID NO: | SEQ ID NO: | SEQ ID NO: |
| | 67 | 92 | 78 | 5 | 92 | 79 |
| 13 | SEQ ID NO: | SEQ ID NO: | SEQ ID NO: | SEQ ID NO: | SEQ ID NO: | SEQ ID NO: |
| | 67 | 92 | 79 | 5 | 92 | 79 |
| 14 | SEQ ID NO: | SEQ ID NO: | SEQ ID NO: | SEQ ID NO: | SEQ ID NO: | SEQ ID NO: |
| | 67 | 92 | 80 | 5 | 92 | 79 |
| 15 | SEQ ID NO: | SEQ ID NO: | SEQ ID NO: | SEQ ID NO: | SEQ ID NO: | SEQ ID NO: |
| | 5 | 92 | 78 | 5 | 92 | 80 |
| 16 | SEQ ID NO: | SEQ ID NO: | SEQ ID NO: | SEQ ID NO: | SEQ ID NO: | SEQ ID NO: |
| | 5 | 92 | 79 | 5 | 92 | 80 |
| 17 | SEQ ID NO: | SEQ ID NO: | SEQ ID NO: | SEQ ID NO: | SEQ ID NO: | SEQ ID NO: |
| | 5 | 92 | 80 | 5 | 92 | 80 |
| 18 | SEQ ID NO: | SEQ ID NO: | SEQ ID NO: | SEQ ID NO: | SEQ ID NO: | SEQ ID NO: |
| | 67 | 92 | 78 | 5 | 92 | 80 |
| 19 | SEQ ID NO: | SEQ ID NO: | SEQ ID NO: | SEQ ID NO: | SEQ ID NO: | SEQ ID NO: |
| | 67 | 92 | 79 | 5 | 92 | 80 |
| 20 | SEQ ID NO: | SEQ ID NO: | SEQ ID NO: | SEQ ID NO: | SEQ ID NO: | SEQ ID NO: |
| | 67 | 92 | 80 | 5 | 92 | 80 |
| 21 | SEQ ID NO: | SEQ ID NO: | SEQ ID NO: | SEQ ID NO: | SEQ ID NO: | SEQ ID NO: |
| | 5 | 92 | 78 | 67 | 92 | 78 |
| 22 | SEQ ID NO: | SEQ ID NO: | SEQ ID NO: | SEQ ID NO: | SEQ ID NO: | SEQ ID NO: |
| | 5 | 92 | 79 | 67 | 92 | 78 |
| 23 | SEQ ID NO: | SEQ ID NO: | SEQ ID NO: | SEQ ID NO: | SEQ ID NO: | SEQ ID NO: |
| | 5 | 92 | 80 | 67 | 92 | 78 |
| 24 | SEQ ID NO: | SEQ ID NO: | SEQ ID NO: | SEQ ID NO: | SEQ ID NO: | SEQ ID NO: |
| | 67 | 92 | 78 | 67 | 92 | 78 |
| 25 | SEQ ID NO: | SEQ ID NO: | SEQ ID NO: | SEQ ID NO: | SEQ ID NO: | SEQ ID NO: |
| | 67 | 92 | 79 | 67 | 92 | 78 |
| 26 | SEQ ID NO: | SEQ ID NO: | SEQ ID NO: | SEQ ID NO: | SEQ ID NO: | SEQ ID NO: |
| | 67 | 92 | 80 | 67 | 92 | 78 |
| 27 | SEQ ID NO: | SEQ ID NO: | SEQ ID NO: | SEQ ID NO: | SEQ ID NO: | SEQ ID NO: |
| | 5 | 92 | 79 | 67 | 92 | 79 |
| 28 | SEQ ID NO: | SEQ ID NO: | SEQ ID NO: | SEQ ID NO: | SEQ ID NO: | SEQ ID NO: |
| | 5 | 92 | 80 | 67 | 92 | 79 |
| 29 | SEQ ID NO: | SEQ ID NO: | SEQ ID NO: | SEQ ID NO: | SEQ ID NO: | SEQ ID NO: |
| | 67 | 92 | 78 | 67 | 92 | 79 |
| 30 | SEQ ID NO: | SEQ ID NO: | SEQ ID NO: | SEQ ID NO: | SEQ ID NO: | SEQ ID NO: |
| | 67 | 92 | 79 | 67 | 92 | 79 |
| 31 | SEQ ID NO: | SEQ ID NO: | SEQ ID NO: | SEQ ID NO: | SEQ ID NO: | SEQ ID NO: |
| | 67 | 92 | 80 | 67 | 92 | 79 |
| 32 | SEQ ID NO: | SEQ ID NO: | SEQ ID NO: | SEQ ID NO: | SEQ ID NO: | SEQ ID NO: |
| | 5 | 92 | 78 | 67 | 92 | 80 |
| 33 | SEQ ID NO: | SEQ ID NO: | SEQ ID NO: | SEQ ID NO: | SEQ ID NO: | SEQ ID NO: |
| | 5 | 92 | 80 | 67 | 92 | 80 |
| 34 | SEQ ID NO: | SEQ ID NO: | SEQ ID NO: | SEQ ID NO: | SEQ ID NO: | SEQ ID NO: |
| | 67 | 92 | 78 | 67 | 92 | 80 |
| 35 | SEQ ID NO: | SEQ ID NO: | SEQ ID NO: | SEQ ID NO: | SEQ ID NO: | SEQ ID NO: |
| | 67 | 92 | 79 | 67 | 92 | 80 |
| 36 | SEQ ID NO: | SEQ ID NO: | SEQ ID NO: | SEQ ID NO: | SEQ ID NO: | SEQ ID NO: |
| | 67 | 92 | 80 | 67 | 92 | 80 |

In some embodiments, the first expression cassette and the second expression cassette as set out in TABLE 2 each independently comprise a DNA sequence encoding an engineered guide RNA (gRNA) sequence under transcriptional control of the promoter sequence. In some embodiments, the first expression cassette and the second expression cassette as set out in TABLE 2 are orientated in a tandem read orientation. In some embodiments, the DNA sequence encoding the promoter sequence, comprises at least 80%, at least 85%, at least 90%, at least 95%, or 100% sequence identity to the sequences identified in TABLE 2. In some embodiments, the DNA sequence encoding the SmOPT and a small nuclear RNA (snRNA) processing hairpin sequence comprises at least 80%, at least 85%, at least 90%, at least 95%, or 100% sequence identity to the sequences identified in TABLE 2. In some embodiments, the transcription termination sequence comprises at least 80%, at least 85%, at least 90%, at least 95%, or 100% sequence identity to the sequences identified in TABLE 2.

In some embodiments, the present disclosure provides a vector comprising three expression cassettes. In such a vector, the first expression cassette has a first promoter selected from TABLE 1 and a first terminator selected from TABLE 1, the second expression cassette has a second promoter selected from TABLE 1, which is different from the first promoter, and a second terminator selected from TABLE 1, which is different from the first terminator, and the third expression cassette has a third promoter selected from TABLE 1, which is different from the first promoter and the second promoter, and a third terminator selected from TABLE 1, which is different from the first terminator and the second terminator. For example, a vector comprising four expression cassettes may have a first expression cassette with a first promoter comprising SEQ ID NO: 5 and a first terminator comprising SEQ ID NO: 11; a second expression cassette with a second promoter comprising SEQ ID NO: 6 and a second terminator comprising SEQ ID NO: 13; a third expression cassette with a third promoter comprising SEQ ID NO: 8 and a third terminator comprising SEQ ID NO: 15. In another example, a vector comprising four expression cassettes may have a first expression cassette with a first promoter comprising SEQ ID NO: 5 and a first terminator comprising SEQ ID NO: 11; a second expression cassette with a second promoter comprising SEQ ID NO: 6 and a second terminator comprising SEQ ID NO: 13; a third expression cassette with a third promoter comprising SEQ ID NO: 9 and a third terminator comprising SEQ ID NO: 16.

In some embodiments, the present disclosure provides a vector comprising four expression cassettes. In such a vector, the first expression cassette has a first promoter selected from TABLE 1 and a first terminator selected from TABLE 1, the second expression cassette has a second promoter selected from TABLE 1, which is different from the first promoter, and a second terminator selected from TABLE 1, which is different from the first terminator, the third expression cassette has a third promoter, which is different from the first promoter and the second promoter, and a third terminator selected from TABLE 1, which is different from the first terminator and the second terminator, and the fourth expression cassette has a fourth promoter selected from TABLE 1, which is different from the first promoter, the second promoter, and the third promoter, and a fourth terminator selected from TABLE 1, which is different from the first terminator, the second terminator, and the third terminator. For example, a vector comprising four expression cassettes may have a first expression cassette with a first promoter comprising SEQ ID NO: 5 and a first terminator comprising SEQ ID NO: 11; a second expression cassette with a second promoter comprising SEQ ID NO: 6 and a second terminator comprising SEQ ID NO: 13; a third expression cassette with a third promoter comprising SEQ ID NO: 8 and a third terminator comprising SEQ ID NO: 15; and a fourth expression cassette with a fourth promoter comprising SEQ ID NO: 9 and a fourth terminator comprising SEQ ID NO: 16.

In some embodiments, the present disclosure provides extended sequence elements (e.g., extended promoter sequences, extended termination sequences, or extended hairpin sequences) for engineering of ideal expression cassette size, vector genome size, or both. A sequence element (e.g., a promoter sequence, a termination sequence, or a hairpin sequence) may be extended by the addition of nucleotides on the 5′ end of the sequence element, the 3′ end of the sequence element, or a combination thereof. In some embodiments, a sequence element (e.g., a promoter sequence, a termination sequence, or a hairpin sequence) may be extended by the addition of nucleotides on the 5′ end of the sequence element. In some embodiments, a sequence element (e.g., a promoter sequence, a termination sequence, or a hairpin sequence) may be extended by the addition of nucleotides on the 3′ end of the sequence element. In some embodiments, a sequence element (e.g., a promoter sequence, a termination sequence, or a hairpin sequence) may be extended by the addition of nucleotides on the 5′ end and 3′ end of the sequence element.

A sequence element (e.g., a promoter sequence, a termination sequence, or a hairpin sequence) may be extended for engineering of ideal vector size (e.g., an AAV genome size such as a scAAV genome size) of the present disclosure. In some embodiments, a sequence element (e.g., a promoter sequence, a termination sequence, or a hairpin sequence) may be extended to achieve a final vector genome size of at least 1.2 kb. In some embodiments, a sequence element (e.g., a promoter sequence, a termination sequence, or a hairpin sequence) may be extended to achieve a final vector genome size of at least 1.0 kb and no greater than 1.4 kb, at least 1.1 kb and no greater than 1.4 kb, at least 1.2 kb and no greater than 1.4 kb, or at least 1.2 kb and no greater than 1.3 kb.

In some embodiments a promoter sequence (e.g., any one of SEQ ID NO: 1-SEQ ID NO: 9, SEQ ID NO: 66-SEQ ID NO: 76, SEQ ID NO: 90, or SEQ ID NO: 91) may be extended for engineering of ideal expression cassette size, vector genome size, or both. In some embodiments, a promoter sequence (e.g., any one of SEQ ID NO: 1-SEQ ID NO: 9, SEQ ID NO: 66-SEQ ID NO: 76, SEQ ID NO: 90, or SEQ ID NO: 91) may be extended to a total sequence length of 250 nucleotides, 300 nucleotides, 350 nucleotides, 400 nucleotides, or 450 nucleotides. In some embodiments, a promoter sequence (e.g., any one of SEQ ID NO: 1-SEQ ID NO: 9, SEQ ID NO: 66-SEQ ID NO: 76, SEQ ID NO: 90, or SEQ ID NO: 91) may be extended to a total sequence length of 300 nucleotides. In some embodiments, a promoter sequence (e.g., any one of SEQ ID NO: 1-SEQ ID NO: 9, SEQ ID NO: 66-SEQ ID NO: 76, SEQ ID NO: 90, or SEQ ID NO: 91) may be extended to a total sequence length of 350 nucleotides. In some embodiments, a promoter sequence (e.g., any one of SEQ ID NO: 1-SEQ ID NO: 9, SEQ ID NO: 66-SEQ ID NO: 76, SEQ ID NO: 90, or SEQ ID NO: 91) may be extended to a total sequence length of 400 nucleotides. For example, a promoter sequence of SEQ ID NO: 6 or SEQ ID NO: 66 may be extended to a total sequence length of 400 nucleotides (e.g., a promoter sequence of SEQ ID NO: 67).

In some embodiments a termination sequence (e.g., any one of SEQ ID NO: 10-SEQ ID NO: 16, SEQ ID NO: 34, SEQ ID NO: 35, SEQ ID NO: 77-SEQ ID NO: 82, or SEQ ID NO: 85-SEQ ID NO: 89) may be extended for engineering of ideal expression cassette size, vector genome size, or both. In some embodiments, a termination sequence (e.g., any one of SEQ ID NO: 10-SEQ ID NO: 16, SEQ ID NO: 34, SEQ ID NO: 35, SEQ ID NO: 77-SEQ ID NO: 82, or SEQ ID NO: 85-SEQ ID NO: 89) may be extended to a total sequence length of 50 nucleotides, 100 nucleotides, 150 nucleotides, 200 nucleotides, 250 nucleotides, or 300 nucleotides. In some embodiments, a termination sequence (e.g., any one of SEQ ID NO: 10-SEQ ID NO: 16, SEQ ID NO: 34, SEQ ID NO: 35, SEQ ID NO: 77-SEQ ID NO: 82, or SEQ ID NO: 85-SEQ ID NO: 89) may be extended to a total sequence length of 250 nucleotides. In some embodiments, a termination sequence (e.g., any one of SEQ ID NO: 10-SEQ ID NO: 16, SEQ ID NO: 34, SEQ ID NO: 35, SEQ ID NO: 77-SEQ ID NO: 82, or SEQ ID NO: 85-SEQ ID NO: 89) may be extended to a total sequence length of 200 nucleotides. For example, a terminator sequence of SEQ ID NO: 34 may be extended to a total sequence length of 200 nucleotides (e.g., a terminator sequence of SEQ ID NO: 78). For example, a terminator sequence of SEQ ID NO: 35 may be extended to a total sequence length of 200 nucleotides (e.g., a terminator sequence of SEQ ID NO: 79). For example, a terminator sequence of SEQ ID NO: 82 may be extended to a total sequence length of 200 nucleotides (e.g., a terminator sequence of SEQ ID NO: 80). For example, a terminator sequence of SEQ ID NO: 11 or SEQ ID NO: 12 may be extended to a total sequence length of 199 nucleotides (e.g., a terminator sequence of SEQ ID NO: 81).

A sequence element (e.g., a promoter sequence, a termination sequence, or a hairpin sequence) may be extended for engineering of ideal size of an expression cassette of the present disclosure. In some embodiments, a sequence element (e.g., a promoter sequence, a termination sequence, or a hairpin sequence) may be extended to achieve a final expression cassette length of at least 600 nucleotides. In some embodiments, a sequence element (e.g., a promoter sequence, a termination sequence, or a hairpin sequence) may be extended to achieve a final expression cassette length of at least 500 and no greater than 700 nucleotides, at least 550 and no greater than 750 nucleotides, at least 550 and no greater than 650 nucleotides, or at least 575 and no greater than 625 nucleotides. For example, an expression cassette of the current disclosure may comprise an extended promoter sequence with a length of 300 nucleotides, a guide RNA sequence with a length of 100 nucleotides, and an extended termination sequence with a length of 250 nucleotides resulting in a total expression cassette length of 650 nucleotides.

A vector comprising a plurality of expression cassettes may comprise one or more extended sequence elements (e.g., extended promoter sequences, extended termination sequences, or extended hairpin sequences) for engineering of ideal expression cassette size, vector genome size, or both. In some embodiments, a vector may comprise one or more extended promoter sequences and one or more extended terminator sequences. In some embodiments, a vector may comprise two extended promoter sequences and two extended termination sequences. For example, a vector comprising two expression cassettes may comprise two extended promoter sequences each with a sequence length of 300 nucleotides, two guide RNA sequences each with a sequence length of 100 nucleotides, and two extended termination sequences each with a sequence length of 250 nucleotides resulting in a total vector genome size of greater than 1.2 kb.

### Payloads

The vectors comprising a plurality of expression cassettes of the present disclosure may encode an RNA payload under transcriptional control of a promoter (e.g., an engineered promoter). In some embodiments, the RNA payload may encode a small RNA payload such as a guide sequence (e.g., for RNA or DNA editing), a tracrRNA, an siRNA, an shRNA, a miRNA, an antisense oligonucleotide (e.g., for expression knockdown), a structural element (e.g., an RNA hairpin), or combinations thereof. Provided herein are engineered RNA payloads and polynucleotides encoding the same; as well as compositions comprising said engineered RNA payloads or said polynucleotides. As used herein, the term “engineered” in reference to an RNA payload or polynucleotide encoding the same refers to a non-naturally occurring RNA or polynucleotide encoding the same. For example, the present disclosure provides for engineered polynucleotides encoding engineered guide RNAs. In some embodiments, the engineered guide comprises RNA. In some embodiments, the engineered guide comprises DNA. In some examples, the engineered guide comprises modified RNA bases or unmodified RNA bases. In some embodiments, the engineered guide comprises modified DNA bases or unmodified DNA bases. In some examples, the engineered guide comprises both DNA and RNA bases.

### Guide RNA Payloads for RNA Editing

The vectors comprising a plurality of expression cassettes described herein may be used to enhance expression of engineered guide RNAs and engineered polynucleotides encoding the same for site-specific, selective editing of a target RNA via an RNA editing entity or a biologically active fragment thereof. An engineered guide RNA of the present disclosure can comprise latent structures, such that when the engineered guide RNA is hybridized to the target RNA to form a guide-target RNA scaffold, at least a portion of the latent structure manifests as at least a portion of a structural feature as described herein.

An engineered guide RNA, as described herein, may comprise a targeting domain with complementarity to a target RNA described herein. As such, a guide RNA can be engineered to site-specifically/selectively target and hybridize to a particular target RNA, thus facilitating editing of specific nucleotide in the target RNA via an RNA editing entity or a biologically active fragment thereof. The targeting domain can include a nucleotide that is positioned such that, when the guide RNA is hybridized to the target RNA, the nucleotide opposes a base to be edited by the RNA editing entity or biologically active fragment thereof and does not base pair, or does not fully base pair, with the base to be edited. This mismatch can help to localize editing of the RNA editing entity to the desired base of the target RNA. However, in some instances there can be some, and in some cases significant, off target editing in addition to the desired edit.

Hybridization of the target RNA and the targeting domain of the guide RNA may produce specific secondary structures in the guide-target RNA scaffold that manifest upon hybridization, which are referred to herein as “latent structures.” Latent structures, when manifested, may become structural features described herein, including mismatches, bulges, internal loops, and hairpins. Without wishing to be bound by theory, the presence of structural features described herein that are produced upon hybridization of the guide RNA with the target RNA configure the guide RNA to facilitate a specific, or selective, targeted edit of the target RNA via the RNA editing entity or biologically active fragment thereof. Further, the structural features in combination with the mismatch described above generally facilitate an increased amount of editing of a target residue (e.g., an adenosine residue), fewer off target edits, or both, as compared to a construct comprising the mismatch alone or a construct having perfect complementarity to a target RNA. Accordingly, rational design of latent structures in engineered guide RNAs of the present disclosure to produce specific structural features in a guide-target RNA scaffold can be a powerful tool to promote editing of the target RNA with high specificity, selectivity, and robust activity.

In some examples, the engineered guides provided herein comprise an engineered guide that can be configured, upon hybridization to a target RNA molecule, to form, at least in part, a guide-target RNA scaffold with at least a portion of the target RNA molecule, wherein the guide-target RNA scaffold comprises at least one structural feature, and wherein the guide-target RNA scaffold recruits an RNA editing entity and facilitates a chemical modification of a base of a nucleotide in the target RNA molecule by the RNA editing entity.

In some examples, a target RNA of an engineered guide RNA of the present disclosure can be a pre-mRNA or mRNA. In some embodiments, the engineered guide RNA of the present disclosure hybridizes to a sequence of the target RNA. In some embodiments, part of the engineered guide RNA (e.g., a targeting domain) hybridizes to the sequence of the target RNA. The part of the engineered guide RNA that hybridizes to the target RNA is of sufficient complementary to the sequence of the target RNA for hybridization to occur.

### Targeting Domain

Engineered guide RNAs disclosed herein can be engineered in any way suitable for RNA editing. In some examples, an engineered guide RNA generally comprises at least a targeting sequence that allows it to hybridize to a region of a target RNA molecule. A targeting sequence can also be referred to as a “targeting domain” or a “targeting region.”

In some cases, a targeting domain of an engineered guide allows the engineered guide to target an RNA sequence through base pairing, such as Watson Crick base pairing. In some examples, the targeting sequence can be located at either the N-terminus or C-terminus of the engineered guide. In some cases, the targeting sequence can be located at both termini. The targeting sequence can be of any length. In some cases, the targeting sequence can be at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In some cases, the targeting sequence can be no greater than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or 200 nucleotides in length. In some examples, an engineered guide comprises a targeting sequence that can be from about 60 to about 500, from about 60 to about 200, from about 75 to about 100, from about 80 to about 200, from about 90 to about 120, or from about 95 to about 115 nucleotides in length. In some examples, an engineered guide RNA comprises a targeting sequence that can be about 100 nucleotides in length.

In some cases, a targeting domain comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence can have a single base mismatch.

The targeting sequence can have sufficient complementarity to a target RNA to allow for hybridization of the targeting sequence to the target RNA. In some embodiments, the targeting sequence has a minimum antisense complementarity of about 50 nucleotides or more to the target RNA. In some embodiments, the targeting sequence has a minimum antisense complementarity of about 60 nucleotides or more to the target RNA. In some embodiments, the targeting sequence has a minimum antisense complementarity of about 70 nucleotides or more to the target RNA. In some embodiments, the targeting sequence has a minimum antisense complementarity of about 80 nucleotides or more to the target RNA. In some embodiments, the targeting sequence has a minimum antisense complementarity of about 90 nucleotides or more to the target RNA. In some embodiments, the targeting sequence has a minimum antisense complementarity of about 100 nucleotides or more to the target RNA. In some embodiments, antisense complementarity refers to non-contiguous stretches of sequence. In some embodiments, antisense complementarity refers to contiguous stretches of sequence.

In some embodiments, hybridization of the targeting sequence to the target RNA to form a guide-target RNA scaffold may manifest a latent structural feature. For example, a latent structural feature may comprise a symmetric bulge, an asymmetric bulge, a symmetric internal loop, an asymmetric internal loop, or combinations thereof. In some embodiments, the latent structural feature may comprise 1, 2, 3, 4, 5, 6, 7, 8, 9, 10, 11, 12, 13, 14, 15, 16, 17, 18, 19, or 20 unpaired nucleotides on the target RNA side. In some embodiments, the latent structural feature may comprise 1, 2, 3, 4, 5, 6, 7, 8, 9, 10, 11, 12, 13, 14, 15, 16, 17, 18, 19, or 20 unpaired nucleotides on the guide RNA side.

In some embodiments an engineered guide RNA for RNA editing may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 24 or SEQ ID NO: 25. For example, an engineered guide RNA of SEQ ID NO: 24 may be used to target PMP22. In another example, an engineered guide RNA of SEQ ID NO: 25 may be used to target SNCA.

### Engineered Guide RNAs Having a Recruitment Domain

In some examples, a subject engineered guide RNA comprises a recruiting domain that recruits an RNA editing entity (e.g., ADAR), where in some instances, the recruiting domain is formed and present in the absence of binding to the target RNA. A “recruiting domain” can be referred to herein as a “recruiting sequence” or a “recruiting region”. In some examples, a subject engineered guide can facilitate editing of a base of a nucleotide of in a target sequence of a target RNA that results in modulating the expression of a polypeptide encoded by the target RNA. In some instances, modulation can be increased or decrease expression of the polypeptide. In some cases, an engineered guide can be configured to facilitate an editing of a base of a nucleotide or polynucleotide of a region of an RNA by an RNA editing entity (e.g., ADAR or APOBEC). In order to facilitate editing, an engineered polynucleotide of the disclosure can recruit an RNA editing entity (e.g., ADAR or APOBEC). Various RNA editing entity recruiting domains can be utilized. In some examples, a recruiting domain comprises: Glutamate ionotropic receptor AMPA type subunit 2 (GluR2), an Alu sequence, or, in the case of recruiting APOBEC, an APOBEC recruiting domain.

In some examples, more than one recruiting domain can be included in an engineered guide of the disclosure. In examples where a recruiting domain can be present, the recruiting domain can be utilized to position the RNA editing entity to effectively react with a subject target RNA after the targeting sequence hybridizes to a target sequence of a target RNA. In some cases, a recruiting domain can allow for transient binding of the RNA editing entity to the engineered guide. In some examples, the recruiting domain allows for permanent binding of the RNA editing entity to the engineered guide. A recruiting domain can be of any length. In some cases, a recruiting domain can be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting domain can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting domain can be about 45 nucleotides in length. In some cases, at least a portion of a recruiting domain comprises at least 1 to about 75 nucleotides. In some cases, at least a portion of a recruiting domain comprises about 45 nucleotides to about 60 nucleotides.

In some embodiments, a recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some examples, a recruiting domain comprises a GluR2 sequence, or a sequence having at least about 70%, 80%, 85%, 90%, 95%, 98%, 99%, or 100% identity and/or length to: GUGGAAUAGUAUAACAAUAUGCUAAAUGUUGUUAUAGUAUCCCAC (SEQ ID NO: 93). In some cases, a recruiting domain can comprise at least about 80% sequence homology to at least about 10, 15, 20, 25, or 30 nucleotides of SEQ ID NO: 51. In some examples, a recruiting domain can comprise at least about 90%, 95%, 96%, 97%, 98%, or 99% sequence homology and/or length to SEQ ID NO: 51.

Additional, RNA editing entity recruiting domains are also contemplated. In an embodiment, a recruiting domain comprises an apolipoprotein B mRNA editing enzyme, catalytic polypeptide-like (APOBEC) domain. In some cases, an APOBEC domain can comprise a non-naturally occurring sequence or naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence. In another embodiment, a recruiting domain can be from an Alu domain.

Any number of recruiting domains can be found in an engineered guide of the present disclosure. In some examples, at least about 1, 2, 3, 4, 5, 6, 7, 8, 9, or up to about 10 recruiting domains can be included in an engineered guide. Recruiting domains can be located at any position of engineered guide RNAs. In some cases, a recruiting domain can be on an N-terminus, middle, or C-terminus of an engineered guide RNA. A recruiting domain can be upstream or downstream of a targeting sequence. In some cases, a recruiting domain flanks a targeting sequence of a subject guide. A recruiting sequence can comprise all ribonucleotides or deoxyribonucleotides, although a recruiting domain comprising both ribo- and deoxyribonucleotides can in some cases not be excluded.

Engineered Guide RNAs with Latent Structure

In some examples, an engineered guide disclosed herein useful for facilitating editing of a target RNA by an RNA editing entity can be an engineered latent guide RNA. An “engineered latent guide RNA” refers to an engineered guide RNA that comprises latent structure. “Latent structure” refers to a structural feature that substantially forms upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed, and the latent structure provided in the guide RNA is, thus, unmasked.

A double stranded RNA (dsRNA) substrate may be formed upon hybridization of an engineered guide RNA of the present disclosure to a target RNA. The resulting dsRNA substrate is also referred to herein as a “guide-target RNA scaffold.”

Unless otherwise noted, the number of participating nucleotides in a given structural feature is indicated as the nucleotides on the target RNA side over nucleotides on the guide RNA side. Also shown in this legend is a key to the positional annotation of each figure. For example, the target nucleotide to be edited is designated as the 0 position. Downstream (3′) of the target nucleotide to be edited, each nucleotide is counted in increments of +1. Upstream (5′) of the target nucleotide to be edited, each nucleotide is counted in increments of −1. Thus, the example 2/2 symmetric bulge in this legend is at the +12 to +13 position in the guide-target RNA scaffold. Similarly, the 2/3 asymmetric bulge in this legend is at the −36 to −37 position in the guide-target RNA scaffold. As used herein, positional annotation is provided with respect to the target nucleotide to be edited and on the target RNA side of the guide-target RNA scaffold. As used herein, if a single position is annotated, the structural feature extends from that position away from position 0 (target nucleotide to be edited). For example, if a latent guide RNA is annotated herein as forming a 2/3 asymmetric bulge at position-36, then the 2/3 asymmetric bulge forms from −36 position to the −37 position with respect to the target nucleotide to be edited (position 0) on the target RNA side of the guide-target RNA scaffold. As another example, if a latent guide RNA is annotated herein as forming a 2/2 symmetric bulge at position +12, then the 2/2 symmetric bulge forms from the +12 to the +13 position with respect to the target nucleotide to be edited (position 0) on the target RNA side of the guide-target RNA scaffold.

In some examples, the engineered guides disclosed herein lack a recruiting region and recruitment of the RNA editing entity can be effectuated by structural features of the guide-target RNA scaffold formed by hybridization of the engineered guide RNA and the target RNA. In some examples, the engineered guide, when present in an aqueous solution and not bound to the target RNA molecule, does not comprise structural features that recruit the RNA editing entity (e.g., ADAR or APOBEC). The engineered guide RNA, upon hybridization to a target RNA, form with the target RNA molecule, one or more structural features that recruits an RNA editing entity (e.g., ADAR or APOBEC).

In cases where a recruiting sequence can be absent, an engineered guide RNA can be still capable of associating with a subject RNA editing entity (e.g., ADAR or APOBEC) to facilitate editing of a target RNA and/or modulate expression of a polypeptide encoded by a subject target RNA. This can be achieved through structural features formed in the guide-target RNA scaffold formed upon hybridization of the engineered guide RNA and the target RNA. Structural features can comprise any one of a: mismatch, symmetrical bulge, asymmetrical bulge, symmetrical internal loop, asymmetrical internal loop, hairpins, wobble base pairs, or any combination thereof.

Described herein are structural features which can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA).

A guide-target RNA scaffold may be formed upon hybridization of an engineered guide RNA of the present disclosure to a target RNA. As disclosed herein, a mismatch refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch is an A/C mismatch. An A/C mismatch can comprise a C in an engineered guide RNA of the present disclosure opposite an A in a target RNA. An A/C mismatch can comprise an A in an engineered guide RNA of the present disclosure opposite a C in a target RNA. A G/G mismatch can comprise a G in an engineered guide RNA of the present disclosure opposite a G in a target RNA.

In some embodiments, a mismatch positioned 5′ of the edit site can facilitate base-flipping of the target A to be edited. A mismatch can also help confer sequence specificity. Thus, a mismatch can be a structural feature formed from latent structure provided by an engineered latent guide RNA.

In another aspect, a structural feature comprises a wobble base. A wobble base pair refers to two bases that weakly base pair. For example, a wobble base pair of the present disclosure can refer to a G paired with a U. Thus, a wobble base pair can be a structural feature formed from latent structure provided by an engineered latent guide RNA.

In some cases, a structural feature can be a hairpin. As disclosed herein, a hairpin includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin can have from 10 to 500 nucleotides in length of the entire duplex structure. The loop portion of a hairpin can be from 3 to 15 nucleotides long. A hairpin can be present in any of the engineered guide RNAs disclosed herein. The engineered guide RNAs disclosed herein can have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin can include a recruitment hairpin or a non-recruitment hairpin. A hairpin can be located anywhere within the engineered guide RNAs of the present disclosure. In some embodiments, one or more hairpins is proximal to or present at the 3′ end of an engineered guide RNA of the present disclosure, proximal to or at the 5′ end of an engineered guide RNA of the present disclosure, proximal to or within the targeting domain of the engineered guide RNAs of the present disclosure, or any combination thereof.

In some aspects, a structural feature comprises a non-recruitment hairpin. A non-recruitment hairpin, as disclosed herein, does not have a primary function of recruiting an RNA editing entity. A non-recruitment hairpin, in some instances, does not recruit an RNA editing entity. In some instances, a non-recruitment hairpin has a dissociation constant for binding to an RNA editing entity under physiological conditions that is insufficient for binding. For example, a non-recruitment hairpin has a dissociation constant for binding an RNA editing entity at 25° C. that is greater than about 1 mM, 10 mM, 100 mM, or 1 M, as determined in an in vitro assay. A non-recruitment hairpin can exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be present in constructs comprising engineered guide RNA constructs, not a structural feature formed by latent structure provided in an engineered latent guide RNA.

A hairpin of the present disclosure can be of any length. In an aspect, a hairpin can be from about 10-500 or more nucleotides. In some cases, a hairpin can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more nucleotides. In other cases, a hairpin can also comprise 10 to 20, 10 to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190, 10 to 200, 10 to 210, 10 to 220, 10 to 230, 10 to 240, 10 to 250, 10 to 260, 10 to 270, 10 to 280, 10 to 290, 10 to 300, 10 to 310, 10 to 320, 10 to 330, 10 to 340, 10 to 350, 10 to 360, 10 to 370, 10 to 380, 10 to 390, 10 to 400, 10 to 410, 10 to 420, 10 to 430, 10 to 440, 10 to 450, 10 to 460, 10 to 470, 10 to 480, 10 to 490, or 10 to 500 nucleotides.

A guide-target RNA scaffold is formed upon hybridization of an engineered guide RNA of the present disclosure to a target RNA. As disclosed herein, a bulge refers to the structure substantially formed only upon formation of the guide-target RNA scaffold, where contiguous nucleotides in either the engineered guide RNA or the target RNA are not complementary to their positional counterparts on the opposite strand. A bulge can change the secondary or tertiary structure of the guide-target RNA scaffold. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, a bulge can be a structural feature formed from latent structure provided by an engineered latent guide RNA.

In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A.

A guide-target RNA scaffold is formed upon hybridization of an engineered guide RNA of the present disclosure to a target RNA. A bulge can be a symmetrical bulge or an asymmetrical bulge. A symmetrical bulge is formed when the same number of nucleotides is present on each side of the bulge. For example, a symmetrical bulge in a guide-target RNA scaffold of the present disclosure can have the same number of nucleotides on the engineered guide RNA side and the target RNA side of the guide-target RNA scaffold. A symmetrical bulge of the present disclosure can be formed by 2 nucleotides on the engineered guide RNA side of the guide-target RNA scaffold target and 2 nucleotides on the target RNA side of the guide-target RNA scaffold. A symmetrical bulge of the present disclosure can be formed by 3 nucleotides on the engineered guide RNA side of the guide-target RNA scaffold target and 3 nucleotides on the target RNA side of the guide-target RNA scaffold. A symmetrical bulge of the present disclosure can be formed by 4 nucleotides on the engineered guide RNA side of the guide-target RNA scaffold target and 4 nucleotides on the target RNA side of the guide-target RNA scaffold. Thus, a symmetrical bulge can be a structural feature formed from latent structure provided by an engineered latent guide RNA.

A guide-target RNA scaffold is formed upon hybridization of an engineered guide RNA of the present disclosure to a target RNA. A bulge can be a symmetrical bulge or an asymmetrical bulge. An asymmetrical bulge is formed when a different number of nucleotides is present on each side of the bulge. For example, an asymmetrical bulge in a guide-target RNA scaffold of the present disclosure can have different numbers of nucleotides on the engineered guide RNA side and the target RNA side of the guide-target RNA scaffold. An asymmetrical bulge of the present disclosure can be formed by 0 nucleotides on the engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the engineered guide RNA side of the guide-target RNA scaffold. An asymmetrical bulge of the present disclosure can be formed by 0 nucleotides on the engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the engineered guide RNA side of the guide-target RNA scaffold. An asymmetrical bulge of the present disclosure can be formed by 0 nucleotides on the engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the engineered guide RNA side of the guide-target RNA scaffold. An asymmetrical bulge of the present disclosure can be formed by 0 nucleotides on the engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the engineered guide RNA side of the guide-target RNA scaffold. An asymmetrical bulge of the present disclosure can be formed by 1 nucleotide on the engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the engineered guide RNA side of the guide-target RNA scaffold. An asymmetrical bulge of the present disclosure can be formed by 1 nucleotide on the engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the engineered guide RNA side of the guide-target RNA scaffold. An asymmetrical bulge of the present disclosure can be formed by 1 nucleotide on the engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the engineered guide RNA side of the guide-target RNA scaffold. An asymmetrical bulge of the present disclosure can be formed by 2 nucleotides on the engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the engineered guide RNA side of the guide-target RNA scaffold. An asymmetrical bulge of the present disclosure can be formed by 2 nucleotides on the engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the engineered guide RNA side of the guide-target RNA scaffold. An asymmetrical bulge of the present disclosure can be formed by 3 nucleotides on the engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the engineered guide RNA side of the guide-target RNA scaffold. Thus, an asymmetrical bulge can be a structural feature formed from latent structure provided by an engineered latent guide RNA.

In some cases, a structural feature can be an internal loop. As disclosed herein, an internal loop refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. Internal loops present in the vicinity of the edit site can help with base flipping of the target A in the target RNA to be edited.

One side of the internal loop, either on the target RNA side or the engineered guide RNA side of the guide-target RNA scaffold, can be formed by from 5 to 150 nucleotides. One side of the internal loop can be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop can be formed by 5 nucleotides. One side of the internal loop can be formed by 10 nucleotides. One side of the internal loop can be formed by 15 nucleotides. One side of the internal loop can be formed by 20 nucleotides. One side of the internal loop can be formed by 25 nucleotides. One side of the internal loop can be formed by 30 nucleotides. One side of the internal loop can be formed by 35 nucleotides. One side of the internal loop can be formed by 40 nucleotides. One side of the internal loop can be formed by 45 nucleotides. One side of the internal loop can be formed by 50 nucleotides. One side of the internal loop can be formed by 55 nucleotides. One side of the internal loop can be formed by 60 nucleotides. One side of the internal loop can be formed by 65 nucleotides. One side of the internal loop can be formed by 70 nucleotides. One side of the internal loop can be formed by 75 nucleotides. One side of the internal loop can be formed by 80 nucleotides. One side of the internal loop can be formed by 85 nucleotides. One side of the internal loop can be formed by 90 nucleotides. One side of the internal loop can be formed by 95 nucleotides. One side of the internal loop can be formed by 100 nucleotides. One side of the internal loop can be formed by 110 nucleotides. One side of the internal loop can be formed by 120 nucleotides. One side of the internal loop can be formed by 130 nucleotides. One side of the internal loop can be formed by 140 nucleotides. One side of the internal loop can be formed by 150 nucleotides. One side of the internal loop can be formed by 200 nucleotides. One side of the internal loop can be formed by 250 nucleotides. One side of the internal loop can be formed by 300 nucleotides. One side of the internal loop can be formed by 350 nucleotides. One side of the internal loop can be formed by 400 nucleotides. One side of the internal loop can be formed by 450 nucleotides. One side of the internal loop can be formed by 500 nucleotides. One side of the internal loop can be formed by 600 nucleotides. One side of the internal loop can be formed by 700 nucleotides. One side of the internal loop can be formed by 800 nucleotides. One side of the internal loop can be formed by 900 nucleotides. One side of the internal loop can be formed by 1000 nucleotides. Thus, an internal loop can be a structural feature formed from latent structure provided by an engineered latent guide RNA.

An internal loop can be a symmetrical internal loop or an asymmetrical internal loop. A symmetrical internal loop is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 15 nucleotides on the engineered guide RNA side of the guide-target RNA scaffold target and 15 nucleotides on the target RNA side of the guide-target RNA scaffold. A symmetrical internal loop of the present disclosure can be formed by 20 nucleotides on the engineered guide RNA side of the guide-target RNA scaffold target and 20 nucleotides on the target RNA side of the guide-target RNA scaffold. A symmetrical internal loop of the present disclosure can be formed by 30 nucleotides on the engineered guide RNA side of the guide-target RNA scaffold target and 30 nucleotides on the target RNA side of the guide-target RNA scaffold. A symmetrical internal loop of the present disclosure can be formed by 40 nucleotides on the engineered guide RNA side of the guide-target RNA scaffold target and 40 nucleotides on the target RNA side of the guide-target RNA scaffold. A symmetrical internal loop of the present disclosure can be formed by 50 nucleotides on the engineered guide RNA side of the guide-target RNA scaffold target and 50 nucleotides on the target RNA side of the guide-target RNA scaffold. A symmetrical internal loop of the present disclosure can be formed by 60 nucleotides on the engineered guide RNA side of the guide-target RNA scaffold target and 60 nucleotides on the target RNA side of the guide-target RNA scaffold. A symmetrical internal loop of the present disclosure can be formed by 70 nucleotides on the engineered guide RNA side of the guide-target RNA scaffold target and 70 nucleotides on the target RNA side of the guide-target RNA scaffold. A symmetrical internal loop of the present disclosure can be formed by 80 nucleotides on the engineered guide RNA side of the guide-target RNA scaffold target and 80 nucleotides on the target RNA side of the guide-target RNA scaffold. A symmetrical internal loop of the present disclosure can be formed by 90 nucleotides on the engineered guide RNA side of the guide-target RNA scaffold target and 90 nucleotides on the target RNA side of the guide-target RNA scaffold. A symmetrical internal loop of the present disclosure can be formed by 100 nucleotides on the engineered guide RNA side of the guide-target RNA scaffold target and 100 nucleotides on the target RNA side of the guide-target RNA scaffold. A symmetrical internal loop of the present disclosure can be formed by 110 nucleotides on the engineered guide RNA side of the guide-target RNA scaffold target and 110 nucleotides on the target RNA side of the guide-target RNA scaffold. A symmetrical internal loop of the present disclosure can be formed by 120 nucleotides on the engineered guide RNA side of the guide-target RNA scaffold target and 120 nucleotides on the target RNA side of the guide-target RNA scaffold. A symmetrical internal loop of the present disclosure can be formed by 130 nucleotides on the engineered guide RNA side of the guide-target RNA scaffold target and 130 nucleotides on the target RNA side of the guide-target RNA scaffold. A symmetrical internal loop of the present disclosure can be formed by 140 nucleotides on the engineered guide RNA side of the guide-target RNA scaffold target and 140 nucleotides on the target RNA side of the guide-target RNA scaffold. A symmetrical internal loop of the present disclosure can be formed by 150 nucleotides on the engineered guide RNA side of the guide-target RNA scaffold target and 150 nucleotides on the target RNA side of the guide-target RNA scaffold. A symmetrical internal loop of the present disclosure can be formed by 200 nucleotides on the engineered guide RNA side of the guide-target RNA scaffold target and 200 nucleotides on the target RNA side of the guide-target RNA scaffold. A symmetrical internal loop of the present disclosure can be formed by 250 nucleotides on the engineered guide RNA side of the guide-target RNA scaffold target and 250 nucleotides on the target RNA side of the guide-target RNA scaffold. A symmetrical internal loop of the present disclosure can be formed by 300 nucleotides on the engineered guide RNA side of the guide-target RNA scaffold target and 300 nucleotides on the target RNA side of the guide-target RNA scaffold. A symmetrical internal loop of the present disclosure can be formed by 350 nucleotides on the engineered guide RNA side of the guide-target RNA scaffold target and 350 nucleotides on the target RNA side of the guide-target RNA scaffold. A symmetrical internal loop of the present disclosure can be formed by 400 nucleotides on the engineered guide RNA side of the guide-target RNA scaffold target and 400 nucleotides on the target RNA side of the guide-target RNA scaffold. A symmetrical internal loop of the present disclosure can be formed by 450 nucleotides on the engineered guide RNA side of the guide-target RNA scaffold target and 450 nucleotides on the target RNA side of the guide-target RNA scaffold. A symmetrical internal loop of the present disclosure can be formed by 500 nucleotides on the engineered guide RNA side of the guide-target RNA scaffold target and 500 nucleotides on the target RNA side of the guide-target RNA scaffold. A symmetrical internal loop of the present disclosure can be formed by 600 nucleotides on the engineered guide RNA side of the guide-target RNA scaffold target and 600 nucleotides on the target RNA side of the guide-target RNA scaffold. A symmetrical internal loop of the present disclosure can be formed by 700 nucleotides on the engineered guide RNA side of the guide-target RNA scaffold target and 700 nucleotides on the target RNA side of the guide-target RNA scaffold. A symmetrical internal loop of the present disclosure can be formed by 800 nucleotides on the engineered guide RNA side of the guide-target RNA scaffold target and 800 nucleotides on the target RNA side of the guide-target RNA scaffold. A symmetrical internal loop of the present disclosure can be formed by 900 nucleotides on the engineered guide RNA side of the guide-target RNA scaffold target and 900 nucleotides on the target RNA side of the guide-target RNA scaffold. A symmetrical internal loop of the present disclosure can be formed by 1000 nucleotides on the engineered guide RNA side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

An asymmetrical internal loop is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetrical internal loop of the present disclosure can be formed by from 5 to 150 nucleotides on the engineered guide RNA side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered guide RNA side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered guide RNA side of the guide-target RNA scaffold. An asymmetrical internal loop of the present disclosure can be formed by 5 nucleotides on the target RNA side of the guide-target RNA scaffold and 100 nucleotides on the engineered guide RNA side of the guide-target RNA scaffold. An asymmetrical internal loop of the present disclosure can be formed by 5 nucleotides on the target RNA side of the guide-target RNA scaffold and 150 nucleotides on the engineered guide RNA side of the guide-target RNA scaffold. An asymmetrical internal loop of the present disclosure can be formed by 5 nucleotides on the target RNA side of the guide-target RNA scaffold and 200 nucleotides on the engineered guide RNA side of the guide-target RNA scaffold. An asymmetrical internal loop of the present disclosure can be formed by 5 nucleotides on the target RNA side of the guide-target RNA scaffold and 300 nucleotides on the engineered guide RNA side of the guide-target RNA scaffold. An asymmetrical internal loop of the present disclosure can be formed by 5 nucleotides on the target RNA side of the guide-target RNA scaffold and 400 nucleotides on the engineered guide RNA side of the guide-target RNA scaffold. An asymmetrical internal loop of the present disclosure can be formed by 5 nucleotides on the target RNA side of the guide-target RNA scaffold and 500 nucleotides on the engineered guide RNA side of the guide-target RNA scaffold. An asymmetrical internal loop of the present disclosure can be formed by 5 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 5 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 5 nucleotides on the engineered guide RNA side of the guide-target RNA scaffold. An asymmetrical internal loop of the present disclosure can be formed by 300 nucleotides on the target RNA side of the guide-target RNA scaffold and 5 nucleotides on the engineered guide RNA side of the guide-target RNA scaffold. An asymmetrical internal loop of the present disclosure can be formed by 200 nucleotides on the target RNA side of the guide-target RNA scaffold and 5 nucleotides on the engineered guide RNA side of the guide-target RNA scaffold. An asymmetrical internal loop of the present disclosure can be formed by 150 nucleotides on the target RNA side of the guide-target RNA scaffold and 5 nucleotides on the engineered guide RNA side of the guide-target RNA scaffold. An asymmetrical internal loop of the present disclosure can be formed by 100 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 100 nucleotides on the engineered guide RNA side of the guide-target RNA scaffold. An asymmetrical internal loop of the present disclosure can be formed by 50 nucleotides on the target RNA side of the guide-target RNA scaffold and 150 nucleotides on the engineered guide RNA side of the guide-target RNA scaffold. An asymmetrical internal loop of the present disclosure can be formed by 50 nucleotides on the target RNA side of the guide-target RNA scaffold and 200 nucleotides on the engineered guide RNA side of the guide-target RNA scaffold. An asymmetrical internal loop of the present disclosure can be formed by 50 nucleotides on the target RNA side of the guide-target RNA scaffold and 300 nucleotides on the engineered guide RNA side of the guide-target RNA scaffold. An asymmetrical internal loop of the present disclosure can be formed by 50 nucleotides on the target RNA side of the guide-target RNA scaffold and 400 nucleotides on the engineered guide RNA side of the guide-target RNA scaffold. An asymmetrical internal loop of the present disclosure can be formed by 50 nucleotides on the target RNA side of the guide-target RNA scaffold and 500 nucleotides on the engineered guide RNA side of the guide-target RNA scaffold. An asymmetrical internal loop of the present disclosure can be formed by 50 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 50 nucleotides on the engineered guide RNA side of the guide-target RNA scaffold. An asymmetrical internal loop of the present disclosure can be formed by 500 nucleotides on the target RNA side of the guide-target RNA scaffold and 50 nucleotides on the engineered guide RNA side of the guide-target RNA scaffold. An asymmetrical internal loop of the present disclosure can be formed by 400 nucleotides on the target RNA side of the guide-target RNA scaffold and 50 nucleotides on the engineered guide RNA side of the guide-target RNA scaffold. An asymmetrical internal loop of the present disclosure can be formed by 300 nucleotides on the target RNA side of the guide-target RNA scaffold and 50 nucleotides on the engineered guide RNA side of the guide-target RNA scaffold. An asymmetrical internal loop of the present disclosure can be formed by 200 nucleotides on the target RNA side of the guide-target RNA scaffold and 50 nucleotides on the engineered guide RNA side of the guide-target RNA scaffold. An asymmetrical internal loop of the present disclosure can be formed by 150 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 150 nucleotides on the engineered guide RNA side of the guide-target RNA scaffold. An asymmetrical internal loop of the present disclosure can be formed by 100 nucleotides on the target RNA side of the guide-target RNA scaffold and 200 nucleotides on the engineered guide RNA side of the guide-target RNA scaffold. An asymmetrical internal loop of the present disclosure can be formed by 100 nucleotides on the target RNA side of the guide-target RNA scaffold and 300 nucleotides on the engineered guide RNA side of the guide-target RNA scaffold. An asymmetrical internal loop of the present disclosure can be formed by 100 nucleotides on the target RNA side of the guide-target RNA scaffold and 400 nucleotides on the engineered guide RNA side of the guide-target RNA scaffold. An asymmetrical internal loop of the present disclosure can be formed by 100 nucleotides on the target RNA side of the guide-target RNA scaffold and 500 nucleotides on the engineered guide RNA side of the guide-target RNA scaffold. An asymmetrical internal loop of the present disclosure can be formed by 100 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 100 nucleotides on the engineered guide RNA side of the guide-target RNA scaffold. An asymmetrical internal loop of the present disclosure can be formed by 500 nucleotides on the target RNA side of the guide-target RNA scaffold and 100 nucleotides on the engineered guide RNA side of the guide-target RNA scaffold. An asymmetrical internal loop of the present disclosure can be formed by 400 nucleotides on the target RNA side of the guide-target RNA scaffold and 100 nucleotides on the engineered guide RNA side of the guide-target RNA scaffold. An asymmetrical internal loop of the present disclosure can be formed by 300 nucleotides on the target RNA side of the guide-target RNA scaffold and 100 nucleotides on the engineered guide RNA side of the guide-target RNA scaffold. An asymmetrical internal loop of the present disclosure can be formed by 200 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 200 nucleotides on the engineered guide RNA side of the guide-target RNA scaffold. An asymmetrical internal loop of the present disclosure can be formed by 150 nucleotides on the target RNA side of the guide-target RNA scaffold and 300 nucleotides on the engineered guide RNA side of the guide-target RNA scaffold. An asymmetrical internal loop of the present disclosure can be formed by 150 nucleotides on the target RNA side of the guide-target RNA scaffold and 400 nucleotides on the engineered guide RNA side of the guide-target RNA scaffold. An asymmetrical internal loop of the present disclosure can be formed by 150 nucleotides on the target RNA side of the guide-target RNA scaffold and 500 nucleotides on the engineered guide RNA side of the guide-target RNA scaffold. An asymmetrical internal loop of the present disclosure can be formed by 150 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 150 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 150 nucleotides on the engineered guide RNA side of the guide-target RNA scaffold. An asymmetrical internal loop of the present disclosure can be formed by 300 nucleotides on the target RNA side of the guide-target RNA scaffold and 150 nucleotides on the engineered guide RNA side of the guide-target RNA scaffold. An asymmetrical internal loop of the present disclosure can be formed by 200 nucleotides on the target RNA side of the guide-target RNA scaffold and 300 nucleotides on the engineered guide RNA side of the guide-target RNA scaffold. An asymmetrical internal loop of the present disclosure can be formed by 200 nucleotides on the target RNA side of the guide-target RNA scaffold and 400 nucleotides on the engineered guide RNA side of the guide-target RNA scaffold. An asymmetrical internal loop of the present disclosure can be formed by 200 nucleotides on the target RNA side of the guide-target RNA scaffold and 500 nucleotides on the engineered guide RNA side of the guide-target RNA scaffold. An asymmetrical internal loop of the present disclosure can be formed by 200 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 200 nucleotides on the engineered guide RNA side of the guide-target RNA scaffold. An asymmetrical internal loop of the present disclosure can be formed by 500 nucleotides on the target RNA side of the guide-target RNA scaffold and 200 nucleotides on the engineered guide RNA side of the guide-target RNA scaffold. An asymmetrical internal loop of the present disclosure can be formed by 400 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 400 nucleotides on the engineered guide RNA side of the guide-target RNA scaffold. An asymmetrical internal loop of the present disclosure can be formed by 300 nucleotides on the target RNA side of the guide-target RNA scaffold and 500 nucleotides on the engineered guide RNA side of the guide-target RNA scaffold. An asymmetrical internal loop of the present disclosure can be formed by 300 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 300 nucleotides on the engineered guide RNA side of the guide-target RNA scaffold. An asymmetrical internal loop of the present disclosure can be formed by 500 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 500 nucleotides on the engineered guide RNA side of the guide-target RNA scaffold. An asymmetrical internal loop of the present disclosure can be formed by 400 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 500 nucleotides on the engineered guide RNA side of the guide-target RNA scaffold. Thus, an asymmetrical internal loop can be a structural feature formed from latent structure provided by an engineered latent guide RNA.

As described herein, a “micro-footprint” sequence refers to a sequence with latent structures that, when manifested, facilitate editing of the adenosine of a target RNA via an adenosine deaminase enzyme. A macro-footprint can serve to guide or focus an RNA editing entity (e.g., ADAR) and direct its activity towards a micro-footprint. In some embodiments, included within the micro-footprint sequence is a nucleotide that is positioned such that, when the guide RNA is hybridized to the target RNA, said nucleotide is opposite the adenosine to be edited by the ADAR enzyme and does not base pair with the adenosine to be edited. This nucleotide is referred to herein as the “mismatched position” or “mismatch” and can be a cytosine. Micro-footprint sequences as described herein have, upon hybridization of the engineered guide RNA and target RNA, at least one structural feature selected from the group consisting of: a bulge, an internal loop, a mismatch, a hairpin, and any combination thereof. Engineered guide RNAs with superior micro-footprint sequences can be selected based on their ability to facilitate editing of a specific target RNA. Engineered guide RNAs selected for their ability to facilitate editing of a specific target are capable of adopting various micro-footprint latent structures, which can vary on a target-by-target basis.

Guide RNAs of the present disclosure may further comprise a macro-footprint. In some embodiments, the macro-footprint comprises a barbell macro-footprint. A micro-footprint can serve to guide or focus an RNA editing enzyme and direct its activity towards the target adenosine to be edited. A “barbell” as described herein refers to a pair of internal loop latent structural features that manifest upon hybridization of the guide RNA to the target RNA. In some embodiments, each internal loop is positioned towards the 5′ end or the 3′ end of the guide-target RNA scaffold formed upon hybridization of the guide RNA and the target RNA. In some embodiments, each internal loop flanks opposing sides of the micro-footprint sequence. Insertion of a barbell macro-footprint sequence flanking opposing sides of the micro-footprint sequence, upon hybridization of the guide RNA to the target RNA, results in formation of barbell internal loops on opposing sides of the micro-footprint, which in turn comprises at least one structural feature that facilitates editing of a specific target RNA.

In some embodiments, the presence of barbells flanking the micro-footprint can improve one or more aspects of editing. For example, the presence of a barbell macro-footprint in addition to a micro-footprint can result in a higher amount of on target adenosine editing, relative to an otherwise comparable guide RNA lacking the barbells. Additionally, and or alternatively, the presence of a barbell macro-footprint in addition to a micro-footprint can result in a lower amount of local off-target adenosine editing, relative to an otherwise comparable guide RNA lacking the barbells. Further, while the effect of various micro-footprint structural features can vary on a target-by-target basis based on selection in a high throughput screen, the increase in the one or more aspects of editing provided by the barbell macro-footprint structures can be independent of the particular target RNA. Thus, inclusion of barbell structures can provide a facile method of improving editing of guide RNAs previously selected to facilitate editing of a target RNA of interest. For example, macro-footprints (e.g., barbell macro-footprints) and micro-footprints can provide an increased amount of on target adenosine editing relative to an otherwise comparable guide RNA lacking the barbells. In other embodiments, the presence of the barbell macro-footprint in addition to the micro-footprint can result in a lower amount of local off-target adenosine editing, relative to an otherwise comparable guide RNA, upon hybridization of the guide RNA and target RNA to form a guide-target RNA scaffold lacking the barbells.

As disclosed herein, a “macro-footprint” sequence can be positioned such that it flanks a micro-footprint sequence. Further, while a macro-footprint sequence can flank a micro-footprint sequence, additional latent structures can be incorporated that flank either end of the macro-footprint as well. In some embodiments, such additional latent structures are included as part of the macro-footprint. In some embodiments, such additional latent structures are separate, distinct, or both separate and distinct from the macro-footprint. In some embodiments, a macro-footprint sequence can comprise a barbell macro-footprint sequence comprising latent structures that, when manifested, produce a first internal loop and a second internal loop.

In some embodiments, the first internal loop of the barbell or the second internal loop of the barbell is positioned at least about 5 bases (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases) away from the A/C mismatch with respect to the base of the first internal loop or the second internal loop that is the most proximal to the A/C mismatch. In some embodiments, the first internal loop of the barbell or the second internal loop of the barbell is positioned at most about 50 bases away from the A/C mismatch (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5) with respect to the base of the first internal loop or the second internal loop that is the most proximal to the A/C mismatch.

In some embodiments, a first internal loop or a second internal loop independently comprises a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the engineered guide RNA and a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the target RNA.

As disclosed herein, a “base paired (bp) region” refers to a region of the guide-target RNA scaffold in which bases in the guide RNA (e.g., the bases in the targeting sequence of the guide RNA) are paired with opposing bases in the target polynucleotide. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold. In some embodiments, a base paired region has from 1 to 50, 1 to 75, 1 to 100, 1 to 125, 1 to 150, 1 to 175, 1 to 200, 1 to 225, 1 to 250, 1 to 275, 1 to 300, 50 to 75, 50 to 100, 50 to 125, 50 to 150, 50 to 175, 50 to 200, 50 to 225, 50 to 250, 50 to 275, 50 to 300, 60 to 75, 60 to 100, 60 to 125, 60 to 150, 60 to 175, 60 to 200, 60 to 225, 60 to 250, 60 to 275, 60 to 300, 70 to 100, 70 to 125, 70 to 150, 70 to 175, 70 to 200, 70 to 225, 70 to 250, 70 to 275, 70 to 300, 80 to 100, 80 to 125, 80 to 150, 80 to 175, 80 to 200, 80 to 225, 80 to 250, 80 to 275, 80 to 300, 90 to 125, 90 to 150, 90 to 175, 90 to 200, 90 to 225, 90 to 250, 90 to 275, 90 to 300, 100 to 125, 100 to 150, 100 to 175, 100 to 200, 100 to 225, 100 to 250, 100 to 275, 100 to 300, 150 to 200, 150 to 225, 150 to 250, 150 to 275, or 150 to 300 base pairs. In some embodiments, a base paired region has at least 1, 2, 3, 4, 5, 6, 7, 8, 9, 10, 12, 14, 16, 18, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 base pairs.

### Additional Engineered Guide RNA Components

The present disclosure provides for engineered guide RNAs with additional structural features and components. For example, an engineered guide RNA described herein can be circular. In another example, an engineered guide RNA described herein can comprise a U7, an SmOPT sequence, or a combination of both sequences.

In some cases, an engineered guide RNA can be circularized. In some cases, an engineered guide RNA provided herein can be circularized or in a circular configuration. In some aspects, an at least partially circular guide RNA lacks a 5′ hydroxyl or a 3′ hydroxyl.

In some examples, an engineered guide RNA can comprise a backbone comprising a plurality of sugar and phosphate moieties covalently linked together. In some examples, a backbone of an engineered guide RNA can comprise a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an engineered guide RNA can lack a 5′ reducing hydroxyl, a 3′ reducing hydroxyl, or both, capable of being exposed to a solvent. In some embodiments, a backbone of an engineered guide can lack a 5′ reducing hydroxyl, a 3′ reducing hydroxyl, or both, capable of being exposed to nucleases. In some embodiments, a backbone of an engineered guide can lack a 5′ reducing hydroxyl, a 3′ reducing hydroxyl, or both, capable of being exposed to hydrolytic enzymes. In some instances, a backbone of an engineered guide can be represented as a polynucleotide sequence in a circular 2-dimensional format with one nucleotide after the other. In some instances, a backbone of an engineered guide can be represented as a polynucleotide sequence in a looped 2-dimensional format with one nucleotide after the other. In some cases, a 5′ hydroxyl, a 3′ hydroxyl, or both, can be joined through a phosphorus-oxygen bond. In some cases, a 5′ hydroxyl, a 3′ hydroxyl, or both, can be modified into a phosphoester with a phosphorus-containing moiety.

As described herein, an engineered guide can comprise a circular structure. An engineered polynucleotide can be circularized from a precursor engineered polynucleotide. Such a precursor engineered polynucleotide can be a precursor engineered linear polynucleotide. In some cases, a precursor engineered linear polynucleotide can be a precursor for a circular engineered guide RNA. For example, a precursor engineered linear polynucleotide can be a linear mRNA transcribed from a plasmid, which can be configured to circularize within a cell using the techniques described herein. A precursor engineered linear polynucleotide can be constructed with domains such as a ribozyme domain and a ligation domain that allow for circularization when inserted into a cell. A ribozyme domain can include a domain that is capable of cleaving the linear precursor RNA at specific sites (e.g., adjacent to the ligation domain). A precursor engineered linear polynucleotide can comprise, from 5′ to 3′: a 5′ ribozyme domain, a 5′ ligation domain, a circularized region, a 3′ ligation domain, and a 3′ ribozyme domain. In some cases, a circularized region can comprise a guide RNA described herein. In some cases, the precursor polynucleotide can be specifically processed at both sites by the 5′ and the 3′ ribozymes, respectively, to free exposed ends on the 5′ and 3′ ligation domains. The free exposed ends can be ligation competent, such that the ends can be ligated to form a mature circularized structure. For instance, the free ends can include a 5′-OH and a 2′, 3′-cyclic phosphate that are ligated via RNA ligation in the cell. The linear polynucleotide with the ligation and ribozyme domains can be transfected into a cell where it can circularize via endogenous cellular enzymes. In some cases, a polynucleotide can encode an engineered guide RNA comprising the ribozyme and ligation domains described herein, which can circularize within a cell. For example, PCT/US2021/034301 provides a description of circular guide RNAs and their structures, sequences of circular guide RNAs, and methods of engineering circularized polynucleotide domains, and each of these descriptions in PCT/US2021/034301 is herein incorporated by reference.

An engineered polynucleotide as described herein (e.g., a circularized guide RNA) can include spacer domains. As described herein, a spacer domain can refer to a domain that provides space between other domains. A spacer domain can be used to between a region to be circularized and flanking ligation sequences to increase the overall size of the mature circularized guide RNA. Where the region to be circularized includes a targeting domain as described herein that is configured to associate to a target sequence, the addition of spacers can provide improvements (e.g., increased specificity, enhanced editing efficiency, etc.) for the engineered polynucleotide to the target polynucleotide, relative to a comparable engineered polynucleotide that lacks a spacer domain. In some instances, the spacer domain is configured to not hybridize with the target RNA. In some embodiments, a precursor engineered polynucleotide or a circular engineered guide, can comprise, in order of 5′ to 3′: a first ribozyme domain; a first ligation domain; a first spacer domain; a targeting domain that can be at least partially complementary to a target RNA, a second spacer domain, a second ligation domain, and a second ribozyme domain. In some cases, the first spacer domain, the second spacer domain, or both are configured to not bind to the target RNA when the targeting domain binds to the target RNA.

A circular or looped RNA can be formed by employing a self-cleaving entity, such as a ribozyme, tRNA, aptamer, catalytically active fragment of any of these, or any combination thereof. For example, a ribozyme, a tRNA, an aptamer, a catalytically active fragment of any of these, or any combination thereof can be added to a 3′ end, a 5′ end, or both of a precursor engineered RNA. In another example, a ribozyme, a tRNA, an aptamer, a catalytically active fragment of any of these, or any combination thereof can be added to a 3′ terminal end, a 5′ terminal end, or both of a precursor engineered RNA. A self-cleaving ribozyme can comprise, for example, an RNase P RNA a Hammerhead ribozyme (e.g., a Schistosoma mansoni ribozyme), a glmS ribozyme, an HDV-like ribozyme, an R2 element, a peptidyl transferase 23S rRNA, a GIR1 branching ribozyme, a leadzyme, a group II intron, a hairpin ribozyme, a VS ribozyme, a CPEB3 ribozyme, a CoTC ribozyme, or a group I intron. In some cases, the self-cleaving ribozyme can be a trans-acting ribozyme that joins one RNA end on which it is present to a separate RNA end. In some embodiments, an aptamer can be added to each end of the engineered guide RNA. A ligase can be contacted with the aptamers at each end of the engineered guide RNA to form a covalent linkage between the aptamers thereby forming a circular engineered guide RNA. In some cases, a self-cleaving element or an aptamer can be configured to facilitate self-circularization of an engineered polynucleotide or a pro-polynucleotide (e.g., from a precursor engineered polypeptide) after transcription in a cell. In some instances, circularization of a guide RNA can be shown by PCR. For example, primers can by developed that bind to the end of a guide RNA and are directed outward such that a product is only formed when guides are circularized.

In some cases, circularization can occur by back-slicing and ligation of an exon. For example, an RNA can be engineered from 5′ to 3′ to comprise a forward complementary sequence intron, an exon (which can comprise the guide sequence), followed by a reverse complementary sequence intron. Once transcribed, the complementary sequence introns can hybridize and form dsRNA. The internal exon containing the guide sequence can be removed by splicing and ligated by an endogenous ligase to form a circular guide. In one example, an engineered guide RNA can initiate circularization in a cell by autocatalytic reactions of encoded ribozymes. After cleavage by one or more ribozymes, the linear polynucleotide will undergo intracellular RNA ligation of the 5′ and the 3′ end of ligation sequences by an endogenous ligase to circularize the guide RNA.

A suitable self-cleaving molecule can include a ribozyme. For example, a ribozyme domain can create an autocatalytic RNA. A ribozyme can comprise an RNase P, an rRNA (such as a Peptidyl transferase 23S rRNA), Leadzyme, Group I intron ribozyme, Group II intron ribozyme, a GIR1 branching ribozyme, a glmS ribozyme, a hairpin ribozyme, a Hammerhead ribozyme, an HDV ribozyme, a Twister ribozyme, a Twister sister ribozyme, a VS ribozyme, a Pistol ribozyme, a Hatchet ribozyme, a viroid, or any combination thereof. A ribozyme can include a P3 twister U2A ribozyme.

A ligation domain can facilitate a linkage, covalent or non-covalent, of a first nucleotide to a second nucleotide. In some embodiments, a ligation domain can recruit a ligating entity to facilitate a ligation reaction. In some cases, a ligation domain can recruit a recombining entity to facilitate a homologous recombination. In some instances, a first ligation domain can facilitate a linkage, covalent or non-covalent, to a second ligation domain. In some embodiments, a first ligation domain can facilitate the complementary pairing of a second ligation domain.

The compositions and methods of the present disclosure provide engineered polynucleotides encoding for guide RNAs that are operably linked to a portion of a small nuclear ribonucleic acid (snRNA) sequence. The engineered polynucleotide can include at least a portion of a small nuclear ribonucleic acid (snRNA) sequence. The U7 and U1 small nuclear RNAs, whose natural role is in spliceosomal processing of pre-mRNA, have for decades been re-engineered to alter splicing at desired disease targets. Replacing a portion of the U7 snRNA which naturally hybridizes to the spacer element of histone pre-mRNA (e.g., the first 18 nucleotides of the U7 snRNA) with a short targeting (or antisense) sequence of a disease gene, may redirect the splicing machinery to alter splicing around that target site. Furthermore, converting the wild type U7 Sm-domain binding site to an optimized consensus Sm-binding sequence (SmOPT) can increase the expression level, activity, and subcellular localization of the artificial antisense-engineered U7 snRNA. Many subsequent groups have adapted this modified U7 SmOPT snRNA chassis with antisense sequences of other genes to recruit spliceosomal elements and modify RNA splicing for additional disease targets.

An snRNA is a class of small RNA molecules found within the nucleus of eukaryotic cells. They are involved in a variety of important processes such as RNA splicing (removal of introns from pre-mRNA), regulation of transcription factors (7SK RNA) or RNA polymerase II (B2 RNA), and maintaining the telomeres. They are always associated with specific proteins, and the resulting RNA-protein complexes are referred to as small nuclear ribonucleoproteins (snRNP) or sometimes as snurps. There are many snRNAs, which are denominated U1, U2, U3, U4, U5, U6, U7, U8, U9, and U10.

The snRNA of the U7 type is normally involved in the maturation of histone mRNA. This snRNA has been identified in a great number of eukaryotic species (56 so far) and the U7 snRNA of each of these species should be regarded as equally convenient for this disclosure.

Wild type U7 snRNA includes a stem-loop structure, the U7-specific Sm sequence, and a sequence antisense to the 3′ end of histone pre-mRNA.

In addition to the SmOPT domain, U7 comprises a sequence antisense to the 3′ end of histone pre-mRNA. When this sequence is replaced by a targeting sequence that is antisense to another target pre-mRNA, U7 is redirected to the new target pre-mRNA. Accordingly, the stable expression of modified U7 snRNAs containing the SmOPT domain and a targeting antisense sequence has resulted in specific alteration of mRNA splicing. While AAV-2/1 based vectors expressing an appropriately modified murine U7 gene along with its natural promoter and 3′ elements have enabled high efficiency gene transfer into the skeletal muscle and complete dystrophin rescue by covering and skipping mouse Dmd exon 23, the engineered polynucleotides as described herein (whether directly administered or administered via, for example, AAV vectors) can facilitate editing of target RNA by a deaminase.

The engineered polynucleotide can comprise at least in part an snRNA sequence. The snRNA sequence can be U1, U2, U3, U4, U5, U6, U7, U8, U9, or a U10 snRNA sequence.

In some instances, an engineered polynucleotide that comprises at least a portion of an snRNA sequence (e.g., an snRNA promoter, an snRNA hairpin, and the like) can have superior properties for treating or preventing a disease or condition, relative to a comparable polynucleotide lacking such features. For example, as described herein an engineered polynucleotide that comprises at least a portion of an snRNA sequence can facilitate exon skipping of an exon at a greater efficiency than a comparable polynucleotide lacking such features. Further, as described herein an engineered polynucleotide that comprises at least a portion of an snRNA sequence can facilitate an editing of a base of a nucleotide in a target RNA (e.g., a pre-mRNA or a mature RNA) at a greater efficiency than a comparable polynucleotide lacking such features. Promoters and snRNA components are described in PCT/US2021/028618 and PCT/US2022/078801, and each of these descriptions in PCT/US2021/028618 and PCT/US2022/078801 are herein incorporated by reference.

Disclosed herein are engineered RNAs comprising (a) an engineered guide RNA as described herein, and (b) a U7 snRNA hairpin sequence, a SmOPT sequence, or a combination thereof. An expression cassette or polynucleotide as described herein may comprise one or more U7 snRNA hairpin sequences, SmOPT sequences, or combinations thereof. For example, an expression cassette or polynucleotide as described herein may comprise two U7 snRNA hairpin sequences and two SmOPT sequences. In some embodiments, the U7 hairpin comprises a human U7 Hairpin sequence, or a mouse U7 hairpin sequence. In some cases, a human U7 hairpin sequence comprises TAGGCTTTCTGGCTTTTTACCGGAAAGCCCCT (SEQ ID NO: 17) or RNA: UAGGCUUUCUGGCUUUUUACCGGAAAGCCCCU (SEQ ID NO: 18). In some cases, a mouse U7 hairpin sequence comprises CAGGTTTTCTGACTTCGGTCGGAAAACCCCT (SEQ ID NO: 19) or RNA: CAGGUUUUCUGACUUCGGUCGGAAAACCCCU (SEQ ID NO: 20). In some cases, a hairpin sequence may comprise SEQ ID NO: 83 or SEQ ID NO: 84. In some embodiments, the SmOPT sequence has a sequence of AATTTTTGGAG (SEQ ID NO: 21) or RNA: AAUUUUUGGAG (SEQ ID NO: 22). In some embodiments, an RNA payload may comprise a guide RNA, a U7 hairpin sequence (e.g., a human or a mouse U7 hairpin sequence), an SmOPT sequence, or a combination thereof. For example, an RNA payload may comprise a sequence of AATTTTTGGAGCAGGTTTTCTGACTTCGGTCGGAAAACCCCTCCCAATTTCACTGGT CTACAATGAAAGCAAAACAGTTCTCTTCCCCGCTCCCCGGTGTGTGAGAGGGGCTTT GATCCTTCTCTGGTTTCCTAGGAAACGCGTATGTG (SEQ ID NO: 23). In some cases, a combination of a U7 hairpin sequence and a SmOPT sequence can comprise a SmOPT U7 hairpin sequence, wherein the SmOPT sequence is linked to the U7 sequence. An expression cassette or polynucleotide as described herein may comprise one or more sequences comprising an SmOPT sequence linked to a U7 sequence. For example, an expression cassette or polynucleotide as described herein may comprise two sequences each comprising an SmOPT sequence linked to a U7 sequence. In some cases, a U7 hairpin sequence, an SmOPT sequence, or a combination thereof is downstream (e.g., 3′) of the engineered guide RNA disclosed herein.

### Guide RNA Payloads for DNA Editing

The vectors comprising a plurality of expression cassettes described herein may be used to enhance expression of RNA components for site-specific, selective editing of a target DNA via a DNA editing entity or a biologically active fragment thereof. An RNA component for site-specific DNA editing may comprise a guide RNA, a transactivating CRISPR RNA (tracrRNA), a single guide RNA, or engineered polynucleotides encoding the same. An engineered guide RNA, as described herein, may comprise a sequence with complementarity to a target DNA described herein. As such, a guide RNA can be engineered to site-specifically/selectively target and hybridize to a particular target DNA, thus facilitating editing of specific nucleotide in the target DNA via a DNA editing entity or a biologically active fragment thereof. DNA editing may be facilitated by a nuclease, such as a Cas nuclease. In some embodiments, the Cas nuclease may be a Cas9, a Cas12, or a Cas14.

In some embodiments, an engineered guide RNA hybridizes to a sequence of the target DNA. In some embodiments, part of the engineered guide RNA hybridizes to the sequence of the target DNA. The part of the engineered guide RNA that hybridizes to the target DNA is of sufficient complementary to the sequence of the target DNA for hybridization to occur. In some embodiments, the guide RNA may comprise a sequence having at least about 95%, at least about 96%, at least about 97%, at least about 98%, at least about 99%, or about 100% sequence complementarity to a target DNA. A guide RNA encoded by an expression cassette of the present disclosure may comprise a length of from about 15 to about 70 nucleotides, from about 40 to about 70 nucleotides, or from about 70 to about 100 nucleotides. In some embodiments, the region of the guide RNA that hybridizes to the target may comprise a length of from about 18 to about 44 nucleotides.

In some examples, an engineered guide RNA can facilitate editing of a base of a nucleotide of in a target sequence of a target DNA that results in modulating the expression of a gene encoded by the target DNA. In some instances, modulation can be increased or decrease expression of the gene. In some cases, an engineered guide can be configured to facilitate an editing of a base of a nucleotide or polynucleotide of a region of an DNA by a DNA editing entity (e.g., a Cas nuclease).

In some embodiments, the vectors comprising a plurality of expression cassettes described herein may be used to enhance expression of transactivating crRNAs (tracrRNAs) and engineered polynucleotides encoding the same for editing of a target DNA via a DNA editing entity or a biologically active fragment thereof. The tracrRNA may bind to and activate a DNA editing enzyme (e.g., a Cas nuclease). A tracrRNA encoded by an expression cassette of a vector of the present disclosure may comprise a length of from about 75 to about 100 nucleotides.

In some embodiments, the expression cassettes described herein may be used to enhance expression of a single guide RNA and engineered polynucleotides encoding the same for editing of a target DNA via a DNA editing entity or a biologically active fragment thereof. The single guide RNA may comprise a region that binds to and activates a DNA editing enzyme (e.g., a Cas nuclease) and a region that hybridizes to the sequence of the target DNA. The part of the single guide RNA that hybridizes to the target DNA is of sufficient complementary to the sequence of the target DNA for hybridization to occur. In some embodiments, the single guide RNA may comprise a sequence having at least about 95%, at least about 96%, at least about 97%, at least about 98%, at least about 99%, or about 100% sequence complementarity to a target DNA. A single guide RNA encoded by an expression cassette of the present disclosure may comprise a length of from about 80 to about 120 nucleotides. In some embodiments, the region of the single guide RNA that hybridizes to the target may comprise a length of from about 18 to about 44 nucleotides.

### Other RNA-Targeting Oligonucleotides

The vectors comprising a plurality of expression cassettes described herein may be used to enhance expression of other engineered RNA-targeting oligonucleotides, including antisense oligonucleotides, siRNAs, shRNAs, and miRNAs, and engineered polynucleotides encoding the same that hybridizes to a target RNA (e.g., a target mRNA or a target pre-mRNA). An engineered oligonucleotide, as described herein, may comprise a targeting domain with complementarity to a target RNA described herein. As such, an oligonucleotide can be engineered to target and hybridize to a particular target RNA, thus altering expression of a polypeptide encoded by the target RNA.

In some embodiments, the engineered oligonucleotide (e.g., antisense oligonucleotide, siRNA, shRNA, or miRNA) of the present disclosure hybridizes to a sequence of the target RNA. In some embodiments, part of the engineered oligonucleotide (e.g., a targeting domain) hybridizes to the sequence of the target RNA. The part of the engineered oligonucleotide that hybridizes to the target RNA is of sufficient complementary to the sequence of the target RNA for hybridization to occur. A targeting sequence can also be referred to as a “targeting domain” or a “targeting region.” In some embodiments, binding of the engineered oligonucleotide to the target RNA may recruit additional components, such as RISC components.

In some cases, a targeting domain of an engineered oligonucleotide allows the engineered oligonucleotide to target an RNA sequence through base pairing, such as Watson Crick base pairing. In some examples, the targeting sequence can be located at either the N-terminus or C-terminus of the engineered oligonucleotide. In some cases, the targeting sequence can be located at both termini. The targeting sequence can be of any length. In some cases, the targeting sequence can be at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In some cases, the targeting sequence can be no greater than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or 200 nucleotides in length. In some examples, an engineered oligonucleotide comprises a targeting sequence that can be from about 60 to about 500, from about 60 to about 200, from about 75 to about 100, from about 80 to about 200, from about 90 to about 120, or from about 95 to about 115 nucleotides in length. In some examples, an engineered oligonucleotide comprises a targeting sequence that can be about 100 nucleotides in length.

In some cases, a targeting domain comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence can have a single base mismatch.

The targeting sequence can have sufficient complementarity to a target RNA to allow for hybridization of the targeting sequence to the target RNA. In some embodiments, the targeting sequence has a minimum antisense complementarity of about 50 nucleotides or more to the target RNA. In some embodiments, the targeting sequence has a minimum antisense complementarity of about 60 nucleotides or more to the target RNA. In some embodiments, the targeting sequence has a minimum antisense complementarity of about 70 nucleotides or more to the target RNA. In some embodiments, the targeting sequence has a minimum antisense complementarity of about 80 nucleotides or more to the target RNA. In some embodiments, the targeting sequence has a minimum antisense complementarity of about 90 nucleotides or more to the target RNA. In some embodiments, the targeting sequence has a minimum antisense complementarity of about 100 nucleotides or more to the target RNA. In some embodiments, antisense complementarity refers to non-contiguous stretches of sequence. In some embodiments, antisense complementarity refers to contiguous stretches of sequence.

### Chemical Modifications

An engineered guide RNA as described herein for use in treating a disease or condition in a subject can comprise at least one chemical modification. In some embodiments, the engineered guide RNA can comprise at least one, two, three, four, five, six, seven, eight, nine, ten, 11, 12, 13, 14, 15, 16, 17, 18, 19, 20, 30, 50, 100, or more chemical modifications. In some embodiments, the engineered guide RNA described herein may not comprise a chemical modification. In some cases, the engineered guide RNAs disclosed herein with barbell macro-footprints can be manufactured, chemically modified, and delivered directly to a subject in need thereof as RNA (without a vector, such as an AAV).

Exemplary chemical modifications comprise any one of: 5′ adenylate, 5′ guanosine-triphosphate cap, 5′ N7-Methylguanosine-triphosphate cap, 5′ triphosphate cap, 3′ phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′ deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′ fluoro RNA, 2′ O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3phosphorothioate or any combinations thereof.

A chemical modification can be made at any location of the engineered guide RNA. In some cases, a modification may be located in a 5′ or 3′ end, or both. In some cases, a polynucleotide can comprise a modification at a base selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150. In some cases, more than one modification can be made to the engineered guide RNA. In some cases, a modification can be permanent. In other cases, a modification can be transient. In some cases, multiple modifications may be made to the engineered guide RNA. The engineered guide RNA modification can alter physio-chemical properties of a nucleotide, such as their conformation, polarity, hydrophobicity, chemical reactivity, base-pairing interactions, or any combination thereof.

In some embodiments, a chemical modification can also be a phosphorothioate substitute. In some cases, a natural phosphodiester bond can be susceptible to rapid degradation by cellular nucleases and a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases may be of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

In some embodiments, a chemical modification can occur at 3′OH, group, 5′OH group, at the backbone, at the sugar component, or at the nucleotide base. Chemical modification can include non-naturally occurring linker molecules of interstrand or intrastrand cross links. In one aspect, the chemically modified nucleic acid comprises modification of one or more of the 3′OH or 5′OH group, the backbone, the sugar component, or the nucleotide base, or addition of non-naturally occurring linker molecules. In some embodiments, a chemically modified backbone comprises a backbone other than a phosphodiester backbone. In some embodiments, a modified sugar comprises a sugar other than deoxyribose (in modified DNA) or other than ribose (modified RNA). In some embodiments, a modified base comprises a base other than adenine, guanine, cytosine, thymine or uracil. In some embodiments, the engineered guide RNA comprises at least one chemically modified base. In some instances, an engineered guide RNA can comprise 2, 3, 4, 5, 6, 7, 8, 9, 10, 15, 20, or more modified bases. In some cases, chemical modifications to the base moiety include natural and synthetic modifications of adenine, guanine, cytosine, thymine, or uracil, and purine or pyrimidine bases.

In some embodiments, a chemical modification of the engineered guide RNA can comprise a modification of any one of or any combination of: modification of one or both of the non-linking phosphate oxygens in the phosphodiester backbone linkage; modification of one or more of the linking phosphate oxygens in the phosphodiester backbone linkage; modification of a constituent of the ribose sugar; replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Chemical modifications to the engineered guide RNA include any modification contained herein, while some exemplary modifications are recited in TABLE 3.

TABLE 3
Exemplary Chemical Modification

| Modification of | |
|---|---|
| engineered guide RNA | Examples |
| Modification of one or | sulfur (S), selenium (Se), BR3 (wherein R can be, e.g., |
| both of the non-linking | hydrogen, alkyl, or aryl), C (e.g., an alkyl group, an aryl group, |
| phosphate oxygens in the | and the like), H, NR2, wherein R can be, e.g., hydrogen, alkyl, |
| phosphodiester backbone | or aryl, or wherein R can be, e.g., alkyl or aryl |
| linkage | |
| Modification of one or | sulfur (S), selenium (Se), BR3 (wherein R can be, e.g., |
| more of the linking | hydrogen, alkyl, or aryl), C (e.g., an alkyl group, an aryl group, |
| phosphate oxygens in the | and the like), H, NR2, wherein R can be, e.g., hydrogen, alkyl, |
| phosphodiester backbone | or aryl, or wherein R can be, e.g., alkyl or aryl |
| linkage | |
| Replacement of the | methyl phosphonate, hydroxylamino, siloxane, carbonate, |
| phosphate moiety with | carboxymethyl, carbamate, amide, thioether, ethylene oxide |
| “dephospho” linkers | linker, sulfonate, sulfonamide, thioformacetal, formacetal, |
| | oxime, methyleneimino, methylenemethylimino, |
| | methylenehydrazo, methylenedimethylhydrazo, or |
| | methyleneoxymethylimino |
| Modification or | Nucleic acid analog (examples of nucleotide analogs can be |
| replacement of a naturally | found in PCT/US2015/025175, PCT/US2014/050423, |
| occurring nucleobase | PCT/US2016/067353, PCT/US2018/041503, |
| | PCT/US18/041509, PCT/US2004/011786, or |
| | PCT/US2004/011833, all of which are expressly incorporated |
| | by reference in their entireties |
| Modification of the ribose- | phosphorothioate, phosphonothioacetate, phosphoroselenates, |
| phosphate backbone | boranophosphates, borano phosphate esters, hydrogen |
| | phosphonates, phosphonocarboxylate, phosphoroamidates, alkyl |
| | or aryl phosphonates, phosphonoacetate, or phosphotriesters |
| Modification of 5′ end | 5′ cap or modification of 5′ cap —OH |
| of polynucleotide | |
| Modification of 3′ end | 3′ tail or modification of 3′ end —OH |
| of polynucleotide | |
| Modification of the | phosphorothioate, phosphonothioacetate, phosphoroselenates, |
| deoxyribose phosphate | borano phosphates, borano phosphate esters, hydrogen |
| backbone | phosphonates, phosphoroamidates, alkyl or aryl phosphonates, |
| | or phosphotriesters |
| Substitution of the | methyl phosphonate, hydroxylamino, siloxane, carbonate, |
| phosphate group | carboxymethyl, carbamate, amide, thioether, ethylene oxide |
| | linker, sulfonate, sulfonamide, thioformacetal, formacetal, |
| | oxime, methyleneimino, methylenemethylimino, |
| | methylenehydrazo, methylenedimethylhydrazo, or |
| | methyleneoxymethylimino. |
| Modification of the | morpholino, cyclobutyl, pyrrolidine, or peptide nucleic acid |
| ribophosphate backbone | (PNA) nucleoside surrogates |
| Modifications to the | Locked nucleic acid (LNA), unlocked nucleic acid (UNA), or |
| sugar of a nucleotide | bridged nucleic acid (BNA) |
| Modification of a | 2′-O-methyl, 2′-O-methoxy-ethyl (2′-MOE), 2′-fluoro, |
| constituent of the | 2′-aminoethyl, 2′-deoxy-2′-fuloarabinou-cleic acid, |
| ribose sugar | 2′-deoxy, 2′-O-methyl, 3′-phosphorothioate, |
| | 3′-phosphonoacetate (PACE), or 3′-phosphonothioacetate (thioPACE) |
| Modifications to the base | Modification of A, T, C, G, or U |
| of a nucleotide | |
| Stereopure of nucleotide | S conformation of phosphorothioate or R conformation of |
| | phosphorothioate |

### Modification of the Phosphate Backbone

In some embodiments, the chemical modification can comprise modification of one or both of the non-linking phosphate oxygens in the phosphodiester backbone linkage or modification of one or more of the linking phosphate oxygens in the phosphodiester backbone linkage. As used herein, “alkyl” may be meant to refer to a saturated hydrocarbon group which may be straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” may refer to monocyclic or polycyclic (e.g., having 2, 3, or 4 fused rings) aromatic hydrocarbons such as, for example, phenyl, naphthyl, anthracenyl, phenanthrenyl, indanyl, or indenyl. In some embodiments, aryl groups have from 6 to about 20 carbon atoms. As used herein, “alkenyl” may refer to an aliphatic group containing at least one double bond. As used herein, “alkynyl” may refer to a straight or branched hydrocarbon chain containing 2-12 carbon atoms and characterized in having one or more triple bonds. Examples of alkynyl groups can include ethynyl, propargyl, or 3-hexynyl. “Arylalkyl” or “aralkyl” may refer to an alkyl moiety in which an alkyl hydrogen atom may be replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. “Cycloalkyl” may refer to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl. “Heterocyclyl” may refer to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” may refer to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some embodiments, the phosphate group of a chemically modified nucleotide can be modified by replacing one or more of the oxygens with a different substituent. In some embodiments, the chemically modified nucleotide can include replacement of an unmodified phosphate moiety with a modified phosphate as described herein. In some embodiments, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur(S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way may be a stereogenic center. The stereogenic phosphorous atom can possess either the “R” configuration (herein Rp) or the “S” configuration (herein Sp). In some cases, the engineered guide RNA can comprise stereopure nucleotides comprising S conformation of phosphorothioate or R conformation of phosphorothioate. In some embodiments, the chiral phosphate product may be present in a diastereomeric excess of 50%, 60%, 70%, 80%, 90%, or more. In some embodiments, the chiral phosphate product may be present in a diastereomeric excess of 95%. In some embodiments, the chiral phosphate product may be present in a diastereomeric excess of 96%. In some embodiments, the chiral phosphate product may be present in a diastereomeric excess of 97%. In some embodiments, the chiral phosphate product may be present in a diastereomeric excess of 98%. In some embodiments, the chiral phosphate product may be present in a diastereomeric excess of 99%. In some embodiments, both non-bridging oxygens of phosphorodithioates can be replaced by sulfur. The phosphorus center in the phosphorodithioates can be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl). In some embodiments, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). In some cases, the replacement can occur at either or both of the linking oxygens.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

In some cases, backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) may be attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene (methylimino) linkages; formacetal and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g., a combination of phosphate linkages such as a combination of phosphodiester and phosphorothioate linkages.

In some cases, substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts. It may be also understood in a nucleotide substitute that both the sugar and the phosphate moieties of the nucleotide can be replaced, by for example an amide type linkage (aminoethylglycine) (PNA). It may be also possible to link other types of molecules (conjugates) to nucleotides or nucleotide analogs to enhance for example, cellular uptake. In some cases, conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some embodiments, a chemical modification described herein can comprise modification of a phosphate backbone. In some embodiments, the engineered guide RNA described herein can comprise at least one chemically modified phosphate backbone. Exemplary chemically modification of the phosphate group or backbone can include replacing one or more of the oxygens with a different substituent. Furthermore, the modified nucleotide present in the engineered guide RNA can include the replacement of an unmodified phosphate moiety with a modified phosphate as described herein. In some embodiments, the modification of the phosphate backbone can include alterations resulting in either an uncharged linker or a charged linker with unsymmetrical charge distribution. Exemplary modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur(S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or OR (wherein R can be, e.g., alkyl or aryl). The phosphorous atom in an unmodified phosphate group may be achiral. However, replacement of one of the non-bridging oxygens with one of the above atoms or groups of atoms can render the phosphorous atom chiral; that may be to say that a phosphorous atom in a phosphate group modified in this way may be a stereogenic center. The stereogenic phosphorous atom can possess either the “R” configuration (herein Rp) or the “S” configuration (herein Sp). In such case, the chemically modified engineered guide RNA can be stereopure (e.g., S or R confirmation). In some cases, a chemically modified engineered guide RNA comprises stereopure phosphate modification. For example, the chemically modified engineered guide RNA can comprise S conformation of phosphorothioate or R conformation of phosphorothioate.

Phosphorodithioates have both non-bridging oxygens replaced by sulfur. The phosphorus center in the phosphorodithioates may be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

In some cases, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of Phosphate Moiety

In some embodiments, at least one phosphate group of the engineered guide RNA can be chemically modified. In some embodiments, the phosphate group can be replaced by non-phosphorus containing connectors. In some embodiments, the phosphate moiety can be replaced by dephospho linker. In some embodiments, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In some embodiments, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g., 3′-amino phosphoramidate and aminoalkylphosphoramidates), thionophosphoramidates, thionoalkylphosphonates, thionoalkylphosphotriesters, and boranophosphates. In some cases, the phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

### Substitution of Phosphate Group

In some embodiments, a chemical modification described herein can comprise modification by replacement of a phosphate group. In some embodiments, the engineered guide RNA described herein can comprise at least one chemically modification comprising a phosphate group substitution or replacement. Exemplary phosphate group replacement can include non-phosphorus containing connectors. In some embodiments, the phosphate group substitution or replacement can include replacing charged phosphate group can by a neutral moiety. Exemplary moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

### Modification of the Ribophosphate Backbone

In some embodiments, the chemical modification described herein can comprise modifying ribophosphate backbone of the engineered guide RNA. In some embodiments, the engineered guide RNA described herein can comprise at least one chemically modified ribophosphate backbone. Exemplary chemically modified ribophosphate backbone can include scaffolds that can mimic nucleic acids can also be constructed wherein the phosphate linker and ribose sugar may be replaced by nuclease resistant nucleoside or nucleotide surrogates. In some embodiments, the nucleobases can be tethered by a surrogate backbone. Examples can include morpholino, cyclobutyl, pyrrolidine and peptide nucleic acid (PNA) nucleoside surrogates.

### Modification of Sugar

In some embodiments, the chemical modification described herein can comprise modifying of sugar. In some embodiments, the engineered guide RNA described herein can comprise at least one chemically modified sugar. Exemplary chemically modified sugar can include 2′ hydroxyl group (OH) modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR, wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2′ hydroxyl group modification can include (LNA, in which the 2′ hydroxyl can be connected, e.g., by a Ci-₆ alkylene or Cj-6 heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the “oxy”-2′ hydroxyl group modification can include the methoxyethyl group (MOE), (OCH₂CH₂OCH3, e.g., a PEG derivative). In some cases, the deoxy modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the I position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that may be in the L form, e.g., L-nucleosides. In some aspects, the engineered guide RNA described herein includes the sugar group ribose, which may be a 5-membered ring having an oxygen. Exemplary modified nucleosides and modified nucleotides can include replacement of the oxygen in ribose (e.g., with sulfur(S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose may be replaced by glycol units attached to phosphodiester bonds), threose nucleic acid. In some embodiments, the modifications to the sugar of the engineered guide RNA comprises modifying the engineered guide RNA to include locked nucleic acid (LNA), unlocked nucleic acid (UNA), or bridged nucleic acid (BNA).

### Modification of a Constituent of the Ribose Sugar

In some embodiments, the engineered guide RNA described herein can comprise at least one chemical modification of a constituent of the ribose sugar. In some embodiments, the chemical modification of the constituent of the ribose sugar can include 2′-O-methyl, 2′-O-methoxy-ethyl(2′-MOE), 2′-fluoro, 2′-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE). In some embodiments, the chemical modification of the constituent of the ribose sugar comprises unnatural nucleic acid. In some instances, the unnatural nucleic acids include modifications at the 5′-position and the 2′-position of the sugar ring, such as 5′—CH₂-substituted 2′-O-protected nucleosides. In some cases, unnatural nucleic acids include amide linked nucleoside dimers that can be prepared for incorporation into oligonucleotides. In some cases, the 3′ linked nucleoside in the dimer (5′ to 3′) comprises a 2′-OCH₃ and a 5′-(S)—CH₃. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position may be unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some embodiments, unnatural nucleic acids also include modifications of the sugar moiety. In some cases, nucleic acids can contain one or more nucleosides wherein the sugar group has been modified. Such sugar modified nucleosides may impart enhanced nuclease stability, increased binding affinity, or some other beneficial biological property. In certain embodiments, nucleic acids can comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R₁)(R₂) (R═H, C₁-C₁₂ alkyl or a protecting group); and combinations thereof.

In some instances, the engineered guide RNA described herein can comprise modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

In some cases, modifications to the sugar moiety include natural modifications of the ribose and deoxy ribose as well as unnatural modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl, 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)n CH₃)]₂, where n and m may be from 1 to about 10. Other chemical modifications at the 2′ position include but are not limited to: C1 to C10 lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′—OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein can include one or more bicyclic nucleic acids. In certain such embodiments, the bicyclic nucleic acid comprises a bridge between the 4′ and the 2′ ribosyl ring atoms. In certain embodiments, nucleic acids provided herein can include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof; 4′-C(CH₃)(CH₃)—O-2′ and analogs thereof.

### Modifications on the Base of Nucleotide

In some embodiments, the chemical modification described herein can comprise modification of the base of nucleotide (e.g., the nucleobase). Exemplary nucleobases can include adenine (A), thymine (T), guanine (G), cytosine (C), and uracil (U). These nucleobases can be modified or replaced to in the engineered guide RNA described herein. The nucleobase of the nucleotide can be independently selected from a purine, a pyrimidine, a purine or pyrimidine analog. In some embodiments, the nucleobase can be naturally-occurring or synthetic derivatives of a base.

In some embodiments, the chemical modification described herein can comprise modifying an uracil. In some embodiments, the engineered guide RNA described herein can comprise at least one chemically modified uracil. Exemplary chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine, 1 methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethyl)-2-thio-uridine, a-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino) uridine, pyrazolo[3,4-d]pyrimidines, xanthine, and hypoxanthine.

In some embodiments, the chemical modification described herein can comprise modifying a cytosine. In some embodiments, the engineered guide RNA described herein can comprise at least one chemically modified cytosine. Exemplary chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, a-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some embodiments, the chemical modification described herein can comprise modifying an adenine. In some embodiments, the engineered guide RNA described herein can comprise at least one chemically modified adenine. Exemplary chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N6, N6-dimethyl-adenosine, N6-hydroxynorvalylcarbamoyl-adenosine, 2-methylthio-N6-hydroxynorvalylcarbamoyl-adenosine, N6-acetyl-adenosine, 7-methyl-adenine, 2-methylthio-adenine, 2-methoxy-adenine, a-thio-adenosine, 2′-O-methyl-adenosine, N6, 2′-O-dimethyl-adenosine, N6-Methyl-2′-deoxyadenosine, N6, N6, 2′-O-trimethyl-adenosine, 1,2′-O-dimethyl-adenosine, 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the chemical modification described herein can comprise modifying a guanine. In some embodiments, the engineered guide RNA described herein can comprise at least one chemically modified guanine. Exemplary chemically modified guanine can include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, a-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2, 7-dimethyl-2′-O-methyl-guanosine, 2′-O-methyl-inosine, 1, 2′-O-dimethyl-inosine, 6-O-phenyl-2′-deoxyinosine, 2′-O-ribosylguanosine, 1-thio-guanosine, 6-O-methylguanosine, 06-Methyl-2′-deoxyguanosine, 2′-F-ara-guanosine, and 2′-F-guanosine.

In some cases, the chemical modification of the engineered guide RNA can include introducing or substituting a nucleic acid analog or an unnatural nucleic acid into the engineered guide RNA. In some embodiments, nucleic acid analog can be any one of the chemically modified nucleic acid described herein. Exemplary nucleic acid analog can be found in PCT/US2021/034272, PCT/US2015/025175, PCT/US2014/050423, PCT/US2016/067353, PCT/US2018/041503, PCT/US18/041509, PCT/US2004/011786, or PCT/US2004/011833, all of which are expressly incorporated by reference in their entireties. In some cases, the chemically modified nucleotide described herein can include a variant of guanosine, uridine, adenosine, thymidine, and cytosine, including any natively occurring or non-natively occurring guanosine, uridine, adenosine, thymidine or cytidine that has been altered chemically, for example by acetylation, methylation, hydroxylation. Exemplary chemically modified nucleotide can include 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2,-O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. In some embodiments, the chemically modified nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 6-methylguanosine-5′-triphosphate, pseudouridine-5′-triphosphate, puromycin-5′-triphosphate, or xanthosine-5′-triphosphate. In some embodiments, the chemically modified nucleic acid as described herein can comprise at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the chemically modified nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, the chemically modified nucleic acid as described herein can comprise at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In certain embodiments, the chemically modified nucleic acid as described herein can comprise at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytidine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

In some embodiments, a modified base of a unnatural nucleic acid includes, but may be not limited to, uracil-5-yl, hypoxanthine-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, O-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and O-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine ([5,4-b][1,4]benzoxazin-2 (3H)-one), phenothiazine cytidine (1H-pyrimido[5,4-b][1,4]benzothiazin-2 (3H)-one), G-clamps, phenoxazine cytidine (e.g. 9-(2-aminoethoxy)-H-pyrimido[5,4-b][1,4]benzoxazin-2 (3H)-one), carbazole cytidine (2H-pyrimido[4,5-b]indol-2-one), pyridoindole cytidine (H-pyrido[3′,2′: 4,5]pyrrolo[2,3-d]pyrimidin-2-one), those in which the purine or pyrimidine base may be replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, or 2-amino-2′-deoxyadenosine.

In some cases, the at least one chemical modification can comprise chemically modifying the 5′ or 3′ end such as 5′ cap or 3′ tail of the engineered guide RNA. In some embodiments, the engineered guide RNA can comprise a chemical modification comprising 3′ nucleotides which can be stabilized against degradation, e.g., by incorporating one or more of the modified nucleotides described herein. In this embodiment, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some embodiments, deaza nucleotides, e.g., 7-deaza-adenosine, can be incorporated into the gRNA. In some embodiments, O- and N-alkylated nucleotides, e.g., N6-methyladenosine, can be incorporated into the gRNA. In some embodiments, sugar-modified ribonucleotides can be incorporated, e.g., wherein the 2′ OH-group may be replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate group. In some embodiments, the nucleotides in the overhang region of the gRNA can each independently be a modified or unmodified nucleotide including, but not limited to 2′-sugar modified, such as, 2-F 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), or any combinations thereof.

### Therapeutic Applications

The vectors comprising one or more expression cassettes of the present disclosure encoding an RNA payload under transcriptional control of an engineered promoter may have a variety of therapeutic applications. The engineered promoters described herein may facilitate the therapeutic use by increasing payload expression and enhancing a therapeutic effect produced by the payload. For example, increased guide RNA payload expression may enhance editing efficiency of a target DNA or RNA. In another example, increased antisense oligonucleotide expression may enhance target knockdown efficiency.

### RNA Editing

RNA editing can refer to a process by which RNA can be enzymatically modified post synthesis at specific nucleosides. RNA editing can comprise any one of an insertion, deletion, or substitution of a nucleotide(s). Examples of RNA editing include chemical modifications, such as pseudouridylation (the isomerization of uridine residues) and deamination (removal of an amine group from: cytidine to give rise to uridine, or C-to-U editing; or from adenosine to inosine, or A-to-I editing). RNA editing can be used to correct mutations (e.g., correction of a missense mutation) to restore protein expression, or to introduce mutations or edit coding or non-coding regions of RNA to inhibit RNA translation and effect protein knockdown. A vector comprising a plurality of expression cassettes of the present disclosure may be used to express an engineered guide RNA to facilitate RNA editing by an RNA entity (e.g., an adenosine Deaminase Acting on RNA (ADAR)) or biologically active fragments thereof.

Described herein are engineered guide RNAs that facilitate RNA editing by an RNA editing entity (e.g., an adenosine Deaminase Acting on RNA (ADAR)) or biologically active fragments thereof. In some instances, ADARs can be enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets. In general, ADAR enzymes share a common domain architecture comprising a variable number of amino-terminal dsRNA binding domains (dsRBDs) and a single carboxy-terminal catalytic deaminase domain. Human ADARs possess two or three dsRBDs. Evidence suggests that ADARs can form homodimer as well as heterodimer with other ADARs when bound to double-stranded RNA, however it can be currently inconclusive if dimerization is needed for editing to occur. The engineered guide RNAs disclosed herein can facilitate RNA editing by any of or any combination of the three human ADAR genes that have been identified (ADARs 1-3). ADARs have a typical modular domain organization that includes at least two copies of a dsRNA binding domain (dsRBD; ADAR1 with three dsRBDs; ADAR2 and ADAR3 each with two dsRBDs) in their N-terminal region followed by a C-terminal deaminase domain.

The engineered guide RNAs of the present disclosure facilitate RNA editing by endogenous ADAR enzymes. In some embodiments, exogenous ADAR can be delivered alongside the engineered guide RNAs disclosed herein to facilitate RNA editing. In some embodiments, the ADAR is human ADAR1. In some embodiments, the ADAR is human ADAR2. In some embodiments, the ADAR is human ADAR3. In some embodiments, the ADAR is human ADAR1, human ADAR2, human ADAR2, or any combination thereof.

The present disclosure, in some embodiments, provides engineered guide RNAs that facilitate edits at particular regions in a target RNA (e.g., mRNA or pre-mRNA). For example, the engineered guide RNAs disclosed herein can target a coding sequence or a non-coding sequence of an RNA. For example, a target region in a coding sequence of an RNA can be a translation initiation site (TIS). In some embodiments, the target region in a non-coding sequence of an RNA can be a polyadenylation (polyA) signal sequence. In some embodiments, engineered guide RNAs of the present disclosure can target a target sequence encoding α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2). In some embodiments, the target sequence encodes α-synuclein (SNCA). In some embodiments, the target sequence encodes peripheral myelin protein 22 (PMP22). In some embodiments, the target sequence encodes double homeobox 4 (DUX4). In some embodiments, the target sequence encodes leucine rich repeat kinase 2 (LRRK2). In some embodiments, the target sequence encodes Tau (MAPT). In some embodiments, the target sequence encodes ATP-binding cassette sub-family A member 4 (ABCA4). In some embodiments, the target sequence encodes alpha-1 antitrypsin (SERPINA1). In some embodiments, the target sequence encodes methyl CpG binding protein 2 (MECP2).

### i) Missense Mutations

In some embodiments, the engineered guide RNAs of the present disclosure may target a missense mutation in a target RNA sequence. The engineered guide RNAs may facilitate ADAR-mediated RNA editing of a target adenosine (A) to convert to an inosine (I), which may be read as a guanosine (G). Conversion of A to I via ADAR-mediated RNA editing may correct G to A missense mutations. For example, ADAR-mediated editing may correct a valine to isoleucine or valine to methionine mutation by converting an isoleucine codon (AUU, AUC, or AUA) or methionine codon (AUG) to a valine codon (AUA, GUC, GUU, or GUG). In another example, ADAR-mediated editing may correct a cysteine to tyrosine or mutation by converting a tyrosine codon (AUA or UAC) to a cysteine codon (UGU or UGC). Alternatively, or in addition, the engineered guide RNAs may facilitate APOBEC-mediated RNA editing of a target cytosine (C) to convert to a uracil (U). Conversion of C to U via APOBEC-mediated RNA editing may correct U to C missense mutations. Engineered guide RNAs of the present disclosure can target one or any combination of missense mutations of a target sequence (e.g., SNCA, PMP22, DUX4, LRRK2, MAPT, GRN, ABCA4, APP, SERPINA1, HEXA, CFTR, LIPA, GBA, PINK1, or MECP2).

### ii) Nonsense Mutations

In some embodiments, the engineered guide RNAs of the present disclosure may target a nonsense mutation in a target RNA sequence. The engineered guide RNAs may facilitate ADAR-mediated RNA editing of a target adenosine (A) to convert to an inosine (I), which may be read as a guanosine (G). Conversion of A to I via ADAR-mediated RNA editing may correct G to A nonsense mutations. For example, ADAR-mediated editing may correct a tryptophan to stop nonsense mutation by converting a UAG stop codon to a tryptophan codon (UGG). In another example, ADAR-mediated editing may correct a tryptophan to stop nonsense mutation by converting a UGA stop codon to a tryptophan codon (UGG). Correction of nonsense mutations via ADAR-mediated editing may increase expression of the target sequence. Engineered guide RNAs of the present disclosure can target one or any combination of missense mutations of a target sequence (e.g., SNCA, PMP22, DUX4, LRRK2, MAPT, GRN, ABCA4, APP, SERPINA1, HEXA, CFTR, LIPA, GBA, PINK1, or MECP2).

iii) Translation Initiation Sites (TIS)

In some embodiments, the engineered guide RNAs of the present disclosure target the adenosine at a translation initiation site (TIS). The engineered guide RNAs may facilitate ADAR-mediated RNA editing of the TIS (AUG) to GUG. This results in inhibition of RNA translation and, thereby, protein knockdown. Protein knockdown can also be referred to as reduced expression of wild type protein. Engineered guide RNAs of the present disclosure can target one or any combination of the TISs of a target sequence (e.g., SNCA, PMP22, DUX4, LRRK2, MAPT, GRN, ABCA4, APP, SERPINA1, HEXA, CFTR, LIPA, GBA, PINK1, or MECP2).

### iv) 3′ Untranslated Region (3′UTR)

In some embodiments, the engineered guide RNAs of the present disclosure target one or more adenosines in the 3′ untranslated region (3′UTR). In some embodiments, an engineered guide RNA facilitates ADAR-mediated RNA editing of the one or more adenosines in the 3′UTR, thereby reducing mRNA export from the nucleus and inhibiting translation, thereby resulting protein knockdown. In some embodiments, the target sequence may be SNCA, PMP22, DUX4, LRRK2, MAPT, GRN, ABCA4, APP, SERPINA1, HEXA, CFTR, LIPA, GBA, PINK1, or MECP2.

### v) PolyA Signal Sequence

In some embodiments, the engineered guide RNAs of the present disclosure target one or more adenosines in the polyA signal sequence. In some embodiments, an engineered guide RNA facilitates ADAR-mediated RNA editing of the one or more adenosines in the polyA signal sequence, thereby resulting in disruption of RNA processing and degradation of the target mRNA and, thereby, protein knockdown. In some embodiments, a target can have one or more polyA signal sequences. In these instances, one or more engineered guide RNAs, varying in their respective sequences, of the present disclosure can be multiplexed to target adenosines in the one or more polyA signal sequences. In both cases, the engineered guide RNAs of the present disclosure facilitated ADAR-mediated RNA editing of adenosines to inosines (read as guanosines by cellular machinery) in the polyA signal sequence, resulting in protein knockdown. In some embodiments, the target sequence may be SNCA, PMP22, DUX4, LRRK2, MAPT, GRN, ABCA4, APP, SERPINA1, HEXA, CFTR, LIPA, GBA, PINK1, or MECP2.

DNA editing can refer to a process by which DNA can be enzymatically (e.g., by an RNA-guided endonuclease). DNA editing can comprise any one of an insertion, deletion, or substitution of a nucleotide(s). DNA editing can be used to correct mutations (e.g., correction of a missense mutation) to restore protein expression, or to introduce mutations or edit coding or non-coding regions of DNA to inhibit DNA transcription and effect protein knockdown. A vector comprising a plurality of expression cassettes of the present disclosure may be used to express an engineered guide RNA to facilitate DNA editing by a DNA entity (e.g., CRISPR/Cas endonuclease) or biologically active fragments thereof. Described herein are engineered guide RNAs that facilitate DNA editing by a DNA editing entity (e.g., CRISPR/Cas endonuclease) or biologically active fragments thereof.

The engineered guide RNAs of the present disclosure may facilitate DNA editing by endogenous Cas enzymes. In some embodiments, exogenous Cas enzymes can be delivered alongside the engineered guide RNAs disclosed herein to facilitate DNA editing. In some embodiments, the Cas nuclease is Cas9. In some embodiments, the Cas nuclease is Cas12. In some embodiments, the Cas nuclease is Cas14.

The present disclosure, in some embodiments, provides engineered guide RNAs that facilitate edits at particular regions in a target DNA. For example, the engineered guide RNAs disclosed herein can target a coding sequence or a non-coding sequence of a DNA.

An engineered guide RNA of the present disclosure may recruit a CRISPR/Cas endonuclease (e.g., a Cas9 nuclease) to form a ribonucleoprotein (RNP) complex that is targeted to a particular site in a target polynucleotide (e.g., a target DNA) via base pairing between the guide RNA and a target region within the target polynucleotide. The engineered guide RNA may include a targeting sequence that is complementary to a target site of the target polynucleotide. Thus, an engineered guide RNA forms a complex with a Cas nuclease, and the guide RNA provides sequence specificity to the RNP complex via the targeting sequence. Upon recruitment to the target polynucleotide, the Cas nuclease may site-specifically edit the target polynucleotide (e.g., the target DNA). In some embodiments, the target polynucleotide may encode SNCA, PMP22, DUX4, LRRK2, MAPT, GRN, ABCA4, APP, SERPINA1, HEXA, CFTR, LIPA, GBA, PINK1, or MECP2.

### Expression Knockdown

A vector comprising a plurality of expression cassettes of the present disclosure may be used to express an engineered RNA-targeting oligonucleotide (e.g., an antisense oligonucleotide, an siRNA, an shRNA, or a miRNA) to facilitate knockdown expression of the target RNA. In some embodiments, binding of the RNA-targeting oligonucleotide to the target RNA may recruit additional components (e.g., RISC complex components) to the target RNA that may reduce expression of a peptide encoded by the target RNA. For example, binding of an siRNA may recruit RISC and facilitate cleavage of the target RNA. In another example, binding of a miRNA or an shRNA may recruit RISC and inhibit translation of the target RNA. In some embodiments, the target RNA may encode SNCA, PMP22, DUX4, LRRK2, MAPT, GRN, ABCA4, APP, SERPINA1, HEXA, CFTR, LIPA, GBA, PINK1, or MECP2.

### Targets and Methods of Treatment

A small RNA payload, such as an engineered guide RNA, of the present disclosure can be used in a method of treating a disorder in a subject in need thereof. A disorder can be a disease, a condition, a genotype, a phenotype, or any state associated with an adverse effect. In some embodiments, treating a disorder can comprise preventing, slowing progression of, reversing, or alleviating symptoms of the disorder. A method of treating a disorder can comprise delivering an engineered polynucleotide encoding one or more engineered guide RNAs to a cell of a subject in need thereof and expressing an engineered guide RNA in the cell. In some embodiments, an engineered guide RNA of the present disclosure can be used to treat a genetic disorder (e.g., a Tauopathy such as AD, FTD, Parkinson's disease). In some embodiments, an engineered guide RNA of the present disclosure can be used to treat a condition associated with one or more mutations.

The present disclosure provides a composition comprising a polynucleotide comprising a plurality of expression cassette sequences as described herein for use as a medicament. In various aspects, the present disclosure provides a composition comprising a polynucleotide comprising a plurality of expression cassette sequences as described herein for use in a method of treating a disease or disorder. In various aspects, the present disclosure provides use of a substance or composition, wherein the substance or composition is a polynucleotide as described herein, a viral vector as described herein, or a pharmaceutical composition as described herein for the manufacture of a medicament for therapeutic applications.

The present disclosure provides for compositions of vectors comprising a plurality of expression cassettes encoding engineered payloads (e.g., engineered guide RNAs) and methods of use thereof, such as methods of treatment. In some embodiments, the vectors comprising a plurality of expression cassettes of the present disclosure encode guide RNAs targeting a coding sequence of an RNA (e.g., an RNA encoding α-synuclein, PMP22, DUX4, LRRK2, tau, progranulin, ABCA4, amyloid precursor protein, or alpha-1 antitrypsin). In some embodiments, the engineered polynucleotides of the present disclosure encode guide RNAs targeting a non-coding sequence of an RNA (e.g., a polyA sequence). In some embodiments, the present disclosure provides compositions of one or more than one engineered polynucleotides encoding more than one engineered guide RNAs targeting the TIS, the polyA sequence, or any other part of a coding sequence or non-coding sequence. The engineered guide RNAs disclosed herein facilitate ADAR-mediated RNA editing of adenosines in the TIS, the polyA sequence, any part of a coding sequence of an RNA, any part of a non-coding sequence of an RNA, or any combination thereof.

Examples of target genes that may be targeted by engineered RNA payloads encoded by the vectors comprising a plurality of expression cassettes of the present disclosure are provided in TABLE 4. The target gene may be a wild type gene, or the target gene may be a mutated gene. Targeting the gene using an engineered RNA payload may treat a condition associated with the target gene.

TABLE 4
Exemplary Gene Targets and Associated Conditions

| Target | |
|---|---|
| Gene (Protein) | Associated Conditions |
| SNCA (α-synuclein) | Synucleinopathies, Parkinson's disease, |
| | Lewy body dementia, multiple system atrophy |
| PMP22 (peripheral | Charcot-Marie-Tooth disease, Hereditary |
| myelin protein 22) | neuropathy with liability to pressure palsies, |
| | Yuan-Harel-Lupski syndrome |
| MAPT (Tau) | Tauopathies, Alzheimer's disease frontotemporal |
| | dementia, Parkinson's disease, progressive |
| | supranuclear palsy, corticobasal degeneration, |
| | chronic traumatic encephalopathy, autism, |
| | traumatic brain injury, Dravet syndrome |
| LRRK2 (leucine rich repeat kinase 2) | Parkinson's disease, Crohn's disease |
| DUX4 (double homeobox 4) | Muscular dystrophy, B-cell leukemia |
| CMT1A (duplication of PMP22 | Charcot-Marie-Tooth disease, Dejerine-Sottas |
| associated with Charcot-Marie-Tooth | disease, hereditary neuropathy with liability |
| disease type 1A) | to pressure palsy |
| GRN (progranulin) | Frontotemporal dementia |
| ABCA4 (ATP-binding cassette sub- | Stargardt disease |
| family A member 4) | |
| APP (amyloid precursor protein) | Alzheimer's disease |
| SERPINA1 (alpha-1 antitrypsin) | Alpha-1 antitrypsin deficiency |
| HEXA (hexosaminidase A) | Tay-Sachs disease |
| CFTR (cystic fibrosis transmembrane | Cystic fibrosis |
| conductance regulator) | |
| LIPA (lipase A) | Liposomal acid lipase deficiency |
| GBA (glucosylceramidase beta) | Gaucher disease, Parkinson's disease |
| PINK1 (PTEN-induced kinase 1) | Parkinson's disease |
| MECP2 (methyl CpG binding protein 2) | Rett syndrome |

The vectors comprising a plurality of expression cassettes of the present disclosure may express payloads to target, modify, and/or express any sequence of interest. Select targets of interest that may be targeted by the payloads described herein for treatment of an associated condition are discussed below by way of example.

### MAPT

The present disclosure provides for vectors comprising a plurality of expression cassettes encoding engineered guide RNAs that facilitate RNA editing MAPT to knockdown expression of Tau protein. Tau pathology can be a key driver of a broad spectrum of neurodegenerative diseases, collectively known as Tauopathies. For example, diseases where Tau can play a primary role include, but are not limited to, Alzheimer's disease (AD), frontotemporal dementia (FTD), Parkinson's disease, progressive supranuclear palsy (PSP), corticobasal degeneration (CBD), and chronic traumatic encephalopathy. Tauopathies are characterized by the intracellular accumulation of neurofibrillary tangles (NFTs) composed of aggregated, misfolded Tau (MAPT gene). Thus, engineered guide RNAs of the present disclosure targeting MAPT RNA for ADAR-mediated editing to knockdown Tau protein can be capable of preventing or ameliorating disease progression in a number of diseases, including, but not limited to, AD, FTD, autism, traumatic brain injury, Parkinson's disease, and Dravet syndrome.

Thus, the engineered guide RNAs of the present disclosure can target MAPT for RNA editing, thereby, driving a reduction in Tau protein expression. In some embodiments, Tau protein expression is reduced in human neurons. In some embodiments, the present disclosure provides compositions of engineered guide RNAs that target MAPT and facilitated ADAR-mediated RNA editing of MAPT to reduce pathogenic levels of Tau by targeting key adenosines for deamination that are present in the translational initiation sites (TISs). In some embodiments, the engineered guide RNAs of the present disclosure target a coding sequence in MAPT. For example, the coding sequence can be a translation initiation site (TIS) (AUG) of MAPT, and the engineered guide RNA can facilitate ADAR-mediated RNA editing of AUG to GUG. Engineered guide RNAs of the present disclosure can target one or more of the TISs in MAPT to reduce or completely inhibit Tau protein expression.

For example, in some embodiments, an engineered guide RNA targets the AUG at the 18th nucleotide in Exon 1 (c.1, Nm_005910.5; GRCh37/Hg19; also referred to as “c.1” for coding nucleotide 1), referred to as the conventional TIS. In some embodiments, an engineered guide RNA targets the AUG at the 48th nucleotide in Exon 1 (c.31). In some embodiments, an engineered guide RNA targets the AUG at the 6th nucleotide in Exon 5 (c.379). With reference to the 2N4R Tau isoform containing 441 amino acids (Np_005901; GRCh37/Hg19), these three TISs correspond to methionines (Met) 1, 11 and 127, respectively. In some embodiments, an engineered guide RNA targets the AUG at the 108th nucleotide in Exon 1 (c.91). In some embodiments, one or more than one engineered guide RNAs of the present disclosure target any one or any combination of said four TISs. For example, a single engineered guide RNA of the present disclosure can be designed to target more than one of the above four TISs. In some embodiments, more than one engineered guide RNAs are designed to each independently target more than one of the above four TISs. In some embodiments, engineered guide RNAs of the present disclosure can target any one or any combination of the TISs in Exon 1 (c.1, c.31, and c.91). Targeting these sites in MAPT facilitate edits that result in inhibition of translation and a reduction in expression of the Tau protein. In some embodiments, the ratio of 3R to 4R isoforms of Tau can be measured by protein analysis (e.g., using an ELISA or flow cytometry) to evaluate the effect of RNA editing, with a 1 to 1 ratio representing the ratio in healthy adult brain. In some embodiments, any of the engineered guide RNAs disclosed herein are packaged in an AAV vector and are virally delivered.

In some embodiments, the engineered guide RNAs target a non-coding sequence in MAPT. The non-coding sequence can be a polyA signal sequence and the engineered guide RNA can facilitate ADAR-mediated RNA editing of one or more adenosines in the polyA signal sequence of MAPT. In some embodiments, engineered guide RNAs of the present disclosure can be multiplexed to target more than one polyA signal sequences in MAPT. In some embodiments, engineered guide RNAs of the present disclosure can be multiplexed to target the TIS and one or more polyA signal sequences in MAPT. In some embodiments, engineered guide RNAs can be multiplexed to target a non-coding sequence and a coding sequence in MAPT. The engineered guide RNAs of the present disclosure facilitated ADAR-mediated RNA editing of MAPT, thereby, effecting protein knockdown.

In some embodiments, the engineered guide RNAs of the present disclosure facilitated ADAR-mediated RNA editing of from 1 to 100% of a target adenosine. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of MAPT, which results in knockdown of protein levels. The knockdown in protein levels is quantitated as a reduction in expression of the Tau protein. The engineered guide RNAs of the present disclosure can facilitate from 1% to 100% Tau protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% Tau protein knockdown. In some embodiments, the engineered guide RNAs of the present disclosure facilitate from 30% to 60% Tau protein knockdown. Tau protein knockdown can be measured by an assay comparing a sample or subject treated with the engineered guide RNA to a control sample or subject not treated with the engineered guide RNA.

### α-Synuclein

The alpha-synuclein gene is made up of 5 exons and encodes a 140 amino-acid protein with a predicted molecular mass of ~14.5 kDa. The encoded product is an intrinsically disordered protein with unknown functions. Usually, Alpha-synuclein is a monomer. Under certain stress conditions or other unknown causes, α-synuclein self-aggregates into oligomers. Lewy-related pathology (LRP), primarily comprised of Alpha-synuclein in more than 50% of autopsy-confirmed Alzheimer's disease patients' brains. While the molecular mechanism of how Alpha-synuclein affects the development of Alzheimer's disease is unclear, experimental evidence has shown that Alpha-synuclein interacts with Tau-p and may seed the intracellular aggregation of Tau-p. Moreover, Alpha-synuclein could regulate the activity of GSK3B, which can mediate Tau-hyperphosphorylation. Alpha-synuclein can also self-assemble into pathogenic aggregates (Lewy bodies). Both Tau and α-synuclein can be released into the extracellular space and spread to other cells. Vascular abnormalities impair the supply of nutrients and removal of metabolic byproducts, cause microinfarcts, and promote the activation of glial cells. Therefore, a multiplex strategy to substantially reduce Tau formation, alpha-synuclein formation, or a combination thereof can be important in effectively treating neurodegenerative diseases.

The domain structure of Alpha-synuclein comprises an N-terminal A2 lipid-binding alpha-helix domain, a Non-amyloid β component (NAC) domain, and a C-terminal acidic domain. Molecularly, Alpha-synuclein is suggested to play a role in neuronal transmission and DNA repair. In some cases, a region of Alpha-synuclein can be targeted utilizing compositions provided herein. In some cases, a region of the Alpha-synuclein mRNA can be targeted with the engineered polynucleotides disclosed herein for knockdown. In some cases, a region of the exon or intron of the Alpha-synuclein mRNA can be targeted. In some embodiments, a region of the non-coding sequence of the Alpha-synuclein mRNA, such as the 5′UTR and 3′UTR, can be targeted. In other cases, a region of the coding sequence of the Alpha-synuclein mRNA can be targeted. Suitable regions include but are not limited to a N-terminal A2 lipid-binding alpha-helix domain, a non-amyloid β component (NAC) domain, or a C-terminal acidic domain.

In some aspects, an alpha-synuclein mRNA sequence is targeted. In some cases, any one of the 3,177 residues of the sequence may be targeted utilizing the compositions and method provided herein. In some cases, a target residue may be located among residues 1 to 100, from 99 to 200, from 199 to 300, from 299 to 400, from 399 to 500, from 499 to 600, from 599 to 700, from 699 to 800, from 799 to 900, from 899 to 1000, from 999 to 1100, from 1099 to 1200, from 1199 to 1300, from 1299 to 1400, from 1399 to 1500, from 1499 to 1600, from 1599 to 1700, from 1699 to 1800, from 1799 to 1900, from 1899 to 2000, from 1999 to 2100, from 2099 to 2200, from 2199 to 2300, from 2299 to 2400, from 2399 to 2500, from 2499 to 2600, from 2599 to 2700, from 2699 to 2800, from 2799 to 2900, from 2899 to 3000, from 2999 to 3100, from 3099 to 3177, or any combination thereof.

In some embodiments, the present disclosure provides vectors comprising a plurality of expression cassettes that may comprise an expression cassette encoding engineered guide RNAs that target SNCA. The engineered guide RNAs may target SNCA to modify or alter expression of SNCA. In some embodiments, targeting SNCA with the engineered guide RNAs of the present disclosure may treat a disease associated with SNCA, such as synucleinopathies, Parkinson's disease, Lewy body dementia, or multiple system atrophy. In some embodiments, the engineered guide RNAs may facilitate ADAR-mediated RNA editing of SNCA to correct G to A mutations by targeting adenosines for deamination. The engineered guide RNAs of the present disclosure may target a coding sequence in SNCA. For example, the coding sequence can be a translation initiation site (TIS) (AUG) of AUG, and the engineered guide RNA can facilitate ADAR-mediated RNA editing of AUG to GUG. Editing of the TIS may affect protein knockdown of SNCA. In another example, the guide RNA can facilitate ADAR-mediated correction of missense mutations in the coding sequence. Correcting a missense mutation may increase expression of functional SNCA protein. In another example, the guide RNA can facilitate ADAR-mediated correction of nonsense mutations in the coding sequence. Correcting a nonsense mutation may increase expression of SNCA protein.

In some embodiments, the engineered guide RNAs target a non-coding sequence in SNCA. The non-coding sequence can be a polyA signal sequence and the engineered guide RNA can facilitate ADAR-mediated RNA editing of one or more adenosines in the polyA signal sequence of SNCA. In some embodiments, engineered guide RNAs of the present disclosure can be multiplexed to target more than one polyA signal sequences in SNCA. In some embodiments, engineered guide RNAs of the present disclosure can be multiplexed to target the TIS and one or more polyA signal sequences in SNCA. In some embodiments, engineered guide RNAs can be multiplexed to target a non-coding sequence and a coding sequence in SNCA. The engineered guide RNAs of the present disclosure facilitated ADAR-mediated RNA editing of SNCA, thereby, affecting protein knockdown.

In some embodiments, the engineered guide RNAs of the present disclosure facilitated ADAR-mediated RNA editing of from 1 to 100% of a target adenosine in SNCA. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of SNCA, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of SNCA, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### PMP22

Peripheral myelin protein 22, encoded by PMP22, is involved in myelinating Schwann cells of the peripheral nervous system. Duplication or deletion of PMP22, and corresponding alteration of gene expression levels, is associated with a variety of diseases, including Charcot-Marie-Tooth type 1A (CMT1A), Dejerine-Sottas disease, and Hereditary Neuropathy with Liability to Pressure Palsy (HNPP). Described herein are methods of editing or modifying expression of PMP22 using a vector comprising a plurality of expression cassettes that may comprise an expression cassette encoding an engineered RNA payload to treat a disease (e.g., Charcot-Marie-Tooth disease, Dejerine-Sottas disease, or hereditary neuropathy).

In some embodiments, the present disclosure provides vectors comprising a plurality of expression cassettes that comprise an expression cassette encoding engineered guide RNAs that target PMP22. The engineered guide RNAs may target PMP22 to modify or alter expression of PMP22. In some embodiments, targeting PMP22 with the engineered guide RNAs of the present disclosure may treat a disease associated with PMP22, such as Charcot-Marie-Tooth disease, Dejerine-Sottas disease, or hereditary neuropathy. In some embodiments, the engineered guide RNAs may facilitate ADAR-mediated RNA editing of PMP22 to correct G to A mutations by targeting adenosines for deamination. The engineered guide RNAs of the present disclosure may target a coding sequence in PMP22. For example, the coding sequence can be a translation initiation site (TIS) (AUG) of AUG, and the engineered guide RNA can facilitate ADAR-mediated RNA editing of AUG to GUG. Editing of the TIS may affect protein knockdown of PMP22. In another example, the guide RNA can facilitate ADAR-mediated correction of missense mutations in the coding sequence. Correcting a missense mutation may increase expression of functional PMP22 protein. In another example, the guide RNA can facilitate ADAR-mediated correction of nonsense mutations in the coding sequence. Correcting a nonsense mutation may increase expression of PMP22 protein.

In some embodiments, the engineered guide RNAs target a non-coding sequence in PMP22. The non-coding sequence can be a polyA signal sequence and the engineered guide RNA can facilitate ADAR-mediated RNA editing of one or more adenosines in the polyA signal sequence of PMP22. In some embodiments, engineered guide RNAs of the present disclosure can be multiplexed to target more than one polyA signal sequences in PMP22. In some embodiments, engineered guide RNAs of the present disclosure can be multiplexed to target the TIS and one or more polyA signal sequences in PMP22. In some embodiments, engineered guide RNAs can be multiplexed to target a non-coding sequence and a coding sequence in PMP22. The engineered guide RNAs of the present disclosure facilitated ADAR-mediated RNA editing of PMP22, thereby, affecting protein knockdown.

In some embodiments, the engineered guide RNAs of the present disclosure facilitated ADAR-mediated RNA editing of from 1 to 100% of a target adenosine in PMP22. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of PMP22, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of PMP22, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### LRRK2

Leucine-rich repeat kinase 2 (LRRK2) has been associated with familial and sporadic cases of Parkinson's Disease and immune-related disorders like Crohn's disease. Its aliases include LRRK2, AURA17, DARDARIN, PARK8, RIPK7, ROCO2, or leucine-rich repeat kinase 2. The LRRK2 gene is made up of 51 exons and encodes a 2527 amino acid protein with a predicted molecular mass of about 286 kDa. The encoded product is a multi-domain protein with kinase and GTPase activities. LRRK2 can be found in various tissues and organs including but not limited to adrenal, appendix, bone marrow, brain, colon, duodenum, endometrium, esophagus, fat, gall bladder, heart, kidney, liver, lung, lymph node, ovary, pancreas, placenta, prostate, salivary gland, skin, small intestine, spleen, stomach, testis, thyroid, and urinary bladder. LRRK2 can be ubiquitously expressed but is generally more abundant in the brain, kidney, and lung tissue. Cellularly, LRRK2 has been found in astrocytes, endothelial cells, microglia, neurons, and peripheral immune cells.

Over 100 mutations have been identified in LRRK2; six of them (G2019S, R1441C/G/H, Y1699C, and I2020T) have been shown to cause Parkinson's Disease through segregation analysis. G2019S and R1441C are the most common disease-causing mutations in inherited cases. In sporadic cases, these mutations have shown age-dependent penetrance: The percentage of individuals carrying the G2019S mutation that develops the disease jumps from 17% to 85% when the age increases from 50 to 70 years old. In some cases, mutation carrying individuals never develop the disease.

At its catalytic core, LRRK2 contains the Ras of complex proteins (Roc), C-terminal of ROC (COR), and kinase domains. Multiple protein-protein interaction domains flank this core: an armadillo repeats (ARM) region, an ankyrin repeat (ANK) region, a leucine-rich repeat (LRR) domain are found in the N-terminus joined by a C-terminal WD40 domain. The G2019S mutation is located within the kinase domain. It has been shown to increase the kinase activity; for R1441C/G/H and Y1699C, these mutations can decrease the GTPase activity of the Roc domain. Genome-wide association study has found that common variations in LRRK2 increase the risk of developing sporadic Parkinson's Disease. While some of these variations are nonconservative mutations that affect the protein's binding or catalytic activities, others modulate its expression. These results suggest that specific alleles or haplotypes can regulate LRRK2 expression.

Pro-inflammatory signals upregulate LRRK2 expression in various immune cell types, suggesting that LRRK2 is a critical regulator in the immune response. Studies have found that both systemic and central nervous system (CNS) inflammation are involved in Parkinson's Disease's symptoms. Moreover, LRRK2 mutations associated with Parkinson's Disease modulate its expression levels in response to inflammatory stimuli. Many mutations in LRRK2 are associated with immune-related disorders such as inflammatory bowel disease such as Crohn's Disease. For example, both G2019S and N2081D increase LRRK2's kinase activity and are over-represented in Crohn's Disease patients in specific populations. Because of its critical role in these disorders, LRRK2 is an important therapeutic target for Parkinson's Disease and Crohn's Disease. In particular, many mutations, such as point mutations including G2019S, play roles in developing these diseases, making LRRK2 an attractive for therapeutic strategy such as RNA editing.

In some embodiments, the present disclosure provides vectors comprising a plurality of expression cassettes that may comprise an expression cassette encoding guide RNAs that are capable of facilitating RNA editing of LRRK2. In some embodiments, a guide RNA of the present disclosure can target the following mutations in LRRK2: E10L, A30P, S52F, E46K, A53T, L119P, A211V, C228S, E334K, N363S, V366M, A419V, R506Q, N544E, N551K, A716V, M712V, I723V, P755L, R793M, 1810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, or Q2490NfsX3. Said guide RNAs targeting a site in LRRK2 can be encoded by an engineered polynucleotide construct of the present disclosure.

In some examples, hybridization of a latent guide RNA targeting LRRK2 to a target LRRK2 mRNA produces a guide-target RNA scaffold that comprises a structural features selected from the group consisting of: (i) one or more X1/X2 bulges, wherein Xi is the number of nucleotides of the target RNA in the bulge and X2 is the number of nucleotides of the engineered guide RNA in the bulge, and wherein the one or more bulges is a 0/1 asymmetric bulge, a 2/2 symmetric bulge, a 3/3 symmetric bulge, or a 4/4 symmetric bulge; (ii) one or more X1/X2 internal loops, wherein Xi is the number of nucleotides of the target RNA in the internal loop and X2 is the number of nucleotides of the engineered guide RNA in the internal loop, and wherein the one or more internal loops is a 5/0 asymmetric internal loop, a 5/4 asymmetric internal loop, a 5/5 symmetric internal loop, a 6/6 symmetric internal loop, a 7/7 symmetric internal loop, or a 10/10 symmetric internal loop; (iii) one or more mismatches, wherein the one or more mismatches is an A/C mismatch, an A/G mismatch, a C/U mismatch, a G/A mismatch, or a C/C mismatch, (iv) a G/U wobble base pair or a U/G wobble base pair, and (v) any combination thereof. Said engineered guide RNAs can be delivered via viral vector (e.g., encoded for and delivered via AAV) as disclosed herein and can be administered via any route of administration disclosed herein to a subject in need thereof. The subject can be human and may be at risk of developing or has developed a disease or condition associated with mutations in LRRK2 (e.g., diseases of the central nervous system (CNS) or gastrointestinal (GI) tract). For example, such diseases of conditions can include Crohn's disease or Parkinson's disease. Such CNS or GI tract diseases (e.g., Crohn's disease or Parkinson's disease) can be at least partially caused by a mutation of LRRK2, for which an engineered guide RNA described herein can facilitate editing in, thus correcting the mutation in LRRK2 and reducing the incidence of the CNS or GI tract disease in the subject. Thus, the guide RNAs of the present disclosure can be used in a method of treatment of diseases such as Crohn's disease or Parkinson's disease.

In some embodiments, the present disclosure provides vectors comprising a plurality of expression cassettes that may comprise an expression cassette encoding engineered guide RNAs that target LRRK2. The engineered guide RNAs may target LRRK2 to modify or alter expression of LRRK2. In some embodiments, targeting LRRK2 with the engineered guide RNAs of the present disclosure may treat a disease associated with LRRK2, such as Parkinson's disease or Crohn's disease. In some embodiments, the engineered guide RNAs may facilitate ADAR-mediated RNA editing of LRRK2 to correct G to A mutations by targeting adenosines for deamination. The engineered guide RNAs of the present disclosure may target a coding sequence in LRRK2. For example, the coding sequence can be a translation initiation site (TIS) (AUG) of AUG, and the engineered guide RNA can facilitate ADAR-mediated RNA editing of AUG to GUG. Editing of the TIS may affect protein knockdown of LRRK2. In another example, the guide RNA can facilitate ADAR-mediated correction of missense mutations in the coding sequence. Correcting a missense mutation may increase expression of functional LRRK2 protein. In another example, the guide RNA can facilitate ADAR-mediated correction of nonsense mutations in the coding sequence. Correcting a nonsense mutation may increase expression of LRRK2 protein.

In some embodiments, the engineered guide RNAs target a non-coding sequence in LRRK2. The non-coding sequence can be a polyA signal sequence and the engineered guide RNA can facilitate ADAR-mediated RNA editing of one or more adenosines in the polyA signal sequence of LRRK2. In some embodiments, engineered guide RNAs of the present disclosure can be multiplexed to target more than one polyA signal sequences in LRRK2. In some embodiments, engineered guide RNAs of the present disclosure can be multiplexed to target the TIS and one or more polyA signal sequences in LRRK2. In some embodiments, engineered guide RNAs can be multiplexed to target a non-coding sequence and a coding sequence in LRRK2. The engineered guide RNAs of the present disclosure facilitated ADAR-mediated RNA editing of LRRK2, thereby, affecting protein knockdown.

In some embodiments, the engineered guide RNAs of the present disclosure facilitated ADAR-mediated RNA editing of from 1 to 100% of a target adenosine in LRRK2. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of LRRK2, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of LRRK2, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### DUX4

Double homeobox, 4 (DUX4) functions as a transcriptional activator of a variety of genes, including PITX1, and regulates expression of small RNAs in muscle cells. In some embodiments, overexpression of DUX4 can cause B-cell leukemia. Described herein are methods of editing or modifying expression of DUX4 using a vector comprising a plurality of expression cassettes that may comprise an expression cassette encoding an engineered RNA payload to treat a disease (e.g., B-cell leukemia or facioscapulohumeral muscular dystrophy).

In some embodiments, the present disclosure provides vectors comprising a plurality of expression cassettes that may comprise an expression cassette encoding engineered guide RNAs that target DUX4. The engineered guide RNAs may target DUX4 to modify or alter expression of DUX4. In some embodiments, targeting DUX4 with the engineered guide RNAs of the present disclosure may treat a disease associated with DUX4, such as B-cell leukemia or facioscapulohumeral muscular dystrophy. In some embodiments, the engineered guide RNAs may facilitate ADAR-mediated RNA editing of DUX4 to correct G to A mutations by targeting adenosines for deamination. The engineered guide RNAs of the present disclosure may target a coding sequence in DUX4. For example, the coding sequence can be a translation initiation site (TIS) (AUG) of AUG, and the engineered guide RNA can facilitate ADAR-mediated RNA editing of AUG to GUG. Editing of the TIS may affect protein knockdown of DUX4. In another example, the guide RNA can facilitate ADAR-mediated correction of missense mutations in the coding sequence. Correcting a missense mutation may increase expression of functional DUX4 protein. In another example, the guide RNA can facilitate ADAR-mediated correction of nonsense mutations in the coding sequence. Correcting a nonsense mutation may increase expression of DUX4 protein.

In some embodiments, the engineered guide RNAs target a non-coding sequence in DUX4. The non-coding sequence can be a polyA signal sequence and the engineered guide RNA can facilitate ADAR-mediated RNA editing of one or more adenosines in the polyA signal sequence of DUX4. In some embodiments, engineered guide RNAs of the present disclosure can be multiplexed to target more than one polyA signal sequences in DUX4. In some embodiments, engineered guide RNAs of the present disclosure can be multiplexed to target the TIS and one or more polyA signal sequences in DUX4. In some embodiments, engineered guide RNAs can be multiplexed to target a non-coding sequence and a coding sequence in DUX4. The engineered guide RNAs of the present disclosure facilitated ADAR-mediated RNA editing of DUX4, thereby, affecting protein knockdown.

In some embodiments, the engineered guide RNAs of the present disclosure facilitated ADAR-mediated RNA editing of from 1 to 100% of a target adenosine in DUX4. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of DUX4, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of DUX4, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### Progranulin

Progranulin, encoded by GRN, is a precursor protein cleaved to form granulin. GRN is expressed in peripheral and central nervous system tissues and is upregulated in microglia following injury. Both granulin and progranulin are implicated in a wide variety of functions, including development, inflammation, cell proliferation, and protein homeostasis. Mutations in GRN are implicated in frontotemporal dementia. Described herein are methods of editing or modifying expression of GRN using a vector comprising a plurality of expression cassettes that may comprise an expression cassette encoding an engineered RNA payload to treat a disease (e.g., frontotemporal dementia).

In some embodiments, the present disclosure provides vectors comprising a plurality of expression cassettes that may comprise an expression cassette encoding engineered guide RNAs that target GRN. The engineered guide RNAs may target GRN to modify or alter expression of GRN. In some embodiments, targeting GRN with the engineered guide RNAs of the present disclosure may treat a disease associated with GRN, such as frontotemporal dementia. In some embodiments, the engineered guide RNAs may facilitate ADAR-mediated RNA editing of GRN to correct G to A mutations by targeting adenosines for deamination. The engineered guide RNAs of the present disclosure may target a coding sequence in GRN. For example, the coding sequence can be a translation initiation site (TIS) (AUG) of AUG, and the engineered guide RNA can facilitate ADAR-mediated RNA editing of AUG to GUG. Editing of the TIS may affect protein knockdown of GRN. In another example, the guide RNA can facilitate ADAR-mediated correction of missense mutations in the coding sequence. Correcting a missense mutation may increase expression of functional GRN protein. In another example, the guide RNA can facilitate ADAR-mediated correction of nonsense mutations in the coding sequence. Correcting a nonsense mutation may increase expression of GRN protein.

In some embodiments, the engineered guide RNAs target a non-coding sequence in GRN. The non-coding sequence can be a polyA signal sequence and the engineered guide RNA can facilitate ADAR-mediated RNA editing of one or more adenosines in the polyA signal sequence of GRN. In some embodiments, engineered guide RNAs of the present disclosure can be multiplexed to target more than one polyA signal sequences in GRN. In some embodiments, engineered guide RNAs of the present disclosure can be multiplexed to target the TIS and one or more polyA signal sequences in GRN. In some embodiments, engineered guide RNAs can be multiplexed to target a non-coding sequence and a coding sequence in GRN. The engineered guide RNAs of the present disclosure facilitated ADAR-mediated RNA editing of GRN, thereby, affecting protein knockdown.

In some embodiments, the engineered guide RNAs of the present disclosure facilitated ADAR-mediated RNA editing of from 1 to 100% of a target adenosine in GRN. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of GRN, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of GRN, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### ABCA4

In some embodiments, the present disclosure provides vectors comprising a plurality of expression cassettes that may comprise an expression cassette encoding guide RNAs that are capable of facilitating RNA editing of ATP binding cassette subfamily A member 4 (ABCA4). In some examples, the disease or condition can be associated with a mutation in an ABCA4 gene. In some examples, the disease or condition can be Stargardt macular degeneration. In some examples, the Stargardt macular degeneration can be caused, at least in part, by a mutation in an ABCA4 gene. In some examples, the mutation comprises a substitution of a G with an A at nucleotide position 5882 in a wild type ABCA4 gene. In some examples, the mutation comprises a G with an A at nucleotide position 5714 in a wild type ABCA4 gene. In some examples, the mutation comprises a substitution of a G with an A at nucleotide position 6320 in a wild type ABCA4 gene. In some examples, the double stranded substrate mimics one or more structural features of the naturally occurring ADAR substrate and comprises a target mRNA molecule encoded by the ABCA4 gene and an engineered guide that can be complementary, at least in part, to a portion of the target mRNA molecule.

In some examples, hybridization of a latent guide RNA targeting ABCA4 to a target ABCA4 mRNA produces a guide-target RNA scaffold that comprises a structural features selected from the group consisting of: (i) one or more X1/X2 bulges, wherein Xi is the number of nucleotides of the target RNA in the bulge and X2 is the number of nucleotides of the engineered guide RNA in the bulge, and wherein the one or more bulges is a 2/1 asymmetric bulge, a I/O asymmetric bulge, a 2/2 symmetric bulge, a 3/3 symmetric bulge, or a 4/4 symmetric bulge; (ii) an X1/X2 internal loop, wherein Xi is the number of nucleotides of the target RNA in the internal loop and X2 is the number of nucleotides of the engineered guide RNA in the internal loop, and wherein the internal loop is a 5/5 symmetric loop (iii) one or more mismatches, wherein the one or more mismatches is a G/G mismatch, an A/C mismatch, or a G/A mismatch, (iv) a G/U wobble base pair or a U/G wobble base pair, and (v) any combination thereof. In some embodiments, the guide-target RNA scaffold comprises a 2/1 asymmetric bulge, a I/O asymmetric bulge, a G/G mismatch, an A/C mismatch, and a 3/3 symmetric bulge. In some instances, the engineered latent guide RNA targeting ABCA4 comprises a G/G mismatch, a U/U mismatch, and a G/G mismatch. Said engineered guide RNAs can be delivered via viral vector (e.g., encoded for and delivered via AAV) as disclosed herein and can be administered via any route of administration disclosed herein to a subject in need thereof. The subject can be human and may be at risk of developing or has developed Stargardt macular degeneration (or Stargardt's disease). Such Stargardt macular degeneration can be at least partially caused by a mutation of ABCA4, for which an engineered guide RNA described herein can facilitate editing in, thus correcting the mutation in ABCA4 and reducing the incidence of Stargardt macular degeneration in the subject. Thus, the guide RNAs of the present disclosure can be used in a method of treatment of Stargardt macular degeneration.

In some embodiments, the present disclosure provides vectors comprising a plurality of expression cassettes that may comprise an expression cassette encoding engineered guide RNAs that target ABCA4. The engineered guide RNAs may target ABCA4 to modify or alter expression of ABCA4. In some embodiments, targeting ABCA4 with the engineered guide RNAs of the present disclosure may treat a disease associated with ABCA4, such as Stargardt disease. In some embodiments, the engineered guide RNAs may facilitate ADAR-mediated RNA editing of ABCA4 to correct G to A mutations by targeting adenosines for deamination. The engineered guide RNAs of the present disclosure may target a coding sequence in ABCA4. For example, the coding sequence can be a translation initiation site (TIS) (AUG) of AUG, and the engineered guide RNA can facilitate ADAR-mediated RNA editing of AUG to GUG. Editing of the TIS may affect protein knockdown of ABCA4. In another example, the guide RNA can facilitate ADAR-mediated correction of missense mutations in the coding sequence. Correcting a missense mutation may increase expression of functional ABCA4 protein. In another example, the guide RNA can facilitate ADAR-mediated correction of nonsense mutations in the coding sequence. Correcting a nonsense mutation may increase expression of ABCA4 protein.

In some embodiments, the engineered guide RNAs target a non-coding sequence in ABCA4. The non-coding sequence can be a polyA signal sequence and the engineered guide RNA can facilitate ADAR-mediated RNA editing of one or more adenosines in the polyA signal sequence of ABCA4. In some embodiments, engineered guide RNAs of the present disclosure can be multiplexed to target more than one polyA signal sequences in ABCA4. In some embodiments, engineered guide RNAs of the present disclosure can be multiplexed to target the TIS and one or more polyA signal sequences in ABCA4. In some embodiments, engineered guide RNAs can be multiplexed to target a non-coding sequence and a coding sequence in ABCA4. The engineered guide RNAs of the present disclosure facilitated ADAR-mediated RNA editing of ABCA4, thereby, affecting protein knockdown.

In some embodiments, the engineered guide RNAs of the present disclosure facilitated ADAR-mediated RNA editing of from 1 to 100% of a target adenosine in ABCA4. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of ABCA4, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of ABCA4, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### Amyloid Precursor Protein

A vector comprising a plurality of expression cassettes of the present disclosure can be used to express an engineered polynucleotide payload sequence targeting an amyloid precursor protein (APP). In some embodiments, the engineered polynucleotides can target a secretase enzyme cleavage site in APP and edit said cleavage site in order to modulate processing and cleavage of APP by secretase enzymes (e.g., a beta secretase such as BACE1, cathepsin B or Meprin beta). In some embodiments, the engineered polynucleotides can modulate the expression of APP. In some cases, the engineered polynucleotides can modulate the transcription or post-transcriptional regulation of the APP mRNA or pre-mRNA. In other cases, the engineered polynucleotides can correct aberrant expression of splice variants generated by a mutation in APP. In some cases, the engineered polynucleotides can modulate the gene or protein translation of APP. In some embodiments, the engineered polynucleotides can decrease, down-regulate, or knock down the expression of APP by decreasing the abundance of the APP transcript. In some instances, the engineered polynucleotides can decrease or down-regulate the processing, splicing, turnover or stability of the APP transcript; or the accessibility of the APP transcript by translational machinery such as ribosome. In some cases, an engineered polynucleotide can facilitate a knockdown of APP. A knockdown can reduce the expression of APP. In some cases, a knockdown can be accompanied by editing of the APP mRNA or pre-mRNA. In some cases, a knockdown can occur with substantially little to no editing of the APP mRNA or pre-mRNA. In some instances, a knockdown can occur by targeting an untranslated region of the APP mRNA or pre-mRNA, such as a 3′ UTR, a 5′ UTR or both. In some cases, a knockdown can occur by targeting a coding region of the APP mRNA or pre-mRNA.

Compositions described herein can edit the cleavage site in APP, so that B/y secretases exhibit reduced cleavage of APP or can no longer cut APP, and therefore reduced levels of Abeta 40/Abeta 42 or no Abetas can be produced. Compositions consistent with the present disclosure may combine compositions for target APP cleavage site editing with compositions for Tau (e.g., a microtubule-associated protein Tau (MAPT) encoded from a MAPT gene) knockdown or compositions for Alpha-synuclein (SNCA) knockdown and can have synergistic effects to prevent and/or cure a neurodegenerative disease. The compositions and methods disclosed herein can yield results in editing and/or knockdown of targets without any of the resulting issues seen in small molecule or antibody therapy. Compositions can knockdown APP (instead of target cleavage site editing). Editing at the target cleavage site in APP and knockdown can be deployed singly or in combination.

In some cases, a targeting sequence of an engineered polynucleotide provided herein can at least partially hybridize to a region of a target RNA. A region of a target RNA can comprise: (a) a sequence that at least partially encodes for a suitable target provided herein, (b) a sequence that is proximal to a sequence that at least partially encodes for a suitable target provided herein, (c) comprises (a) and (b). For example, a region of a target RNA can comprise (a) a sequence that at least partially encodes for an APP, (b) a sequence that is proximal to a sequence that at least partially encodes for an APP, or (c) comprises (a) and (b). Other suitable targets can be targeted with engineered polynucleotides disclosed herein. Amyloid precursor protein (APP)

Pathogenic cleavage of amyloid precursor protein (APP) can create Amyloid beta (Abeta) fragments, which has been implicated in Alzheimer's disease. The accumulation of Abeta fragments can: impair synaptic functions and related signaling pathways, change neuronal activities, trigger the release of neurotoxic mediators from glial cells, or any combination thereof. Abeta can alter kinase function, leading to Tau hyperphosphorylation.

The generation of Abeta by enzymatic cleavages of the α-amyloid precursor protein (APP) is an important player in Alzheimer's disease. The non-amyloidogenic APP processing pathway involves cleavages by alpha- and gamma-secretase. The cleavage by alpha-secretase generates a long form of secreted APP (APPs alpha) and a C-terminal fragment (alpha-CTF). Further processing of alpha-CTF by gamma-secretase generates a p3 and AICD fragment. The amyloidogenic APP processing pathway instead involves cleavages by beta- and gamma-secretase. The cleavage by beta-secretase generates a short form of secreted APP (APPs beta) and a C-terminal fragment (beta-CTF). Further processing of beta-CTF by gamma-secretase generates an Abeta and AICD fragment. The oligomerization and fibrillization of Abeta fragments lead to AD pathology. In some cases, amyloid precursor protein (APP) can be cut by a beta secretase (e.g., BACE1, cathepsin B or Meprin beta) or gamma secretase, and the fragment resulting from such cuts can be Abeta peptides of 36-43 amino acids. Certain Abeta peptide metabolites of this cleavage can be crucially involved in Alzheimer's disease pathology and progression.

In some embodiments, the present disclosure provides vectors comprising a plurality of expression cassettes that may comprise an expression cassette encoding engineered guide RNAs that target APP. The engineered guide RNAs may facilitate ADAR-mediated RNA editing of APP to correct G to A mutations by targeting adenosines for deamination. In some embodiments, the engineered guide RNAs of the present disclosure target a coding sequence in APP. For example, the coding sequence can be a translation initiation site (TIS) (AUG) of AUG, and the engineered guide RNA can facilitate ADAR-mediated RNA editing of AUG to GUG. Editing of the TIS may affect protein knockdown of APP. In another example, the guide RNA can facilitate ADAR-mediated correction of missense mutations in the coding sequence. Correcting a missense mutation may increase expression of functional AAP protein. In another example, the guide RNA can facilitate ADAR-mediated correction of nonsense mutations in the coding sequence. Correcting a nonsense mutation may increase expression of AAP protein.

In some embodiments, the engineered guide RNAs target a non-coding sequence in APP. The non-coding sequence can be a polyA signal sequence and the engineered guide RNA can facilitate ADAR-mediated RNA editing of one or more adenosines in the polyA signal sequence of APP. In some embodiments, engineered guide RNAs of the present disclosure can be multiplexed to target more than one polyA signal sequences in APP. In some embodiments, engineered guide RNAs of the present disclosure can be multiplexed to target the TIS and one or more polyA signal sequences in APP. In some embodiments, engineered guide RNAs can be multiplexed to target a non-coding sequence and a coding sequence in APP. The engineered guide RNAs of the present disclosure facilitated ADAR-mediated RNA editing of APP, thereby, affecting protein knockdown.

In some embodiments, the engineered guide RNAs of the present disclosure facilitated ADAR-mediated RNA editing of from 1 to 100% of a target adenosine in APP. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of APP, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of APP, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### SERPINA1

In some embodiments, the present disclosure provides vectors comprising a plurality of expression cassettes that may comprise an expression cassette encoding guide RNAs that are capable of facilitating RNA editing of serpin family A member 1 (SERPINA1). In some examples, the disease or condition can be an AAT deficiency or an associated lung or liver pathology (e.g., chronic obstructive pulmonary disease, cirrhosis, hepatocellular carcinoma) caused, at least in part, by a mutation in a SERPINA1 gene. In some examples, the mutation can be a substitution of a G with an A at nucleotide position 9989 within a wild type SERPINA1 gene. In some examples, administration of the engineered guides disclosed herein restores expression of a normal AAT protein (e.g., as compared to an inactive or defective AAT protein) in a subject with an AAT deficiency. In some examples, a double stranded RNA (dsRNA) substrate (a guide-target RNA scaffold) is formed upon hybridization of an engineered guide of the present disclosure to a target RNA. In some examples, the target RNA forming the double stranded substrate comprises a portion of a mRNA or pre-mRNA molecule encoded by the SERPINA1 gene. In some examples the targeting region of the engineered guide forming the double stranded substrate is, at least in part, complementary to a portion of a mRNA or pre-mRNA molecule encoded by the SERPINA1 gene. In some examples the double stranded substrate comprises a single mismatch. In some examples, the engineered substrate additionally comprises one or two bulges. In some examples, the double stranded substrate can be formed by a target RNA comprising a mRNA or pre-mRNA encoded by the SERPINA1 gene and an engineered guide complementary to a portion of the mRNA encoded by the SERPINA1 gene, wherein the engineered substrate comprises a single mismatch. In some examples, the double stranded substrate can be formed by a target RNA comprising a mRNA or pre-mRNA encoded by the SERPINA1 gene and an engineered guide complementary to a portion of the mRNA or pre-mRNA encoded by the SERPINA1 gene, wherein the engineered substrate comprises a single mismatch, and wherein the engineered substrate comprises two additional bulges.

Guide RNAs can facilitate correction of a G to A mutation at nucleotide position 9989 of a SERPINA1 gene. In some embodiments, a guide RNA of the present disclosure can target, for example, E342K of SERPINA1. Said guide RNAs targeting a site in SERPINA1 can be encoded for by an engineered polynucleotide construct of the present disclosure.

In some embodiments, the present disclosure provides vectors comprising a plurality of expression cassettes that may comprise an expression cassette encoding engineered guide RNAs that target SERPINA1. The engineered guide RNAs may target SERPINA1 to modify or alter expression of SERPINA1. In some embodiments, targeting SERPINA1 with the engineered guide RNAs of the present disclosure may treat a disease associated with SERPINA1, such as alpha-1 antitrypsin deficiency. In some embodiments, the engineered guide RNAs may facilitate ADAR-mediated RNA editing of SERPINA1 to correct G to A mutations by targeting adenosines for deamination. The engineered guide RNAs of the present disclosure may target a coding sequence in SERPINA1. For example, the coding sequence can be a translation initiation site (TIS) (AUG) of AUG, and the engineered guide RNA can facilitate ADAR-mediated RNA editing of AUG to GUG. Editing of the TIS may affect protein knockdown of SERPINA1. In another example, the guide RNA can facilitate ADAR-mediated correction of missense mutations in the coding sequence. Correcting a missense mutation may increase expression of functional SERPINA1 protein. In another example, the guide RNA can facilitate ADAR-mediated correction of nonsense mutations in the coding sequence. Correcting a nonsense mutation may increase expression of SERPINA1 protein.

In some embodiments, the engineered guide RNAs target a non-coding sequence in SERPINA1. The non-coding sequence can be a polyA signal sequence and the engineered guide RNA can facilitate ADAR-mediated RNA editing of one or more adenosines in the polyA signal sequence of SERPINA1. In some embodiments, engineered guide RNAs of the present disclosure can be multiplexed to target more than one polyA signal sequences in SERPINA1. In some embodiments, engineered guide RNAs of the present disclosure can be multiplexed to target the TIS and one or more polyA signal sequences in SERPINA1. In some embodiments, engineered guide RNAs can be multiplexed to target a non-coding sequence and a coding sequence in SERPINA1. The engineered guide RNAs of the present disclosure facilitated ADAR-mediated RNA editing of SERPINA1, thereby, affecting protein knockdown.

In some embodiments, the engineered guide RNAs of the present disclosure facilitated ADAR-mediated RNA editing of from 1 to 100% of a target adenosine in SERPINA1. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of SERPINA1, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of SERPINA1, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### Recombinant Vectors and Delivery

In some embodiments, the polynucleotide (e.g., vector) comprising a plurality of expression cassettes (e.g., an expression cassette encoding a small RNA payload, such as an engineered guide RNA) of the present disclosure is introduced into a subject via delivery of the vector. In some embodiments the vector is a plasmid, a viral vector, an expression cassette, or a transformed cell. A vector can facilitate delivery of the engineered polynucleotide into a cell to genetically modify the cell. In some examples, the vector comprises DNA, such as double stranded or single stranded DNA. In some examples, the delivery vector can be a eukaryotic vector, a prokaryotic vector (e.g., a bacterial vector or plasmid), a viral vector, or any combination thereof. In some embodiments, the vector comprises a plurality of expression cassettes. In some embodiments, a viral vector comprises a viral capsid, an inverted terminal repeat sequence, and the engineered polynucleotide can be used to deliver the small RNA payload to a cell.

In some embodiments, the viral vector can be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector can be a recombinant vector, a hybrid vector, a chimeric vector, a self-complementary vector, a single-stranded vector, or any combination thereof.

In some embodiments, the viral vector is an adenoviral vector, an adeno-associated viral vector, or a lentiviral vector. Adeno-associated virus (AAV) vectors include vectors derived from any AAV serotype, including, but not limited to AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68, chimeras thereof, variants or derivatives thereof, and combinations thereof.

In some embodiments, a polynucleotide is introduced into a subject by non-viral vector systems. In some embodiments, cationic lipids, polymers, hydrodynamic injection and/or ultrasound may be used in delivering a polynucleotide to a subject in the absence of virus.

In some examples, the vector may be a eukaryotic vector, a prokaryotic vector (e.g., a bacterial vector) a viral vector, or any combination thereof. In some examples, the vector may be a viral vector. In some embodiments, the viral vector may be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector may be a recombinant vector, a hybrid vector, a chimeric vector, a self-complementary vector, a single-stranded vector, or any combination thereof.

In some embodiments, the viral vector may be an adeno-associated virus (AAV). In some embodiments, the AAV may be any AAV known in the art. In some embodiments, the viral vector may be of a specific serotype. In some embodiments, the viral vector may be an AAV1 serotype, AAV2 serotype, AAV3 serotype, AAV4 serotype, AAV5 serotype, AAV6 serotype, AAV7 serotype, AAV8 serotype, AAV9 serotype, AAV10 serotype, AAV11 serotype, AAV 12 serotype, AAV13 serotype, AAV14 serotype, AAV15 serotype, AAV16 serotype, AAV-DJ serotype, AAV-DJ/8 serotype, AAV-DJ/9 serotype, AAV1/2 serotype, AAV.rh8 serotype, AAV.rh10 serotype, AAV.rh20 serotype, AAV.rh39 serotype, AAV.Rh43 serotype, AAV.Rh74 serotype, AAV.v66 serotype, AAV.Oligo001 serotype, AAV.SCH9 serotype, AAV.r3.45 serotype, AAV.RHM4-1 serotype, AAV.hu37 serotype, AAV.Anc80 serotype, AAV.Anc80L65 serotype, AAV.7m8 serotype, AAV.PhP.eB serotype, AAV.PhP.V1 serotype, AAV.PHP.B serotype, AAV.PhB.C1 serotype, AAV.PhB.C2 serotype, AAV.PhB.C3 serotype, AAV.PhB.C6 serotype, AAV.cy5 serotype, AAV2.5 serotype, AAV2tYF serotype, AAV3B serotype, AAV.LK03 serotype, AAV.HSC1 serotype, AAV.HSC2 serotype, AAV.HSC3 serotype, AAV.HSC4 serotype, AAV.HSC5 serotype, AAV.HSC6 serotype, AAV.HSC7 serotype, AAV.HSC8 serotype, AAV.HSC9 serotype, AAV.HSC10 serotype, AAV.HSC11 serotype, AAV.HSC12 serotype, AAV.HSC13 serotype, AAV.HSC14 serotype, AAV.HSC15 serotype, AAV.HSC16 serotype, AAV.HSC17 serotype, or AAVhu68 serotype, chimeras thereof, variants or derivatives thereof, and combinations thereof.

In some embodiments, the AAV vector may be a recombinant vector, a hybrid AAV vector, a chimeric AAV vector, a self-complementary AAV (scAAV) vector, a single-stranded AAV, or any combination thereof.

In some embodiments, the AAV vector may be a recombinant AAV (rAAV) vector. Methods of producing recombinant AAV vectors may be known in the art and generally involve, in some cases, introducing into a producer cell line: (1) DNA necessary for AAV replication and synthesis of an AAV capsid, (b) one or more helper constructs comprising the viral functions missing from the AAV vector, (c) a helper virus, and (d) the plasmid construct containing the genome of the AAV vector, e.g., ITRs, promoter and payload sequences, etc. In some examples, the viral vectors described herein may be engineered through synthetic or other suitable means by references to published sequences, such as those that may be available in the literature. For example, the genomic and protein sequences of various serotypes of AAV, as well as the sequences of the native terminal repeats (TRs), Rep proteins, and capsid subunits may be known in the art and may be found in the literature or in public databases such as GenBank or Protein Data Bank (PDB).

In some examples, methods of producing delivery vectors herein comprising packaging a polynucleotide of the present disclosure in an AAV vector. In some examples, methods of producing the delivery vectors described herein comprise, (a) introducing into a cell: (i) a polynucleotide disclosed herein; and (ii) a viral genome comprising a Replication (Rep) gene and Capsid (Cap) gene that encodes a wild type AAV capsid protein or modified version thereof; (b) expressing in the cell the wild type AAV capsid protein or modified version thereof; (c) assembling an AAV particle; and (d) packaging the polynucleotide disclosed herein in the AAV particle, thereby generating an AAV delivery vector. In some examples, any polynucleotide disclosed herein may be packaged in the AAV vector. In some examples, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site, thereby enabling formation of a self-complementary AAV.

In some examples, a hybrid AAV vector may be produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV5 or AAV9), wherein the first and second AAV serotypes may not be the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some examples, the AAV vector may be a chimeric AAV vector. In some examples, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some examples, the AAV vector comprises a self-complementary AAV genome. Self-complementary AAV genomes may be generally known in the art and contain both DNA strands which can anneal together to form double-stranded DNA.

In some examples, the delivery vector may be a retroviral vector. In some examples, the retroviral vector may be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector may be transfected such that the majority of sequences coding for the structural genes of the virus (e.g., gag, pol, and env) may be deleted and replaced by the gene(s) of interest.

In some examples, the delivery vehicle may be a non-viral vector. Examples of non-viral vectors may include plasmids, lipid nanoparticles, lipoplexes, polymersomes, polyplexes, dendrimers, nanoparticles, and cell-penetrating peptides. The non-viral vector may comprise a polynucleotide, such as a plasmid, encoding for a promoter (e.g., comprising a cell type- or cell state-specific response element and a switchable core promoter) and a payload sequence. In some examples, the delivery vehicle may be a plasmid. In some examples, the plasmid may be a minicircle plasmid. In some embodiments, a vector may comprise naked DNA (e.g., a naked DNA plasmid). In some embodiments, the non-viral vector comprises DNA. In some embodiments, the non-viral vector comprises RNA. In some examples, the non-viral vector comprises circular double-stranded DNA. In some examples, the non-viral vector may comprise a linear polynucleotide. In some examples, the non-viral vector comprises a polynucleotide encoding one or more genes of interest and one or more regulatory elements. In some examples, the non-viral vector comprises a bacterial backbone containing an origin of replication and an antibiotic resistance gene or other selectable marker for plasmid amplification in bacteria. In some examples, the non-viral vector contains one or more genes that provide a selective marker to induce a target cell to retain a polynucleotide (e.g., a plasmid) of the non-viral vector. In some examples, the non-viral vector may be formulated for delivery through injection by a needle carrying syringe. In some examples, the non-viral vector may be formulated for delivery via electroporation. In some examples, a polynucleotide of the non-viral vector may be engineered through synthetic or other suitable means known in the art. For example, in some cases, the genetic elements may be assembled by restriction digest of the desired genetic sequence from a donor plasmid or organism to produce ends of the DNA which may then be readily ligated to another genetic sequence.

In some embodiments, the vector comprising a plurality of expression cassettes is a non-viral vector system. In some embodiments, the non-viral vector system comprises cationic lipids, or polymers. For example, the non-viral vector system comprises can be a liposome or polymeric nanoparticle. In some embodiments, the small RNA payload or a non-viral vector comprising the small RNA payload is delivered to a cell by hydrodynamic injection or ultrasound.

### Vector Genome Integrity

Vector genome integrity may be considered when developing vectors with multiple expression cassettes. Vectors may undergo recombination events resulting in a recombined product that is different from the original vector sequence. The recombined product may be a shorter sequence than the original vector. Comparing the concentration of recombination products to the concentration of the original vector may show the genome integrity of a vector sequence. A vector's genome integrity may be considered when developing the multi-expression cassette vectors as described herein to ensure a given vector sequence is stable and undergoes minimal recombination events thereby keeping the original vector sequence intact. Vector genome integrity referred to herein is described as the ability for a given vector sequence to retain its size and length over a period of time. Vector genome integrity may be reduced by events such as recombination events which result in recombination products that are shortened vector sequences relative to the original vector sequence. Vector genome integrity may be characterized by a percent intact value which can be calculated by the concentration of the original vector sequence divided by the sum of the concentration of the original vector sequence and the concentration of recombination products of the vector sequence.

Recombination events in a vector sequence may be more likely when there are multiple regions in the vector sequence with 100% identity. For example, recombination events may be more likely when using a two-expression cassette vector wherein a first expression cassette and a second expression cassette have a 100% sequence identity to each other. Recombination events in a vector sequence may be minimized by using two or more different expression cassette sequences in a multi-expression cassette vector. For example, a first expression cassette and a second expression cassette may have no more than 99% sequence identity, no more than 98% sequence identity, no more than 97% sequence identity, no more than 96% sequence identity, no more than 95% sequence identity, no more than 94% sequence identity, no more than 93% sequence identity, no more than 92% sequence identity, no more than 91% sequence identity, no more than 90% sequence identity, no more than 85% sequence identity, no more than 80% sequence identity, no more than 75% sequence identity, no more than 70% sequence identity, no more than 65% sequence identity, no more than 60% sequence identity, no more than 55% sequence identity, or no more than 50% sequence identity to each other. In some embodiments, a second expression cassette may have a different promoter sequence than a promoter sequence in a first expression cassette. In some embodiments, a second expression cassette may have a different RNA payload sequence (e.g., an engineered guide RNA sequence) than a RNA payload sequence (e.g., an engineered guide RNA sequence) in a first expression cassette. In some embodiments, a second expression cassette may have a different termination sequence than a termination sequence in a first expression cassette.

Guide RNAs with Sequence Divergence

To increase vector genome integrity of the polynucleotides (e.g., a viral vector) of the present disclosure, guide RNAs with sequence divergence may be developed. Sequence divergence refers to a difference in a nucleotide sequence between two guide RNA sequences (e.g., a first guide RNA sequence and a second guide RNA sequence). Two guide RNA sequences may differ by a percent sequence identity. In some embodiments a first guide RNA sequence and a second guide RNA sequence may have no more than 99% sequence identity, no more than 98% sequence identity, no more than 97% sequence identity, no more than 96% sequence identity, no more than 95% sequence identity, no more than 94% sequence identity, no more than 93% sequence identity, no more than 92% sequence identity, no more than 91% sequence identity, no more than 90% sequence identity, no more than 85% sequence identity, no more than 80% sequence identity, no more than 75% sequence identity, no more than 70% sequence identity, no more than 65% sequence identity, no more than 60% sequence identity, no more than 55% sequence identity, or no more than 50% sequence identity to each other.

Sequence divergence in a guide RNA sequence may be introduced by altering nucleotide identities in a first guide RNA sequence to create a second guide RNA sequence (e.g., a sequence divergent guide RNA sequence). Altering the nucleotide identities may comprise changing an A to a T, C, or G; changing a T to an A, C, or G; changing a C to an A, T, or G; changing a G to an A, T, or C; or a combination thereof in a DNA sequence encoding a guide RNA sequence. In some embodiments an A is changed to a G in a DNA sequence encoding a guide RNA sequence. In some embodiments a C is changed to a T in a DNA sequence encoding a guide RNA sequence.

Sequence divergent guide RNAs may refer to a second guide RNA sequence that is altered from a first guide RNA sequence to decrease sequence identity between two guide RNA sequences (e.g., a first guide RNA sequence and a second guide RNA sequence). In some embodiments a sequence divergent guide RNA sequence may comprise at least one and no more than 30 nucleotide alterations from a first guide RNA sequence. In some embodiments a sequence divergent guide RNA sequence may comprise at least one and no more than 30 nucleotide alterations, at least one and no more than 28 nucleotide alterations, at least one and no more than 26 nucleotide alterations, at least one and no more than 24 nucleotide alterations, at least one and no more than 22 nucleotide alterations, at least one and no more than 20 nucleotide alterations, at least one and no more than 18 nucleotide alterations, at least one and no more than 16 nucleotide alterations, at least one and no more than 14 nucleotide alterations, at least one and no more than 12 nucleotide alterations, at least one and no more than 10 nucleotide alterations, at least one and no more than 8 nucleotide alterations, at least one and no more than 6 nucleotide alterations, or at least one and no more than 5 nucleotide alterations from a first guide RNA sequence.

In some embodiments, a sequence divergent guide RNA sequence may comprise nucleotide alterations from a first guide RNA sequence that are dispersed throughout the sequence divergent guide RNA sequence. In some embodiments, the dispersed nucleotide alterations in a sequence divergent guide RNA sequence may have a pattern of dispersion with a frequency of nucleotide alterations in a sequence divergent guide RNA sequence. In some embodiments, the nucleotide alterations from the first engineered guide RNA sequence are dispersed at a frequency of at least one and no more than 4 nucleotide alterations per every 10 nucleotides in the sequence divergent guide RNA sequence (e.g., a second engineered guide RNA sequence). In some embodiments, the nucleotide alterations from the first engineered guide RNA sequence are dispersed at a frequency of 3 nucleotide alterations per every 10 nucleotides in the second engineered guide RNA sequence.

Sequence divergent guide RNAs may form a similar guide-target RNA scaffold upon hybridization to a target sequence to a guide-target RNA scaffold formed from a first engineered guide RNA hybridizing to a target sequence. In some embodiments, the first engineered guide RNA sequence and the sequence divergent guide RNA (e.g., a second engineered guide RNA sequence) are each independently capable of forming a guide-target RNA scaffold comprising one or more structural features upon hybridization of the small RNA payload to a target sequence. In some embodiments, the guide-target RNA scaffold of the first engineered guide RNA sequence and the guide-target RNA scaffold of the sequence divergent guide RNA sequence (e.g., a second engineered guide RNA sequence) comprise the same one or more structural features. In some embodiments, the one or more structural features comprise a bulge, a mismatch, an internal loop, a hairpin, or combinations thereof. In some embodiments, the one or more structural features comprises the bulge, and wherein the bulge is a symmetric bulge. In some embodiments, the one or more structural features comprises the bulge, and wherein the bulge is an asymmetric bulge. In some embodiments, the one or more structural features comprises the internal loop, and wherein the internal loop is a symmetric internal loop. In some embodiments, the one or more structural features comprises the internal loop, and wherein the internal loop is an asymmetric internal loop. In some embodiments, the one or more structural features comprises the hairpin, and wherein the hairpin is a recruitment hairpin or a non-recruitment hairpin.

Sequence divergent guide RNA sequences may have one or more wobble base pairs in the guide-target RNA scaffold as compared to the guide-target RNA scaffold of a first engineered guide RNA sequence hybridized to a target sequence. In some embodiments, the guide-target RNA scaffold of the sequence divergent guide RNA sequence (e.g., a second engineered guide RNA sequence) has between at least one and no more than 30 additional wobble base pairs than the guide-target RNA scaffold of the first engineered guide RNA sequence. In some embodiments, one or more of the wobble base pairs are GU wobble base pairs. In some embodiments, the introduction of GU wobble base pairs to a guide-target RNA scaffold may over twist the double stranded RNA (dsRNA) helix as compared to the dsRNA helix in a guide-target RNA scaffold without GU wobble base pairs.

An engineered polynucleotide as described herein can comprise one or more polynucleotide sequence(s) that encode one or more engineered guide RNA(s). For example, an engineered polynucleotide can comprise 1, 2, 3, 4, or more than 4 polynucleotide sequence(s) that encode 1, 2, 3, 4, or more than 4 engineered guide RNAs.

In some instances, the engineered polynucleotide can comprise one or more polynucleotide sequence(s) encoding one or more engineered guide RNA(s) that independently hybridize to (target): (1) different target sequences of the same target RNA, or (2) different target sequences of different target RNAs. For example, a first engineered guide RNA encoded by a first polynucleotide sequence can hybridize to a target sequence of a first target RNA while a second engineered guide RNA encoded by a second polynucleotide sequence can hybridize to a target sequence of a second target RNA, in some instances resulting in ADAR-mediated editing of an adenosine in the target sequence of the first target RNA and an adenosine in the target sequence of the second target RNA.

In some instances, the engineered polynucleotide can comprise one or more polynucleotide sequence(s) encoding one or more engineered guide RNA(s) that independently hybridize to (target) the same target sequence of a target RNA. For example, the one or more engineered guide RNA(s) encoded by the one or more polynucleotide sequence(s) can each independently hybridize to a target sequence of a target RNA and/or facilitate editing of the same adenosine in the target sequence of the target RNA via ADAR. In some cases, the one or more engineered guide RNA(s) that hybridize to (target) the same target sequence of a target RNA have identical sequences (i.e., the one or more engineered guide RNAs are copies of each other).

Alternatively, two or more engineered guide RNA(s) that hybridize to (target) the same target sequence of a target RNA can comprise different sequences. For example, a first engineered guide RNA encoded by an engineered polynucleotide can have a sequence identity of less than, greater than, or equal to: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a second engineered guide RNA encoded by the engineered polynucleotide, where the second engineered guide RNA hybridizes to (targets) the same target sequence of a target RNA as the first engineered guide RNA. In some instances, a first engineered guide RNA encoded by an engineered polynucleotide can have at least about 70% to about 99% sequence identity, at least about 60% to about 99% sequence identity, at least about 80% to about 99% sequence identity, at least about 60% to about 70% sequence identity, at least about 70% to about 80% sequence identity, at least about 75% to about 85% sequence identity, at least about 85% to about 99% sequence identity, at least about 85% to about 90% sequence identity, at least about 88% to about 93% sequence identity, at least about 90% to about 95% sequence identity, at least about 92% to about 99% sequence identity, or at least about 95% to about 99% sequence identity to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 60% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 61% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 62% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 63% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 64% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 65% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 66% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 67% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 68% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 69% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 70% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 71% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 72% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 73% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 74% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 75% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 76% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 77% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 78% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 79% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 80% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 81% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 82% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 83% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 84% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 85% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 86% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 87%, to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 88% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 89% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 90% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 91% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 92% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 93% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 94% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 95% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 96% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 97% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 98% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 99% to a second engineered guide RNA encoded by the engineered polynucleotide, where the second engineered guide RNA hybridizes to (targets) the same target sequence of a target RNA as the first engineered guide RNA. In some embodiments, polynucleotides encoding a first engineered guide RNA, a second engineered guide RNA, or both can be delivered via an AAV. In some instances, the AAV can be formulated in a composition, such as any of the pharmaceutical compositions disclosed herein.

### Pharmaceutical Compositions

Methods for treatment of diseases or disorders characterized by genetic mutations or aberrant gene expression are also encompassed by the present disclosure. Said methods include administering a therapeutically effective amount of a payload sequence as part of a recombinant polynucleotide cassette. The vectors comprising a plurality of expression cassettes of the disclosure can be formulated in pharmaceutical compositions. These compositions can comprise, in addition to one or more of the recombinant polynucleotide cassettes, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes.

The compositions described herein (e.g., vectors comprising a plurality of expression cassettes) can be formulated with a pharmaceutically acceptable carrier for administration to a subject (e.g., a human or a non-human animal). A pharmaceutically acceptable carrier can include, but is not limited to, phosphate buffered saline solution, water, emulsions (e.g., an oil/water emulsion or a water/oil emulsions), glycerol, liquid polyethylene glycols, aprotic solvents such (e.g., dimethylsulfoxide, N-methylpyrrolidone, or mixtures thereof), and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. Additional examples of carriers, stabilizers and adjuvants consistent with the compositions of the present disclosure can be found in, for example, Remington's Pharmaceutical Sciences, 21st Ed., Mack Publ. Co., Easton, Pa. (2005), incorporated herein by reference in its entirety.

In some examples, the pharmaceutical composition can be formulated in unit dose forms or multiple-dose forms. In some examples, the unit dose forms can be physically discrete units suitable for administration to human or non-human subjects (e.g., animals). In some examples, the unit dose forms can be packaged individually. In some examples, each unit dose contains a predetermined quantity of an active ingredient(s) that can be sufficient to produce the desired therapeutic effect in association with pharmaceutical carriers, diluents, excipients, or any combination thereof. In some examples, the unit dose forms comprise ampules, syringes, or individually packaged tablets and capsules, or any combination thereof. In some instances, a unit dose form can be comprised in a disposable syringe. In some instances, unit-dosage forms can be administered in fractions or multiples thereof. In some examples, a multiple-dose form comprises a plurality of identical unit dose forms packaged in a single container, which can be administered in segregated a unit dose form. In some examples, multiple dose forms comprise vials, bottles of tablets or capsules, or bottles of pints or gallons. In some instances, a multiple-dose forms comprise the same pharmaceutically active agents. In some instances, a multiple-dose forms comprise different pharmaceutically active agents.

In some examples, the pharmaceutical composition comprises a pharmaceutically acceptable excipient. In some examples, the excipient comprises a buffering agent, a cryopreservative, a preservative, a stabilizer, a binder, a compaction agent, a lubricant, a chelator, a dispersion enhancer, a disintegration agent, a flavoring agent, a sweetener, or a coloring agent, or any combination thereof.

In some examples, an excipient comprises a buffering agent. In some examples, the buffering agent comprises sodium citrate, magnesium carbonate, magnesium bicarbonate, calcium carbonate, calcium bicarbonate, or any combination thereof. In some examples, the buffering agent comprises sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, or calcium hydroxide and other calcium salts, or any combination thereof.

In some examples, an excipient comprises a cryopreservative. In some examples, the cryopreservative comprises DMSO, glycerol, polyvinylpyrrolidone (PVP), or any combination thereof. In some examples, a cryopreservative comprises a sucrose, a trehalose, a starch, a salt of any of these, a derivative of any of these, or any combination thereof. In some examples, an excipient comprises a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. In some examples, an excipient comprises a surfactant, a sugar, an amino acid, an antioxidant, a salt, a non-ionic surfactant, a solubilizer, a triglyceride, an alcohol, or any combination thereof. In some examples, an excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. In some examples, the excipient can be an excipient described in the Handbook of Pharmaceutical Excipients, American Pharmaceutical Association (1986).

In some examples, the excipient comprises a preservative. In some examples, the preservative comprises an antioxidant, such as alpha-tocopherol and ascorbate, an antimicrobial, such as parabens, chlorobutanol, and phenol, or any combination thereof. In some examples, the antioxidant comprises EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol or N-acetyl cysteine, or any combination thereof. In some examples, the preservative comprises validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitors, or any combination thereof.

In some examples, the excipient comprises a binder. In some examples, the binder comprises starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, or any combination thereof.

In some examples, the binder can be a starch, for example a potato starch, corn starch, or wheat starch; a sugar such as sucrose, glucose, dextrose, lactose, or maltodextrin; a natural and/or synthetic gum; a gelatin; a cellulose derivative such as microcrystalline cellulose, hydroxypropyl cellulose, hydroxyethyl cellulose, hydroxypropyl methyl cellulose, carboxymethyl cellulose, methyl cellulose, or ethyl cellulose; polyvinylpyrrolidone (povidone); polyethylene glycol (PEG); a wax; calcium carbonate; calcium phosphate; an alcohol such as sorbitol, xylitol, mannitol, or water, or any combination thereof.

In some examples, the excipient comprises a lubricant. In some examples, the lubricant comprises magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, or light mineral oil, or any combination thereof. In some examples, the lubricant comprises metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate or talc or a combination thereof.

In some examples, the excipient comprises a dispersion enhancer. In some examples, the dispersion enhancer comprises starch, alginic acid, polyvinylpyrrolidones, guar gum, kaolin, bentonite, purified wood cellulose, sodium starch glycolate, isomorphous silicate, or microcrystalline cellulose, or any combination thereof as high HLB emulsifier surfactants.

In some examples, the excipient comprises a disintegrant. In some examples, a disintegrant comprises a non-effervescent disintegrant. In some examples, a non-effervescent disintegrants comprises starches such as corn starch, potato starch, pregelatinized and modified starches thereof, sweeteners, clays, such as bentonite, micro-crystalline cellulose, alginates, sodium starch glycolate, or gums such as agar, guar, locust bean, karaya, pectin, and tragacanth, or any combination thereof. In some examples, a disintegrant comprises an effervescent disintegrant. In some examples, a suitable effervescent disintegrant comprises bicarbonate in combination with citric acid, and sodium bicarbonate in combination with tartaric acid.

In some examples, the excipient comprises a sweetener, a flavoring agent or both. In some examples, a sweetener comprises glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like, or any combination thereof. In some cases, flavoring agents incorporated into a composition comprise synthetic flavor oils and flavoring aromatics; natural oils; extracts from plants, leaves, flowers, and fruits; or any combination thereof. In some embodiments, a flavoring agent comprises a cinnamon oil; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot, or any combination thereof.

In some examples, the excipient comprises a pH agent (e.g., to minimize oxidation or degradation of a component of the composition), a stabilizing agent (e.g., to prevent modification or degradation of a component of the composition), a buffering agent (e.g., to enhance temperature stability), a solubilizing agent (e.g., to increase protein solubility), or any combination thereof. In some examples, the excipient comprises a surfactant, a sugar, an amino acid, an antioxidant, a salt, a non-ionic surfactant, a solubilizer, a trigylceride, an alcohol, or any combination thereof. In some examples, the excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerine, xanthan rubber, soy isoflavones, polysorbate 80, ethyl alcohol, water, teprenone, or any combination thereof. In some examples, the excipient comprises a cryo-preservative. In some examples, the excipient comprises DMSO, glycerol, polyvinylpyrrolidone (PVP), or any combination thereof. In some examples, the excipient comprises a sucrose, a trehalose, a starch, a salt of any of these, a derivative of any of these, or any combination thereof.

In some examples, the pharmaceutical composition comprises a diluent. In some examples, the diluent comprises water, glycerol, methanol, ethanol, or other similar biocompatible diluents, or any combination thereof. In some examples, a diluent comprises an aqueous acid such as acetic acid, citric acid, maleic acid, hydrochloric acid, phosphoric acid, nitric acid, sulfuric acid, or any combination thereof. In some examples, a diluent comprises an alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, choline, lactose, maltose, mannitol, simethicone, sorbitol, starch, pregelatinized starch, talc, xylitol and/or anhydrates, hydrates and/or pharmaceutically acceptable derivatives thereof or combinations thereof.

In some examples, the pharmaceutical composition comprises a carrier. In some examples, the carrier comprises a liquid or solid filler, solvent, or encapsulating material. In some examples, the carrier comprises additives proteins, peptides, amino acids, lipids, and carbohydrates (e.g., sugars, including monosaccharides, di-, tri-, tetra-oligosaccharides, and oligosaccharides; derivatized sugars such as alditols, aldolic acids, esterified sugars and the like; and polysaccharides or sugar polymers), alone or in combination.

### Administration

Administration can refer to methods that can be used to enable the delivery of a composition described herein (e.g., vectors comprising a plurality of expression cassettes) to the desired site of biological action. For example, an engineered guide RNA or an expression cassette can be comprised in a DNA construct, a viral vector, or both and be administered by intravenous administration. Administration disclosed herein to an area in need of treatment or therapy can be achieved by, for example, and not by way of limitation, oral administration, topical administration, intravenous administration, inhalation administration, or any combination thereof. In some embodiments, delivery can include inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration, or a combination thereof. Delivery can include direct application to the affected tissue or region of the body. In some cases, topical administration can comprise administering a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment to an external surface of a surface, such as a skin. Delivery can include a parenchymal injection, an intra-thecal injection, an intra-ventricular injection, or an intra-cisternal injection. A composition provided herein can be administered by any method. A method of administration can be by intra-arterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a liposome, an exosome, an extracellular vesicle, an implant, or a combination thereof. In some cases, delivery can be from a device. In some instances, delivery can be administered by a pump, an infusion pump, or a combination thereof. In some embodiments, delivery can be by an enema, an eye drop, a nasal spray, or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). In some embodiments, a medical professional can administer the composition.

In some cases, administering can be oral ingestion. In some cases, delivery can be a capsule or a tablet. Oral ingestion delivery can comprise a tea, an elixir, a food, a drink, a beverage, a syrup, a liquid, a gel, a capsule, a tablet, an oil, a tincture, or any combination thereof. In some embodiments, a food can be a medical food. In some instances, a capsule can comprise hydroxymethylcellulose. In some embodiments, a capsule can comprise a gelatin, hydroxypropylmethyl cellulose, pullulan, or any combination thereof. In some cases, capsules can comprise a coating, for example, an enteric coating. In some embodiments, a capsule can comprise a vegetarian product or a vegan product such as a hypromellose capsule. In some embodiments, delivery can comprise inhalation by an inhaler, a diffuser, a nebulizer, a vaporizer, or a combination thereof.

In some embodiments, disclosed herein can be a method, comprising administering a composition disclosed herein to a subject (e.g., a human) in need thereof. In some instances, the method can treat (including prevent) a disease in the subject.

In some examples, a pharmaceutical composition disclosed herein can be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

The appropriate dosage and treatment regimen for the methods of treatment described herein vary with respect to the particular disease being treated, the gRNA and/or ADAR (or a vector encoding the gRNA and/or ADAR) being delivered, and the specific condition of the subject. In some examples, the administration can be over a period of time until the desired effect (e.g., reduction in symptoms can be achieved). In some examples, administration can be 1, 2, 3, 4, 5, 6, or 7 times per week. In some examples, administration or application of a composition disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or more. In some examples, administration can be over a period of 1, 2, 3, 4, 5, 6, 7, 8, 9, or 10 weeks. In some examples, administration can be over a period of 2, 3, 4, 5, 6 or more months. In some examples, administration can be performed repeatedly over a lifetime of a subject, such as once a month or once a year for the lifetime of a subject. In some examples, administration can be performed repeatedly over a substantial portion of a subject's life, such as once a month or once a year for at least about 1 year, 5 years, 10 years, 15 years, 20 years, 25 years, 30 years, or more. In some examples, treatment can be resumed following a period of remission.

Pharmaceutical compositions for oral administration can be in tablet, capsule, powder, or liquid form. A tablet can include a solid carrier such as gelatin or an adjuvant. Liquid pharmaceutical compositions generally include a liquid carrier such as water, petroleum, animal or vegetable oils, mineral oil, or synthetic oil. Physiological saline solution, dextrose or other saccharide solution or glycols such as ethylene glycol, propylene glycol or polyethylene glycol can be included.

For intravenous, cutaneous, or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

In some embodiments, the polynucleotide of the present disclosure or the vector comprising a plurality of expression cassettes of the present disclosure may be administered to cells via a lipid nanoparticle. In some embodiments, the lipid nanoparticle may be administered at the appropriate concentration according to standard methods appropriate for the target cells.

In some embodiments, the polynucleotide of the present disclosure or the vector comprising a plurality of expression cassettes of the present disclosure may be administered to cells via a viral vector. In some embodiments, the viral vector may be administered at the appropriate multiplicity of infection according to standard transduction methods appropriate for the target cells. Titers of the virus vector or capsid to administer can vary depending on the target cell type or cell state and number and can be determined by those of skill in the art. In some embodiments, at least about 102 infections units are administered. In some embodiments, at least about 103, 104, 105, 106, 107, 108, 109, 1010, 1011, 1012, or 1013 infectious units are administered.

In some embodiments, the polynucleotide or the vector comprising a plurality of expression cassettes is introduced to cells of any type or state, including, but not limited to neural cells, cells of the eye (including retinal cells, retinal pigment epithelium, and corneal cells), lung cells, epithelial cells, skeletal muscle cells, dendritic cells, hepatic cells, pancreatic cells, bone cells, hematopoietic stem cells, spleen cells, keratinocytes, fibroblasts, endothelial cells, prostate cells, and heart cells. In some embodiments, the polynucleotide or the vector comprising a plurality of expression cassettes is introduced to a target tissue type including central nervous system tissue, liver tissue, muscle tissue, ocular tissue, retinal tissue, heart tissue, skeletal muscle tissue, or kidney tissue.

In some embodiments, the polynucleotide or the vector comprising a plurality of expression cassettes of the disclosure may be introduced to cells in vitro via a viral vector for administration of modified cells to a subject. In some embodiments, the viral vector encoding the polynucleotide or a plurality of recombinant polynucleotide cassettes of the disclosure is introduced to cells that have been removed from a subject. In some embodiments, the modified cells are placed back in the subject following introduction of the viral vector.

In some embodiments, a dose of modified cells is administered to a subject according to the age and species of the subject, disease or disorder to be treated, as well as the cell type or state and mode of administration. In some embodiments, at least about 10²-10⁸ cells are administered per dose. In some embodiments, cells transduced with viral vector are administered to a subject in an effective amount.

In some embodiments, the dose of viral vector administered to a subject will vary according to the age of the subject, the disease or disorder to be treated, and mode of administration. In some embodiments, the dose for achieving a therapeutic effect is a virus titer of at least about 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶ or more transducing units.

Administration of the pharmaceutically useful polynucleotide of the present disclosure or the polynucleotide cassette of the present disclosure is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

Unless defined otherwise, all terms of art, notations and other technical and scientific terms or terminology used herein is intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “complementary” or “complementarity” refers to the ability of a nucleic acid to form one or more bonds with a corresponding nucleic acid sequence by, for example, hydrogen bonding (e.g., traditional Watson-Crick), covalent bonding, or other similar methods. In Watson-Crick base pairing, a double hydrogen bond forms between nucleobases T and A, whereas a triple hydrogen bond forms between nucleobases C and G. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%, 97%, 98%, 99%, or 100% over a region of 10, 15, 20, 25, 30, 35, 40, 45, 50, or more nucleotides, or can refer to two nucleic acids that hybridize under stringent conditions (i.e., stringent hybridization conditions). Nucleic acids can include nonspecific sequences. As used herein, the term “nonspecific sequence” or “not specific” can refer to a nucleic acid sequence that contains a series of residues that can be not designed to be complementary to or can be only partially complementary to any other nucleic acid sequence.

The terms “determining,” “measuring,” “evaluating,” “assessing,” “assaying,” and “analyzing” can be used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of”′ can include determining the amount of something present in addition to determining whether it is present or absent depending on the context.

The term “encode,” as used herein, refers to an ability of a polynucleotide to provide information or instructions sequence sufficient to produce a corresponding gene expression product. In a non-limiting example, mRNA can encode a polypeptide during translation, whereas DNA can encode a mRNA molecule during transcription.

As used herein, the term “engineered guide RNA” can be used interchangeable with “guide RNA” and refers to a designed polynucleotide that is at least partially complementary to a target RNA. An engineered guide RNA of the present disclosure can be used to facilitate modification of the target RNA. Modification of the target RNA includes alteration of RNA splicing, reduction or enhancement of protein translation, target RNA knockdown, target RNA degradation, and/or ADAR mediated RNA editing of the target RNA. In some cases, guide RNAs facilitate ADAR mediated RNA editing for the purpose of target mRNA knockdown, downstream protein translation reduction or inhibition, downstream protein translation enhancement, correction of mutations (including correction of any G to A mutation, such as missense or nonsense mutations), introduction of mutations (e.g., introduction of an A to I (read as a G by cellular machinery) substitution), or alter the function of any adenosine containing a regulatory motif (e.g., polyadenylation signal, miRNA binding site, etc.). In some cases, a guide RNA can effect a functional outcome (e.g., target RNA modulation, downstream protein translation) via a combination of mechanisms, for example, ADAR-mediated RNA editing and binding and/or degrading target RNA. In some cases, a guide RNA can facilitate introduction of mutations at sites targeted by enzymes in order to modify the affinity of such enzymes for targeting and cleaving such sites. The guide RNAs of this disclosure can contain one or more structural features. A structural feature can be formed from latent structure in latent (unbound) guide RNA upon hybridization of the engineered latent guide RNA to a target RNA. Latent structure refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed, and the latent structure provided in the guide RNA is, thus, unmasked. The formation and structure of a latent structural feature upon binding to the target RNA depends on the guide RNA sequence. For example, formation and structure of the latent structural feature may depend on a pattern of complementary and mismatched residues in the guide RNA sequence relative to the target RNA. The guide RNA sequence may be engineered to have a latent structural feature that forms upon binding to the target RNA. In such cases, a structural feature can include, but is not limited to, a mismatch, a wobble base pair, a symmetric internal loop, an asymmetric internal loop, a symmetric bulge, or an asymmetric bulge. In other instances, a structural feature can be a pre-formed structure (e.g., a GluR2 recruitment hairpin, or a hairpin from U7 snRNA).

As used herein, the term “targeting sequence” can be used interchangeable with “targeting domain” or “targeting region” and refers to a polynucleotide sequence within an engineered guide RNA sequence that is at least partially complementary to a target polynucleotide. The target polynucleotide (e.g., a target RNA or a target DNA) may be a region of a polynucleotide of interest, such as a gene or a messenger RNA. As used herein, a “complementary” sequence refers to a sequence that is a reverse complement relative to a second sequence.

A targeting sequence of an engineered guide RNA allows the engineered guide RNA to hybridize to a target polynucleotide (e.g., a target RNA) through base pairing, such as Watson Crick base pairing. A targeting sequence can be located at either the N-terminus or C-terminus of the engineered guide RNA, or both, or the targeting sequence can be within the engineered guide RNA. The targeting sequence can be of any length sufficient to hybridize with the target polynucleotide. In some cases, the targeting sequence is at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or up to about 200 nucleotides in length. In an embodiment, an engineered polynucleotide comprises a targeting sequence that is about 25 to 200, 50 to 150, 75 to 100, 80 to 110, 90 to 120, 95 to 115, 60 to 200, 60 to 180, 60 to 160, 60 to 140, 70 to 200, 70 to 180, 70 to 160, 70 to 140, 80 to 200, 80 to 190, 80 to 170, 80 to 160, 80 to 150, 80 to 140, 80 to 130, 80 to 120, 90 to 200, 90 to 190, 90 to 180, 90 to 170, 90 to 160, 90 to 150, 90 to 140, 90 to 130, 90 to 120, 100 to 200, 100 to 190, 100 to 180, 100 to 170, 100 to 160, 100 to 150, 100 to 140, 100 to 130, 100 to 120, 110 to 200, 110 to 190, 110 to 180, 110 to 170, 110 to 160, 110 to 150, 110 to 140, 110 to 120, 120 to 200, 120 to 190, 120 to 180, 120 to 170, 120 to 160, 120 to 150, 120 to 140, 130 to 200, 130 to 190, 130 to 180, 130 to 170, 130 to 160, 130 to 150, 140 to 200, 140 to 190, 140 to 180, 140 to 170, 140 to 160, 150 to 200, 150 to 190, 150 to 180, 150 to 170, 160 to 200, 160 to 190 or 160 to 180 nucleotides in length.

A targeting sequence comprises at least partial sequence complementarity to a target polynucleotide. The targeting sequence may have a degree of sequence complementarity to the target polynucleotide sufficient to hybridize with the target polynucleotide. In some cases, the targeting sequence comprises 95%, 96%, 97%, 98%, 99%, or 100% sequence complementarity to the target polynucleotide. In some cases, the targeting sequence comprises less than 100% complementarity to the target polynucleotide sequence. For example, the targeting sequence may have a single base mismatch relative to the target polynucleotide when bound to the target polynucleotide. In other cases, the targeting sequence comprises at least about 1, 2, 3, 4, 5, 6, 7, 8, 9, 10, 11, 12, 13, 14, 15, 16, 20, 30, 40 or up to about 50 base mismatches relative to the target polynucleotide when bound to the target polynucleotide. In some aspects, nucleotide mismatches can be associated with structural features provided herein. In some aspects, a targeting sequence comprises at least about 1, 2, 3, 4, 5, 6, 7, 8, 9, 10, 11, 12, 13, 14, or up to about 15 nucleotides that differ in complementarity from a wildtype polynucleotide of a subject target polynucleotide.

A targeting sequence comprises nucleotide residues having complementarity to a target polynucleotide. The targeting sequence may have a number of residues with complementarity to the target polynucleotide sufficient to hybridize with the target polynucleotide. The complementary residues may be contiguous or non-contiguous. In some cases, the targeting sequence comprises at least 50 nucleotides having complementarity to the target polynucleotide. In some cases, the targeting sequence comprises from 50 to 150 nucleotides having complementarity to the target polynucleotide. In some cases, the targeting sequence comprises from 50 to 200 nucleotides having complementarity to the target polynucleotide. In some cases, the targeting sequence comprises from 50 to 250 nucleotides having complementarity to the target polynucleotide. In some cases, the targeting sequence comprises from 50 to 300 nucleotides having complementarity to the target polynucleotide. In some cases, the targeting sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 nucleotides having complementarity to the target polynucleotide. In some cases, the targeting sequence comprises more than 50 nucleotides total and has at least 50 nucleotides having complementarity to the target polynucleotide. In some cases, the targeting sequence comprises from 50 to 400 nucleotides total and has from 50 to 150 nucleotides having complementarity to the target polynucleotide. In some cases, the targeting sequence comprises from 50 to 400 nucleotides total and has from 50 to 200 nucleotides having complementarity to the target polynucleotide. In some cases, the targeting sequence comprises from 50 to 400 nucleotides total and has from 50 to 250 nucleotides having complementarity to the target polynucleotide. In some cases, the targeting sequence comprises from 50 to 400 nucleotides total and has from 50 to 300 nucleotides having complementarity to the target polynucleotide. In some cases, the at least 50 nucleotides having complementarity to the target polynucleotide are separated by one or more mismatches, one or more bulges, or one or more loops, or any combination thereof. In some cases, the from 50 to 150 nucleotides having complementarity to the target polynucleotide are separated by one or more mismatches, one or more bulges, or one or more loops, or any combination thereof. In some cases, the from 50 to 200 nucleotides having complementarity to the target polynucleotide are separated by one or more mismatches, one or more bulges, or one or more loops, or any combination thereof. In some cases, the from 50 to 250 nucleotides having complementarity to the target polynucleotide are separated by one or more mismatches, one or more bulges, or one or more loops, or any combination thereof. In some cases, the from 50 to 300 nucleotides having complementarity to the target polynucleotide are separated by one or more mismatches, one or more bulges, or one or more loops, or any combination thereof. For example, a targeting sequence comprises a total of 54 nucleotides wherein, sequentially, 25 nucleotides are complementarity to the target polynucleotide, 4 nucleotides form a bulge, and 25 nucleotides are complementarity to the target polynucleotide. As another example, a targeting sequence comprises a total of 118 nucleotides wherein, sequentially, 25 nucleotides are complementarity to the target polynucleotide, 4 nucleotides form a bulge, 25 nucleotides are complementarity to the target polynucleotide, 14 nucleotides form a loop, and 50 nucleotides are complementary to the target polynucleotide.

As used herein, the term “facilitates RNA editing” by an engineered guide RNA refers to the ability of the engineered guide RNA when associated with an RNA editing entity and a target RNA to provide a targeted edit of the target RNA by the RNA edited entity. In some instances, the engineered guide RNA can directly recruit or position/orient the RNA editing entity to the proper location for editing of the target RNA. In other instances, the engineered guide RNA when hybridized to the target RNA forms a guide-target RNA scaffold with one or more structural features as described herein, where the guide-target RNA scaffold with structural features recruits or positions/orients the RNA editing entity to the proper location for editing of the target RNA.

A “guide-target RNA scaffold,” as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more structural features selected from a bulge, mismatch, internal loop, hairpin, or wobble base pair.

“Messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As disclosed herein, a “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a “bulge” or an “internal loop,” depending on the size of the structural feature.

The term “structured motif” refers to a combination of two or more structural features in a guide-target RNA scaffold.

The terms “subject,” “individual,” or “patient” can be used interchangeably herein. A “subject” refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some cases, the subject may not be necessarily diagnosed or suspected of being at high risk for the disease. The term “in vivo” refers to an event that takes place in a subject's body.

The term “ex vivo” refers to an event that takes place outside of a subject's body. An ex vivo assay may not be performed on a subject. Rather, it can be performed upon a sample separate from a subject. An example of an ex vivo assay performed on a sample can be an “in vitro” assay.

The term “in vitro” refers to an event that takes places contained in a container for holding laboratory reagent such that it can be separated from the biological source from which the material can be obtained. In vitro assays can encompass cell-based assays in which living or dead cells can be employed. In vitro assays can also encompass a cell-free assay in which no intact cells can be employed.

The term “wobble base pair” refers to two bases that weakly pair. For example, a wobble base pair can refer to a G paired with a U.

The term “substantially forms” as described herein, when referring to a particular secondary structure, refers to formation of at least 80% of the structure under physiological conditions (e.g., physiological pH, physiological temperature, physiological salt concentration, etc.).

As used herein, the term “therapeutic polynucleotide” may to a polynucleotide that is introduced into a cell and is capable of being expressed in the cell or to a polynucleotide that may, in itself, have a therapeutic activity, such as a gRNA or a tRNA.

As used herein, the term “polynucleotide” refers to a single or double-stranded polymer of deoxyribonucleotide (DNA) or ribonucleotide (RNA) bases read from the 5′ to the 3′ end. The term “RNA” is inclusive of dsRNA (double stranded RNA), snRNA (small nuclear RNA), lncRNA (long non-coding RNA), mRNA (messenger RNA), miRNA (microRNA) RNAi (inhibitory RNA), siRNA (small interfering RNA), shRNA (short hairpin RNA), tRNA (transfer RNA), rRNA (ribosomal RNA), snoRNA (small nucleolar RNA), and cRNA (complementary RNA). The term DNA is inclusive of cDNA, genomic DNA, and DNA-RNA hybrids. A sequence of a polynucleotide may be provided interchangeably as an RNA sequence (containing U) or a DNA sequence (containing T). A sequence provided as an RNA sequence is intended to also cover the corresponding DNA sequence and the reverse complement RNA sequence or DNA sequence. A sequence provided as a DNA sequence is intended to also cover the corresponding RNA sequence and the reverse complement RNA sequence or DNA sequence.

The term “protein”, “peptide” and “polypeptide” can be used interchangeably and in their broadest sense can refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation can be placed on the maximum number of amino acids which can comprise a protein's or peptide's sequence. As used herein the term “amino acid” can refer to either natural amino acids, unnatural amino acids, or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that can be used to link these domains together-optionally to preserve the conformation of the fused protein domains, prevent unfavorable interactions between the fused protein domains which can compromise their respective functions, or both.

The term “ameliorating” refers to any therapeutically beneficial result in the treatment of a disease state, e.g., Rett syndrome, including prophylaxis, lessening in the severity or progression, remission, or cure thereof.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence (also called the subject sequence) to which test sequences (also called query sequences) are compared. The percent sequence identity is defined as a test sequence's percent identity to a reference sequence. For example, when stated “Sequence A having a sequence identity of 50% to Sequence B,” Sequence A is the test sequence and Sequence B is the reference sequence. When using a sequence comparison algorithm, test and reference sequences are input into a computer program, subsequence coordinates are designated, if necessary, and sequence algorithm program parameters are designated. The sequence comparison algorithm then aligns the sequences to achieve the maximum alignment, based on the designated program parameters, introducing gaps in the alignment if necessary. The percent sequence identity for the test sequence(s) relative to the reference sequence can then be determined from the alignment of the test sequence to the reference sequence. The equation for percent sequence identity from the aligned sequence is as follows:

[(Number of Identical Positions)/(Total Number of Positions in the Test Sequence)]×100%

For purposes herein, percent identity and sequence similarity calculations are performed using the BLAST algorithm for sequence alignment, which is described in Altschul et al., J. Mol. Biol. 215:403-410 (1990). Software for performing BLAST analyses is publicly available through the National Center for Biotechnology Information (www.ncbi.nlm.nih.gov/). The BLAST algorithm uses a test sequence (also called a query sequence) and a reference sequence (also called a subject sequence) to search against, or in some cases, a database of multiple reference sequences to search against. The BLAST algorithm performs sequence alignment by finding high-scoring alignment regions between the test and the reference sequences by scoring alignment of short regions of the test sequence (termed “words”) to the reference sequence. The scoring of each alignment is determined by the BLAST algorithm and takes factors into account, such as the number of aligned positions, as well as whether introduction of gaps between the test and the reference sequences would improve the alignment. The alignment scores for nucleic acids can be scored by set match/mismatch scores. For protein sequences, the alignment scores can be scored using a substitution matrix to evaluate the significance of the sequence alignment, for example, the similarity between aligned amino acids based on their evolutionary probability of substitution. For purposes herein, the substitution matrix used is the BLOSUM62 matrix. For purposes herein, the public default values of Apr. 6, 2023, are used when using the BLASTN and BLASTP algorithms. The BLASTN and BLASTP algorithms then output a “Percent Identity” output value and a “Query Coverage” output value. The overall percent sequence identity as used herein can then be calculated from the BLASTN or BLASTP output values as follows:

Percent Sequence Identity=(“Percent Identity” output value)×(“Query Coverage” output value)

The following non-limiting examples illustrate the calculation of percent identity between two nucleic acids sequences. The percent identity is calculated as follows: [(number of identical nucleotide positions)/(total number of nucleotides in the test sequence)]×100%. Percent identity is calculated to compare test sequence 1: AAAAAGGGGG (SEQ ID NO: 94) (length=10 nucleotides) to reference sequence 2: AAAAAAAAAA (SEQ ID NO: 95) (length=10 nucleotides). The percent identity between test sequence 1 and reference sequence 2 would be [(5)/(10)]×100%=50%. Test sequence 1 has 50% sequence identity to reference sequence 2. In another example, percent identity is calculated to compare test sequence 3: CCCCCGGGGGGGGGGCCCCC (SEQ ID NO: 96) (length=20 nucleotides) to reference sequence 4: GGGGGGGGGG (SEQ ID NO: 97) (length=10 nucleotides). The percent identity between test sequence 3 and reference sequence 4 would be [(10)/(20)]×100%=50%. Test sequence 3 has 50% sequence identity to reference sequence 4. In another example, percent identity is calculated to compare test sequence 5: GGGGGGGGGG (SEQ ID NO: 97) (length=10 nucleotides) to reference sequence 6: CCCCCGGGGGGGGGGCCCCC (SEQ ID NO: 96) (length=20 nucleotides). The percent identity between test sequence 5 and reference sequence 6 would be [(10)/(10)]×100%=100%. Test sequence 5 has 100% sequence identity to reference sequence 6.

The following non-limiting examples illustrate the calculation of percent identity between two protein sequences. The percent identity is calculated as follows: [(number of identical amino acid positions)/(total number of amino acids in the test sequence)]×100%. Percent identity is calculated to compare test sequence 7: FFFFFYYYYY (SEQ ID NO: 98) (length=10 amino acids) to reference sequence 8: YYYYYYYYYY (SEQ ID NO: 99) (length=10 amino acids). The percent identity between test sequence 7 and reference sequence 8 would be [(5)/(10)]×100%=50%. Test sequence 7 has 50% sequence identity to reference sequence 8. In another example, percent identity is calculated to compare test sequence 9: LLLLLFFFFFYYYYYLLLLL (SEQ ID NO: 100) (length=20 amino acids) to reference sequence 10: FFFFFYYYYY (SEQ ID NO: 98) (length=10 amino acids). The percent identity between test sequence 9 and reference sequence 10 would be [(10)/(20)]×100%=50%. Test sequence 9 has 50% sequence identity to reference sequence 10. In another example, percent identity is calculated to compare test sequence 11: FFFFFYYYYY (SEQ ID NO: 98) (length=10 amino acids) to reference sequence 12: LLLLLFFFFFYYYYYLLLLL (SEQ ID NO: 100) (length=20 amino acids). The percent identity between test sequence 11 and reference sequence 12 would be [(10)/(10)]×100%=100%. Test sequence 11 has 100% sequence identity to reference sequence 12.

As used herein, the term “subject” broadly refers to any animal, including but not limited to, human and non-human animals (e.g., dogs, cats, cows, horses, sheep, pigs, poultry, fish, crustaceans, etc.).

As used herein, the term “effective amount” refers to the amount of a composition (e.g., a synthetic peptide) sufficient to effect beneficial or desired results. An effective amount can be administered in one or more administrations, applications or dosages and is not intended to be limited to a particular formulation or administration route.

As used herein, the term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment (e.g., peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “treatment” or “treating” means an approach to obtaining a beneficial or intended clinical result. The beneficial or intended clinical result can include a therapeutic benefit and/or a prophylactic benefit, alleviation of symptoms, a reduction in the severity of the disease, inhibiting an underlying cause of a disease or condition, steadying diseases in a non-advanced state, delaying the progress of a disease, and/or improvement or alleviation of disease conditions. Also, a therapeutic benefit can be achieved with the eradication or amelioration of one or more of the physiological symptoms associated with the underlying disorder such that an improvement can be observed in the subject, notwithstanding that the subject can still be afflicted with the underlying disorder. A prophylactic effect includes delaying, preventing, or eliminating the appearance of a disease or condition, delaying or eliminating the onset of one or more symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 21st Ed., Mack Publ. Co., Easton, Pa. (2005), incorporated herein by reference in its entirety.

Throughout this application, various embodiments are presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the singular forms “a,” “an” and “the” include plural referents unless the context clearly dictates otherwise.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

### Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A viral vector comprising: a plurality of expression cassettes, wherein each expression cassette independently comprises: a promoter sequence; a payload sequence under transcriptional control of the promoter sequence, the payload sequence comprising a small RNA payload; and a transcription termination sequence, wherein each expression cassette of the plurality of expression cassettes is arranged in a 5′ to 3′ orientation to have a read directionality of forward or reverse. 2. The viral vector of embodiment 1, wherein the plurality of expression cassettes comprises two expression cassettes, three expression cassettes, four expression cassettes, five expression cassettes, six expression cassettes, seven expression cassettes, eight expression cassettes, nine expression cassettes, or ten expression cassettes. 3. The viral vector of embodiment 1 or embodiment 2, wherein the plurality of expression cassettes comprises a first expression cassette and a second expression cassette, wherein: a) the first expression cassette has the read directionality of forward and the second expression cassette has the read directionality of reverse; b) the first expression cassette has the read directionality of reverse and the second expression cassette has the read directionality of forward; c) the first expression cassette has the read directionality of forward and the second expression cassette has the read directionality of forward; or d) the first expression cassette has the read directionality of reverse and the second expression cassette has the read directionality of reverse. 4. The viral vector of embodiment 1 or embodiment 2, wherein the plurality of expression cassettes comprises a first expression cassette, a second expression cassette and a third expression cassette, wherein: a) the first expression cassette has the read directionality of forward, the second expression cassette has the read directionality of forward, and the third expression cassette has the read directionality of reverse; b) the first expression cassette has the read directionality of forward, the second expression cassette has the read directionality of reverse, and the third expression cassette has the read directionality of forward; c) the first expression cassette has the read directionality of forward, the second expression cassette has the read directionality of reverse, and the third expression cassette has the read directionality of reverse; d) the first expression cassette has the read directionality of reverse, the second expression cassette has the read directionality of forward, and the third expression cassette has the read directionality of forward; e) the first expression cassette has the read directionality of reverse, the second expression cassette has the read directionality of forward, and the third expression cassette has the read directionality of reverse; f) the first expression cassette has the read directionality of reverse, the second expression cassette has the read directionality of reverse, and the third expression cassette has the read directionality of forward; g) the first expression cassette has the read directionality of forward, the second expression cassette has the read directionality of forward, and the third expression cassette has the read directionality of forward; or h) the first expression cassette has the read directionality of reverse, the second expression cassette has the read directionality of reverse, and the third expression cassette has the read directionality of reverse. 5. The viral vector of embodiment 1 or embodiment 2, wherein the plurality of expression cassettes comprises a first expression cassette, a second expression cassette, a third expression cassette, and a fourth expression cassette, wherein: a) the first expression cassette has the read directionality of reverse, the second expression cassette has the read directionality of reverse, the third expression cassette has the read directionality of forward, and the fourth expression cassette has the read directionality of forward; b) the first expression cassette has the read directionality of forward, the second expression cassette has the read directionality of forward, the third expression cassette has the read directionality of forward, and the fourth expression cassette has the read directionality of forward; c) the first expression cassette has the read directionality of reverse, the second expression cassette has the read directionality of reverse, the third expression cassette has the read directionality of reverse, and the fourth expression cassette has the read directionality of reverse; d) the first expression cassette has the read directionality of reverse, the second expression cassette has the read directionality of forward, the third expression cassette has the read directionality of forward, and the fourth expression cassette has the read directionality of forward; e) the first expression cassette has the read directionality of reverse, the second expression cassette has the read directionality of reverse, the third expression cassette has the read directionality of reverse, and the fourth expression cassette has the read directionality of forward; f) the first expression cassette has the read directionality of forward, the second expression cassette has the read directionality of forward, the third expression cassette has the read directionality of forward, and the fourth expression cassette has the read directionality of reverse; g) the first expression cassette has the read directionality of forward, the second expression cassette has the read directionality of forward, the third expression cassette has the read directionality of reverse, and the fourth expression cassette has the read directionality of forward; h) the first expression cassette has the read directionality of forward, the second expression cassette has the read directionality of forward, the third expression cassette has the read directionality of reverse, and the fourth expression cassette has the read directionality of reverse; i) the first expression cassette has the read directionality of forward, the second expression cassette has the read directionality of reverse, the third expression cassette has the read directionality of forward, and the fourth expression cassette has the read directionality of forward; j) the first expression cassette has the read directionality of forward, the second expression cassette has the read directionality of reverse, the third expression cassette has the read directionality of forward, and the fourth expression cassette has the read directionality of reverse; k) the first expression cassette has the read directionality of forward, the second expression cassette has the read directionality of reverse, the third expression cassette has the read directionality of reverse, and the fourth expression cassette has the read directionality of forward; 1) the first expression cassette has the read directionality of forward, the second expression cassette has the read directionality of reverse, the third expression cassette has the read directionality of reverse, and the fourth expression cassette has the read directionality of reverse; m) the first expression cassette has the read directionality of reverse, the second expression cassette has the read directionality of forward, the third expression cassette has the read directionality of forward, and the fourth expression cassette has the read directionality of reverse; n) the first expression cassette has the read directionality of reverse, the second expression cassette has the read directionality of forward, the third expression cassette has the read directionality of reverse, and the fourth expression cassette has the read directionality of forward; o) the first expression cassette has the read directionality of reverse, the second expression cassette has the read directionality of forward, the third expression cassette has the read directionality of reverse, and the fourth expression cassette has the read directionality of reverse; or p) the first expression cassette has the read directionality of reverse, the second expression cassette has the read directionality of reverse, the third expression cassette has the read directionality of forward, and the fourth expression cassette has the read directionality of reverse. 6. The viral vector of embodiment 1 or embodiment 2, wherein the plurality of expression cassettes comprises a first expression cassette, a second expression cassette, a third expression cassette, and a fourth expression cassette, wherein: a) the first expression cassette has the read directionality of reverse, the second expression cassette has the read directionality of reverse, the third expression cassette has the read directionality of forward, and the fourth expression cassette has the read directionality of forward; b) the first expression cassette has the read directionality of forward, the second expression cassette has the read directionality of forward, the third expression cassette has the read directionality of forward, and the fourth expression cassette has the read directionality of forward; c) the first expression cassette has the read directionality of reverse, the second expression cassette has the read directionality of reverse, the third expression cassette has the read directionality of reverse, and the fourth expression cassette has the read directionality of reverse; d) the first expression cassette has the read directionality of reverse, the second expression cassette has the read directionality of forward, the third expression cassette has the read directionality of forward, and the fourth expression cassette has the read directionality of forward; or e) the first expression cassette has the read directionality of reverse, the second expression cassette has the read directionality of reverse, the third expression cassette has the read directionality of reverse, and the fourth expression cassette has the read directionality of forward. 7. The viral vector of any one of embodiments 3-6, wherein the first expression cassette and the second expression cassette comprise a different promoter sequence. 8. The viral vector of any one of embodiments 3-6, wherein the first expression cassette and the second expression cassette comprise same promoter sequence. 9. The viral vector of any one of embodiments 4-8, wherein the first expression cassette, the second expression cassette and the third expression cassette each comprise a different promoter sequence. 10. The viral vector of any one of embodiments 4-8, wherein at least two expression cassettes comprise same promoter sequence. 11. The viral vector of any one of embodiments 5-10, wherein the first expression cassette, the second expression cassette, the third expression cassette and fourth expression cassette each comprise a different promoter sequence. 12. The viral vector of embodiment 5-11, wherein at least two of the four expression cassettes comprise same promoter sequence. 13. The viral vector of any one of embodiments 1-12, wherein the viral vector is an adeno-associated viral vector. 14. The viral vector of any one of embodiments 1-13, wherein the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, chimeras thereof, and combinations thereof. 15. The viral vector of any one of embodiments 1-14, wherein the promoter sequence comprises at least 80%, at least 85%, at least 90%, at least 95%, or 100% sequence identity to any one of SEQ ID NO: 1-SEQ ID NO: 9. 16. The viral vector of any one of embodiments 1-15, wherein the transcription termination sequence comprises at least 80%, at least 85%, at least 90%, at least 95%, or 100% sequence identity to any one of SEQ ID NO: 10-SEQ ID NO: 16. 17. The viral vector of any one of embodiments 1-16, wherein the small RNA payload comprises an engineered guide RNA capable of hybridizing to a target sequence. 18. The viral vector of embodiment 17, wherein the engineered guide RNA is at least 70%, at least 75%, at least 80%, at least 85%, at least 90%, at least 95%, or 100% reverse complementary to the target sequence. 19. The viral vector of embodiment 17 or embodiment 18, wherein the engineered guide RNA comprises at least one base pair mismatch relative to the target sequence. 20. The viral vector of any one of embodiments 17-19, wherein the target sequence comprises an adenosine residue. 21. The viral vector of any one of embodiments 17-20, wherein the target sequence is an RNA sequence. 22. The viral vector of embodiment 21, wherein the RNA sequence is a mRNA or a pre-mRNA. 23. The viral vector of any one of embodiments 17-22, wherein the target sequence comprises a G to A mutation relative to a wild type sequence. 24. The viral vector of any one of embodiments 17-23, wherein the target sequence comprises a missense mutation or a nonsense mutation relative to a wild type sequence. 25. The viral vector of any one of embodiments 17-24, wherein the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2). 26. The viral vector of any one of embodiments 1-25, wherein the small RNA payload comprises an antisense oligonucleotide, an siRNA, an shRNA, a miRNA, or a tracrRNA. 27. The viral vector of any one of embodiments 1-26, wherein the small RNA payload is not less than 20 nucleotide residues and not more than 500 nucleotide residues long. 28. The viral vector of any one of embodiments 1-27, wherein the small RNA payload is not less than 60 and not more than 100 residues long. 29. The viral vector of any one of embodiments 1-28, wherein the small RNA payload is not less than 80 and not more than 120 residues long. 30. The viral vector of any one of embodiments 1-29, wherein the small RNA payload is not less than 100 and not more than 140 residues long. 31. The viral vector of any one of embodiments 1-30, wherein the small RNA payload is not less than 130 and not more than 170 residues long. 32. The viral vector of any one of embodiments 1-31, wherein the payload sequence further comprises an Sm binding sequence or a hairpin sequence. 33. The viral vector of embodiment 32, wherein the hairpin sequence comprises a U7 hairpin. 34. The viral vector of any one of embodiments 1-33, wherein each expression cassette of the plurality of expression cassettes independently has a length of not less than 1300 nucleotide residues and not more than 2160 nucleotide residues. 35. The viral vector of any one of embodiments 1-34, wherein each expression cassette of the plurality of expression cassettes independently comprises at least 80% sequence identity to a U1 sequence or a U7 sequence. 36. The viral vector of any one of embodiments 1-35, wherein the U1 sequence is a mouse U1 sequence or a human U1 sequence. 37. The viral vector of any one of embodiments 1-36, wherein the U7 sequence is a mouse U7 sequence or a human U7 sequence. 38. The viral vector of embodiment 17, wherein the engineered guide RNA is capable of forming a guide-target RNA scaffold comprising a structural feature upon hybridization of the small RNA payload to a target sequence. 39. The viral vector of embodiment 38, wherein the structural feature is a bulge, a mismatch, an internal loop, a hairpin, or combinations thereof. 40. The viral vector of embodiment 38 or embodiment 39, wherein the structural feature comprises the bulge, and wherein the bulge is a symmetric bulge. 41. The viral vector of any one of embodiments 38-40, wherein the structural feature comprises the bulge, and wherein the bulge is an asymmetric bulge. 42. The viral vector of any one of embodiments 38-41, wherein the structural feature comprises the internal loop, and wherein the internal loop is a symmetric internal loop. 43. The viral vector of any one of embodiments 38-42, wherein the structural feature comprises the internal loop, and wherein the internal loop is an asymmetric internal loop. 44. The viral vector of any one of embodiments 38-43, wherein the structural feature comprises the hairpin, and wherein the hairpin is a recruitment hairpin or a non-recruitment hairpin. 45. The viral vector of any one of embodiments 38-44, wherein the guide-target RNA scaffold comprises a wobble base pair. 46. A pharmaceutical composition comprising the viral vector of any one of embodiments 1-45 and a pharmaceutically acceptable excipient, carrier, diluent, or combination thereof. 47. A method of expressing a small RNA payload in a cell, the method comprising delivering the viral vector of any one of embodiments 1-45 or the pharmaceutical composition of embodiment 46 to a cell and expressing the small RNA payload encoded by the expression cassette in the cell. 48. A method of editing a target sequence, the method comprising: delivering the viral vector of any one of embodiments 1-45 or the pharmaceutical composition of embodiment 46 to a cell encoding the target sequence, expressing the small RNA payload in the cell; forming a guide-target RNA scaffold upon hybridization of the small RNA payload to the target sequence; recruiting an editing enzyme to the target sequence; and editing the target sequence with the editing enzyme. 49. A method of administering a viral vector to a subject with a disease, the method comprising: administering to the subject a composition comprising the viral vector of any one of embodiments 1-45 or the pharmaceutical composition of embodiment 46; delivering the expression cassette to a cell of the subject; and expressing a small RNA payload in the cell. 50. A method of treating a disease in a subject, the method comprising: administering to the subject a composition comprising the viral vector of any one of embodiments 1-45 or the pharmaceutical composition of embodiment 46; delivering the expression cassette to a cell of the subject; and expressing a small RNA payload in the cell, thereby treating the disease. 51. The method of embodiment 49 or embodiment 50, wherein the disease is a synucleinopathy, Parkinson's disease, Lewy body dementia, multiple system atrophy, Charcot-Marie-Tooth disease, hereditary neuropathy with liability to pressure palsies, Yuan-Harel-Lupski syndrome, a tauopathy, Alzheimer's disease, frontotemporal dementia, progressive supranuclear palsy, corticobasal degeneration, chronic traumatic encephalopathy, autism, traumatic brain injury, Dravet syndrome, Crohn's disease, muscular dystrophy, B-cell leukemia, Dejerine-Sottas disease, Stargardt disease, alpha-1 antitrypsin deficiency, Tay-Sachs disease, cystic fibrosis, liposomal acid lipase deficiency, or Gaucher disease. 52. The method of any one of embodiments 48-51, wherein the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2). 53. The method of any one of embodiments 47-52, wherein the small RNA payload comprises an engineered guide RNA that hybridizes to a target sequence, and wherein the cell encodes the target sequence. 54. The method of embodiment 53, further comprising forming a guide-target RNA scaffold upon hybridization of the engineered guide RNA to the target sequence, recruiting an editing enzyme to the target sequence, and editing the target sequence with the editing enzyme. 55. The method of embodiment 53 or embodiment 54, wherein the target sequence comprises a mutation relative to a wild type sequence. 56. The method of embodiment 55, wherein editing the target sequence corrects the mutation in the target sequence. 57. The method of embodiment 55 or embodiment 56, wherein the mutation is a missense mutation. 58. The method of embodiment 55 or embodiment 56, wherein the mutation is a nonsense mutation. 59. The method of any one of embodiments 55-58, wherein the mutation is a G to A mutation. 60. The method of any one of embodiments 55-59, wherein the mutation is associated with the disease. 61. The method of any one of embodiment 48 or embodiments 54-60, wherein editing the target sequence comprises editing an untranslated region of the target. 62. The method of embodiment 61, wherein the untranslated region is a 5′ untranslated region or a 3′ untranslated region. 63. The method of embodiment 62, wherein the 3′ untranslated region is a polyadenylation sequence. 64. The method of any one of embodiment 48 or embodiments 54-63, wherein editing the target sequence comprises editing a translation initiation site. 65. The method of any one of embodiment 48 or embodiments 54-64, wherein editing the target sequence alters expression of the target sequence. 66. The method of embodiment 65, wherein editing the target sequence increases expression of the target sequence. 67. The method of embodiment 65, wherein editing the target sequence decreases expression of the target sequence. 68. The method of any one of embodiments 48 or 54-67, wherein the editing enzyme comprises an ADAR, an APOBEC, or a Cas nuclease. 69. The method of embodiment 68, wherein the ADAR comprises ADAR1, ADAR2, ADAR3, or combinations thereof. 70. The method of any one of embodiments 48 or 54-69, wherein the target sequence comprises RNA or DNA. 71. The method of any one of embodiments 48 or 54-70, wherein the target sequence is a mRNA or a pre-mRNA. 72. The method of any one of embodiments 48 or 54-71, wherein editing the target sequence comprises deamidating a nucleotide of the target sequence. 73. The method of any one of embodiments 48 or 54-72, wherein the target sequence is edited with an efficiency of at least 10%, at least 20%, or at least 25%.

### Further Numbered Embodiments

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A polynucleotide comprising a plurality of expression cassette sequences comprising a first expression cassette sequence and a second expression cassette sequence, wherein: the first, second, and plurality of expression cassette sequences each independently comprise: a promoter sequence, a payload sequence under transcriptional control of the promoter sequence, the payload sequence comprising a small RNA payload, and a transcription termination sequence; the first, second, and plurality of expression cassette sequences are each independently arranged in a 5′ to 3′ orientation to have a read directionality of forward or reverse; and the first and second expression cassette sequences are different sequences. 2. The polynucleotide of embodiment 1, wherein the first expression cassette sequence has a read directionality of forward and the second expression cassette sequence has a read directionality of reverse. 3. The polynucleotide of embodiment 1, wherein the first expression cassette sequence has a read directionality of reverse and the second expression cassette sequence has a read directionality of forward. 4. The polynucleotide of embodiment 1, wherein the first expression cassette sequence has a read directionality of forward and the second expression cassette sequence has a read directionality of forward. 5. The polynucleotide of any one of embodiments 1-4, wherein the plurality of expression cassette sequences comprises two expression cassette sequences, three expression cassette sequences, four expression cassette sequences, five expression cassette sequences, six expression cassette sequences, seven expression cassette sequences, eight expression cassette sequences, nine expression cassette sequences, or ten expression cassette sequences. 6. The polynucleotide of any one of embodiments 1-5, wherein the small RNA payload comprises an engineered guide RNA sequence capable of hybridizing to a target sequence. 7. The polynucleotide of any one of embodiments 1-6, wherein the first expression cassette sequence comprises a first engineered guide RNA sequence capable of hybridizing to a target sequence and the second expression cassette comprises a second engineered guide RNA sequence capable of hybridizing to the target sequence. 8. The polynucleotide of embodiment 7, wherein the second engineered guide RNA sequence has at least one and no more than 30 nucleotide alterations from the first engineered guide RNA sequence. 9. The polynucleotide of embodiment 8, wherein the nucleotide alterations from the first engineered guide RNA sequence are dispersed at a frequency of at least one and no more than 4 nucleotide alterations per every 10 nucleotides in the second engineered guide RNA sequence. 10. The polynucleotide of embodiment 7 or embodiment 8, wherein the nucleotide alterations from the first engineered guide RNA sequence are dispersed at a frequency of 3 nucleotide alterations per every 10 nucleotides in the second engineered guide RNA sequence. 11. The polynucleotide of any one of embodiments 6-10, wherein the engineered guide RNA sequence, the first engineered guide RNA sequence, or the second engineered guide RNA sequence are each independently at least 70%, at least 75%, at least 80%, at least 85%, at least 90%, at least 95%, or 100% reverse complementary to the target sequence. 12. The polynucleotide of any one of embodiments 6-11, wherein the engineered guide RNA sequence, the first engineered guide RNA sequence, or the second engineered guide RNA sequence are each independently capable of forming a guide-target RNA scaffold comprising one or more structural features upon hybridization of the small RNA payload to a target sequence. 13. The polynucleotide of embodiment 12, wherein the guide-target RNA scaffold of the first engineered guide RNA sequence and the guide-target RNA scaffold of the second engineered guide RNA sequence comprise the same one or more structural features. 14. The polynucleotide of embodiment 12 or embodiment 13, wherein the one or more structural features comprise a bulge, a mismatch, an internal loop, a hairpin, or combinations thereof. 15. The polynucleotide of any one of embodiments 12-14, wherein the one or more structural features comprises the bulge, and wherein the bulge is a symmetric bulge. 16. The polynucleotide of any one of embodiments 12-14, wherein the one or more structural features comprises the bulge, and wherein the bulge is an asymmetric bulge. 17. The polynucleotide of any one of embodiments 12-16, wherein the one or more structural features comprises the internal loop, and wherein the internal loop is a symmetric internal loop. 18. The polynucleotide of any one of embodiments 12-16, wherein the one or more structural features comprises the internal loop, and wherein the internal loop is an asymmetric internal loop. 19. The polynucleotide of any one of embodiments 12-18, wherein the one or more structural features comprises the hairpin, and wherein the hairpin is a recruitment hairpin or a non-recruitment hairpin. 20. The polynucleotide of any one of embodiments 12-19, wherein the guide-target RNA scaffold comprises one or more wobble base pairs. 21. The polynucleotide of embodiment 20, wherein the one or more of the wobble base pairs are GU wobble base pairs. 22. The polynucleotide of embodiment 20 or embodiment 21, wherein the guide-target RNA scaffold of the second engineered guide RNA sequence has between at least one and no more than 15 additional wobble base pairs than the guide-target RNA scaffold of the first engineered guide RNA sequence. 23. The polynucleotide of any one of embodiments 12-22, wherein the engineered guide RNA sequence, the first engineered guide RNA sequence, or the second engineered guide RNA sequence comprise at least one base pair mismatch relative to the target sequence. 24. The polynucleotide of any one of embodiments 12-23, wherein the target sequence comprises an adenosine residue. 25. The polynucleotide of any one of embodiments 12-24, wherein the target sequence is an RNA sequence. 26. The polynucleotide of embodiment 25, wherein the RNA sequence is a mRNA or a pre-mRNA. 27. The polynucleotide of any one of embodiments 12-26, wherein the target sequence comprises a G to A mutation relative to a wild type sequence. 28. The polynucleotide of any one of embodiments 12-27, wherein the target sequence comprises a missense mutation or a nonsense mutation relative to a wild type sequence. 29. The polynucleotide of any one of embodiments 12-28, wherein the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2). 30. The polynucleotide of any one of embodiments 1-29, wherein the small RNA payload comprises an antisense oligonucleotide, an siRNA, an shRNA, a miRNA, or a tracrRNA. 31. The polynucleotide of any one of embodiments 1-30, wherein the small RNA payload is not less than 20 nucleotide residues and not more than 500 nucleotide residues long. 32. The polynucleotide of any one of embodiments 1-31, wherein the small RNA payload is not less than 60 and not more than 100 residues long. 33. The polynucleotide of any one of embodiments 1-32, wherein the small RNA payload is not less than 80 and not more than 120 residues long. 34. The polynucleotide of any one of embodiments 1-33, wherein the small RNA payload is not less than 100 and not more than 140 residues long. 35. The polynucleotide of any one of embodiments 1-34, wherein the small RNA payload is not less than 130 and not more than 170 residues long. 36. The polynucleotide of any one of embodiments 1-35, wherein the promoter sequence comprises at least 80%, at least 85%, at least 90%, at least 95%, or 100% sequence identity to any one of SEQ ID NO: 1-SEQ ID NO: 9 or SEQ ID NO: 66-SEQ ID NO: 76. 37. The polynucleotide of any one of embodiments 1-36, wherein the transcription termination sequence comprises at least 80%, at least 85%, at least 90%, at least 95%, or 100% sequence identity to any one of SEQ ID NO: 10-SEQ ID NO: 16, SEQ ID NO: 34, SEQ ID NO: 35, or SEQ ID NO: 77-SEQ ID NO: 82. 38. The polynucleotide of any one of embodiments 1-37, wherein the payload sequence further comprises an Sm binding sequence or a hairpin sequence. 39. The polynucleotide of embodiment 38, wherein the hairpin sequence comprises a U7 hairpin. 40. The polynucleotide of any one of embodiments 1-39, wherein each expression cassette of the plurality of expression cassettes independently has a length of not less than 1300 nucleotide residues and not more than 2160 nucleotide residues. 41. The polynucleotide of any one of embodiments 1-40, wherein each expression cassette of the plurality of expression cassettes independently comprises at least 80% sequence identity to a U1 sequence or a U7 sequence. 42. The polynucleotide of embodiment 41, wherein the U1 sequence is a mouse U1 sequence or a human U1 sequence. 43. The polynucleotide of embodiment 41 or embodiment 42, wherein the U7 sequence is a mouse U7 sequence or a human U7 sequence. 44. A viral vector encoding the polynucleotide of any one of embodiments 1-43. 45. The viral vector of embodiment 44, wherein the viral vector is an adeno-associated viral vector. 46. The viral vector of embodiment 45, wherein the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, chimeras thereof, and combinations thereof. 47. A pharmaceutical composition comprising the polynucleotide of any one of embodiments 1-43 or the viral vector of any one of embodiments 44-46 and a pharmaceutically acceptable excipient, carrier, diluent, or combination thereof. 48. A method of editing a target sequence, the method comprising: delivering the polynucleotide of any one of embodiments 1-43, the viral vector of any one of embodiments 44-46, or the pharmaceutical composition of embodiment 47 to a cell encoding the target sequence, expressing the small RNA payload in the cell; forming a guide-target RNA scaffold upon hybridization of the small RNA payload to the target sequence; recruiting an editing enzyme to the target sequence; and editing the target sequence with the editing enzyme. 49. A method of administering a therapeutic polynucleotide to a subject with a disease, the method comprising: administering to the subject a composition comprising the polynucleotide of any one of embodiments 1-43, the viral vector of any one of embodiments 44-46, or the pharmaceutical composition of embodiment 47; delivering the therapeutic polynucleotide to a cell of the subject; and expressing a small RNA payload in the cell. 50. A method of treating a disease in a subject, the method comprising: administering to the subject a composition comprising the polynucleotide of any one of embodiments 1-43, the viral vector of any one of embodiments 44-46, or the pharmaceutical composition of embodiment 47; delivering a therapeutic polynucleotide to a cell of the subject; and expressing a small RNA payload in the cell, thereby treating the disease. 51. A method of editing a target sequence in a cell with an increased specificity, the method comprising: delivering the polynucleotide of any one of embodiments 1-43, the viral vector of any one of embodiments 44-46, or the pharmaceutical composition of embodiment 47 to a cell encoding the target sequence; expressing the small RNA payload in the cell, wherein, the small RNA payload comprises an engineered guide RNA sequence capable of hybridizing to the target sequence; forming a guide-target RNA scaffold upon hybridization of the engineered guide RNA sequence to the target sequence, wherein the guide-target RNA scaffold comprises at least one and no more than 15 wobble base pairs; recruiting an editing enzyme to the target sequence; and editing the target sequence with the editing enzyme with the increased specificity as compared to a specificity of a guide-target RNA scaffold comprising 0 wobble base pairs. 52. The method of embodiment 51, wherein the wobble base pairs comprise one or more GU wobble base pairs. 53. The method of embodiment 51 or embodiment 52, further comprising over twisting a helical structure of the guide-target RNA scaffold. 54. A method of expressing a small RNA payload in a cell, the method comprising delivering the polynucleotide of any one of embodiments 1-43, the viral vector of any one of embodiments 44-46, or the pharmaceutical composition of embodiment 47 to a cell and expressing the small RNA payload encoded by the expression cassette in the cell. 55. The method of any one of embodiments 49-50, wherein the disease is a synucleinopathy, Parkinson's disease, Lewy body dementia, multiple system atrophy, Charcot-Marie-Tooth disease, hereditary neuropathy with liability to pressure palsies, Yuan-Harel-Lupski syndrome, a tauopathy, Alzheimer's disease, frontotemporal dementia, progressive supranuclear palsy, corticobasal degeneration, chronic traumatic encephalopathy, autism, traumatic brain injury, Dravet syndrome, Crohn's disease, muscular dystrophy, B-cell leukemia, Dejerine-Sottas disease, Stargardt disease, alpha-1 antitrypsin deficiency, Tay-Sachs disease, cystic fibrosis, liposomal acid lipase deficiency, or Gaucher disease. 56. The method of any one of embodiments 48-55, wherein the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2). 57. The method of any one of embodiments 48-50, 54, or 55, wherein the small RNA payload comprises an engineered guide RNA that hybridizes to a target sequence, and wherein the cell encodes the target sequence. 58. The method of embodiment 57, further comprising forming a guide-target RNA scaffold upon hybridization of the engineered guide RNA to the target sequence, recruiting an editing enzyme to the target sequence, and editing the target sequence with the editing enzyme. 59. The method of embodiment 57 or embodiment 58, wherein the target sequence comprises a mutation relative to a wild type sequence. 60. The method of embodiment 59, wherein editing the target sequence corrects the mutation in the target sequence. 61. The method of embodiment 59 or embodiment 60, wherein the mutation is a missense mutation. 62. The method of embodiment 60 or embodiment 61, wherein the mutation is a nonsense mutation. 63. The method of any one of embodiments 59-62, wherein the mutation is a G to A mutation. 64. The method of any one of embodiments 59-63, wherein the mutation is associated with the disease. 65. The method of any one of embodiment 48 or embodiments 58-64, wherein editing the target sequence comprises editing an untranslated region of the target. 66. The method of embodiment 65, wherein the untranslated region is a 5′ untranslated region or a 3′ untranslated region. 67. The method of embodiment 65, wherein the 3′ untranslated region is a polyadenylation sequence. 68. The method of any one of embodiment 48 or embodiments 58-67, wherein editing the target sequence comprises editing a translation initiation site. 69. The method of any one of embodiment 48 or embodiments 58-68, wherein editing the target sequence alters expression of the target sequence. 70. The method of embodiment 69, wherein editing the target sequence increases expression of the target sequence. 71. The method of embodiment 69, wherein editing the target sequence decreases expression of the target sequence. 72. The method of any one of embodiments 48 or 58-71, wherein the editing enzyme comprises an ADAR, an APOBEC, or a Cas nuclease. 73. The method of embodiment 72, wherein the ADAR comprises ADAR1, ADAR2, ADAR3, or combinations thereof. 74. The method of any one of embodiments 48 or 58-73, wherein the target sequence comprises RNA or DNA. 75. The method of any one of embodiments 48 or 58-74, wherein the target sequence is a mRNA or a pre-mRNA. 76. The method of any one of embodiments 48 or 58-75, wherein editing the target sequence comprises deamidating a nucleotide of the target sequence. 77. The method of any one of embodiments 48 or 58-76, wherein the target sequence is edited with an efficiency of at least 10%, at least 20%, or at least 25%, 78. A method of increasing a vector genome integrity of a vector with a first expression cassette and a second expression cassette, the method comprising: a) generating a sequence of the second expression cassette by altering a sequence of the first expression cassette, wherein the first expression cassette and the second expression cassette each independently comprise: a promoter sequence, a payload sequence under transcriptional control of the promoter sequence, the payload sequence comprising a small RNA payload, and a transcription termination sequence; b) generating the multiple payload vector by combining the sequence of the first expression cassette sequence and the sequence of the second expression cassette; and c) increasing the vector genome integrity as compared to a vector comprising a first expression cassette and a second expression cassette that each have the sequence of the first expression cassette. 79. The method of embodiment 78, wherein the sequence of the first expression cassette and the sequence of the second expression cassette have different promoter sequences. 80. The method of embodiment 78 or embodiment 79, wherein the sequence of the first expression cassette and the sequence of the second expression cassette have different transcription termination sequences. 81. The method of any one of embodiments 78-80, wherein the sequence of the first expression cassette and the sequence of the second expression cassette have different payload sequences. 82. The method of any one of embodiments 78-81, wherein the promoter sequence comprises at least 80%, at least 85%, at least 90%, at least 95%, or 100% sequence identity to any one of SEQ ID NO: 1-SEQ ID NO: 9 or SEQ ID NO: 66-SEQ ID NO: 76. 83. The method of any one of embodiments 78-82, wherein the transcription termination sequence comprises at least 80%, at least 85%, at least 90%, at least 95%, or 100% sequence identity to any one of SEQ ID NO: 10-SEQ ID NO: 16, SEQ ID NO: 34, SEQ ID NO: 35, or SEQ ID NO: 77-SEQ ID NO: 82. 84. The method of any one of embodiments 78-83, the method further comprising: a) altering a sequence of a first payload comprising a first engineered guide RNA sequence to generate a second engineered guide RNA sequence to be comprised by a second payload by: i) hybridizing the first engineered guide RNA sequence to a target sequence; ii) forming a first guide-target RNA scaffold comprising one or more structural features; iv) altering at least 20 and no more than 40 nucleotides in the first engineered guide RNA sequence to a different nucleotide resulting in the second engineered guide RNA sequence, wherein hybridizing the second engineered guide RNA sequence to the target sequence forms a second guide-target RNA scaffold comprising the same one or more features at the first guide-target RNA scaffold; b) encoding the first engineered guide RNA sequence in the first payload and the second engineered guide RNA sequence in the second payload in the multiple payload vector; and c) increasing the vector genome integrity of the multiple payload vector as compared to a multiple payload vector comprising a first and second payload each comprising the first engineered guide RNA sequence. 85. The method of embodiment 84, wherein the one or more structural features comprise a bulge, a mismatch, an internal loop, a hairpin, or combinations thereof. 86. The method of embodiment 85, wherein the one or more structural features comprises the bulge, and wherein the bulge is a symmetric bulge. 87. The method of embodiment 85, wherein the one or more structural features comprises the bulge, and wherein the bulge is an asymmetric bulge. 88. The method of any one of embodiments 85-87, wherein the one or more structural features comprises the internal loop, and wherein the internal loop is a symmetric internal loop. 89. The method of any one of embodiments 85-87, wherein the one or more structural features comprises the internal loop, and wherein the internal loop is an asymmetric internal loop. 90. The method of any one of embodiments 85-89, wherein the one or more structural features comprises the hairpin, and wherein the hairpin is a recruitment hairpin or a non-recruitment hairpin. 91. The method of any one of embodiments 84-90, wherein the second guide-target RNA scaffold comprises at least one and no more than 15 additional wobble base pairs as compared to the first guide-target RNA scaffold. 92. The method of embodiment 91, wherein the one or more of the additional wobble base pairs is a GU wobble base pair. 93. The method of any one of embodiments 84-92, wherein the structure of the first guide-target RNA scaffold and the structure of the second guide-target RNA scaffold comprise a helical structure. 94. The method of embodiment 93, wherein the helical structure is over twisted in the structure of the second guide-target RNA scaffold compared to the structure of the first guide-target RNA scaffold.

### EXAMPLES

The invention is further illustrated by the following non-limiting examples.

### Example 1

### Design and Screening of Expression Cassette Arrangement

This example describes engineering and analyzing vectors with a plurality of expression cassettes in various orientations and arrangements to identify the effect of cassette orientation on guide RNA expression.

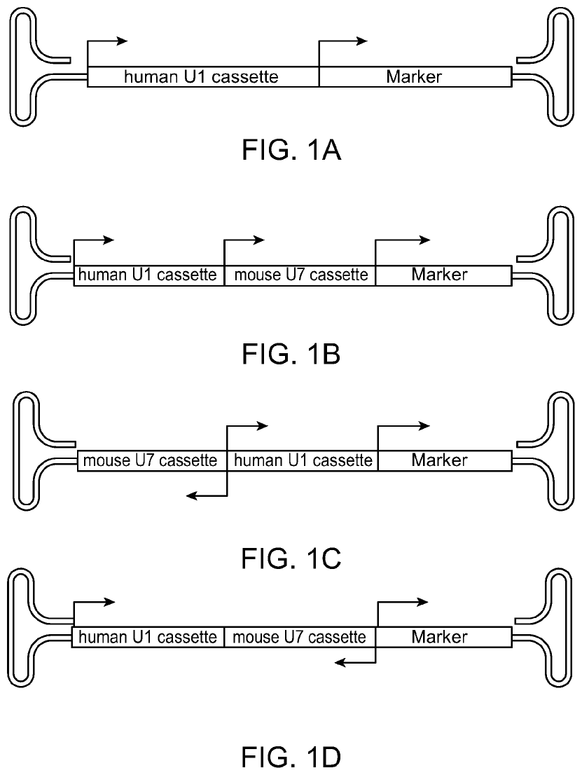

Constructs were screened for engineered guide RNA expression using a luciferase reporter assay. A Kozak competition reporter construct containing an ATG initiation site that is deaminated to ITG, which is read as GTG, in the presence of expressed engineered guide RNA was used as an assay readout. The engineered guide RNA and reporter systems included Reporter 1 which comprised a PMP22 guide RNA payload (SEQ ID NO: 24; GACCGCACCAGCACCGCGACGTGGAGGACGATGATACTCAGCAACAGGAGGAGCC CACTGGCGGCAAGTTCTGCTCAGCGGAGTTTCTGCCCGGCCAAACAGCGTGTGGAA TTTTTGGAGCAGGTTTTCTGACTTCGGTCGGAAAACCCCTCCCAATTTCACTGGTTTC AAAAACAGAAAAACAGTTCTCTTCCCCGCTCCCCGGTGTGTGAGAGGGGCTTTGAT CCTTCTCTGGTTTCCTAGGAAACGCGTATGTG) and Reporter 2 which comprised an SNCA guide RNA payload (SEQ ID NO: 25; GACCGGCCACAACTCCCTCCTTGGCCTTTGAAAGTCCTTTCATGAATACATCCACGG CTAATGAATTCCTTTACACCACACTGGAAAACATAAAATACACTTTGAGTGGAATTT TTGGAGCAGGTTTTCTGACTTCGGTCGGAAAACCCCTCCCAATTTCACTGGTTTCAA AAACAGAAAAACAGTTCTCTTCCCCGCTCCCCGGTGTGTGAGAGGGGCTTTGATCCT TCTCTGGTTTCCTAGGAAACGCGTATGTG). In the absence of start codon deamination, the CDS1 was translated. Deamination of the start codon from ATG to ITG, facilitated by the expressed engineered guide RNA, disrupted CDS1 translation. Instead, a luciferase (“NanoLuc”) was translated. Luciferase activity was used as a readout of engineered guide RNA expression and engineered guide RNA-dependent editing. To identify cassette orientations in a viral vector that enhanced expression, small nucleotide RNA (snRNA) promoters including the mU7 promoter (mU7, SEQ ID NO: 4) and the human U1 promoter (hU1, SEQ ID NO: 1) were positioned upstream of a payload including Reporter 1 with a PMP22 guide RNA (SEQ ID NO: 24) and Reporter 2 with a SNCA guide RNA (SEQ ID NO: 25) in a viral vector and terminator sequences including mU7 terminator (mU7, SEQ ID NO: 10). These included a viral vector with a human U1 cassette (FIG. 1A) in the forward read direction, a viral vector with a human U1 cassette having a forward read directionality and a mouse U7 cassette having a forward read directionality (FIG. 1B), a viral vector with mouse U7 cassette having a reverse read directionality and a human U1 cassette having a forward read directionality (FIG. 1C), a viral vector with human U1 cassette having a forward read directionality and a mouse U7 cassette having a reverse read directionality (FIG. 1D). All four designs were transfected via plasmid or delivered in an AAV virus in cells and evaluated for expression of a guide RNA payload (e.g., Reporter 1 with a PMP22 guide RNA (SEQ ID NO: 24) and Reporter 2 with a SNCA guide RNA (SEQ ID NO: 25)). The vector sequences tested are provided in TABLE 5, and include: a viral vector with a human U1 cassette in the forward read direction with a PMP22 guide RNA (SEQ ID NO: 26); a viral vector with a human U1 cassette having a forward read directionality and a mouse U7 cassette having a forward read directionality each having a PMP22 guide RNA payload (SEQ ID NO: 27); a viral vector with mouse U7 cassette having a reverse read directionality and a human U1 cassette having a forward read directionality each having a PMP22 guide RNA payload (SEQ ID NO: 28); a viral vector with human U1 cassette having a forward read directionality and a mouse U7 cassette having a reverse read directionality each having a PMP22 guide RNA payload (SEQ ID NO: 29); a viral vector with a human U1 cassette in the forward read direction with an SNCA guide RNA (SEQ ID NO: 30); a viral vector with a human U1 cassette having a forward read directionality and a mouse U7 cassette having a forward read directionality each having an SNCA guide RNA payload (SEQ ID NO: 31); a viral vector with mouse U7 cassette having a reverse read directionality and a human U1 cassette having a forward read directionality each having an SNCA guide RNA payload (SEQ ID NO: 32); and a viral vector with human U1 cassette having a forward read directionality and a mouse U7 cassette having a reverse read directionality each having an SNCA guide RNA payload (SEQ ID NO: 33).

TABLE 5
Examples of Multi-Expression Cassette Vector Sequences

| SEQ ID | |
|---|---|
| NO: | Sequence |
| SEQ ID | GGATCCTATCAAGAACCAACCTAGAACAGGATACCACAGTGAATATCACTGTTAGAT |
| NO: 26 | AAGGACCAGCTTCTTTGGGAGAGAACAGACGCAGGGGGGGGAGGGAAAAAGGGAGA |
| | GGCAGACGTCACTTCCTCTTGGCGACTCTGGCAGCAGATTGGTCGGTTGAGTGGCAGA |
| | AAGGCAGACGGGGACTGGGCAAGGCACTGTCGGTGACATCACGGACAGGGCGACTT |
| | CTATGTAGATGAGGCAGCGCAGAGGCTGCTGCTTCGCCACTTGCTGCTTCGCCACGAA |
| | GGGAGTTCCCGTGCCCTGGGAGCGGGTTCAGGACCGCTGATCGGAAGTGAGAATCCC |
| | AGCTGTGTGTCAGGGCTGGAAAGGGCTCGGGAGTGCGCGGGGCAAGTGACCGTGTGT |
| | GTAAAGAGTGAGGCGTATGAGGCTGTGTCGGGGCAGAGCCCGAAGATCTCACCGCAC |
| | CAGCACCGCGACGTGGAGGACGATGATACTCAGCAACAGGAGGAGCCCACTGGCGG |
| | CAAGTTCTGCTCAGCGGAGTTTCTGCCCGGCCAAACAGCGTGTGGAATTTTTGGAGCA |
| | GGTTTTCTGACTTCGGTCGGAAAACCCCTCCCAATTTCACTGGTCTACAATGAAAGCA |
| | AAACAGTTCTCTTCCCCGCTCCCCGGTGTGTGAGAGGGGCTTTGATCCTTCTCTGGTTT |
| | CCTAGGAAACGCGTATGTGACTGTTAACAAGACACAGTGTGTTGTCTATTCGGTTGGA |
| | ATACCTAACGCCCTGGCATTATGCCCAGTACATGACCTTATGGGACTTTCCTACTTGG |
| | CAGTACATCTACGTATTAGTCATCGCTATTACCATGGTGATGCGGTTTTGGCAGTACA |
| | TCAATGGGCGTGGATAGCGGTTTGACTCACGGGGATTTCCAAGTCTCCACCCCATTGA |
| | CGTCAATGGGAGTTTGTTTTGGCACCAAAATCAACGGGACTTTCCAAAATGTCGTAAC |
| | AACTCCGCCCCATTGACGCAAATGGGCGGTAGGCGTGTACGGTGGGAGGTCTATATA |
| | AGCAGAGCTCGTTTAGTGAACCGTCAGATCGCCTGGAGACGCCATCCACGCTGTTTTG |
| | ACCTCCATAGTAGAGCCGCCACCATGGCCACAACCATGGGTCTTTTCTGCAATCACCG |
| | TCCTCGAGGCACCATGAACCCAGCCATCAGCGTCGCTCTCCTGCTCTCAGTCTTGCGG |
| | GTGTCCCGAGGGCAGAAGGTGACCAGCCTGACAGCCTGCCTGGTGAACCAAAACCTT |
| | CGCCTGGACTGCCGCCATGAGAATAACACCAAGGATAACTCCATCCAGCATGAGTTC |
| | AGCCTGACCCGAGAGAAGAGGAAGCACGTGCTCTCAGGCACCCTTGGGATACCCGAG |
| | CACACGTACCGCTCCCGCGTCACCCTCTCCAACCAGCCCTATATCAAGGTCCTTACCC |
| | TAGCCAACTTCACCACCAAGGATGAGGGCGACTACTTTTGTGAGCTTCGCGTCTCGGG |
| | CGCGAATCCCATGAGCTCCAATAAAAGTATCAGTGTGTATAGAGACAAGCTGGTCAA |
| | GTGTGGCGGCATAAGCCTGCTGGTTCAGAACACATCCTGGATGCTGCTGCTGCTGCTT |
| | TCCCTCTCCCTCCTCCAAGCCCTGGACTTCATTTCTCTGTAAAATAAAGGAAATTTATT |
| | TTCATTGCAATAGTGTGTTGGAATTTTTTGTGTCTCTCAGTACTAAGATTGCGGCCTGG |
| | CCACTGACTAAGATAGAAGCTGTAACTTGCCTCAGATTGTGGCCGCTGATCTGCAG |
| SEQ ID | GGATCCTAAGGACCAGCTTCTTTGGGAGAGAACAGACGCAGGGGGGGGAGGGAAAA |
| NO: 27 | AGGGAGAGGCAGACGTCACTTCCTCTTGGCGACTCTGGCAGCAGATTGGTCGGTTGA |
| | GTGGCAGAAAGGCAGACGGGGACTGGGCAAGGCACTGTCGGTGACATCACGGACAG |
| | GGCGACTTCTATGTAGATGAGGCAGCGCAGAGGCTGCTGCTTCGCCACTTGCTGCTTC |
| | GCCACGAAGGGAGTTCCCGTGCCCTGGGAGCGGGTTCAGGACCGCTGATCGGAAGTG |
| | AGAATCCCAGCTGTGTGTCAGGGCTGGAAAGGGCTCGGGAGTGCGCGGGGCAAGTGA |
| | CCGTGTGTGTAAAGAGTGAGGCGTATGAGGCTGTGTCGGGGCAGAGCCCGAAGATCT |
| | CACCGCACCAGCACCGCGACGTGGAGGACGATGATACTCAGCAACAGGAGGAGCCC |
| | ACTGGCGGCAAGTTCTGCTCAGCGGAGTTTCTGCCCGGCCAAACAGCGTGTGGAATTT |
| | TTGGAGCAGGTTTTCTGACTTCGGTCGGAAAACCCCTCCCAATTTCACTGGTCTACAA |
| | TGAAAGCAAAACAGTTCTCTTCCCCGCTCCCCGGTGTGTGAGAGGGGCTTTGATCCTT |
| | CTCTGGTTTCCTAGGAAACGCGTATGTGTAACAACATAGGAGCTGTGATTGGCTGTTT |
| | TCAGCCAATCAGCACTGACTCATTTGCATAGCCTTTACAAGCGGTCACAAACTCAAGA |
| | AACGAGCGGTTTTAATAGTCTTTTAGAATATTGTTTATCGAACCGAATAAGGAACTGT |
| | GCTTTGTGATTCACATATCAGTGGAGGGGTGTGGAAATGGCACCTTGATCTCACCCTC |
| | ATCGAAAGTGGAGTTGATGTCCTTCCCTGGCTCGCTACAGACGCACTTCCGCACCGCA |
| | CCAGCACCGCGACGTGGAGGACGATGATACTCAGCAACAGGAGGAGCCCACTGGCG |
| | GCAAGTTCTGCTCAGCGGAGTTTCTGCCCGGCCAAACAGCGTGTGGAATTTTTGGAGC |
| | AGGTTTTCTGACTTCGGTCGGAAAACCCCTCCCAATTTCACTGGTCTACAATGAAAGC |
| | AAAACAGTTCTCTTCCCCGCTCCCCGGTGTGTGAGAGGGGCTTTGATCCTTCTCTGGTT |
| | TCCTAGGAAACGCGTATGTGCCTGGCATTATGCCCAGTACATGACCTTATGGGACTTT |
| | CCTACTTGGCAGTACATCTACGTATTAGTCATCGCTATTACCATGGTGATGCGGTTTTG |
| | GCAGTACATCAATGGGCGTGGATAGCGGTTTGACTCACGGGGATTTCCAAGTCTCCAC |
| | CCCATTGACGTCAATGGGAGTTTGTTTTGGCACCAAAATCAACGGGACTTTCCAAAAT |
| | GTCGTAACAACTCCGCCCCATTGACGCAAATGGGCGGTAGGCGTGTACGGTGGGAGG |
| | TCTATATAAGCAGAGCTCGTTTAGTGAACCGTCAGATCGCCTGGAGACGCCATCCACG |
| | CTGTTTTGACCTCCATAGTAGAGCCGCCACCATGGCCACAACCATGGGTCTTTTCTGC |
| | AATCACCGTCCTCGAGGCACCATGAACCCAGCCATCAGCGTCGCTCTCCTGCTCTCAG |
| | TCTTGCGGGTGTCCCGAGGGCAGAAGGTGACCAGCCTGACAGCCTGCCTGGTGAACC |
| | AAAACCTTCGCCTGGACTGCCGCCATGAGAATAACACCAAGGATAACTCCATCCAGC |
| | ATGAGTTCAGCCTGACCCGAGAGAAGAGGAAGCACGTGCTCTCAGGCACCCTTGGGA |
| | TACCCGAGCACACGTACCGCTCCCGCGTCACCCTCTCCAACCAGCCCTATATCAAGGT |
| | CCTTACCCTAGCCAACTTCACCACCAAGGATGAGGGCGACTACTTTTGTGAGCTTCGC |
| | GTCTCGGGCGCGAATCCCATGAGCTCCAATAAAAGTATCAGTGTGTATAGAGACAAG |
| | CTGGTCAAGTGTGGCGGCATAAGCCTGCTGGTTCAGAACACATCCTGGATGCTGCTGC |
| | TGCTGCTTTCCCTCTCCCTCCTCCAAGCCCTGGACTTCATTTCTCTGTAAAATAAAGGA |
| | AATTTATTTTCATTGCAATAGTGTGTTGGAATTTTTTGTGTCTCTCAGTACTAAGATTG |
| | CGGCCTGGCCACTGACTAAGATAGAAGCTGTAACTTGCCTCAGATTGTGGCCGCTGAT |
| | CTGCAG |
| SEQ ID | GGATCCCACATACGCGTTTCCTAGGAAACCAGAGAAGGATCAAAGCCCCTCTCACAC |
| NO: 28 | ACCGGGGAGCGGGGAAGAGAACTGTTTTGCTTTCATTGTAGACCAGTGAAATTGGGA |
| | GGGGTTTTCCGACCGAAGTCAGAAAACCTGCTCCAAAAATTCCACACGCTGTTTGGCC |
| | GGGCAGAAACTCCGCTGAGCAGAACTTGCCGCCAGTGGGCTCCTCCTGTTGCTGAGT |
| | ATCATCGTCCTCCACGTCGCGGTGCTGGTGCGGTGCGGAAGTGCGTCTGTAGCGAGCC |
| | AGGGAAGGACATCAACTCCACTTTCGATGAGGGTGAGATCAAGGTGCCATTTCCACA |
| | CCCCTCCACTGATATGTGAATCACAAAGCACAGTTCCTTATTCGGTTCGATAAACAAT |
| | ATTCTAAAAGACTATTAAAACCGCTCGTTTCTTGAGTTTGTGACCGCTTGTAAAGGCT |
| | ATGCAAATGAGTCAGTGCTGATTGGCTGAAAACAGCCAATCACAGCTCCTATGTTGTT |
| | ATAAGGACCAGCTTCTTTGGGAGAGAACAGACGCAGGGGGGGGAGGGAAAAAGGGA |
| | GAGGCAGACGTCACTTCCTCTTGGCGACTCTGGCAGCAGATTGGTCGGTTGAGTGGCA |
| | GAAAGGCAGACGGGGACTGGGCAAGGCACTGTCGGTGACATCACGGACAGGGCGAC |
| | TTCTATGTAGATGAGGCAGCGCAGAGGCTGCTGCTTCGCCACTTGCTGCTTCGCCACG |
| | AAGGGAGTTCCCGTGCCCTGGGAGCGGGTTCAGGACCGCTGATCGGAAGTGAGAATC |
| | CCAGCTGTGTGTCAGGGCTGGAAAGGGCTCGGGAGTGCGCGGGGCAAGTGACCGTGT |
| | GTGTAAAGAGTGAGGCGTATGAGGCTGTGTCGGGGCAGAGCCCGAAGATCTCACCGC |
| | ACCAGCACCGCGACGTGGAGGACGATGATACTCAGCAACAGGAGGAGCCCACTGGC |
| | GGCAAGTTCTGCTCAGCGGAGTTTCTGCCCGGCCAAACAGCGTGTGGAATTTTTGGAG |
| | CAGGTTTTCTGACTTCGGTCGGAAAACCCCTCCCAATTTCACTGGTCTACAATGAAAG |
| | CAAAACAGTTCTCTTCCCCGCTCCCCGGTGTGTGAGAGGGGCTTTGATCCTTCTCTGG |
| | TTTCCTAGGAAACGCGTATGTGCCTGGCATTATGCCCAGTACATGACCTTATGGGACT |
| | TTCCTACTTGGCAGTACATCTACGTATTAGTCATCGCTATTACCATGGTGATGCGGTTT |
| | TGGCAGTACATCAATGGGCGTGGATAGCGGTTTGACTCACGGGGATTTCCAAGTCTCC |
| | ACCCCATTGACGTCAATGGGAGTTTGTTTTGGCACCAAAATCAACGGGACTTTCCAAA |
| | ATGTCGTAACAACTCCGCCCCATTGACGCAAATGGGCGGTAGGCGTGTACGGTGGGA |
| | GGTCTATATAAGCAGAGCTCGTTTAGTGAACCGTCAGATCGCCTGGAGACGCCATCC |
| | ACGCTGTTTTGACCTCCATAGTAGAGCCGCCACCATGGCCACAACCATGGGTCTTTTC |
| | TGCAATCACCGTCCTCGAGGCACCATGAACCCAGCCATCAGCGTCGCTCTCCTGCTCT |
| | CAGTCTTGCGGGTGTCCCGAGGGCAGAAGGTGACCAGCCTGACAGCCTGCCTGGTGA |
| | ACCAAAACCTTCGCCTGGACTGCCGCCATGAGAATAACACCAAGGATAACTCCATCC |
| | AGCATGAGTTCAGCCTGACCCGAGAGAAGAGGAAGCACGTGCTCTCAGGCACCCTTG |
| | GGATACCCGAGCACACGTACCGCTCCCGCGTCACCCTCTCCAACCAGCCCTATATCAA |
| | GGTCCTTACCCTAGCCAACTTCACCACCAAGGATGAGGGCGACTACTTTTGTGAGCTT |
| | CGCGTCTCGGGCGCGAATCCCATGAGCTCCAATAAAAGTATCAGTGTGTATAGAGAC |
| | AAGCTGGTCAAGTGTGGCGGCATAAGCCTGCTGGTTCAGAACACATCCTGGATGCTG |
| | CTGCTGCTGCTTTCCCTCTCCCTCCTCCAAGCCCTGGACTTCATTTCTCTGTAAAATAA |
| | AGGAAATTTATTTTCATTGCAATAGTGTGTTGGAATTTTTTGTGTCTCTCAGTACTAAG |
| | ATTGCGGCCTGGCCACTGACTAAGATAGAAGCTGTAACTTGCCTCAGATTGTGGCCGC |
| | TGATCTGCAG |
| SEQ ID | GGATCCTAAGGACCAGCTTCTTTGGGAGAGAACAGACGCAGGGGGGGGAGGGAAAA |
| NO: 29 | AGGGAGAGGCAGACGTCACTTCCTCTTGGCGACTCTGGCAGCAGATTGGTCGGTTGA |
| | GTGGCAGAAAGGCAGACGGGGACTGGGCAAGGCACTGTCGGTGACATCACGGACAG |
| | GGCGACTTCTATGTAGATGAGGCAGCGCAGAGGCTGCTGCTTCGCCACTTGCTGCTTC |
| | GCCACGAAGGGAGTTCCCGTGCCCTGGGAGCGGGTTCAGGACCGCTGATCGGAAGTG |
| | AGAATCCCAGCTGTGTGTCAGGGCTGGAAAGGGCTCGGGAGTGCGCGGGGCAAGTGA |
| | CCGTGTGTGTAAAGAGTGAGGCGTATGAGGCTGTGTCGGGGCAGAGCCCGAAGATCT |
| | CACCGCACCAGCACCGCGACGTGGAGGACGATGATACTCAGCAACAGGAGGAGCCC |
| | ACTGGCGGCAAGTTCTGCTCAGCGGAGTTTCTGCCCGGCCAAACAGCGTGTGGAATTT |
| | TTGGAGCAGGTTTTCTGACTTCGGTCGGAAAACCCCTCCCAATTTCACTGGTCTACAA |
| | TGAAAGCAAAACAGTTCTCTTCCCCGCTCCCCGGTGTGTGAGAGGGGCTTTGATCCTT |
| | CTCTGGTTTCCTAGGAAACGCGTATGTGCACATACGCGTTTCCTAGGAAACCAGAGAA |
| | GGATCAAAGCCCCTCTCACACACCGGGGAGCGGGGAAGAGAACTGTTTTGCTTTCAT |
| | TGTAGACCAGTGAAATTGGGAGGGGTTTTCCGACCGAAGTCAGAAAACCTGCTCCAA |
| | AAATTCCACACGCTGTTTGGCCGGGCAGAAACTCCGCTGAGCAGAACTTGCCGCCAG |
| | TGGGCTCCTCCTGTTGCTGAGTATCATCGTCCTCCACGTCGCGGTGCTGGTGCGGTGC |
| | GGAAGTGCGTCTGTAGCGAGCCAGGGAAGGACATCAACTCCACTTTCGATGAGGGTG |
| | AGATCAAGGTGCCATTTCCACACCCCTCCACTGATATGTGAATCACAAAGCACAGTTC |
| | CTTATTCGGTTCGATAAACAATATTCTAAAAGACTATTAAAACCGCTCGTTTCTTGAG |
| | TTTGTGACCGCTTGTAAAGGCTATGCAAATGAGTCAGTGCTGATTGGCTGAAAACAGC |
| | CAATCACAGCTCCTATGTTGTTACCTGGCATTATGCCCAGTACATGACCTTATGGGAC |
| | TTTCCTACTTGGCAGTACATCTACGTATTAGTCATCGCTATTACCATGGTGATGCGGTT |
| | TTGGCAGTACATCAATGGGCGTGGATAGCGGTTTGACTCACGGGGATTTCCAAGTCTC |
| | CACCCCATTGACGTCAATGGGAGTTTGTTTTGGCACCAAAATCAACGGGACTTTCCAA |
| | AATGTCGTAACAACTCCGCCCCATTGACGCAAATGGGCGGTAGGCGTGTACGGTGGG |
| | AGGTCTATATAAGCAGAGCTCGTTTAGTGAACCGTCAGATCGCCTGGAGACGCCATC |
| | CACGCTGTTTTGACCTCCATAGTAGAGCCGCCACCATGGCCACAACCATGGGTCTTTT |
| | CTGCAATCACCGTCCTCGAGGCACCATGAACCCAGCCATCAGCGTCGCTCTCCTGCTC |
| | TCAGTCTTGCGGGTGTCCCGAGGGCAGAAGGTGACCAGCCTGACAGCCTGCCTGGTG |
| | AACCAAAACCTTCGCCTGGACTGCCGCCATGAGAATAACACCAAGGATAACTCCATC |
| | CAGCATGAGTTCAGCCTGACCCGAGAGAAGAGGAAGCACGTGCTCTCAGGCACCCTT |
| | GGGATACCCGAGCACACGTACCGCTCCCGCGTCACCCTCTCCAACCAGCCCTATATCA |
| | AGGTCCTTACCCTAGCCAACTTCACCACCAAGGATGAGGGCGACTACTTTTGTGAGCT |
| | TCGCGTCTCGGGCGCGAATCCCATGAGCTCCAATAAAAGTATCAGTGTGTATAGAGA |
| | CAAGCTGGTCAAGTGTGGCGGCATAAGCCTGCTGGTTCAGAACACATCCTGGATGCT |
| | GCTGCTGCTGCTTTCCCTCTCCCTCCTCCAAGCCCTGGACTTCATTTCTCTGTAAAATA |
| | AAGGAAATTTATTTTCATTGCAATAGTGTGTTGGAATTTTTTGTGTCTCTCAGTACTAA |
| | GATTGCGGCCTGGCCACTGACTAAGATAGAAGCTGTAACTTGCCTCAGATTGTGGCCG |
| | CTGATCTGCAG |
| SEQ ID | GGATCCTATCAAGAACCAACCTAGAACAGGATACCACAGTGAATATCACTGTTAGAT |
| NO: 30 | AAGGACCAGCTTCTTTGGGAGAGAACAGACGCAGGGGGGGGAGGGAAAAAGGGAGA |
| | GGCAGACGTCACTTCCTCTTGGCGACTCTGGCAGCAGATTGGTCGGTTGAGTGGCAGA |
| | AAGGCAGACGGGGACTGGGCAAGGCACTGTCGGTGACATCACGGACAGGGCGACTT |
| | CTATGTAGATGAGGCAGCGCAGAGGCTGCTGCTTCGCCACTTGCTGCTTCGCCACGAA |
| | GGGAGTTCCCGTGCCCTGGGAGCGGGTTCAGGACCGCTGATCGGAAGTGAGAATCCC |
| | AGCTGTGTGTCAGGGCTGGAAAGGGCTCGGGAGTGCGCGGGGCAAGTGACCGTGTGT |
| | GTAAAGAGTGAGGCGTATGAGGCTGTGTCGGGGCAGAGCCCGAAGATCTCACCGGCC |
| | ACAACTCCCTCCTTGGCCTTTGAAAGTCCTTTCATGAATACATCCACGGCTAATGAAT |
| | TCCTTTACACCACACTGGAAAACATAAAATACACTTTGAGTGGAATTTTTGGAGCAGG |
| | TTTTCTGACTTCGGTCGGAAAACCCCTCCCAATTTCACTGGTCTACAATGAAAGCAAA |
| | ACAGTTCTCTTCCCCGCTCCCCGGTGTGTGAGAGGGGCTTTGATCCTTCTCTGGTTTCC |
| | TAGGAAACGCGTATGTGACTGTTAACAAGACACAGTGTGTTGTCTATTCGGTTGGAAT |
| | ACCTAACGCCCTGGCATTATGCCCAGTACATGACCTTATGGGACTTTCCTACTTGGCA |
| | GTACATCTACGTATTAGTCATCGCTATTACCATGGTGATGCGGTTTTGGCAGTACATC |
| | AATGGGCGTGGATAGCGGTTTGACTCACGGGGATTTCCAAGTCTCCACCCCATTGACG |
| | TCAATGGGAGTTTGTTTTGGCACCAAAATCAACGGGACTTTCCAAAATGTCGTAACAA |
| | CTCCGCCCCATTGACGCAAATGGGCGGTAGGCGTGTACGGTGGGAGGTCTATATAAG |
| | CAGAGCTCGTTTAGTGAACCGTCAGATCGCCTGGAGACGCCATCCACGCTGTTTTGAC |
| | CTCCATAGTAGAGCCGCCACCATGGCCACAACCATGGGTCTTTTCTGCAATCACCGTC |
| | CTCGAGGCACCATGAACCCAGCCATCAGCGTCGCTCTCCTGCTCTCAGTCTTGCGGGT |
| | GTCCCGAGGGCAGAAGGTGACCAGCCTGACAGCCTGCCTGGTGAACCAAAACCTTCG |
| | CCTGGACTGCCGCCATGAGAATAACACCAAGGATAACTCCATCCAGCATGAGTTCAG |
| | CCTGACCCGAGAGAAGAGGAAGCACGTGCTCTCAGGCACCCTTGGGATACCCGAGCA |
| | CACGTACCGCTCCCGCGTCACCCTCTCCAACCAGCCCTATATCAAGGTCCTTACCCTA |
| | GCCAACTTCACCACCAAGGATGAGGGCGACTACTTTTGTGAGCTTCGCGTCTCGGGCG |
| | CGAATCCCATGAGCTCCAATAAAAGTATCAGTGTGTATAGAGACAAGCTGGTCAAGT |
| | GTGGCGGCATAAGCCTGCTGGTTCAGAACACATCCTGGATGCTGCTGCTGCTGCTTTC |
| | CCTCTCCCTCCTCCAAGCCCTGGACTTCATTTCTCTGTAAAATAAAGGAAATTTATTTT |
| | CATTGCAATAGTGTGTTGGAATTTTTTGTGTCTCTCAGTACTAAGATTGCGGCCTGGCC |
| | ACTGACTAAGATAGAAGCTGTAACTTGCCTCAGATTGTGGCCGCTGATCTGCAG |
| SEQ ID | GGATCCTAAGGACCAGCTTCTTTGGGAGAGAACAGACGCAGGGGGGGGAGGGAAAA |
| NO: 31 | AGGGAGAGGCAGACGTCACTTCCTCTTGGCGACTCTGGCAGCAGATTGGTCGGTTGA |
| | GTGGCAGAAAGGCAGACGGGGACTGGGCAAGGCACTGTCGGTGACATCACGGACAG |
| | GGCGACTTCTATGTAGATGAGGCAGCGCAGAGGCTGCTGCTTCGCCACTTGCTGCTTC |
| | GCCACGAAGGGAGTTCCCGTGCCCTGGGAGCGGGTTCAGGACCGCTGATCGGAAGTG |
| | AGAATCCCAGCTGTGTGTCAGGGCTGGAAAGGGCTCGGGAGTGCGCGGGGCAAGTGA |
| | CCGTGTGTGTAAAGAGTGAGGCGTATGAGGCTGTGTCGGGGCAGAGCCCGAAGATCT |
| | CACCGGCCACAACTCCCTCCTTGGCCTTTGAAAGTCCTTTCATGAATACATCCACGGC |
| | TAATGAATTCCTTTACACCACACTGGAAAACATAAAATACACTTTGAGTGGAATTTTT |
| | GGAGCAGGTTTTCTGACTTCGGTCGGAAAACCCCTCCCAATTTCACTGGTCTACAATG |
| | AAAGCAAAACAGTTCTCTTCCCCGCTCCCCGGTGTGTGAGAGGGGCTTTGATCCTTCT |
| | CTGGTTTCCTAGGAAACGCGTATGTGTAACAACATAGGAGCTGTGATTGGCTGTTTTC |
| | AGCCAATCAGCACTGACTCATTTGCATAGCCTTTACAAGCGGTCACAAACTCAAGAA |
| | ACGAGCGGTTTTAATAGTCTTTTAGAATATTGTTTATCGAACCGAATAAGGAACTGTG |
| | CTTTGTGATTCACATATCAGTGGAGGGGTGTGGAAATGGCACCTTGATCTCACCCTCA |
| | TCGAAAGTGGAGTTGATGTCCTTCCCTGGCTCGCTACAGACGCACTTCCGCACCGGCC |
| | ACAACTCCCTCCTTGGCCTTTGAAAGTCCTTTCATGAATACATCCACGGCTAATGAAT |
| | TCCTTTACACCACACTGGAAAACATAAAATACACTTTGAGTGGAATTTTTGGAGCAGG |
| | TTTTCTGACTTCGGTCGGAAAACCCCTCCCAATTTCACTGGTCTACAATGAAAGCAAA |
| | ACAGTTCTCTTCCCCGCTCCCCGGTGTGTGAGAGGGGCTTTGATCCTTCTCTGGTTTCC |
| | TAGGAAACGCGTATGTGCCTGGCATTATGCCCAGTACATGACCTTATGGGACTTTCCT |
| | ACTTGGCAGTACATCTACGTATTAGTCATCGCTATTACCATGGTGATGCGGTTTTGGC |
| | AGTACATCAATGGGCGTGGATAGCGGTTTGACTCACGGGGATTTCCAAGTCTCCACCC |
| | CATTGACGTCAATGGGAGTTTGTTTTGGCACCAAAATCAACGGGACTTTCCAAAATGT |
| | CGTAACAACTCCGCCCCATTGACGCAAATGGGCGGTAGGCGTGTACGGTGGGAGGTC |
| | TATATAAGCAGAGCTCGTTTAGTGAACCGTCAGATCGCCTGGAGACGCCATCCACGCT |
| | GTTTTGACCTCCATAGTAGAGCCGCCACCATGGCCACAACCATGGGTCTTTTCTGCAA |
| | TCACCGTCCTCGAGGCACCATGAACCCAGCCATCAGCGTCGCTCTCCTGCTCTCAGTC |
| | TTGCGGGTGTCCCGAGGGCAGAAGGTGACCAGCCTGACAGCCTGCCTGGTGAACCAA |
| | AACCTTCGCCTGGACTGCCGCCATGAGAATAACACCAAGGATAACTCCATCCAGCAT |
| | GAGTTCAGCCTGACCCGAGAGAAGAGGAAGCACGTGCTCTCAGGCACCCTTGGGATA |
| | CCCGAGCACACGTACCGCTCCCGCGTCACCCTCTCCAACCAGCCCTATATCAAGGTCC |
| | TTACCCTAGCCAACTTCACCACCAAGGATGAGGGCGACTACTTTTGTGAGCTTCGCGT |
| | CTCGGGCGCGAATCCCATGAGCTCCAATAAAAGTATCAGTGTGTATAGAGACAAGCT |
| | GGTCAAGTGTGGCGGCATAAGCCTGCTGGTTCAGAACACATCCTGGATGCTGCTGCTG |
| | CTGCTTTCCCTCTCCCTCCTCCAAGCCCTGGACTTCATTTCTCTGTAAAATAAAGGAAA |
| | TTTATTTTCATTGCAATAGTGTGTTGGAATTTTTTGTGTCTCTCAGTACTAAGATTGCG |
| | GCCTGGCCACTGACTAAGATAGAAGCTGTAACTTGCCTCAGATTGTGGCCGCTGATCT |
| | GCAG |
| SEQ ID | GGATCCCACATACGCGTTTCCTAGGAAACCAGAGAAGGATCAAAGCCCCTCTCACAC |
| NO: 32 | ACCGGGGAGCGGGGAAGAGAACTGTTTTGCTTTCATTGTAGACCAGTGAAATTGGGA |
| | GGGGTTTTCCGACCGAAGTCAGAAAACCTGCTCCAAAAATTCCACTCAAAGTGTATTT |
| | TATGTTTTCCAGTGTGGTGTAAAGGAATTCATTAGCCGTGGATGTATTCATGAAAGGA |
| | CTTTCAAAGGCCAAGGAGGGAGTTGTGGCCGGTGCGGAAGTGCGTCTGTAGCGAGCC |
| | AGGGAAGGACATCAACTCCACTTTCGATGAGGGTGAGATCAAGGTGCCATTTCCACA |
| | CCCCTCCACTGATATGTGAATCACAAAGCACAGTTCCTTATTCGGTTCGATAAACAAT |
| | ATTCTAAAAGACTATTAAAACCGCTCGTTTCTTGAGTTTGTGACCGCTTGTAAAGGCT |
| | ATGCAAATGAGTCAGTGCTGATTGGCTGAAAACAGCCAATCACAGCTCCTATGTTGTT |
| | ATAAGGACCAGCTTCTTTGGGAGAGAACAGACGCAGGGGGGGGAGGGAAAAAGGGA |
| | GAGGCAGACGTCACTTCCTCTTGGCGACTCTGGCAGCAGATTGGTCGGTTGAGTGGCA |
| | GAAAGGCAGACGGGGACTGGGCAAGGCACTGTCGGTGACATCACGGACAGGGCGAC |
| | TTCTATGTAGATGAGGCAGCGCAGAGGCTGCTGCTTCGCCACTTGCTGCTTCGCCACG |
| | AAGGGAGTTCCCGTGCCCTGGGAGCGGGTTCAGGACCGCTGATCGGAAGTGAGAATC |
| | CCAGCTGTGTGTCAGGGCTGGAAAGGGCTCGGGAGTGCGCGGGGCAAGTGACCGTGT |
| | GTGTAAAGAGTGAGGCGTATGAGGCTGTGTCGGGGCAGAGCCCGAAGATCTCACCGG |
| | CCACAACTCCCTCCTTGGCCTTTGAAAGTCCTTTCATGAATACATCCACGGCTAATGA |
| | ATTCCTTTACACCACACTGGAAAACATAAAATACACTTTGAGTGGAATTTTTGGAGCA |
| | GGTTTTCTGACTTCGGTCGGAAAACCCCTCCCAATTTCACTGGTCTACAATGAAAGCA |
| | AAACAGTTCTCTTCCCCGCTCCCCGGTGTGTGAGAGGGGCTTTGATCCTTCTCTGGTTT |
| | CCTAGGAAACGCGTATGTGCCTGGCATTATGCCCAGTACATGACCTTATGGGACTTTC |
| | CTACTTGGCAGTACATCTACGTATTAGTCATCGCTATTACCATGGTGATGCGGTTTTG |
| | GCAGTACATCAATGGGCGTGGATAGCGGTTTGACTCACGGGGATTTCCAAGTCTCCAC |
| | CCCATTGACGTCAATGGGAGTTTGTTTTGGCACCAAAATCAACGGGACTTTCCAAAAT |
| | GTCGTAACAACTCCGCCCCATTGACGCAAATGGGCGGTAGGCGTGTACGGTGGGAGG |
| | TCTATATAAGCAGAGCTCGTTTAGTGAACCGTCAGATCGCCTGGAGACGCCATCCACG |
| | CTGTTTTGACCTCCATAGTAGAGCCGCCACCATGGCCACAACCATGGGTCTTTTCTGC |
| | AATCACCGTCCTCGAGGCACCATGAACCCAGCCATCAGCGTCGCTCTCCTGCTCTCAG |
| | TCTTGCGGGTGTCCCGAGGGCAGAAGGTGACCAGCCTGACAGCCTGCCTGGTGAACC |
| | AAAACCTTCGCCTGGACTGCCGCCATGAGAATAACACCAAGGATAACTCCATCCAGC |
| | ATGAGTTCAGCCTGACCCGAGAGAAGAGGAAGCACGTGCTCTCAGGCACCCTTGGGA |
| | TACCCGAGCACACGTACCGCTCCCGCGTCACCCTCTCCAACCAGCCCTATATCAAGGT |
| | CCTTACCCTAGCCAACTTCACCACCAAGGATGAGGGCGACTACTTTTGTGAGCTTCGC |
| | GTCTCGGGCGCGAATCCCATGAGCTCCAATAAAAGTATCAGTGTGTATAGAGACAAG |
| | CTGGTCAAGTGTGGCGGCATAAGCCTGCTGGTTCAGAACACATCCTGGATGCTGCTGC |
| | TGCTGCTTTCCCTCTCCCTCCTCCAAGCCCTGGACTTCATTTCTCTGTAAAATAAAGGA |
| | AATTTATTTTCATTGCAATAGTGTGTTGGAATTTTTTGTGTCTCTCAGTACTAAGATTG |
| | CGGCCTGGCCACTGACTAAGATAGAAGCTGTAACTTGCCTCAGATTGTGGCCGCTGAT |
| | CTGCAG |
| SEQ ID | GGATCCTAAGGACCAGCTTCTTTGGGAGAGAACAGACGCAGGGGGGGGAGGGAAAA |
| NO: 33 | AGGGAGAGGCAGACGTCACTTCCTCTTGGCGACTCTGGCAGCAGATTGGTCGGTTGA |
| | GTGGCAGAAAGGCAGACGGGGACTGGGCAAGGCACTGTCGGTGACATCACGGACAG |
| | GGCGACTTCTATGTAGATGAGGCAGCGCAGAGGCTGCTGCTTCGCCACTTGCTGCTTC |
| | GCCACGAAGGGAGTTCCCGTGCCCTGGGAGCGGGTTCAGGACCGCTGATCGGAAGTG |
| | AGAATCCCAGCTGTGTGTCAGGGCTGGAAAGGGCTCGGGAGTGCGCGGGGCAAGTGA |
| | CCGTGTGTGTAAAGAGTGAGGCGTATGAGGCTGTGTCGGGGCAGAGCCCGAAGATCT |
| | CACCGGCCACAACTCCCTCCTTGGCCTTTGAAAGTCCTTTCATGAATACATCCACGGC |
| | TAATGAATTCCTTTACACCACACTGGAAAACATAAAATACACTTTGAGTGGAATTTTT |
| | GGAGCAGGTTTTCTGACTTCGGTCGGAAAACCCCTCCCAATTTCACTGGTCTACAATG |
| | AAAGCAAAACAGTTCTCTTCCCCGCTCCCCGGTGTGTGAGAGGGGCTTTGATCCTTCT |
| | CTGGTTTCCTAGGAAACGCGTATGTGCACATACGCGTTTCCTAGGAAACCAGAGAAG |
| | GATCAAAGCCCCTCTCACACACCGGGGAGCGGGGAAGAGAACTGTTTTGCTTTCATT |
| | GTAGACCAGTGAAATTGGGAGGGGTTTTCCGACCGAAGTCAGAAAACCTGCTCCAAA |
| | AATTCCACTCAAAGTGTATTTTATGTTTTCCAGTGTGGTGTAAAGGAATTCATTAGCC |
| | GTGGATGTATTCATGAAAGGACTTTCAAAGGCCAAGGAGGGAGTTGTGGCCGGTGCG |
| | GAAGTGCGTCTGTAGCGAGCCAGGGAAGGACATCAACTCCACTTTCGATGAGGGTGA |
| | GATCAAGGTGCCATTTCCACACCCCTCCACTGATATGTGAATCACAAAGCACAGTTCC |
| | TTATTCGGTTCGATAAACAATATTCTAAAAGACTATTAAAACCGCTCGTTTCTTGAGT |
| | TTGTGACCGCTTGTAAAGGCTATGCAAATGAGTCAGTGCTGATTGGCTGAAAACAGC |
| | CAATCACAGCTCCTATGTTGTTACCTGGCATTATGCCCAGTACATGACCTTATGGGAC |
| | TTTCCTACTTGGCAGTACATCTACGTATTAGTCATCGCTATTACCATGGTGATGCGGTT |
| | TTGGCAGTACATCAATGGGCGTGGATAGCGGTTTGACTCACGGGGATTTCCAAGTCTC |
| | CACCCCATTGACGTCAATGGGAGTTTGTTTTGGCACCAAAATCAACGGGACTTTCCAA |
| | AATGTCGTAACAACTCCGCCCCATTGACGCAAATGGGCGGTAGGCGTGTACGGTGGG |
| | AGGTCTATATAAGCAGAGCTCGTTTAGTGAACCGTCAGATCGCCTGGAGACGCCATC |
| | CACGCTGTTTTGACCTCCATAGTAGAGCCGCCACCATGGCCACAACCATGGGTCTTTT |
| | CTGCAATCACCGTCCTCGAGGCACCATGAACCCAGCCATCAGCGTCGCTCTCCTGCTC |
| | TCAGTCTTGCGGGTGTCCCGAGGGCAGAAGGTGACCAGCCTGACAGCCTGCCTGGTG |
| | AACCAAAACCTTCGCCTGGACTGCCGCCATGAGAATAACACCAAGGATAACTCCATC |
| | CAGCATGAGTTCAGCCTGACCCGAGAGAAGAGGAAGCACGTGCTCTCAGGCACCCTT |
| | GGGATACCCGAGCACACGTACCGCTCCCGCGTCACCCTCTCCAACCAGCCCTATATCA |
| | AGGTCCTTACCCTAGCCAACTTCACCACCAAGGATGAGGGCGACTACTTTTGTGAGCT |
| | TCGCGTCTCGGGCGCGAATCCCATGAGCTCCAATAAAAGTATCAGTGTGTATAGAGA |
| | CAAGCTGGTCAAGTGTGGCGGCATAAGCCTGCTGGTTCAGAACACATCCTGGATGCT |
| | GCTGCTGCTGCTTTCCCTCTCCCTCCTCCAAGCCCTGGACTTCATTTCTCTGTAAAATA |
| | AAGGAAATTTATTTTCATTGCAATAGTGTGTTGGAATTTTTTGTGTCTCTCAGTACTAA |
| | GATTGCGGCCTGGCCACTGACTAAGATAGAAGCTGTAACTTGCCTCAGATTGTGGCCG |
| | CTGATCTGCAG |

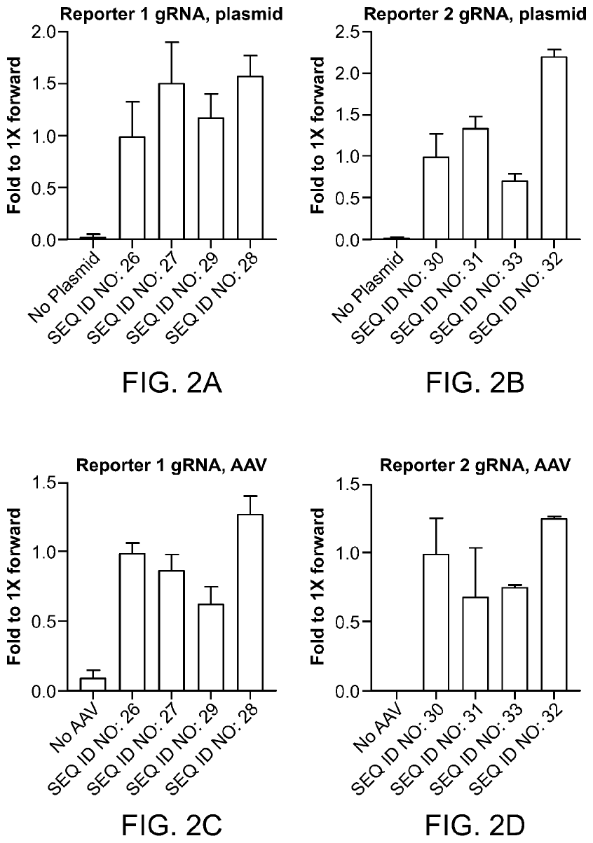

FIG. 2A and FIG. 2C show Reporter 1 gRNA expression (relative to gRNA expression from the 1× forward cassette) for each experimental group as delivered via plasmid transfection and AAV, respectively. FIG. 2B and FIG. 2D show Reporter 2 gRNA expression (relative to gRNA expression from the 1× forward cassette) for each experimental group as delivered via plasmid transfection and AAV, respectively.

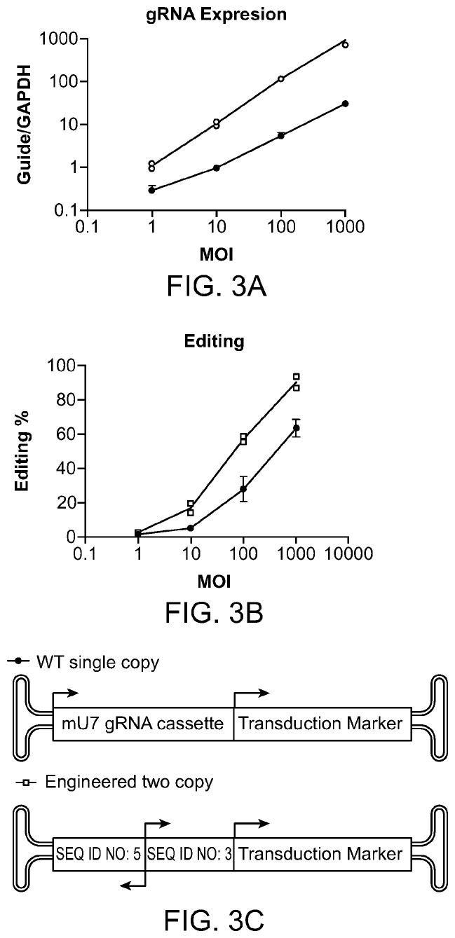

These data demonstrated the benefit to gRNA expression in vectors having two expression cassettes, one in which the first cassette has a reverse read directionality and the second cassette has a forward read directionality (e.g., SEQ ID NO: 28 and SEQ ID NO: 32). The read directionality of the vector with two expression cassettes with the first cassette reverse and the second cassette forward (“reverse, forward” shown in FIG. 1C) was further evaluated in a vector with two expression cassettes with a first expression cassette comprising a first promoter of an engineered mU7 promoter (SEQ ID NO: 5) and a second expression cassette with a second promoter of an engineered hU1 Promoter (SEQ ID NO: 3) and a transduction marker (as shown in the “engineered two copy” vector shown in FIG. 3C. Guide RNA expression (“gRNA expression” in FIG. 3A) and guide RNA editing (“Editing” in FIG. 3B) were tested for a vector with a single expression cassette (“WT single copy” in FIG. 3C, grey lines in FIG. 3A and FIG. 3B) and a vector with two expression cassettes with the first cassette reverse and the second cassette forward (“engineered two copy” in FIG. 3C, blue lines in FIG. 3A and FIG. 3B). AAV expressed gRNAs were delivered to HEK293 cells at different multiplicity of infection (MOI) and RNA was collected at 48 hours. As shown in FIG. 3A, an about 10-fold increase in expression of the guide RNA normalized to a GAPDH control (Guide/GAPDH) was seen with the engineered two copy vector (top blue line) compared to the wild type single copy vector (bottom grey line), over increasing MOI. Sanger analysis of an ATG to GTG change was performed to evaluate editing activity facilitated by the gRNAs expressed from the vector constructs. As shown in FIG. 3B, the engineered two copy vector (top blue line) showed a higher percentage of editing compared to the wild type single copy vector (bottom grey line) at all MOIs ≥10.

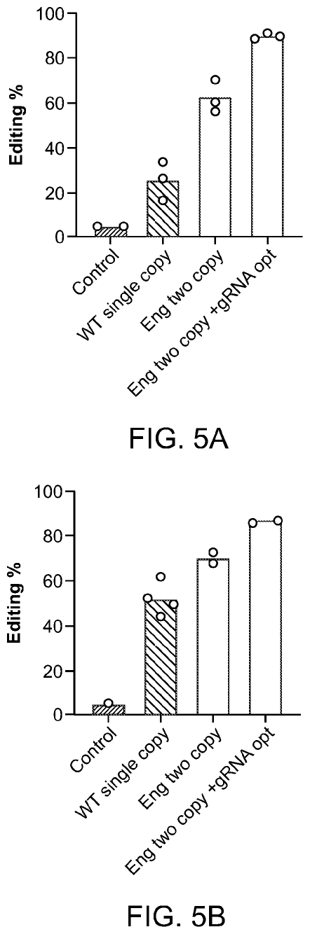

In addition to HEK293 cells, editing was also evaluated in central nervous system (CNS) cell models including mouse primary neurons and SH-SY5Y cells for both the vector with a single expression cassette (“WT single copy” in FIG. 3C, FIG. 5A, and FIG. 5B) and the vector with two expression cassettes with the first cassette reverse and the second cassette forward (“Engineered two copy” in FIG. 3C, and “Eng two copy” in FIG. 5A and FIG. 5B). A vector with two expression cassettes, with the first cassette reverse and the second cassette forward, with additional engineered sequence elements, (“Eng two copy+gRNA opt” in FIG. 5A and FIG. 5B). Sanger analysis of an ATG to GTG change was performed to evaluate editing activity facilitated by the gRNAs expressed from the vector constructs. As shown in FIG. 5A, the editing in mouse primary neurons was enhanced with both the vector with two expression cassettes with the first cassette reverse and the second cassette forward (“Eng two copy”) and the vector with two expression cassettes, with the first cassette reverse and the second cassette forward, with additional engineered sequence elements, including an hnRNP motif, an OPT sequence, and a hairpin sequence (a non-recruitment hairpin sequence), was also evaluated (“Eng two copy+gRNA opt”) compared to the vector with a single expression cassette (“WT single copy”). As shown in FIG. 5B, the editing in SH-SY5Y cells was also enhanced with both the vector with two expression cassettes with the first cassette reverse and the second cassette forward (“Eng two copy”) and the vector with two expression cassettes, with the first cassette reverse and the second cassette forward, with additional engineered sequence elements, including an hnRNP motif, an OPT sequence, and a hairpin sequence (a non-recruitment hairpin sequence), was also evaluated (“Eng two copy+gRNA opt”) compared to the vector with a single expression cassette (“WT single copy”).

### Example 2

### Design and Screening of Four Expression Cassette Vector Systems

This example describes engineering and analyzing vectors with four expression cassettes in various orientations and arrangements to identify the effect of cassette orientation on guide RNA expression. Constructs are screened for engineered guide RNA expression using a luciferase reporter assay. The constructs comprise four expression cassettes with each cassette having a pair of a promoter and terminator sequence. The four pairs of promoter and terminator sequences in the vector with four expression cassettes include a promoter sequence of SEQ ID NO: 5 paired with a terminator sequence of SEQ ID NO: 11; SEQ ID NO: 6 paired with a terminator sequence of SEQ ID NO: 13; SEQ ID NO: 8 paired with a terminator sequence of SEQ ID NO: 15; or SEQ ID NO: 6 paired with a terminator sequence of SEQ ID NO: 13. The four expression cassettes are each independently arranged in either a forward or reverse read orientation creating the 16 permutations of read directionality shown in FIG. 4A. As shown in FIG. 4B, permutations include a vector with reverse, reverse, forward, forward read orientation (1), a vector with forward, forward, forward, forward read orientation (2), a vector with reverse, reverse, reverse, reverse read orientation (3), a vector with reverse, forward, forward, forward read orientation (4), and a vector with reverse, reverse, reverse, forward read orientation (5). Each vector read directionality permutation is tested for guide RNA expression. Read orientation of a multi-expression cassette vector is then designed for increased guide RNA expression and editing.

### Example 3

### Further Screening of Multi-Expression Cassette Vector Systems

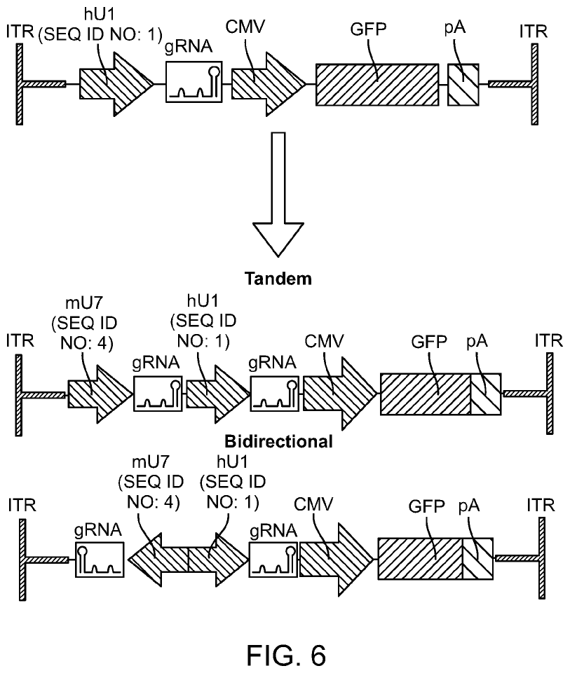

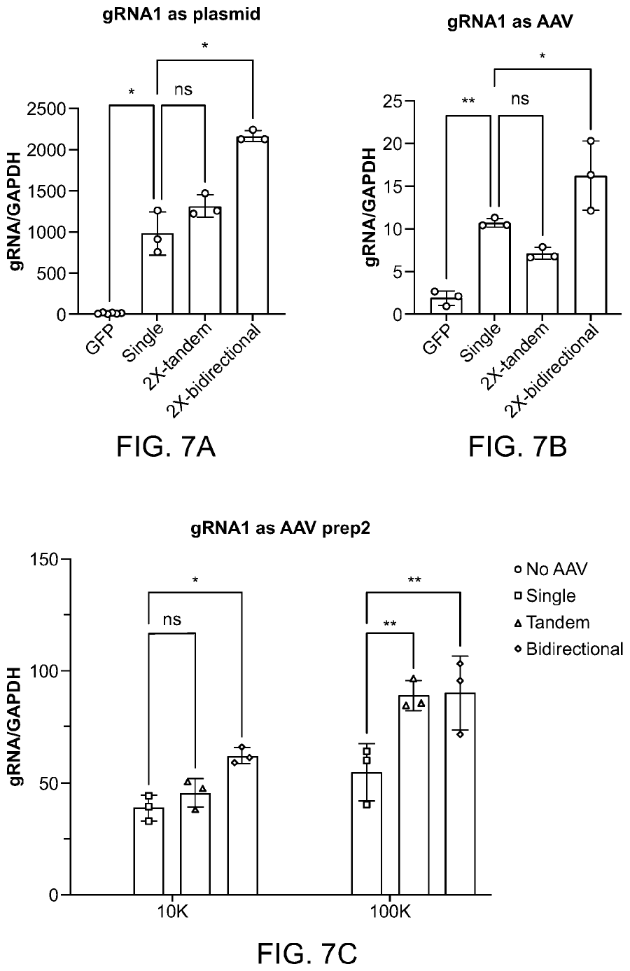

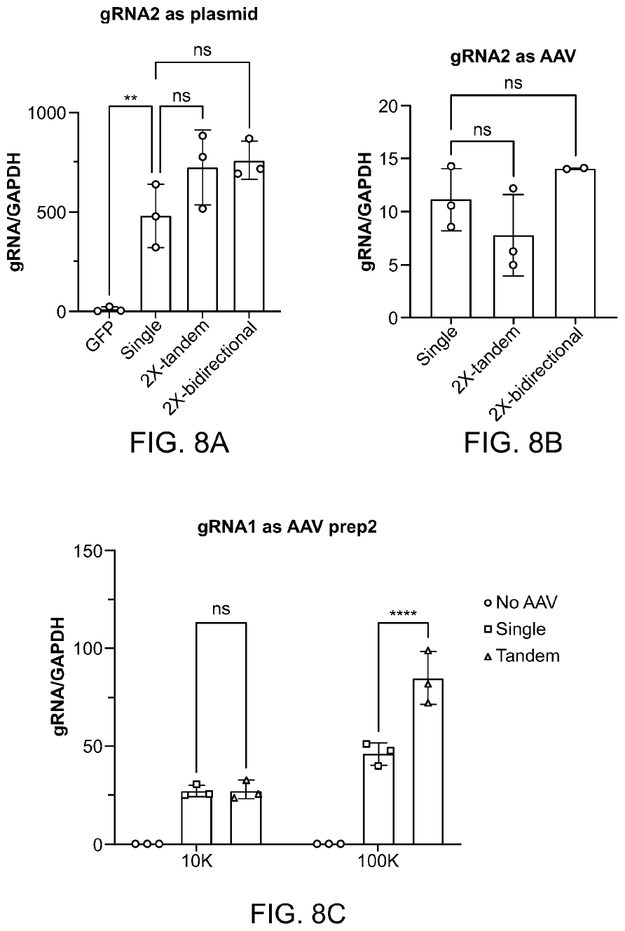

This example describes engineering and analyzing vectors with two expression cassettes in various orientations to identify the effect of cassette orientation on guide RNA expression. Constructs are screened for engineered guide RNA expression via plasmid and AAV transduction using a fluorescence reporting assay to measure the expression of the guide RNA normalized to a GAPDH control (Guide/GAPDH). As shown in FIG. 6, the two-copy vectors were tested in two orientations including a tandem orientation wherein the guide RNAs have the same read directionality of forward and a bidirectional orientation wherein the guide RNAs have different read directionalities. Specifically, the bidirectional orientation has a first guide RNA with a read directionality of reverse, and a second guide RNA with a read directionality of forward. As shown in FIG. 7A and FIG. 7B, the bidirectional orientation (2×-bidirectional) had increased expression of the guide RNA (gRNA1) in plasmid (FIG. 7A) and AAV (FIG. 7B) transfection compared to either the tandem orientation (2×-tandem) or a vector with a single guide RNA (single). Further shown in FIG. 7C, AAV transfection at a multiplicity of infection (MOI) of 10K also had increased expression of the guide RNA (gRNA1) with a bidirectional orientation (shaded diamonds) compared to either the tandem orientation (shaded triangles) or the vector with a single guide RNA (shaded squares). Also shown in FIG. 7C, AAV transfection at a multiplicity of infection (MOI) of 100K had increased expression of the guide RNA (gRNA1) with the bidirectional orientation (shaded diamonds) and the tandem orientation (shaded triangles) compared to the vector with a single guide RNA (shaded squares). As shown in FIG. 8A and FIG. 8B, the bidirectional orientation (2×-bidirectional), the tandem orientation (2×-tandem), and the vector with a single guide RNA (single) showed similar expression of a guide RNA (gRNA2) in plasmid (FIG. 8A) and AAV (FIG. 8B) transfection. Further shown in FIG. 8C, AAV transfection at a multiplicity of infection (MOI) of 10K showed similar expression of the guide RNA (gRNA2) with the tandem orientation (shaded triangles) and the vector with a single guide RNA (shaded squares). Also shown in FIG. 8C, AAV transfection at a multiplicity of infection (MOI) of 100K had increased expression of the guide RNA (gRNA2) with the tandem orientation (shaded triangles) compared to the vector with a single guide RNA (shaded squares).

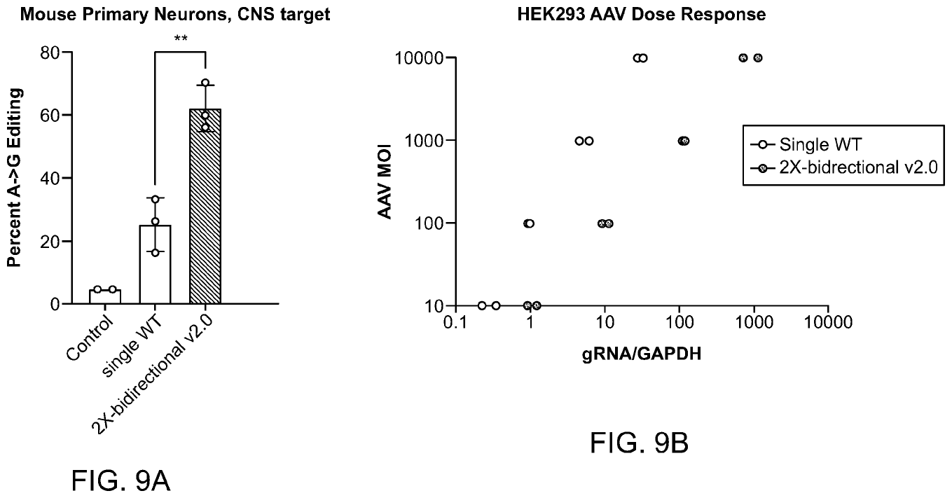

The bidirectional orientation was then tested for editing of a CNS target in mouse primary neuron cells. The percent editing of the CNS target was quantified by Sanger sequencing. As shown in FIG. 9A, the bidirectional orientation (2× bidirectional v2.0) resulted in increased percent A to G editing compared to a vector with a single guide RNA (single WT). The bidirectional orientation was also tested at varied multiplicity of infection (MOI) in HEK 293 cells. Each AAV transduction was replicated in triplicate at each MOI. The RNA was collected at 48 hours and quantified by ddPCR for guide RNA expression relative to GAPDH. As shown in FIG. 9B, the bidirectional orientation (2× bidirectional v2.0) resulted in increased guide expression (gRNA/GAPDH) compared to a vector with a single guide RNA (single WT) across all tested MOIs.

### Example 4

### Vector Genome Integrity of Multi-Expression Cassette Vector Systems

This example describes the characterization of the vector genome integrity of vectors with multiple expression cassettes. The recombination of multi-expression cassette vectors was assessed with alkaline gels that denature AAV genomes. In an alkaline gel, denatured AAV genomes will run as single stranded molecules and therefore can show if the AAV genome has undergone recombination which will result in AAV genomes of multiple sizes and multiple bands on the alkaline gel.

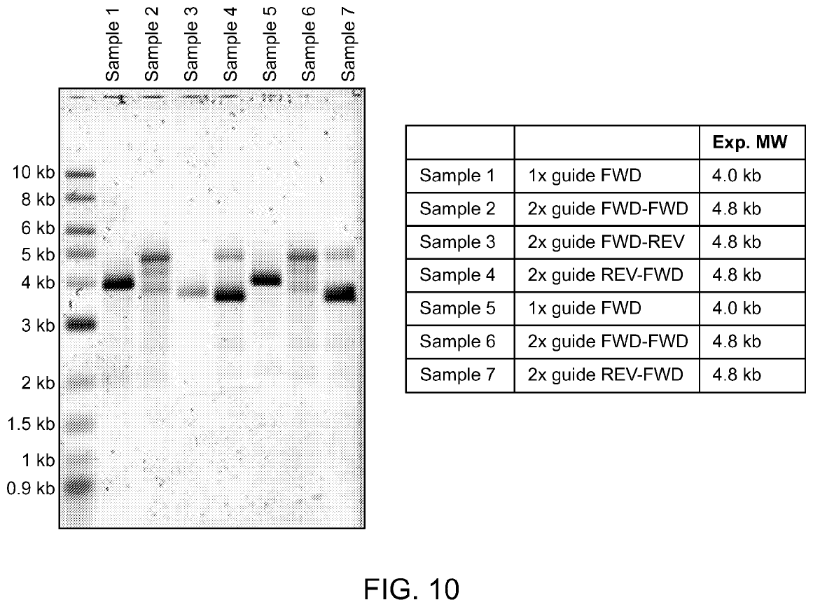

The genome integrity of two-copy vectors comprising two expression cassettes each with the same guide RNA, hairpin structure (e.g., a non-recruitment hairpin), and terminator sequence (about 250 nucleotides of sequence homology) was evaluated. The two-copy vectors had varied orientations including forward, forward; forward reverse; and reverse, forward and were compared to one-copy vectors with a forward orientation. The size of each of the two-copy vector genomes were 4.8 kb and the size of the single-copy vector genomes were 4.0 kb. As seen in FIG. 10, the single-copy vectors had a band around 4.0 kb. For the two-copy vectors, the vectors with a forward, forward orientation showed a band around 4.8 kb with two less intense bands around 4.5 kb and 3.9 kb. For the two-copy vector with a forward, reverse read orientation, one band was seen around 3.9 kb. For the two-copy vectors with a reverse, forward read orientation, an intense band was seen around 3.9 kb and a less intense band was seen around 4.8 kb. The two-copy vectors that showed a band less than 4.8 kb likely underwent a recombination event and therefore had a product with a lower molecular weight than the intact vector genome.

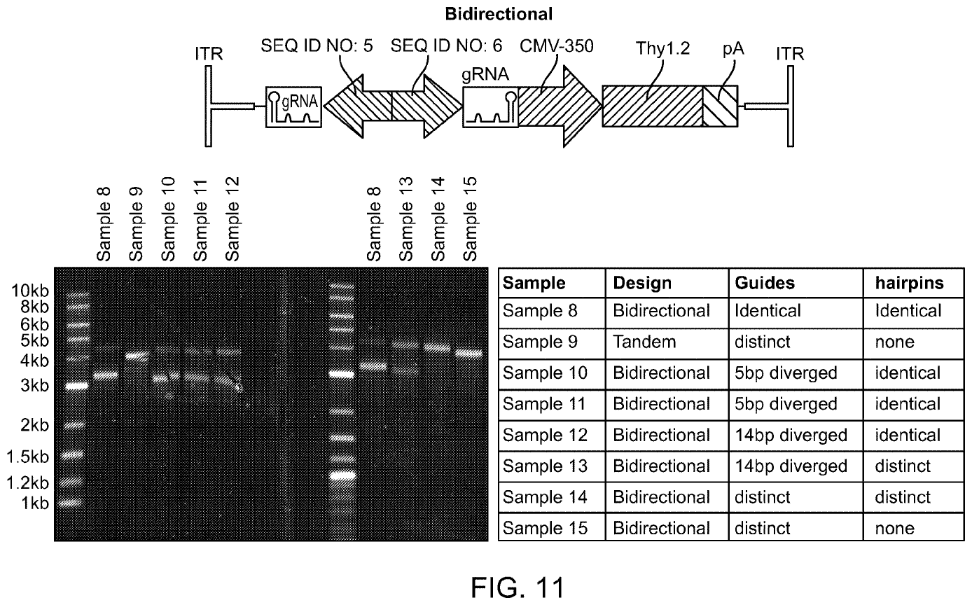

The genome integrity of multiple expression cassette vectors was also assessed for vectors with two guide RNAs with different sequences (distinct sequences), vectors with two guide RNAs with similar sequences (diverged guide RNAs), and vectors with two guide RNAs with the same sequence (identical guide RNAs). As shown in FIG. 11, additional bands were seen in the lanes of the bidirectional vectors with two identical guide RNAs and two diverged guide RNAs. Only one band was seen in the lanes of the bidirectional vectors with two distinct guide RNAs and two distinct guide RNAs and no hairpins and the tandem vector with two distinct guide RNAs and no hairpins. These data show that vector genome integrity was increased in two-copy vectors as sequence divergence increased from identical to diverged (e.g., 5 bp or 14 bp diverged) to distinct guide RNA sequences in each expression cassette of the two-copy vector. These data suggest that increased sequence homology may promote the propensity for recombination in two-copy vectors.

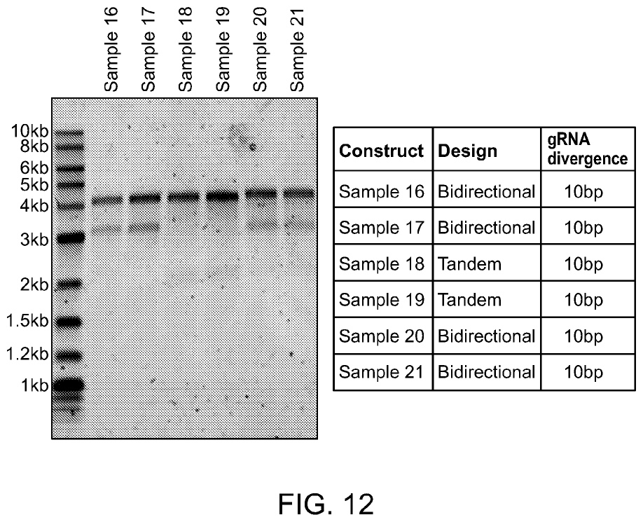

The genome integrity of multiple expression cassette vectors was also assessed for two-copy vectors with guide RNAs with 10 bp of divergence with each expression cassette also comprising distinct hairpin (e.g., non-recruitment hairpin) and terminator sequences. The two-copy vectors were tested in both bidirectional and tandem orientations. As shown in FIG. 12, the two-copy vectors with a tandem read orientation had an intense band around 4.0 kb whereas the two-copy vectors with a bidirectional had an intense band around 4.0 kb but also showed an additional less intense band around 3.0 kb. These data suggest that the two-copy vectors with a tandem read orientation had less recombination than the two-copy vectors with a bidirectional read orientation.

### Example 5

### Enhancing Vector Genome Integrity by Using Divergent Guide RNAs in a Two-Copy Vector

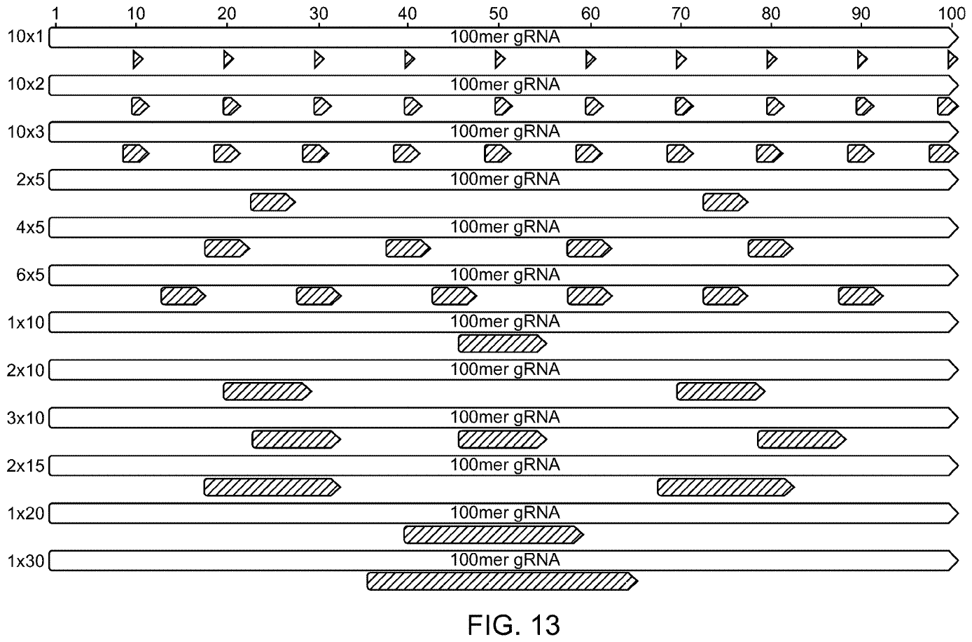

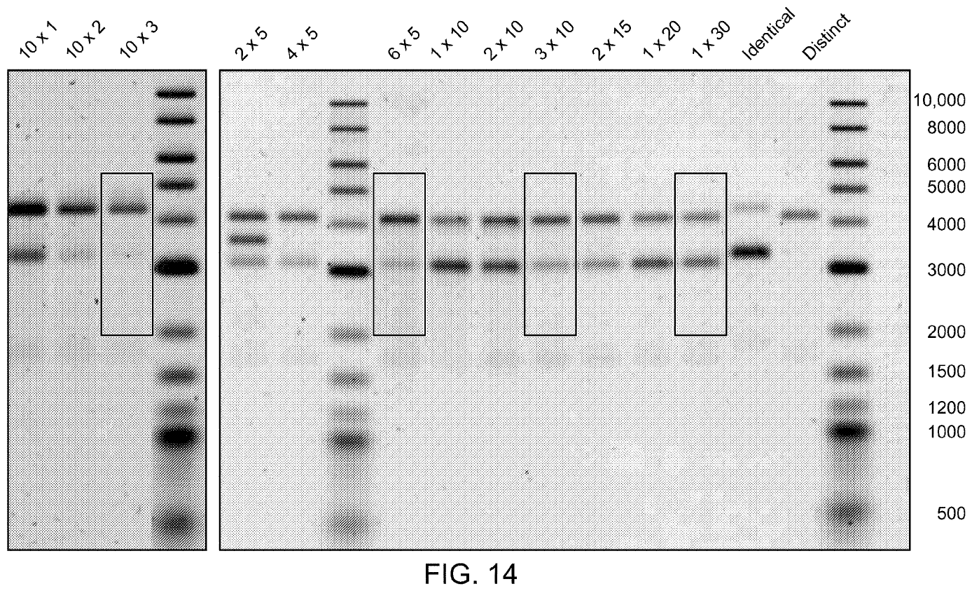

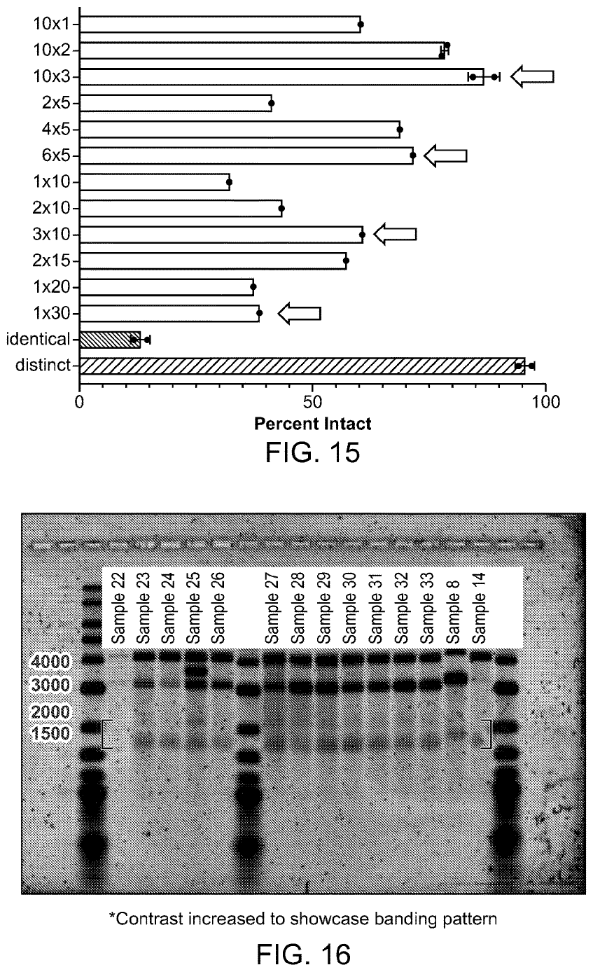

This example describes the characterization of the genome integrity of vectors with multiple expression cassettes wherein the guide RNA sequences vary in sequence homology and have dispersed regions of nucleotide divergence from a first guide RNA. The guide RNA sequences were varied by a total of 10 nucleotides, 20 nucleotides, or 30 nucleotides from a first guide RNA sequence that was 100 nucleotides in length. These sequence divergent guide RNAs were then incorporated into a two-copy vector also comprising the first guide RNA and the vector genome integrity resulting was compared to a two-copy vector design that had two guide RNAs with different sequences (distinct) and a two-copy vector design that had two guide RNAs that were the same sequence (identical). Multiple 100 nucleotide guide RNAs (100mer gRNA) were varied at regions of divergence that were 1 nucleotide alterations, 2 nucleotide alterations, 3 nucleotide alterations, 5 nucleotide alterations, 10 nucleotide alterations, 15 nucleotide alterations, 20 nucleotide alterations, or 30 nucleotide alterations from a guide RNA sequence wherein the nucleotide alterations were dispersed in 10 regions, 2 regions, 4 regions, 6 regions, 1 region, or 3 regions over the 100-nucleotide sequence. For example, as shown in FIG. 13, 100mer gRNAs were tested that had 10 regions of 1 nucleotide alteration (10×1), 10 regions of 2 nucleotide alterations (10×2), 10 regions of 3 nucleotide alterations (10×3), 2 regions of 5 nucleotide alterations (2×5), 4 regions of 5 nucleotide alterations (4×5), 6 regions of 5 nucleotide alterations (6×5), 1 region of 10 nucleotide alterations (1×10), 2 regions of 10 nucleotide alterations (2×10), 3 regions of 10 nucleotide alterations (3×10), 2 regions of 15 nucleotide alterations (2×15), 1 region of 20 nucleotide alterations (1×20), or 1 region of 30 nucleotide alterations (1×20). As shown in FIG. 14, the 100mer sequences with more dispersed regions of nucleotide alterations resulted in less recombination observed by fainter or absent second gel bands. For example, by comparing a multi-expression cassette vector comprising a first 100mer guide RNA and a second 100mer guide RNA with 30 total nucleotide alterations from the first 100mer guide RNA, a trend was seen of a less intense band from more dispersed regions of nucleotide alterations to less dispersed regions of nucleotide regions (i.e., 10×3<6×5<3×10<1×30). This trend is further seen by calculating the vector genome integrity by calculating a percent intact value. The percent intact values for each of the 100mer gRNAs were calculated using the intensities of each gel band as measured by ImageJ analysis software and the following equation: Percent Intact=(Intensity (Full length vector)/(Intensity (Full length vector)+Intensity (Vector truncations)))*100%; wherein the Intensity (Full length vector) is the intensity of the highest molecular weight band in the gel and the Intensity (Vector truncations) is the intensity of the lower molecular weight band. The percent intact values for each of the 100mer gRNAs are provided in FIG. 15. As shown in FIG. 15, the 100mer sequence with 30 total nucleotide alterations from the first guide RNA sequence dispersed over 10 regions of 3 nucleotide alterations (10×3) had the highest percent intact value of the two-copy vector designs and was near the percent intact value of a two-copy vector design with two different guide RNA sequences (distinct). Also seen in FIG. 15, all sequence divergent guide RNAs with between 10 nucleotide and 30 nucleotide alterations had increased vector genome integrity as compared to a two-copy vector design with two guide RNAs of the same sequence (identical). As shown in FIG. 16, all vector constructs also were seen to have a distinct lower band around 1.3 kb, as seen when the contrast is increased from FIG. 14.

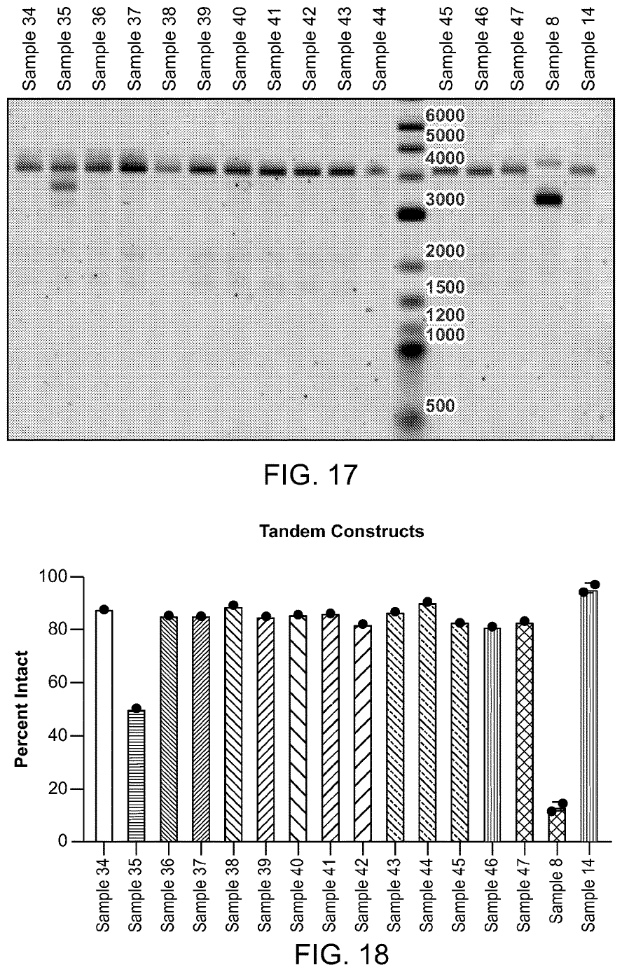

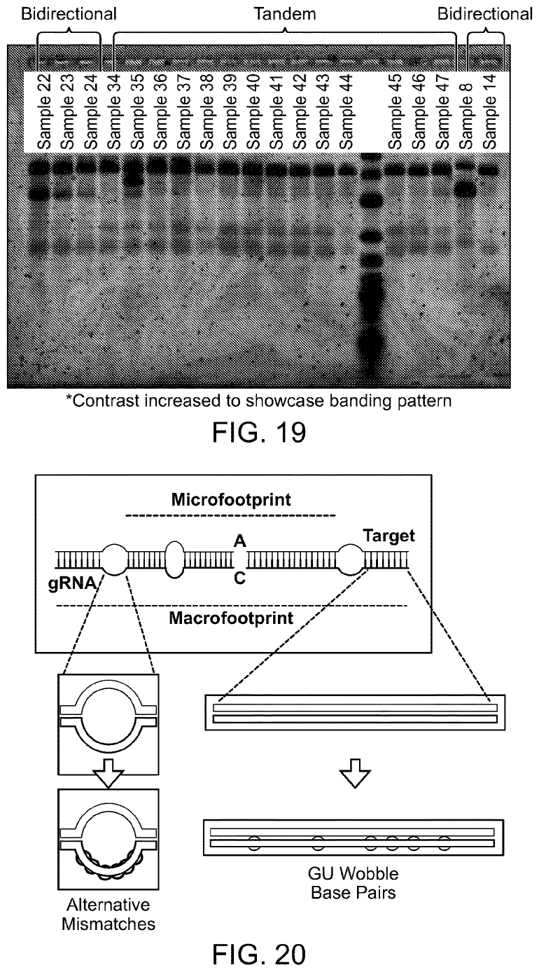

The 100mer sequence divergent guide RNAs were also tested in a tandem orientation. As shown in FIG. 17, the tandem orientation vectors showed less recombination than the bidirectional orientation. The percent intact value was also calculated for the tandem orientation vectors as described above and is provided in FIG. 18. As seen in FIG. 18, most of the tandem orientation vectors were seen to have around 85 percent intact vector genomes. As seen in FIG. 19, all tandem vector constructs also were seen to have a distinct lower band, as seen when the contrast is increased, comparable to the bidirectional vector constructs, and further have an additional lower band that is directly above the low band seen in both orientations.

### Example 6

### Developing Sequence Divergent Guide RNAs

This example describes the development of sequence divergent guide RNAs for use in a multi-copy vector. As discussed in EXAMPLE 4 and EXAMPLE 5, vector genome integrity may decline with increased sequence homology between two expression cassettes in a multi-copy vector due to recombination events. To minimize recombination events and increase vector genome integrity, guide RNA sequences were altered to introduce sequence divergence (e.g., nucleotide alterations between two guide RNA sequences) for use in a two-copy vector. While sequence divergence between two guide RNAs may reduce recombination, guide RNA structure can be mostly retained in the guide-target RNA scaffold. Accordingly, nucleotides were altered in the guide RNA sequence that had a minimal impact on the guide-target RNA scaffold including alternative mismatches (nucleotides that are not base paired within the guide-target RNA scaffold) and GU wobble base pairs (which retain association and base pairing within the guide-target RNA scaffold). Guide RNA sequences were diverged by taking a first guide RNA sequence and introducing nucleotide alterations in the sequence. Nucleotide alterations were introduced at nucleotide positions in the guide RNA sequence that would either be an alternative mismatch position in the guide-target RNA scaffold or would introduce a GU wobble base pair in the guide-target RNA scaffold as shown in FIG. 20. As shown in FIG. 20, alternative mismatches were introduced in regions of internal structure in the guide-target RNA scaffold (e.g., internal loops) and GU wobble base pairs were introduced at A and C nucleotides by replacement with G and T nucleotides, respectively.

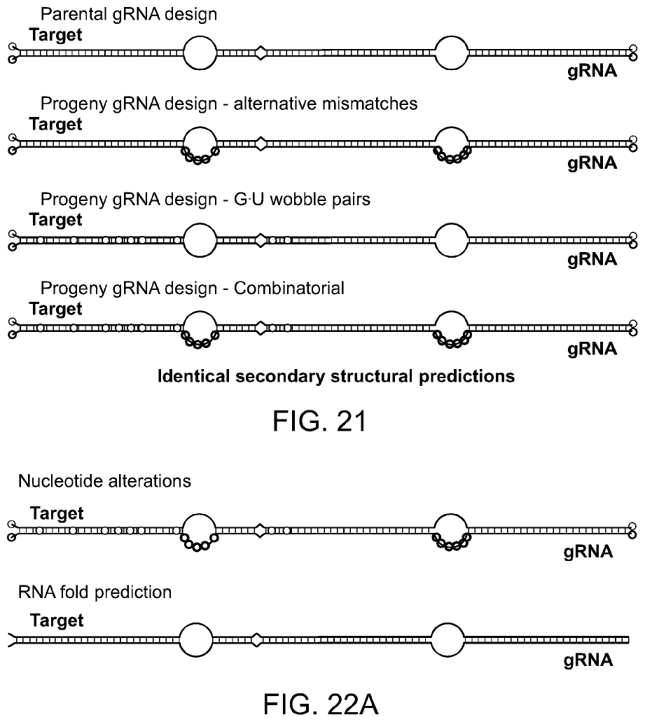

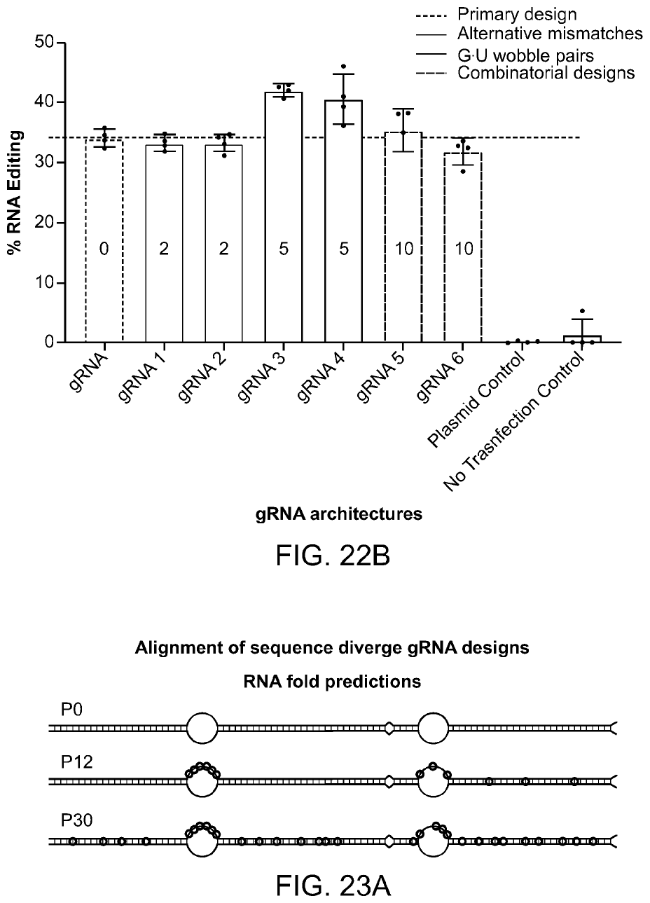

Sequence divergent guide RNAs were designed with i) only alternative mismatches, ii) only GU wobble base pairs and iii) combinatorial design with both alternative mismatches and GU wobble base pairs, as shown in FIG. 21. The guide RNA sequence was altered at the nucleotides indicated in FIG. 22A and confirmed to have the same RNA fold prediction as the original guide RNA. As shown in FIG. 22A, the sequence divergent guide RNA had both GU wobble base pairs introduced into the guide-target RNA scaffold (dark blue circles) and alternative mismatches in the internal loops of the guide-target RNA scaffold (light blue circles). The sequence divergent guide RNAs were then tested for RNA editing activity, provided in FIG. 22B. As shown in FIG. 22B, the sequence divergent guide RNAs with alternative mismatches, GU wobble base pairs, and the combinatorial designs of both alternative mismatches and GU wobble base pairs had similar RNA editing levels to the original guide RNA sequence (“primary design”). Also shown in FIG. 22B, the guide RNA sequence with the GU wobble base pairs had increased RNA editing as compared to the original guide RNA sequence (“primary design”).

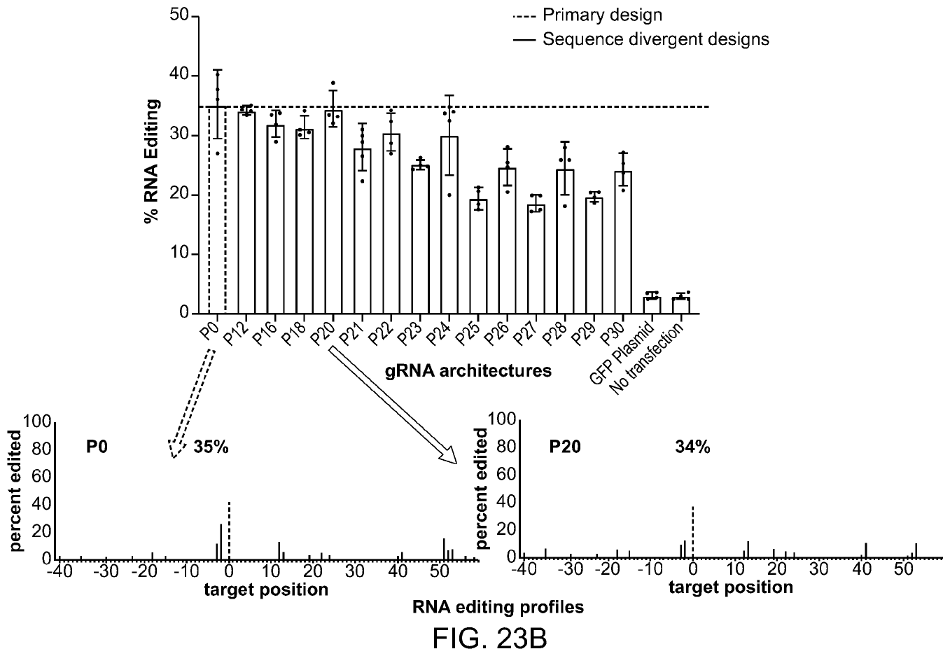

Sequence divergent guide RNAs were also developed with varying numbers of total nucleotide alterations to evaluate the impact on number of nucleotide alterations on RNA editing. As shown in FIG. 23A and FIG. 23B, sequence divergent guide RNAs were developed by introducing alternative mismatches and GU wobble base pairs for a total of 12 (“P12”), 16 (“P16”), 18 (“P18”), 20 (“P20”), 21 (“P21”), 22 (“P22”), 23 (“P23”), 24 (“P24”), 25 (“P25”), 26 (“P26”), 27 (“P27”), 28 (“P28”), 29 (“P29”), and 30 (“P30”) total nucleotide alterations from the original guide RNA sequence (“P0”). The sequence divergent guide RNA sequences are provided in TABLE 6.

TABLE 6
Sequence Divergent Guide RNAs

| Guide Name in | | |
|---|---|---|
| FIGURE | RNA Sequence | DNA Sequence |
| PO | (SEQ ID NO: 36) | (SEQ ID NO: 37) |
| | GUGUCCUUUAGCAGGGCCUU | GTGTCCTTTAGCAGGGCCTTGGA |
| | GGAGCCACCUAGGACUCUCC | GCCACCTAGGACTCTCCTGGCCC |
| | UGGCCCUUACCACAGGGGCC | TTACCACAGGGGCCATTCACCTT |
| | AUUCACCUUGUGCCUCCUGC | GTGCCTCCTGCCGTGGCACTGG |
| | CGUGGCACUGGAGCUGCGGU | AGCTGCGGT |
| P12 | (SEQ ID NO: 38) | (SEQ ID NO: 39) |
| | GUGUCCUUUAGCAGGGCCUU | GTGTCCTTTAGCAGGGCCTTGGA |
| | GGAGAGCAACAGGACUCUCC | GAGCAACAGGACTCTCCTGGCC |
| | UGGCCCUUACCACAGGGGCC | CTTACCACAGGGGCCATTCAAC |
| | AUUCAACCUUUGCCUCCUGU | CTTTGCCTCCTGTCGTGGTACTG |
| | CGUGGUACUGGAGUUGCGGU | GAGTTGCGGT |
| P16 | (SEQ ID NO: 40) | (SEQ ID NO: 41) |
| | GUGUCCUUUAGCAGGGCCUU | GTGTCCTTTAGCAGGGCCTTGGA |
| | GGAGAGCAACAGGACUCUCU | GAGCAACAGGACTCTCTTGGCT |
| | UGGCUCUUAUCACAGGGGCC | CTTATCACAGGGGCCATTCAAC |
| | AUUCAACCUUUGCCUUCUGU | CTTTGCCTTCTGTCGTGGTACTG |
| | CGUGGUACUGGAGUUGCGGU | GAGTTGCGGT |
| P18 | (SEQ ID NO: 42) | (SEQ ID NO: 43) |
| | GUGUCCUUUAGCAGGGCCUU | GTGTCCTTTAGCAGGGCCTTGGA |
| | GGAGAGCAACAGGGCUCUCU | GAGCAACAGGGCTCTCTTGGCT |
| | UGGCUCUUAUCGCAGGGGCC | CTTATCGCAGGGGCCATTCAAC |
| | AUUCAACCUUUGCCUUCUGU | CTTTGCCTTCTGTCGTGGTACTG |
| | CGUGGUACUGGAGUUGCGGU | GAGTTGCGGT |
| P20 | (SEQ ID NO: 44) | (SEQ ID NO: 45) |
| | GUGUCCUUUGGCAGGGUCUU | GTGTCCTTTGGCAGGGTCTTGGA |
| | GGAGAGCAACAGGGCUCUCU | GAGCAACAGGGCTCTCTTGGCT |
| | UGGCUCUUAUCGCAGGGGCC | CTTATCGCAGGGGCCATTCAAC |
| | AUUCAACCUUUGCCUUCUGU | CTTTGCCTTCTGTCGTGGTACTG |
| | CGUGGUACUGGAGUUGCGGU | GAGTTGCGGT |
| P21 | (SEQ ID NO: 46) | (SEQ ID NO: 47) |
| | GUGUCCUUUGGCAGGGUCUU | GTGTCCTTTGGCAGGGTCTTGGA |
| | GGAGAGCAACAGGGCUCUCU | GAGCAACAGGGCTCTCTTGGCT |
| | UGGCUCUUAUCGCAGGGGCC | CTTATCGCAGGGGCCATTCAAC |
| | AUUCAACCUUUGUCUUCUGU | CTTTGTCTTCTGTCGTGGTACTG |
| | CGUGGUACUGGAGUUGCGGU | GAGTTGCGGT |
| P22 | (SEQ ID NO: 48) | (SEQ ID NO: 49) |
| | GUGUCCUUUGGCAGGGUCUU | GTGTCCTTTGGCAGGGTCTTGGA |
| | GGAGAGCAACAGGGCUCUCU | GAGCAACAGGGCTCTCTTGGCT |
| | UGGCUCUUAUCGCAGGGGCC | CTTATCGCAGGGGCCATTCAAC |
| | AUUCAACCUUUGUCUUCUGU | CTTTGTCTTCTGTCGTGGTACTG |
| | CGUGGUACUGGGGUUGCGGU | GGGTTGCGGT |
| P23 | (SEQ ID NO: 50) | (SEQ ID NO: 51) |
| | GUGUCCUUUGGCAGGGUCUU | GTGTCCTTTGGCAGGGTCTTGGA |
| | GGAGAGCAACAGGGCUCUCU | GAGCAACAGGGCTCTCTTGGCT |
| | UGGCUCUUAUCGCAGGGGCC | CTTATCGCAGGGGCCATTTAACC |
| | AUUUAACCUUUGUCUUCUGU | TTTGTCTTCTGTCGTGGTACTGG |
| | CGUGGUACUGGGGUUGCGGU | GGTTGCGGT |
| P24 | (SEQ ID NO: 52) | (SEQ ID NO: 53) |
| | GUGUCCUUUGGCAGGGUCUU | GTGTCCTTTGGCAGGGTCTTGGA |
| | GGAGAGCAACAGGGCUCUCU | GAGCAACAGGGCTCTCTTGGCT |
| | UGGCUCUUAUCGCAGGGGCC | CTTATCGCAGGGGCCATTTAACC |
| | AUUUAACCUUUGUCUUCUGU | TTTGTCTTCTGTCGTGGTACTGG |
| | CGUGGUACUGGGGUUGUGGU | GGTTGTGGT |
| P25 | (SEQ ID NO: 54) | (SEQ ID NO: 55) |
| | GUGUCCUUUGGCAGGGUCUU | GTGTCCTTTGGCAGGGTCTTGGA |
| | GGAGAGCAACAGGGCUUUCU | GAGCAACAGGGCTTTCTTGGCT |
| | UGGCUCUUAUCGCAGGGGCC | CTTATCGCAGGGGCCATTTAACC |
| | AUUUAACCUUUGUCUUCUGU | TTTGTCTTCTGTCGTGGTACTGG |
| | CGUGGUACUGGGGUUGUGGU | GGTTGTGGT |
| P26 | (SEQ ID NO: 56) | (SEQ ID NO: 57) |
| | GUGUUCUUUGGCAGGGUCUU | GTGTTCTTTGGCAGGGTCTTGGA |
| | GGAGAGCAACAGGGCUUUCU | GAGCAACAGGGCTTTCTTGGCT |
| | UGGCUCUUAUCGCAGGGGCC | CTTATCGCAGGGGCCATTTAACC |
| | AUUUAACCUUUGUCUUCUGU | TTTGTCTTCTGTCGTGGTACTGG |
| | CGUGGUACUGGGGUUGUGGU | GGTTGTGGT |
| P27 | (SEQ ID NO: 58) | (SEQ ID NO: 59) |
| | GUGUUCUUUGGCAGGGUCUU | GTGTTCTTTGGCAGGGTCTTGGA |
| | GGAGAGCAACAGGGCUUUCU | GAGCAACAGGGCTTTCTTGGCT |
| | UGGCUCUUAUCGCAGGGGCC | CTTATCGCAGGGGCCATTTAACC |
| | AUUUAACCUUUGUCUUCUGU | TTTGTCTTCTGTCGTGGTATTGG |
| | CGUGGUAUUGGGGUUGUGGU | GGTTGTGGT |
| P28 | (SEQ ID NO: 60) | (SEQ ID NO: 61) |
| | GUGUUCUUUGGCAGGGUCUU | GTGTTCTTTGGCAGGGTCTTGGA |
| | GGAGAGCAACAGGGCUUUCU | GAGCAACAGGGCTTTCTTGGCT |
| | UGGCUCUUAUCGCAGGGGCC | CTTATCGCAGGGGCCATTTAACC |
| | AUUUAACCUUUGUCUUCUGU | TTTGTCTTCTGTTGTGGTATTGG |
| | UGUGGUAUUGGGGUUGUGGU | GGTTGTGGT |
| P29 | (SEQ ID NO: 62) | (SEQ ID NO: 63) |
| | GUGUUCUUUGGCAGGGUCUU | GTGTTCTTTGGCAGGGTCTTGGA |
| | GGAGAGCAACAGGGCUUUCU | GAGCAACAGGGCTTTCTTGGCT |
| | UGGCUCUUGUCGCAGGGGCC | CTTGTCGCAGGGGCCATTTAACC |
| | AUUUAACCUUUGUCUUCUGU | TTTGTCTTCTGTTGTGGTATTGG |
| | UGUGGUAUUGGGGUUGUGGU | GGTTGTGGT |
| P30 | (SEQ ID NO: 64) | (SEQ ID NO: 65) |
| | GUGUUCUUUGGCGGGGUCUU | GTGTTCTTTGGCGGGGTCTTGGA |
| | GGAGAGCAACAGGGCUUUCU | GAGCAACAGGGCTTTCTTGGCT |
| | UGGCUCUUGUCGCAGGGGCC | CTTGTCGCAGGGGCCATTTAACC |
| | AUUUAACCUUUGUCUUCUGU | TTTGTCTTCTGTTGTGGTATTGG |
| | UGUGGUAUUGGGGUUGUGGU | GGTTGTGGT |

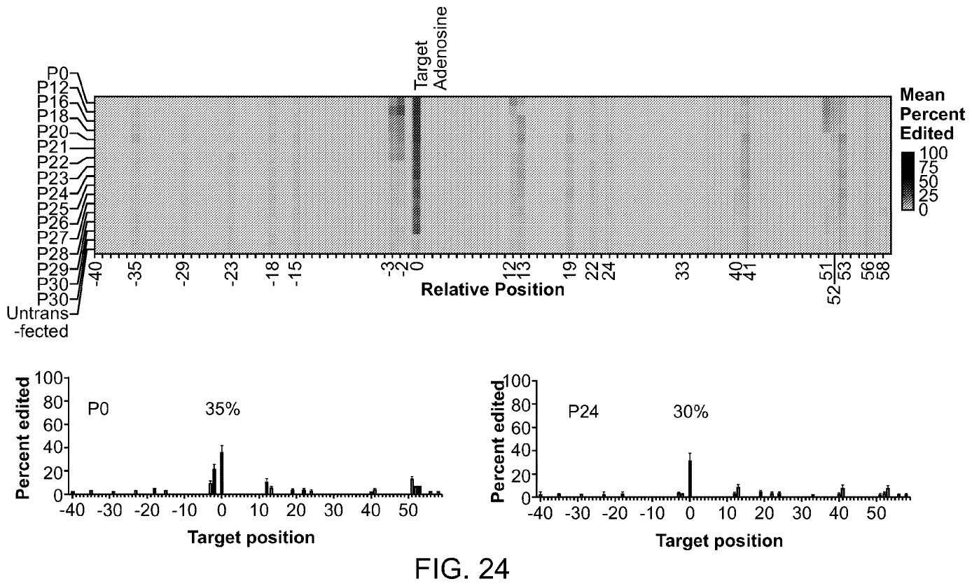

The introduction of the nucleotide alterations was confirmed to not change the guide-target RNA scaffold as shown for 12 (“P12”) and 30 (“P30”) nucleotide alterations as shown in FIG. 23A. As shown in FIG. 23B, 12 (“P12”), 16 (“P16”), 18 (“P18”), 20 (“P20”), 21 (“P21”), 22 (“P22”), and 24 (“P24”) total nucleotide alterations were seen to have similar RNA editing to that of the original guide sequence (“P0”). Also shown in FIG. 23B, the sequence divergent guide RNAs (e.g., 20 nucleotide alterations (“P20”)) were also shown to have similar specificity to the original guide RNA sequence (“P0”) by showing minimal RNA editing of off-target positions. FIG. 24 also shows the sequence divergent guide RNA have similar or increased specificity of editing a target position. As shown in FIG. 24, as the total number of nucleotide alterations increases in the sequence divergent guide RNA sequences, the off-target editing at −2 and −3 relative position decreases.

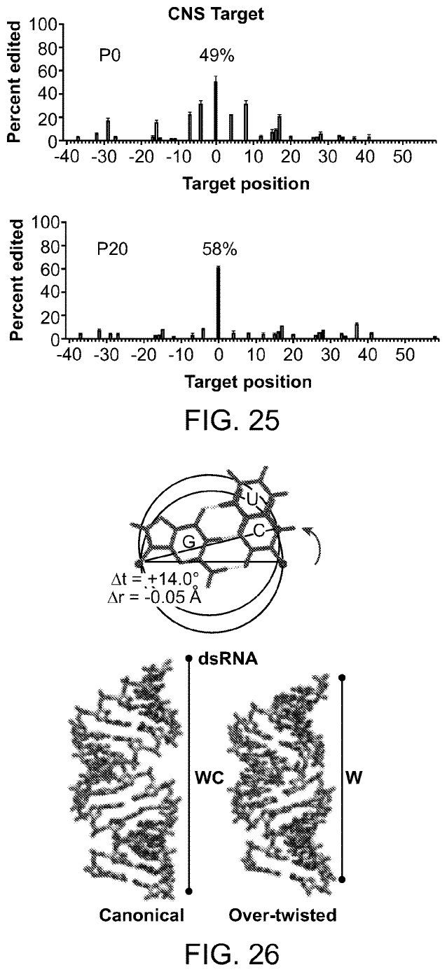

A CNS guide sequence was also used for the development of a sequence divergent guide RNA. As shown in FIG. 25, a sequence diverged guide RNA with 20 total nucleotide alterations (“P20”) showed 58% RNA editing as compared to the original guide RNA sequence (“P0”) that showed a 49% RNA editing. Further as shown in FIG. 25, the sequence diverged guide RNA (“P20”) had increased editing specificity and show less off-target RNA editing than the original guide RNA sequence (“P0”). The increase in RNA editing specificity may be due to the presence of GU wobble base pairs in the sequence diverged guide RNA sequence that would over twist the helix of the guide-target RNA scaffold (FIG. 26) and thereby increase the editing specificity.

### Example 7

### In-Vitro Assessments of Clinical Two-Copy Vector Designs

This example describes the evaluation of clinical two-copy vector designs. Using the tested multi-expression cassette vector designs as described in EXAMPLE 1, and EXAMPLE 3-EXAMPLE 6, vector designs without a transduction marker cassette were further evaluated for their vector genome integrity and RNA editing. The promoter and terminator sequence lengths were extended from 300 base pairs to 400 base pairs and 100 base pairs to 200 base pairs, respectively, for the optimal vector size. Each of the two-copy vectors evaluated are provided in TABLE 7.

TABLE 7
Two-Copy Vector Sequences

| | | Cassette 1 | | | | Cassette 2 | | | | | | |
|---|---|---|---|---|---|---|---|---|---|---|---|---|
| | | | | Small | | | | Small | | | | |
| | | | | nuclear | | | | nuclear | | | | |
| | | | | RNA | | | | RNA | | | | |
| | | | | (snRNA) | | | | (snRNA) | | | | |
| | | | | processing | Tran- | | | processing | Tran- | | | |
| | | | | hairpin | scription | | | hairpin | scription | Marker | | |
| Construct | Configuration | Promoter | gRNA | sequence | terminator | Promoter | gRNA | sequence | terminator | Promoter | marker | polyA |
| Sample 8 | Bidirectional | SEQ ID | gRNA | SEQ ID | SEQ ID | SEQ ID | gRNA | SEQ ID | SEQ ID | CMV350 | GFP | RBG |
| | | NO: 5 | 7 | NO: 83 | NO: 12 | NO: 66 | 7 | NO: 83 | NO: 77 | | | polyA |
| Sample 14 | Bidirectional | SEQ ID | gRNA | SEQ ID | SEQ ID | SEQ ID | gRNA | SEQ ID | SEQ ID | CMV350 | GFP | RBG |
| | | NO: 5 | 8 | NO: 84 | NO: 12 | NO: 66 | 7 | NO: 83 | NO: 77 | | | polyA |
| Sample 48 | Bidirectional | SEQ ID | gRNA | SEQ ID | SEQ ID | SEQ ID | gRNA | SEQ ID | SEQ ID | — | — | — |
| | | NO: 5 | 9 | NO: 84 | NO:78 | NO: 67 | 7 | NO: 83 | NO: 79 | | | |
| Sample 49 | Bidirectional | SEQ ID | gRNA | SEQ ID | SEQ ID | SEQ ID | gRNA | SEQ ID | SEQ ID | — | — | — |
| | | NO: 5 | 10 | NO: 84 | NO: 78 | NO: 67 | 7 | NO: 83 | NO: 79 | | | |
| Sample 50 | Bidirectional | SEQ ID | gRNA | SEQ ID | SEQ ID | SEQ ID | gRNA | SEQ ID | SEQ ID | — | — | — |
| | | NO: 5 | 9 | NO: 84 | NO: 78 | NO: 67 | 10 | NO: 83 | NO: 79 | | | |
| Sample 51 | Tandem | SEQ ID | gRNA | SEQ ID | SEQ ID | SEQ ID | gRNA | SEQ ID | SEQ ID | — | — | — |
| | | NO: 5 | 9 | NO: 84 | NO: 78 | NO: 67 | 7 | NO: 83 | NO: 79 | | | |
| Sample 52 | Tandem | SEQ ID | gRNA | SEQ ID | SEQ ID | SEQ ID | gRNA | SEQ ID | SEQ ID | — | — | — |
| | | NO: 5 | 10 | NO: 84 | NO: 78 | NO: 67 | 7 | NO: 83 | NO: 79 | | | |
| Sample 53 | Tandem | SEQ ID | gRNA | SEQ ID | SEQ ID | SEQ ID | gRNA | SEQ ID | SEQ ID | — | — | — |
| | | NO: 5 | 9 | NO: 84 | NO: 78 | NO: 67 | 10 | NO: 83 | NO: 79 | | | |
| Sample 54 | Tandem | SEQ ID | gRNA | SEQ ID | SEQ ID | SEQ ID | gRNA | SEQ ID | SEQ ID | — | — | — |
| | | NO: 5 | 7 | NO: 84 | NO: 78 | NO: 67 | 7 | NO: 83 | NO: 79 | | | |
| Sample 55 | Bidirectional | SEQ ID | gRNA | SEQ ID | SEQ ID | SEQ ID | gRNA | SEQ ID | SEQ ID | — | — | — |
| | | NO: 5 | 7 | NO: 84 | NO: 78 | NO: 67 | 7 | NO: 83 | NO: 79 | | | |
| Sample 56 | single | SEQ ID | gRNA | SEQ ID | SEQ ID | — | — | — | — | — | — | — |
| | | NO: 67 | 7 | NO: 83 | NO: 79 | | | | | | | |
| Sample 57 | single | SEQ ID | gRNA | SEQ ID | SEQ ID | — | — | — | — | — | — | — |
| | | NO: 67 | 10 | NO: 83 | NO: 79 | | | | | | | |
| Sample 58 | single | SEQ ID | gRNA | SEQ ID | SEQ ID | — | — | — | — | — | — | — |
| | | NO: 67 | 9 | NO: 83 | NO: 79 | | | | | | | |

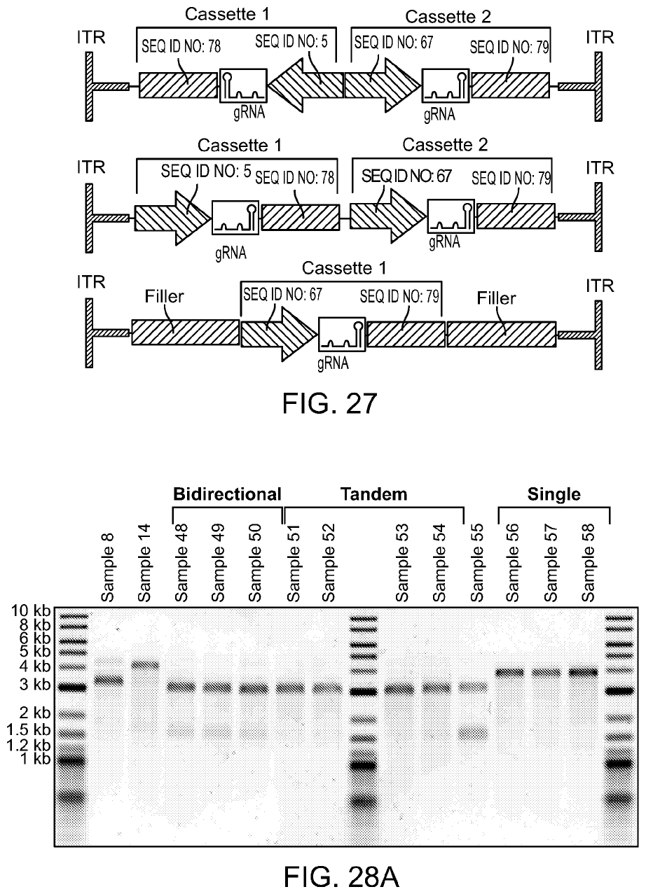

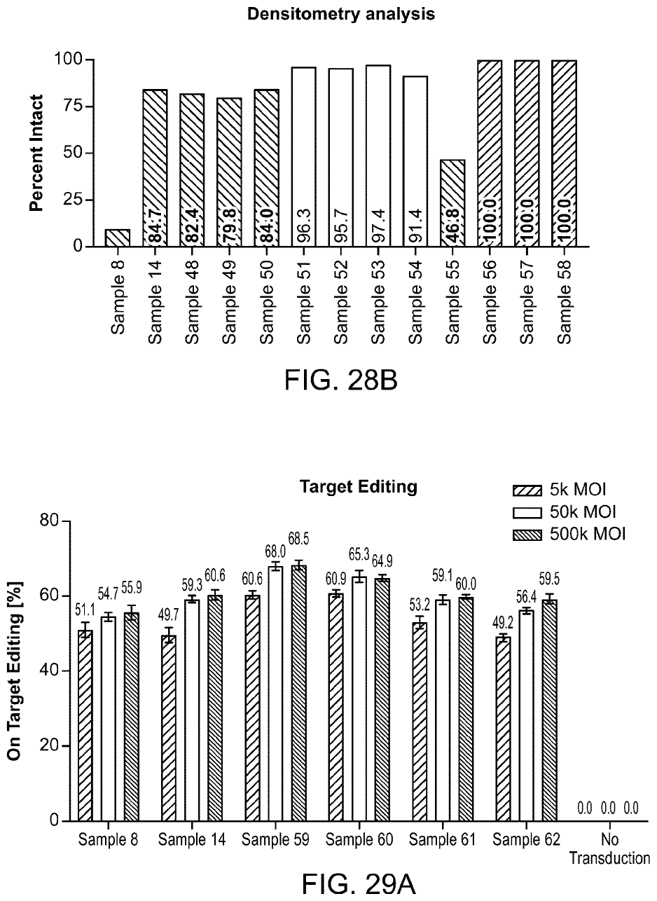

The two-copy vector design included bidirectional vector designs (e.g., top of FIG. 27) and tandem vector designs (e.g., middle of FIG. 27) that were compared to single copy vector designs (e.g., bottom of FIG. 27). Each of the vector designs were tested for vector genome integrity as shown in FIG. 28A and FIG. 28B. As shown in FIG. 28A and FIG. 28B, the tandem vector design showed increased vector genome integrity as compared to the bidirectional vector design.

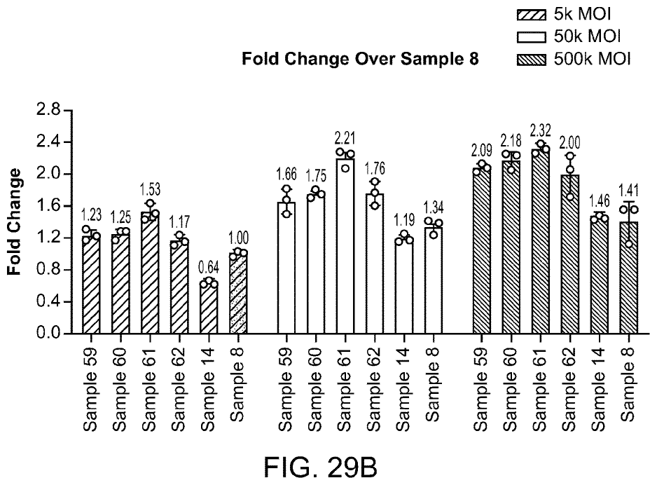

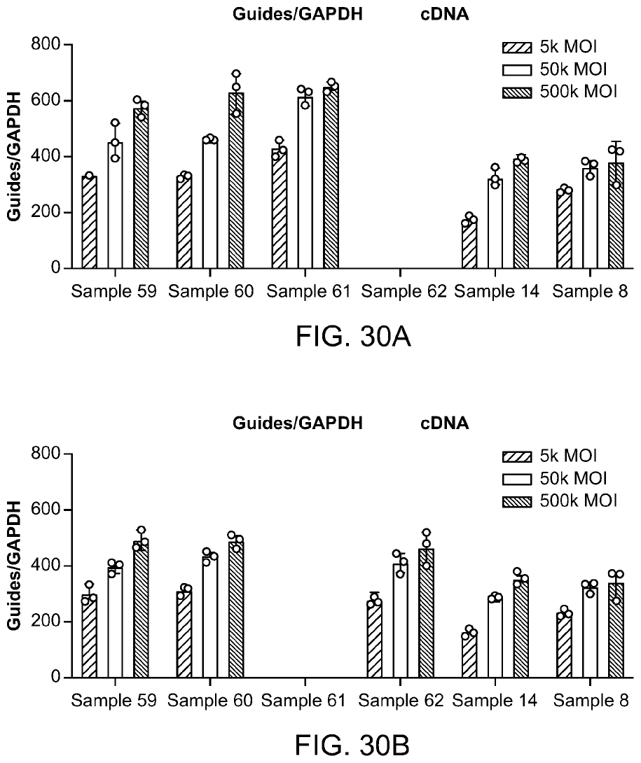

The two-copy vector designs in TABLE 7 were then tested for RNA editing with an SNCA guide RNA against a target sequence. As shown in FIG. 29A, extending the promoter and terminator sequences for the development of transduction marker-free two-copy vectors did not reduce on-target RNA editing. The transduction marker-free two-copy vectors were shown to have similar or increased editing than they two-copy vector with a transduction marker as shown in FIG. 29B. Further, as shown in FIG. 30A and FIG. 30B, the transduction marker-free two-copy vectors produced more guide RNA than the two-copy vectors with a transduction marker.

### Example 8

### Extended Termination Sequences for Clinical Two-Copy Vector Designs

This example describes the development of extended termination sequences for use in a clinical vector without a transduction marker. Termination sequences were extended for optimal vector genome size. The termination sequence of SEQ ID NO: 11 (CCCAATTTCACTGGTTTCAAAAACAGAAAAACAGTTCTCTTCCCCGCTCCCCGGTGT GTGAGAGGGGCTTTGATCCTTCTCTGGTTTCCTAGGAAACGCGTATGTG) was extended by 93 nucleotides to generate the extended termination sequence of SEQ ID NO: 81 (CCCAATTTCACTGGTTTCAAAAACAGAAAAACAGTTCTCTTCCCCGCTCCCCGGTGT GTGAGAGGGGCTTTGATCCTTCTCTGGTTTCCTAGGAAACGCGTATGTGCTAGAGCC ACGCTCTGAGACTTCCGCCTCGTGCGGTCCCGCTTCCTTTCTGCCTCCTCTGGCCTGC ATCCGTGGGGGAGGTGGCTGGCTGCAG) having a total of 199 nucleotides. The termination sequence of SEQ ID NO: 82 (CTTAGTAAGTTTAAAAACAGAAAAAAAACCGTGTTGCTACAGCTATAAACTTCAAA CATGCAGTTTATAGCAGTGGGCAACACGTCTCATCTCAAAAATT) was extended by 100 nucleotides to generate the extended termination sequence of SEQ ID NO: 80 (CTTAGTAAGTTTAAAAACAGAAAAAAAACCGTGTTGCTACAGCTATAAACTTCAAA CATGCAGTTTATAGCAGTGGGCAACACGTCTCATCTCAAAAATTCAGCACTCAAAC ATTACACAGGAAGAGGATGTAATTTTTTAAAATGAAAGCTCTGAAGTAATTTGAGT ATTCTCTGTCCTTTTTGTAAAAAAAATTACGA) having a total of 200 nucleotides. The extended termination sequences are used in a transduction-marker free vector design to maintain vector genome size in absence of the transduction marker sequence.

### Example 9

### Promoter Sequences for Enhancing Guide RNA Expression

This example describes the variation of promoter sequences to enhance guide RNA expression. Sequence variations were introduced into a promoter sequence of SEQ ID NO: 66 to screen for promoter sequence variants with enhanced expression. The sequence variations were introduced in the proximal sequence element (PSE) region, known to be important for promoter activity. The sequence variants of SEQ ID NO: 66 are provided below in TABLE 8.

TABLE 8
Promoter Sequence Variants

| SEQ ID NO | Sequence |
|---|---|
| SEQ ID NO: 68 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCACAAGTCACCATGAGTGTAAAGGGAGTTTCTGTTACCC |
| | GAGAAACGAGTTATGAAATTAACAAGC |
| SEQ ID NO: 69 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCAGATCTCACCATGAGTGTAAAGGGAGTTTCTGTTACCC |
| | GAGAAACGAGTTATGAAATTAACAAGC |
| SEQ ID NO: 70 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCACAAATCACCATAAACGTGAAATGAGTTTCTGTTACCC |
| | GAGAAACGAGTTATGAAATTAACAAGC |
| SEQ ID NO: 71 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCAGATCTCACCATAAACGTGAAATGAGTTTCTGTTACCCG |
| | AGAAACGAGTTATGAAATTAACAAGC |
| SEQ ID NO: 72 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCAGATCTCACCATCACTATACTAGGAGTTTCTGTTACCCG |
| | AGAAACGAGTTATGAAATTAACAAGC |
| SEQ ID NO: 73 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCACAATTTGCCATGAGTATGTTGTGAGTTTCTGTTACCCG |
| | AGAAACGAGTTATGAAATTAACAAGC |
| SEQ ID NO: 74 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCAGATCTTGCCATGAGTATGTTGTGAGTTTCTGTTACCCG |
| | AGAAACGAGTTATGAAATTAACAAGC |
| SEQ ID NO: 75 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCACTAGACACCATCAGTGTACTAGGAGTTTCTGTTACCCG |
| | AGAAACGAGTTATGAAATTAACAAGC |
| SEQ ID NO: 76 | TCGCCCCCTAACGGTGACATAAGGCACTCTGTGAAATGCTCT |
| | GTTCCGGAATCAAAAGATTGATCCGATTATTTGCATACCCAT |
| | AATGCACTGCTCACAGTACAAATTTAAAAAGGCAAAATCAAA |
| | CATTTTTATTCTAAGCATATTCTGTGAAAGTTAGACTTTTGTTT |
| | AAACAATACTCTTAAAATTTTTTTCTAGGTATAGAACCTTGGC |
| | ATTCAGATCACACCATCAGTGTACTAGGAGTTTCTGTTACCCG |
| | AGAAACGAGTTATGAAATTAACAAGC |

The promoter sequence variants provided in TABLE 8 are screened for promoter activity to evaluate their effect on guide RNA expression.

### Example 10

### Increasing Vector Size by Inclusion of Synthetic Filler Sequence

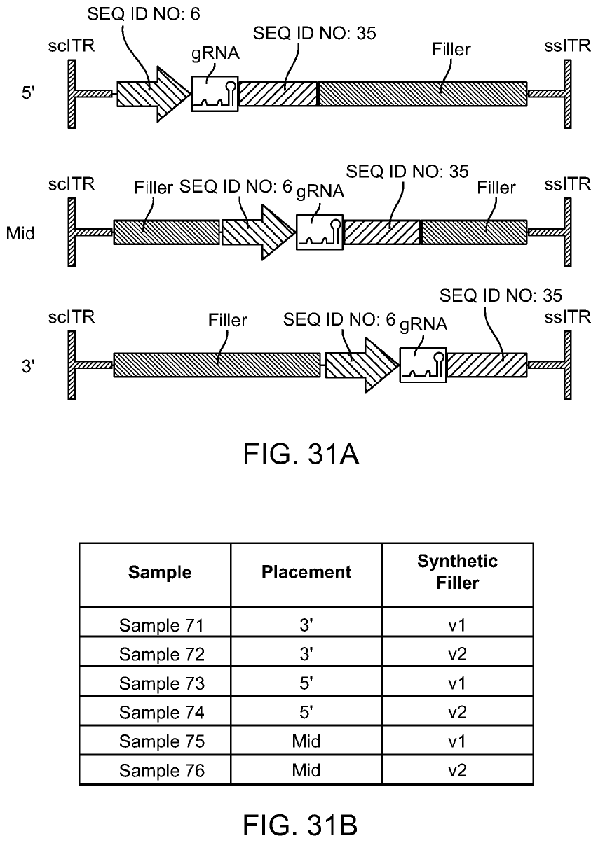

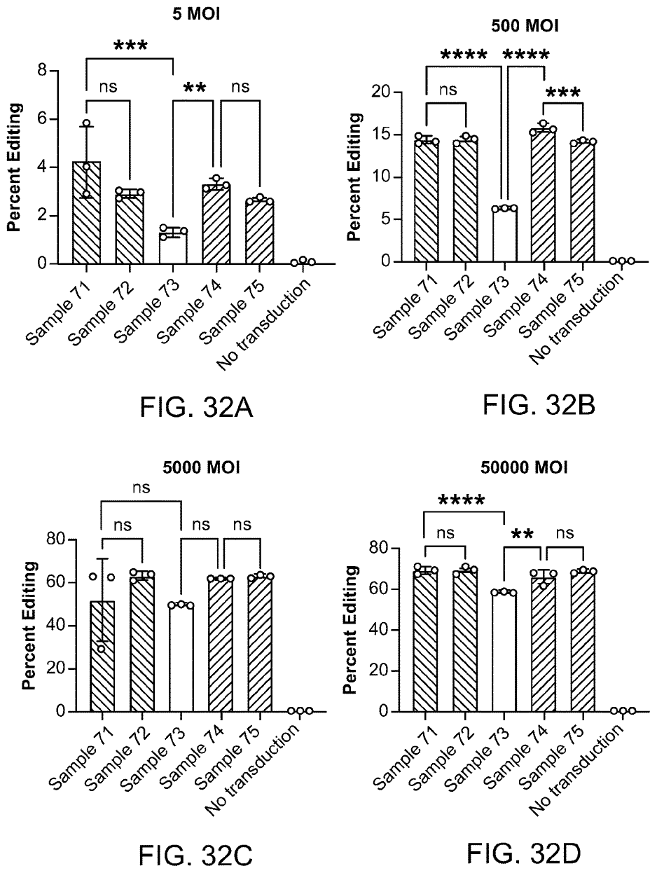

This example describes the addition of synthetic filler sequence to increase the size of a vector. Vectors with expression cassettes for the expression of an engineered guide RNA were cloned with two excerpts of synthetic filler sequence in three different positions, addition on the 5′ end of the expression cassette (5′), addition on both the 5′ and 3′ end of the expression cassette (Mid), and addition on the 3′ end of the expression cassette (3′), as shown in FIG. 31A and FIG. 31B. The vectors contained an expression cassette with an SNCA-TIS guide RNA and were transduced into HEK293 cells. The editing from the expression of the SNCA-TIS guide RNA was then measured. As shown in FIG. 32A, FIG. 32B, FIG. 32C, and FIG. 32D, the vectors with synthetic filler addition on the 3′ end (3′) and both the 3′ and 5′ end (Mid) showed similar level of percent editing indicating that the synthetic filler placement did not impact SNCA-TIS editing. The vector constructs with synthetic filler placement on the 5′ end (5′) showed lower editing compared to the other placements.

### Example 11

### Increasing Vector Size by Extension of Sequence Elements

This example describes the extension of sequence elements in a vector to increase the size of a vector genome since vector genomes less than half of the 2.3 kb scAAV limit may result in concatemers or other unintended genomic rearrangements. Vectors containing two expression cassettes were increased by extension of the termination sequences or the promoter sequences to increase the overall vector size. Each vector construct had two expression cassettes each having their own promoter sequence and termination sequences. Vector constructs were tested that either had two extended termination sequences or two extended promoter sequences. The promoter and termination sequences were each extended by about 100 base pairs and compared for function assessment by both a GFP reporter guide RNA and an SNCA-TIS guide RNA.

The extended termination sequences were as follows: SEQ ID NO: 81 (100 bp extension of SEQ ID NO: 11); SEQ ID NO: 79 (100 bp extension of SEQ ID NO: 35); SEQ ID NO: 85 (100 bp extension of SEQ ID NO: 13); SEQ ID NO: 87 (100 bp extension of SEQ ID NO: 86); SEQ ID NO: 89 (100 bp extension of SEQ ID NO: 88); SEQ ID NO: 78 (100 bp extension of SEQ ID NO: 34); and SEQ ID NO: 80 (100 bp extension of SEQ ID NO: 82).

The extended promoter sequences were as follows: SEQ ID NO: 61 (extension of SEQ ID NO: 66); SEQ ID NO: 90 (100 bp extension of SEQ ID NO: 8); and SEQ ID NO: 91 (100 bp extension of SEQ ID NO: 9). These promoters were also compared to the promoters of SEQ ID NO: 5 and SEQ ID NO: 3.

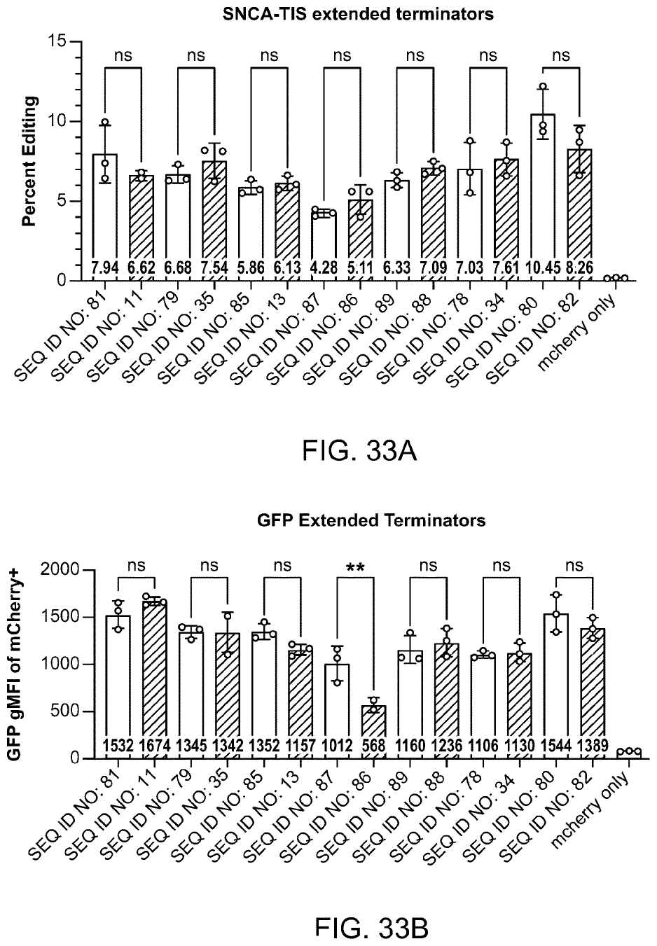

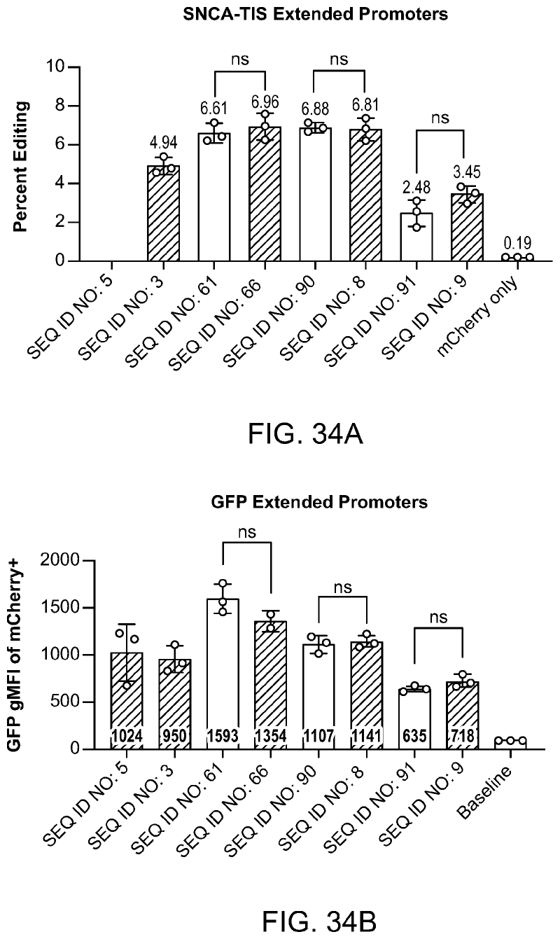

As shown in and FIG. 33B, the extension of the termination sequence to increase vector size did not impact the expression of the guide RNAs shown by similar SNCA-TIS editing values (FIG. 33A) and similar GFP fluorescence (FIG. 33B) between the non-extended termination sequences and the extended termination sequences, expect for one pair of termination sequences. Similarly, as shown in FIG. 34A and FIG. 34B, the extension of the promoter sequences did not impact the expression of the guide RNAs shown by similar SNCA-TIS editing values (FIG. 34A) and similar GFP fluorescence (FIG. 34B) between the non-extended promoter sequences and the extended promoter sequences.

### Example 12

### AAV Dosing Impact on Expression of Multiple Expression Cassettes

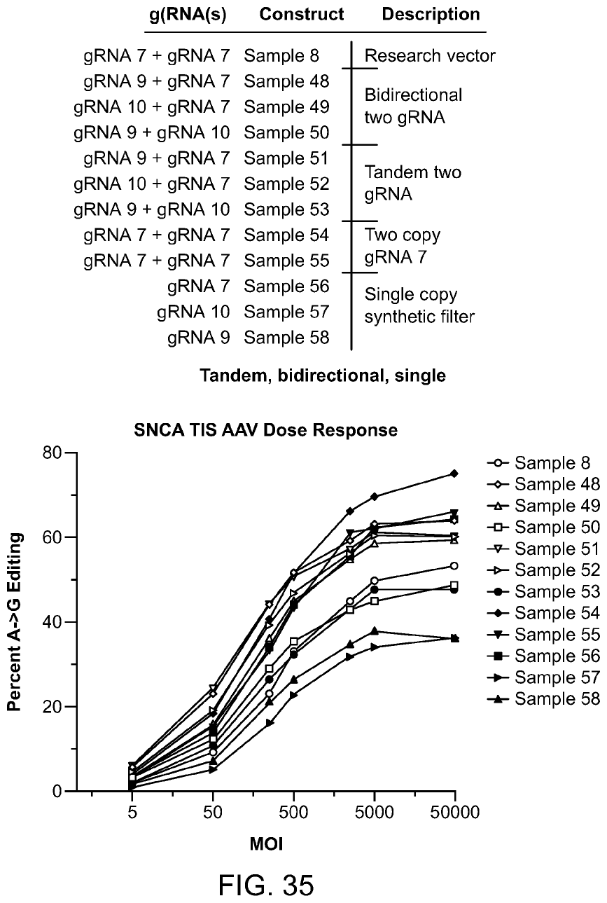

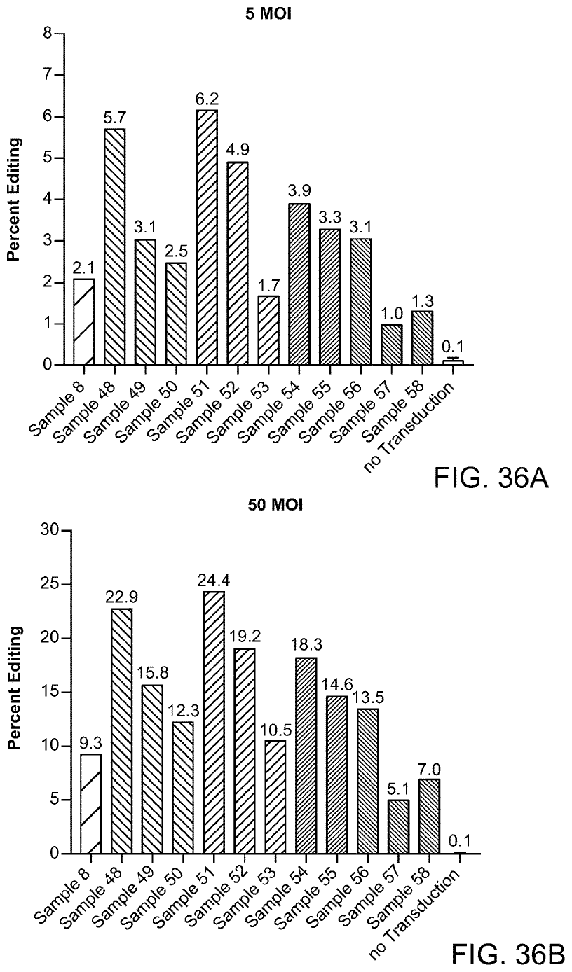

This example describes testing of the AAV dose response of vector constructs with different expression cassette orientations for SNCA-TIS editing. Vector constructs containing two expression cassettes were tested in a tandem and bidirectional orientation (shown in FIG. 27) and compared to a vector construct with a single expression cassette (single). The vector constructs were transfected in HEK293 cells and the percent A→G editing was measured as a function of the multiplicity of infection (MOI) of the AAV dose tested. As shown in FIG. 35, FIG. 36A, and FIG. 36B, the impact of the second expression cassette in the bidirectional tandem vector cassettes had the greatest increase in percent editing at the lower MOIs tested.

### Example 13

### Sequence Divergence of Expression Cassette Elements and Design of a Development Vector

This example describes the design of vector constructs with sequence elements (e.g., promoter sequences, hairpin sequences, and termination sequences) that have sequence divergence for the design of a development vector to be used in gene editing therapies. The developmental vector construct design also investigated removing a transduction marker sequence (previously included for experimental purposes) and accounting for the removed transduction marker sequence by extension of sequence elements to maintain the ideal vector genome size.

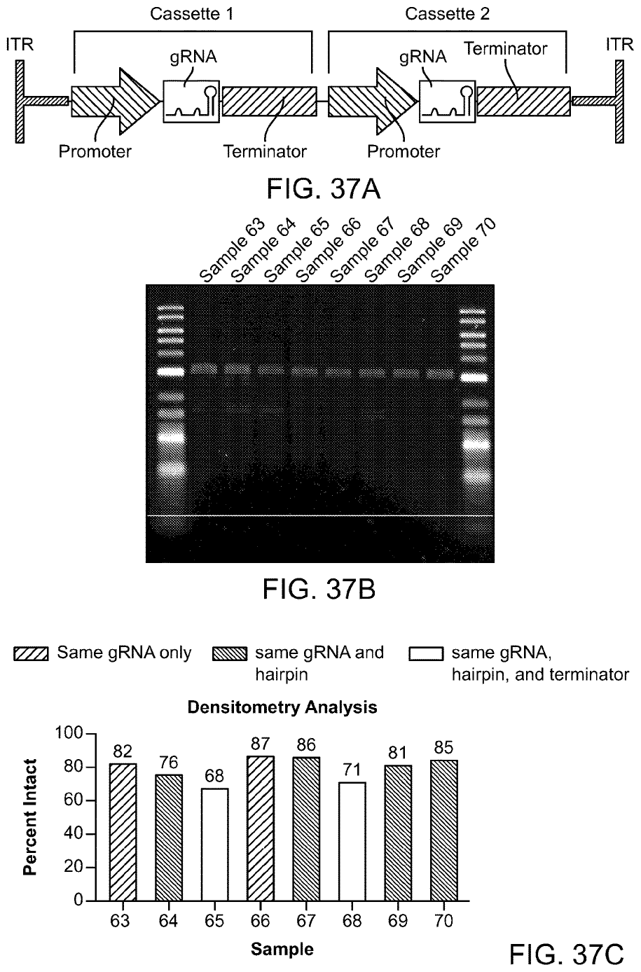

Vector constructs were tested in a tandem read orientation, as shown in FIG. 37A. The vector constructs contained two expression cassettes, with each expression cassette having a promoter sequence, a guide RNA sequence, a hairpin sequence, and a termination sequence. The vector constructs evaluated are provided below in TABLE 9.

TABLE 9
Two-Copy Vector Sequences

| | | Cassette 1 | | | | Cassette 2 | | | |
|---|---|---|---|---|---|---|---|---|---|
| | | | | Small nuclear | | | | Small nuclear | |
| | | | | RNA (snRNA) | | | | RNA (snRNA) | |
| | | | | processing | | | | processing | |
| | Config- | | | hairpin | Transcription | | | hairpin | Transcription |
| Construct | uration | Promoter | gRNA | sequence | terminator | Promoter | gRNA | sequence | terminator |
| Sample 63 | Tandem | SEQ ID NO: | gRNA 11 | SEQ ID NO: | SEQ ID NO: | SEQ ID NO: | gRNA 11 | SEQ ID NO: | SEQ ID NO: |
| | | 5 | | 84 | 78 | 67 | | 83 | 79 |
| Sample 64 | Tandem | SEQ ID NO: | gRNA 11 | SEQ ID NO: | SEQ ID NO: | SEQ ID NO: | gRNA 11 | SEQ ID NO: | SEQ ID NO: |
| | | 5 | | 83 | 78 | 67 | | 83 | 79 |
| Sample 65 | Tandem | SEQ ID NO: | gRNA 11 | SEQ ID NO: | SEQ ID NO: | SEQ ID NO: | gRNA 11 | SEQ ID NO: | SEQ ID NO: |
| | | 5 | | 83 | 78 | 67 | | 83 | 78 |
| Sample 66 | Tandem | SEQ ID NO: | gRNA 12 | SEQ ID NO: | SEQ ID NO: | SEQ ID NO: | gRNA 12 | SEQ ID NO: | SEQ ID NO: |
| | | 5 | | 84 | 78 | 67 | | 83 | 79 |
| Sample 67 | Tandem | SEQ ID NO: | gRNA 12 | SEQ ID NO: | SEQ ID NO: | SEQ ID NO: | gRNA 12 | SEQ ID NO: | SEQ ID NO: |
| | | 5 | | 83 | 78 | 67 | | 83 | 79 |
| Sample 68 | Tandem | SEQ ID NO: | gRNA 12 | SEQ ID NO: | SEQ ID NO: | SEQ ID NO: | gRNA 12 | SEQ ID NO: | SEQ ID NO: |
| | | 5 | | 83 | 78 | 67 | | 83 | 78 |
| Sample 69 | Tandem | SEQ ID NO: | gRNA 11 | SEQ ID NO: | SEQ ID NO: | SEQ ID NO: | gRNA 11 | SEQ ID NO: | SEQ ID NO: |
| | | 5 | | 83 | 78 | 67 | | 83 | 80 |
| Sample 70 | Tandem | SEQ ID NO: | gRNA 12 | SEQ ID NO: | SEQ ID NO: | SEQ ID NO: | gRNA 12 | SEQ ID NO: | SEQ ID NO: |
| | | 5 | | 83 | 78 | 67 | | 83 | 80 |

The vector constructs in TABLE 9 were tested for vector genome integrity. As shown in FIG. 37B and FIG. 37C, the percent intact AAV genome was comparable between vector constructs that had the same guide RNA in both expression cassettes and also was comparable between vector constructs with the same guide RNA and hairpin sequence in both expression cassettes. Also shown in FIG. 37B and FIG. 37C, vector constructs with the same guide RNA, hairpin sequence, and termination sequence resulted in a decrease in vector genome integrity.

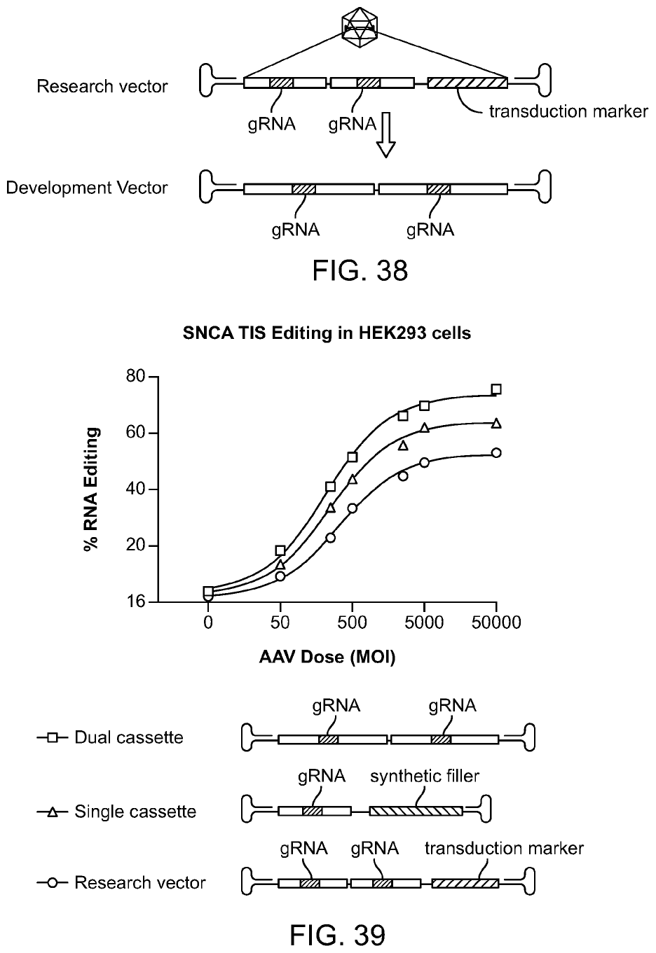

Vector cassettes were also designed to remove the transduction marker sequence originally included for experimental purposes in a vector construct (“Research Vector”) to design a development vector design (“Development Vector”), as shown in FIG. 38. As shown in FIG. 39, the “Development Vector” outperformed both the “Research Vector” and the vector design with a singular expression cassette in RNA editing percentage.

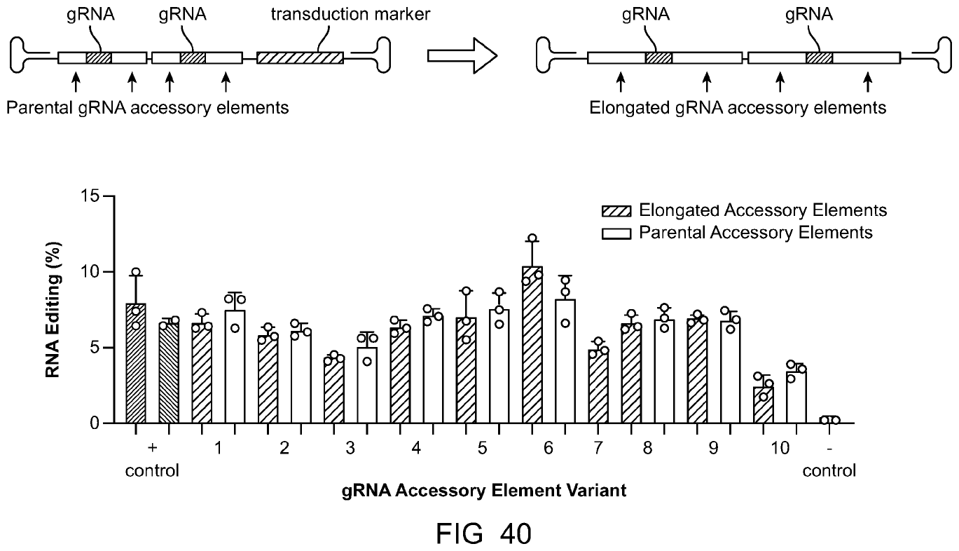

Development vector constructs were designed to maintain ideal vector genome size by elongating parental guide RNA expression cassette elements (parental gRNA accessory elements) to elongated guide RNA accessory elements, as shown in FIG. 40. Also shown in FIG. 40, the elongated guide RNA accessory elements performed had similar RNA editing as compared to the parental guide RNA accessory elements and also to the positive control (a single expression cassette copy integration assay mimicking a single expression cassette vector design).

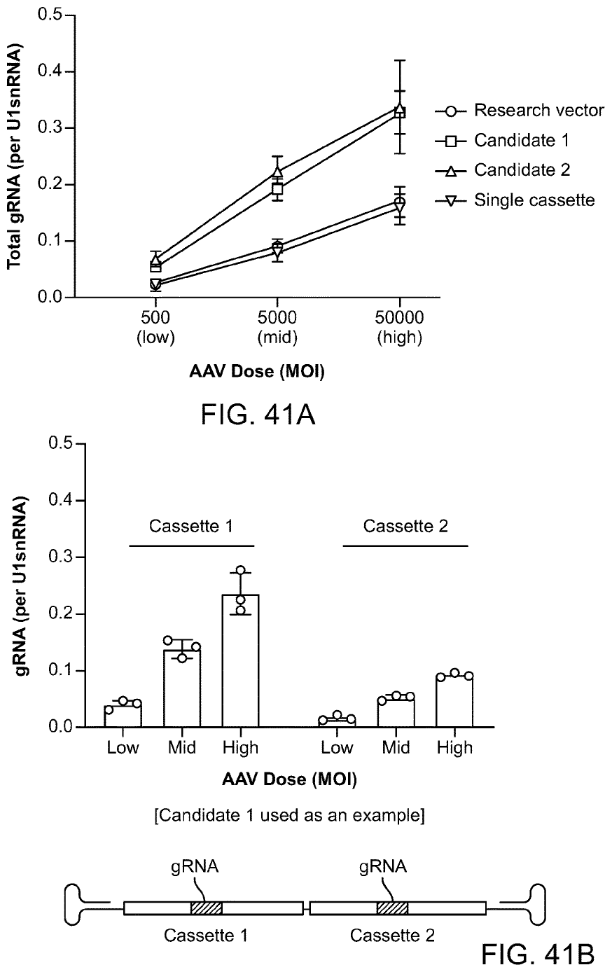

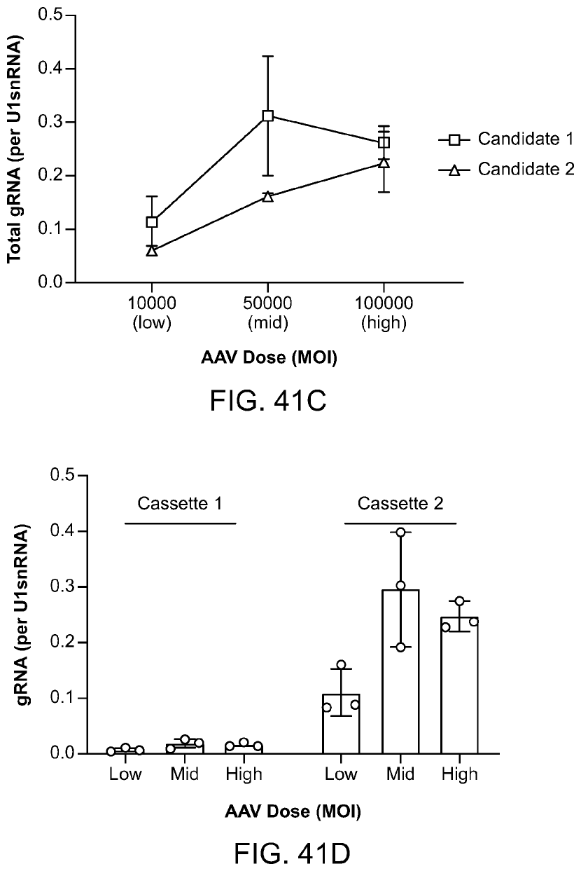

Development vector designs were also tested for guide RNA expression in mouse primary neuron cells. As shown in FIG. 41A and FIG. 41B, the development vector designs showed higher guide RNA expression as compared to both the “Research Vector” design and the single expression cassette vector construct.

Development vector designs were also tested in human iPSC-derived neuron cells. As shown in FIG. 41C and FIG. 41D, guide RNA expression from cassette 1 was decreased as compared to guide RNA expression from cassette 2 in human iPSC-derived neuron cells. This was presumably due to the inclusion of a U1 snRNA hairpin (SEQ ID NO: 84) sequence element in expression cassette 1 of the development vector design. The expression of expression cassette 1 was increased in human iPSC neuron cells by replacing the U1 snRNA hairpin (SEQ ID NO: 84) sequence element with the mU7 SmOPT hairpin variant sequence (SEQ ID NO: 83) in expression cassette 1 in the development vector design.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A polynucleotide comprising a plurality of expression cassette sequences comprising a first expression cassette sequence and a second expression cassette sequence, wherein:
the first expression cassette sequence and the second expression cassette sequence each independently comprise:
a DNA sequence encoding a promoter sequence,
a DNA sequence encoding an engineered guide RNA (gRNA) sequence under transcriptional control of the promoter sequence,
a DNA sequence encoding a SmOPT and a small nuclear RNA (snRNA) processing hairpin sequence, and
a DNA sequence encoding a transcription termination sequence;
at least one of the DNA sequences encoding a promoter sequence comprises at least 80% sequence identity to SEQ ID NO: 5;
the first expression cassette sequence and the second expression cassette sequence are each independently arranged in a 5′ to 3′ orientation to have a read directionality of forward;
the DNA sequence encoding an engineered guide RNA (gRNA) sequence of the first expression cassette sequence and the second expression cassette sequence encodes the same engineered guide RNA (gRNA) sequence, and
the first and second expression cassette sequences are different sequences.

2. The polynucleotide of claim 1, wherein the first expression cassette sequence and the second expression cassette sequence are orientated in a tandem read orientation.

3. The polynucleotide of claim 1 or claim 2, wherein the DNA sequence encoding a promoter sequence comprises at least 80%, at least 85%, at least 90%, at least 95%, or 100% sequence identity to SEQ ID NO: 5 or SEQ ID NO: 67.

4. The polynucleotide of any one of claims 1-3, wherein the DNA sequence encoding a transcription termination comprises at least 80%, at least 85%, at least 90%, at least 95%, or 100% sequence identity to SEQ ID NO: 78, SEQ ID NO: 79, or SEQ ID NO: 80.

5. The polynucleotide of any one of claims 1-4, wherein the DNA sequence encoding a SmOPT and a small nuclear RNA (snRNA) processing hairpin sequence comprises at least 80%, at least 85%, at least 90%, at least 95%, or 100% sequence identity to SEQ ID NO: 92.

6. The polynucleotide of any one of claims 1-5, wherein:
the DNA sequence encoding a promoter sequence of the first expression cassette sequence comprises at least 80%, at least 85%, at least 90%, at least 95%, or 100% sequence identity to SEQ ID NO: 5, and
the DNA sequence encoding a transcription termination sequence of the first expression cassette sequence comprises at least 80%, at least 85%, at least 90%, at least 95%, or 100% sequence identity to SEQ ID NO: 78 or SEQ ID NO: 79.

7. The polynucleotide of any one of claims 1-6, wherein:
the DNA sequence encoding a promoter sequence of the second expression cassette sequence comprises at least 80%, at least 85%, at least 90%, at least 95%, or 100% sequence identity to SEQ ID NO: 67, and
the DNA sequence encoding a transcription termination sequence of the second expression cassette sequence comprises at least 80%, at least 85%, at least 90%, at least 95%, or 100% sequence identity to SEQ ID NO: 79 or SEQ ID NO: 80.

8. The polynucleotide of any one of claims 1-7, wherein:
the DNA sequence encoding a promoter sequence of the first expression cassette sequence comprises at least 80%, at least 85%, at least 90%, at least 95%, or 100% sequence identity to SEQ ID NO: 5, and
the DNA sequence encoding a promoter sequence of the second expression cassette sequence comprises at least 80%, at least 85%, at least 90%, at least 95%, or 100% sequence identity to SEQ ID NO: 67.

9. The polynucleotide of any one of claims 1-8, wherein:
the DNA sequence encoding a transcription termination sequence of the first expression cassette sequence comprises at least 80%, at least 85%, at least 90%, at least 95%, or 100% sequence identity to SEQ ID NO: 78 or SEQ ID NO: 79, and
the DNA sequence encoding a transcription termination sequence of the second expression cassette sequence comprises at least 80%, at least 85%, at least 90%, at least 95%, or 100% sequence identity to SEQ ID NO: 79 or SEQ ID NO: 80.

10. The polynucleotide of any one of claims 1-9, wherein:
the DNA sequence encoding a promoter sequence of the first expression cassette sequence comprises at least 80%, at least 85%, at least 90%, at least 95%, or 100% sequence identity to SEQ ID NO: 5,
the DNA sequence encoding a SmOPT and a small nuclear RNA (snRNA) processing hairpin sequence of the first expression cassette sequence comprises at least 80%, at least 85%, at least 90%, at least 95%, or 100% sequence identity to SEQ ID NO: 92, and
the DNA sequence encoding a transcription termination sequence of the first expression cassette sequence comprises at least 80%, at least 85%, at least 90%, at least 95%, or 100% sequence identity to SEQ ID NO: 78 or SEQ ID NO: 79.

11. The polynucleotide of any one of claims 1-10, wherein:
the DNA sequence encoding a promoter sequence of the second expression cassette sequence comprises at least 80%, at least 85%, at least 90%, at least 95%, or 100% sequence identity to SEQ ID NO: 67,
the DNA sequence encoding a SmOPT and a small nuclear RNA (snRNA) processing hairpin sequence of the second expression cassette sequence comprises at least 80%, at least 85%, at least 90%, at least 95%, or 100% sequence identity to SEQ ID NO: 92, and
the DNA sequence encoding a transcription termination sequence of the second expression cassette sequence comprises at least 80%, at least 85%, at least 90%, at least 95%, or 100% sequence identity to SEQ ID NO: 79 or SEQ ID NO: 80.

12. The polynucleotide of any one of claims 1-11, wherein:
the DNA sequence encoding a promoter sequence of the first expression cassette sequence comprises at least 80%, at least 85%, at least 90%, at least 95%, or 100% sequence identity to SEQ ID NO: 5,
the DNA sequence encoding a SmOPT and a small nuclear RNA (snRNA) processing hairpin sequence of the first expression cassette sequence comprises at least 80%, at least 85%, at least 90%, at least 95%, or 100% sequence identity to SEQ ID NO: 92,
the DNA sequence encoding a transcription termination sequence of the first expression cassette sequence comprises at least 80%, at least 85%, at least 90%, at least 95%, or 100% sequence identity to SEQ ID NO: 78,
the DNA sequence encoding a promoter sequence of the second expression cassette sequence comprises at least 80%, at least 85%, at least 90%, at least 95%, or 100% sequence identity to SEQ ID NO: 67,
the DNA sequence encoding a SmOPT and a small nuclear RNA (snRNA) processing hairpin sequence of the second expression cassette sequence comprises at least 80%, at least 85%, at least 90%, at least 95%, or 100% sequence identity to SEQ ID NO: 92, and
the DNA sequence encoding a transcription termination sequence of the second expression cassette sequence comprises at least 80%, at least 85%, at least 90%, at least 95%, or 100% sequence identity to SEQ ID NO: 79.

13. The polynucleotide of any one of claims 1-12, wherein:
the DNA sequence encoding a promoter sequence of the first expression cassette sequence comprises at least 80%, at least 85%, at least 90%, at least 95%, or 100% sequence identity to SEQ ID NO: 5,
the DNA sequence encoding a SmOPT and a small nuclear RNA (snRNA) processing hairpin sequence of the first expression cassette sequence comprises at least 80%, at least 85%, at least 90%, at least 95%, or 100% sequence identity to SEQ ID NO: 92,
the DNA sequence encoding a transcription termination sequence of the first expression cassette sequence comprises at least 80%, at least 85%, at least 90%, at least 95%, or 100% sequence identity to SEQ ID NO: 79,
the DNA sequence encoding a promoter sequence of the second expression cassette sequence comprises at least 80%, at least 85%, at least 90%, at least 95%, or 100% sequence identity to SEQ ID NO: 67,
the DNA sequence encoding a SmOPT and a small nuclear RNA (snRNA) processing hairpin sequence of the second expression cassette sequence comprises at least 80%, at least 85%, at least 90%, at least 95%, or 100% sequence identity to SEQ ID NO: 92, and
the DNA sequence encoding a transcription termination sequence of the second expression cassette sequence comprises at least 80%, at least 85%, at least 90%, at least 95%, or 100% sequence identity to SEQ ID NO: 80.

14. A polynucleotide comprising a plurality of expression cassette sequences comprising a first expression cassette sequence and a second expression cassette sequence, wherein:
the first, second, and plurality of expression cassette sequences each independently comprise:
a promoter sequence,
a payload sequence under transcriptional control of the promoter sequence, the payload sequence comprising a small RNA payload, and
a transcription termination sequence;
the first, second, and plurality of expression cassette sequences are each independently arranged in a 5′ to 3′ orientation to have a read directionality of forward or reverse; and
the first and second expression cassette sequences are different sequences.

15. The polynucleotide of claim 14, wherein the first expression cassette sequence has a read directionality of forward and the second expression cassette sequence has a read directionality of reverse.

16. The polynucleotide of claim 14, wherein the first expression cassette sequence has a read directionality of reverse and the second expression cassette sequence has a read directionality of forward.

17. The polynucleotide of claim 14, wherein the first expression cassette sequence has a read directionality of forward and the second expression cassette sequence has a read directionality of forward.

18. The polynucleotide of any one of claims 14-17, wherein the plurality of expression cassette sequences comprises two expression cassette sequences, three expression cassette sequences, four expression cassette sequences, five expression cassette sequences, six expression cassette sequences, seven expression cassette sequences, eight expression cassette sequences, nine expression cassette sequences, or ten expression cassette sequences.

19. The polynucleotide of any one of claims 14-18, wherein the small RNA payload comprises an engineered guide RNA sequence.

20. The polynucleotide of any one of claims 1-19, wherein the engineered guide RNA sequence is capable of hybridizing to a target sequence.

21. The polynucleotide of any one of claims 1-20, wherein the engineered guide RNA sequence is at least 70%, at least 75%, at least 80%, at least 85%, at least 90%, at least 95%, or 100% reverse complementary to the target sequence.

22. The polynucleotide of any one of claims 1-21, wherein the engineered guide RNA sequence is capable of forming a guide-target RNA scaffold comprising one or more structural features upon hybridization to a target sequence.

23. The polynucleotide of claim 22, wherein the one or more structural features comprise a bulge, a mismatch, an internal loop, a hairpin, or combinations thereof.

24. The polynucleotide of claim 22 or claim 23, wherein the one or more structural features comprises the bulge, and wherein the bulge is a symmetric bulge.

25. The polynucleotide of any one of claims 22-24, wherein the one or more structural features comprises the bulge, and wherein the bulge is an asymmetric bulge.

26. The polynucleotide of any one of claims 22-25, wherein the one or more structural features comprises the internal loop, and wherein the internal loop is a symmetric internal loop.

27. The polynucleotide of any one of claims 22-26, wherein the one or more structural features comprises the internal loop, and wherein the internal loop is an asymmetric internal loop.

28. The polynucleotide of any one of claims 22-27, wherein the one or more structural features comprises the hairpin, and wherein the hairpin is a recruitment hairpin or a non-recruitment hairpin.

29. The polynucleotide of any one of claims 22-28, wherein the guide-target RNA scaffold comprises one or more wobble base pairs.

30. The polynucleotide of claim 29, wherein the one or more of the wobble base pairs are GU wobble base pairs.

31. The polynucleotide of any one of claims 20-30, wherein the engineered guide RNA sequence comprises at least one base pair mismatch relative to the target sequence.

32. The polynucleotide of any one of claims 20-31, wherein the target sequence comprises an adenosine residue.

33. The polynucleotide of any one of claims 20-32, wherein the target sequence is an RNA sequence.

34. The polynucleotide of claim 33, wherein the RNA sequence is a mRNA or a pre-mRNA.

35. The polynucleotide of any one of claims 20-34, wherein the target sequence comprises a G to A mutation relative to a wild type sequence.

36. The polynucleotide of any one of claims 20-35, wherein the target sequence comprises a missense mutation or a nonsense mutation relative to a wild type sequence.

37. The polynucleotide of any one of claims 20-36, wherein the target sequence encodes α-synuclein (SNCA).

38. The polynucleotide of any one of claims 20-36, wherein the target sequence encodes peripheral myelin protein 22 (PMP22).

39. The polynucleotide of any one of claims 20-36, wherein the target sequence encodes double homeobox 4 (DUX4).

40. The polynucleotide of any one of claims 20-36, wherein the target sequence encodes leucine rich repeat kinase 2 (LRRK2).

41. The polynucleotide of any one of claims 20-36, wherein the target sequence encodes Tau (MAPT).

42. The polynucleotide of any one of claims 20-36, wherein the target sequence encodes ATP-binding cassette sub-family A member 4 (ABCA4).

43. The polynucleotide of any one of claims 20-36, wherein the target sequence encodes alpha-1 antitrypsin (SERPINA1).

44. The polynucleotide of any one of claims 20-36, wherein the target sequence encodes methyl CpG binding protein 2 (MECP2).

45. The polynucleotide of any one of claims 1-44, wherein the engineered guide RNA sequence is not less than 20 nucleotide residues and not more than 500 nucleotide residues long.

46. The polynucleotide of any one of claims 1-45, wherein the engineered guide RNA sequence is not less than 60 and not more than 100 residues long.

47. The polynucleotide of any one of claims 1-46, wherein the engineered guide RNA sequence is not less than 80 and not more than 120 residues long.

48. The polynucleotide of any one of claims 1-47, wherein the engineered guide RNA sequence is not less than 100 and not more than 140 residues long.

49. The polynucleotide of any one of claims 1-48, wherein the engineered guide RNA sequence is not less than 130 and not more than 170 residues long.

50. The polynucleotide of any one of claims 14-49, wherein the promoter sequence comprises at least 80%, at least 85%, at least 90%, at least 95%, or 100% sequence identity to any one of SEQ ID NO: 1-SEQ ID NO: 9, SEQ ID NO: 66-SEQ ID NO: 76, SEQ ID NO: 90, or SEQ ID NO: 91.

51. The polynucleotide of any one of claims 14-50, wherein the transcription termination sequence comprises at least 80%, at least 85%, at least 90%, at least 95%, or 100% sequence identity to any one of SEQ ID NO: 10-SEQ ID NO: 16, SEQ ID NO: 34, SEQ ID NO: 35, SEQ ID NO: 77-SEQ ID NO: 82, or SEQ ID NO: 85-SEQ ID NO: 89.

52. The polynucleotide of any one of claims 14-51, wherein the payload sequence further comprises an Sm binding sequence or a hairpin sequence.

53. The polynucleotide of claim 52, wherein the hairpin sequence comprises a U7 hairpin.

54. The polynucleotide of any one of claims 1-53, wherein the first expression cassette sequence, the second expression cassette sequence, or each expression cassette of the plurality of expression cassette sequences independently has a length of not less than 1300 nucleotide residues and not more than 2160 nucleotide residues.

55. The polynucleotide of any one of claims 14-54, wherein the first expression cassette sequence, the second expression cassette sequence, or each expression cassette of the plurality of expression cassette sequences independently comprises at least 80% sequence identity to a U1 sequence or a U7 sequence.

56. The polynucleotide of claim 55, wherein the U1 sequence is a mouse U1 sequence or a human U1 sequence.

57. The polynucleotide of claim 55 or claim 56, wherein the U7 sequence is a mouse U7 sequence or a human U7 sequence.

58. A viral vector encoding the polynucleotide of any one of claims 1-57.

59. The viral vector of claim 58, wherein the viral vector is an adeno-associated viral vector.

60. The viral vector of claim 59, wherein the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, chimeras thereof, variants or derivatives thereof, and combinations thereof.

61. A pharmaceutical composition comprising the polynucleotide of any one of claims 1-57 or the viral vector of any one of claims 58-60 and a pharmaceutically acceptable excipient, carrier, diluent, or combination thereof.

62. A method of expressing an engineered guide RNA in a cell, the method comprising:
(i) delivering a composition comprising a polynucleotide to a cell, wherein the polynucleotide comprises a plurality of expression cassette sequences comprising a first expression cassette sequence and a second expression cassette sequence, wherein:
the first expression cassette sequence and the second expression cassette sequence each independently comprise:
a DNA sequence encoding a promoter sequence,
a DNA sequence encoding an engineered guide RNA (gRNA) sequence under transcriptional control of the promoter sequence,
a DNA sequence encoding a SmOPT and a small nuclear RNA (snRNA) processing hairpin sequence, and
a DNA sequence encoding a transcription termination sequence;
at least one of the DNA sequences encoding a promoter sequence comprises at least 80% sequence identity to SEQ ID NO: 5;
the first expression cassette sequence and the second expression cassette sequence are each independently arranged in a 5′ to 3′ orientation to have a read directionality of forward;
the DNA sequence encoding an engineered guide RNA (gRNA) sequence of the first expression cassette sequence and the second expression cassette sequence encodes the same engineered guide RNA (gRNA) sequence; and
the first and second expression cassette sequences are different sequences; and
(ii) expressing the engineered guide RNA (gRNA) sequence in the cell.

63. A method of editing a target sequence, the method comprising:
(i) delivering a composition comprising a polynucleotide to a cell encoding a target sequence, wherein the polynucleotide comprises a plurality of expression cassette sequences comprising a first expression cassette sequence and a second expression cassette sequence, wherein:
the first expression cassette sequence and the second expression cassette sequence each independently comprise:
a DNA sequence encoding a promoter sequence,
a DNA sequence encoding an engineered guide RNA (gRNA) sequence under transcriptional control of the promoter sequence,
a DNA sequence encoding a SmOPT and a small nuclear RNA (snRNA) processing hairpin sequence, and
a DNA sequence encoding a transcription termination sequence;
at least one of the DNA sequences encoding a promoter sequence comprises at least 80% sequence identity to SEQ ID NO: 5;
the first expression cassette sequence and the second expression cassette sequence are each independently arranged in a 5′ to 3′ orientation to have a read directionality of forward;
the DNA sequence encoding an engineered guide RNA (gRNA) sequence of the first expression cassette sequence and the second expression cassette sequence encodes the same engineered guide RNA (gRNA) sequence; and
the first and second expression cassette sequences are different sequences;
(ii) expressing the engineered guide RNA (gRNA) sequence in the cell;
(iii) forming a guide-target RNA scaffold upon hybridization of the engineered guide RNA (gRNA) sequence to the target sequence;
(iv) recruiting an editing enzyme to the target sequence; and
(v) editing the target sequence with the editing enzyme.

64. A method of expressing an engineered guide RNA sequence in a cell, the method comprising:
(i) delivering a composition comprising a polynucleotide to a cell, wherein the polynucleotide comprises a plurality of expression cassette sequences comprising a first expression cassette sequence and a second expression cassette sequence, wherein:
the first, second, and plurality of expression cassette sequences each independently comprise:
a promoter sequence,
a payload sequence under transcriptional control of the promoter sequence, the payload sequence comprising an engineered guide RNA sequence, and
a transcription termination sequence;
the first, second, and plurality of expression cassette sequences are each independently arranged in a 5′ to 3′ orientation to have a read directionality of forward or reverse; and
the first and second expression cassette sequences are different sequences; and
(ii) expressing the engineered guide RNA sequence in the cell.

65. A method of editing a target sequence, the method comprising:
(i) delivering a composition comprising a polynucleotide to a cell encoding a target sequence, wherein the polynucleotide comprises a plurality of expression cassette sequences comprising a first expression cassette sequence and a second expression cassette sequence, wherein:
the first, second, and plurality of expression cassette sequences each independently comprise:
a promoter sequence,
a payload sequence under transcriptional control of the promoter sequence, the payload sequence comprising an engineered guide RNA sequence, and
a transcription termination sequence;
the first, second, and plurality of expression cassette sequences are each independently arranged in a 5′ to 3′ orientation to have a read directionality of forward or reverse; and
the first and second expression cassette sequences are different sequences;
(ii) expressing the engineered guide RNA sequence in the cell;
(iii) forming a guide-target RNA scaffold upon hybridization of the engineered guide RNA sequence to the target sequence;
(iv) recruiting an editing enzyme to the target sequence; and
(v) editing the target sequence with the editing enzyme.

66. A method of administering a polynucleotide to a subject with a disease, the method comprising:
(i) administering to the subject a composition comprising the polynucleotide, wherein the polynucleotide comprises a plurality of expression cassette sequences comprising a first expression cassette sequence and a second expression cassette sequence, wherein:
the first expression cassette sequence and the second expression cassette sequence each independently comprise:
a DNA sequence encoding a promoter sequence,
a DNA sequence encoding an engineered guide RNA (gRNA) sequence under transcriptional control of the promoter sequence,
a DNA sequence encoding a SmOPT and a small nuclear RNA (snRNA) processing hairpin sequence, and
a DNA sequence encoding a transcription termination sequence;
at least one of the DNA sequences encoding a promoter sequence comprises at least 80% sequence identity to SEQ ID NO: 5;
the first expression cassette sequence and the second expression cassette sequence are each independently arranged in a 5′ to 3′ orientation to have a read directionality of forward;
the DNA sequence encoding an engineered guide RNA (gRNA) sequence of the first expression cassette sequence and the second expression cassette sequence encodes the same engineered guide RNA (gRNA) sequence; and
the first and second expression cassette sequences are different sequence;
(ii) delivering the polynucleotide to a cell of the subject; and
(iii) expressing the engineered guide RNA (gRNA) sequence in the cell.

67. A method of treating a disease in a subject, the method comprising:
(i) administering to the subject a composition comprising a polynucleotide, wherein the polynucleotide comprises a plurality of expression cassette sequences comprising a first expression cassette sequence and a second expression cassette sequence, wherein:
the first expression cassette sequence and the second expression cassette sequence each independently comprise:
a DNA sequence encoding a promoter sequence,
a DNA sequence encoding an engineered guide RNA (gRNA) sequence under transcriptional control of the promoter sequence,
a DNA sequence encoding a SmOPT and a small nuclear RNA (snRNA) processing hairpin sequence, and
a DNA sequence encoding a transcription termination sequence;
at least one of the DNA sequences encoding a promoter sequence comprises at least 80% sequence identity to SEQ ID NO: 5;
the first expression cassette sequence and the second expression cassette sequence are each independently arranged in a 5′ to 3′ orientation to have a read directionality of forward;
the DNA sequence encoding an engineered guide RNA (gRNA) sequence of the first expression cassette sequence and the second expression cassette sequence encodes the same engineered guide RNA (gRNA) sequence; and
the first and second expression cassette sequences are different sequence;
(ii) delivering the polynucleotide to a cell of the subject; and
(iii) expressing the engineered guide RNA (gRNA) sequence in the cell, thereby treating the disease.

68. A method of administering a polynucleotide to a subject with a disease, the method comprising:
(i) administering to the subject a composition comprising the polynucleotide, wherein the polynucleotide comprises a plurality of expression cassette sequences comprising a first expression cassette sequence and a second expression cassette sequence, wherein:
the first, second, and plurality of expression cassette sequences each independently comprise:
a promoter sequence,
a payload sequence under transcriptional control of the promoter sequence, the payload sequence comprising an engineered guide RNA sequence, and
a transcription termination sequence;
the first, second, and plurality of expression cassette sequences are each independently arranged in a 5′ to 3′ orientation to have a read directionality of forward or reverse; and
the first and second expression cassette sequences are different sequences;
(ii) delivering the therapeutic polynucleotide to a cell of the subject; and
(iii) expressing the engineered guide RNA sequence in the cell.

69. A method of treating a disease in a subject, the method comprising:
(i) administering to the subject a composition comprising a polynucleotide, wherein the polynucleotide comprises a plurality of expression cassette sequences comprising a first expression cassette sequence and a second expression cassette sequence, wherein:
the first, second, and plurality of expression cassette sequences each independently comprise:
a promoter sequence,
a payload sequence under transcriptional control of the promoter sequence, the payload sequence comprising an engineered guide RNA sequence, and
a transcription termination sequence;
the first, second, and plurality of expression cassette sequences are each independently arranged in a 5′ to 3′ orientation to have a read directionality of forward or reverse; and
the first and second expression cassette sequences are different sequences;
(ii) delivering the therapeutic polynucleotide to a cell of the subject; and
(iii) expressing the engineered guide RNA sequence in the cell, thereby treating the disease.

70. The method of any one of claims 62-69, wherein the cell is in a central nervous system tissue.

71. The method of any one of claims 62-69, wherein the cell is in a liver tissue, muscle tissue, ocular tissue, retinal tissue, heart tissue, skeletal muscle tissue, or kidney tissue.

72. The method of any one of claims 62-71, wherein the composition is the pharmaceutical composition of claim 61.

73. The method of any one of claims 62-72, wherein the composition comprises the polynucleotide of any one of claims 1-57 or the viral vector of any one of claims 58-60.

74. The method of any one of claims 66-73, wherein the disease is a synucleinopathy, Parkinson's disease, Lewy body dementia, multiple system atrophy, Charcot-Marie-Tooth disease, hereditary neuropathy with liability to pressure palsies, Yuan-Harel-Lupski syndrome, a tauopathy, Alzheimer's disease, frontotemporal dementia, progressive supranuclear palsy, corticobasal degeneration, chronic traumatic encephalopathy, autism, traumatic brain injury, Dravet syndrome, Crohn's disease, muscular dystrophy, B-cell leukemia, Dejerine-Sottas disease, Stargardt disease, alpha-1 antitrypsin deficiency, Tay-Sachs disease, cystic fibrosis, liposomal acid lipase deficiency, or Gaucher disease.

75. The method of any one of claim 62, 64, or 66-74, wherein the engineered guide RNA sequence hybridizes to a target sequence, and wherein the cell encodes the target sequence.

76. The method of claim 75, further comprising forming a guide-target RNA scaffold upon hybridization of the engineered guide RNA to the target sequence, recruiting an editing enzyme to the target sequence, and editing the target sequence with the editing enzyme.

77. The method of any one of claim 63, 65, or 70-76, wherein the target sequence encodes α-synuclein (SNCA).

78. The method of any one of claim 63, 65, or 70-76, wherein the target sequence encodes peripheral myelin protein 22 (PMP22).

79. The method of any one of claim 63, 65, or 70-76, wherein the target sequence encodes double homeobox 4 (DUX4).

80. The method of any one of claim 63, 65, or 70-76, wherein the target sequence encodes leucine rich repeat kinase 2 (LRRK2).

81. The method of any one of claim 63, 65, or 70-76, wherein the target sequence encodes Tau (MAPT).

82. The method of any one of claim 63, 65, or 70-76, wherein the target sequence encodes ATP-binding cassette sub-family A member 4 (ABCA4).

83. The method of any one of claim 63, 65, or 70-76, wherein the target sequence encodes alpha-1 antitrypsin (SERPINA1).

84. The method of any one of claim 63, 65, or 70-76, wherein the target sequence encodes methyl CpG binding protein 2 (MECP2).

85. The method of any one of claim 63, 65, or 70-84, wherein the target sequence comprises a mutation relative to a wild type sequence.

86. The method of claim 85, wherein editing the target sequence corrects the mutation in the target sequence.

87. The method of claim 85 or claim 86, wherein the mutation is a missense mutation.

88. The method of claim 85 or claim 86, wherein the mutation is a nonsense mutation.

89. The method of any one of claims 85-88, wherein the mutation is a G to A mutation.

90. The method of any one of claims 85-89, wherein the mutation is associated with the disease.

91. The method of any one of claim 63, 65, or 70-76, wherein editing the target sequence comprises editing an untranslated region of the target sequence.

92. The method of claim 91, wherein the untranslated region is a 5′ untranslated region or a 3′ untranslated region.

93. The method of claim 92, wherein the 3′ untranslated region is a polyadenylation sequence.

94. The method of any one of claim 63, 65, or 70-93, wherein editing the target sequence comprises editing a translation initiation site.

95. The method of any one of claim 63, 65, or 70-94, wherein editing the target sequence alters expression of the target sequence.

96. The method of claim 95, wherein editing the target sequence increases expression of the target sequence.

97. The method of claim 95, wherein editing the target sequence decreases expression of the target sequence.

98. The method of any one of claim 63, 65, or 70-97, wherein the editing enzyme comprises an ADAR, an APOBEC, or a Cas nuclease.

99. The method of claim 98, wherein the ADAR comprises ADAR1, ADAR2, or a combination thereof.

100. The method of any one of claim 63, 65, or 70-99, wherein the target sequence comprises RNA or DNA.

101. The method of any one of claim 63, 65, or 70-100, wherein the target sequence is a mRNA or a pre-mRNA.

102. The method of any one of claim 63, 65, or 70-101, wherein editing the target sequence comprises deamidating a nucleotide of the target sequence.

103. The method of any one of claim 63, 65, or 70-102, wherein the target sequence is edited with an efficiency of at least 10%, at least 20%, or at least 25%.

104. A method of increasing a vector genome integrity of a multi-expression cassette vector comprising a first expression cassette and a second expression cassette, the method comprising:
a) generating a sequence of the second expression cassette by altering a sequence of the first expression cassette, wherein the first expression cassette and the second expression cassette each independently comprise:
a DNA sequence encoding a promoter sequence,
a DNA sequence encoding an engineered guide RNA (gRNA) sequence under transcriptional control of the promoter sequence,
a DNA sequence encoding a SmOPT and a small nuclear RNA (snRNA) processing hairpin sequence, and
a DNA sequence encoding a transcription termination sequence;
b) generating the multi-expression cassette vector by combining the sequence of the first expression cassette and the sequence of the second expression cassette; and
c) increasing the vector genome integrity as compared to a vector comprising a first expression cassette and a second expression cassette that each have the sequence of the first expression cassette.

105. The method of claim 104, wherein the sequence of the first expression cassette and the sequence of the second expression cassette have different DNA sequences encoding a promoter sequence.

106. The method of claim 104 or claim 105, wherein the sequence of the first expression cassette and the sequence of the second expression cassette have different DNA sequences encoding a transcription termination sequence.

107. The method of any one of claims 104-106, wherein the sequence of the first expression cassette and the sequence of the second expression cassette have different DNA sequences encoding a SmOPT and a small nuclear RNA (snRNA) processing hairpin sequence.

108. The method of any one of claims 104-107, wherein the DNA sequence encoding a promoter sequence comprises at least 80%, at least 85%, at least 90%, at least 95%, or 100% sequence identity to any one of SEQ ID NO: 1-SEQ ID NO: 9, SEQ ID NO: 66-SEQ ID NO: 76, SEQ ID NO: 90, or SEQ ID NO: 91.

109. The method of any one of claims 104-108, wherein the DNA sequence encoding a transcription termination sequence comprises at least 80%, at least 85%, at least 90%, at least 95%, or 100% sequence identity to any one of SEQ ID NO: 10-SEQ ID NO: 16, SEQ ID NO: 34, SEQ ID NO: 35, SEQ ID NO: 77-SEQ ID NO: 82, or SEQ ID NO: 85-SEQ ID NO: 89.


SEQUENCE LISTING (100 sequences)

SEQ ID NO: 1 - DNA, 393 bp, Homo sapiens
  source 1..393: mol_type = genomic DNA
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctc

SEQ ID NO: 2 - DNA, 263 bp, Homo sapiens
  source 1..263: mol_type = genomic DNA
ttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 3 - DNA, 411 bp, synthetic construct
  source 1..411: mol_type = other DNA
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgcaaatcaagagaaatgcaaatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaaaagtcaccatgagtgtaaagggtgaggcgtatgaggctgtgtcggggcagagcccgaagatctc

SEQ ID NO: 4 - DNA, 256 bp, Mus sp.
  source 1..256: mol_type = genomic DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 5 - DNA, 274 bp, synthetic construct
  source 1..274: mol_type = other DNA
taacaacataggagctgtgattggctgttttcagccaatcagcactgactcatgcaaatcaagagaaatgcaaatagcctttacaagcggtcacaaactcaagaaacgagcggttttaatagtcttttagaatattgtttatcgaaccgaataaggaactgtgctttgtgattcacatatcagtggaggggtgtggaaatggcaccttgataagtcaccatgagtgtaaagggagttgatgtccttccctggctcgctacagacgcacttccgc

SEQ ID NO: 6 - DNA, 300 bp, synthetic construct
  source 1..300: mol_type = other DNA
gccagccagtcttcggcttcgccccctaacggtgacataaggcactctgtgaaatgctctgttccggaatcaaaagattgatccgattatttgcatacccataatgcactgctcacagtacaaatttaaaaaggcaaaatcaaacatttttattctaagcatattctgtgaaagttagacttttgtttaaacaatactcttaaaatttttttctaggtatagaaccttggcattcactagtcaccatcactatactaggagtttctgttacccgagaaacgagttatgaaattaacaagc

SEQ ID NO: 7 - DNA, 300 bp, synthetic construct
  source 1..300: mol_type = other DNA
tggtggcctcaggcgcagcgctaaaacgcatgaaccatttaaggtatttcctgaaactggagcgtgattggtgagactttatttgcatacccacaatgcattgcgcactaaataatgttcgtctttaaaattatttcccctttttccttcaacatatctttctcggaaccgagattgctgtcccagaattgtctgaagaaaaaggctgaagtcaatagctcttttgggccgaaggaaagttaccattacccgtttaggagtagccgttacctgagaactgtagtgtcgacgactgatgtt

SEQ ID NO: 8 - DNA, 316 bp, synthetic construct
  source 1..316: mol_type = other DNA
gtggcctcaggcgcagcgctaaaacgcatgaaccatttaaggtatttcctgaaactggagcgtgattggtgagactttatgcaaatcaagagaaatgcaaatacccacaatgcattgcgcactaaataatgttcgtctttaaaattatttcccctttttccttcaacatatctttctcggaaccgagattgctgtcccagaattgtctgaagaaaaaggctgaagtcaatagctcttttgggccgaaggaaagtcaccatgagtgtaaagggagtagccgttacctgagaactgtagtgtcgacgactgatgttat

SEQ ID NO: 9 - DNA, 316 bp, synthetic construct
  source 1..316: mol_type = other DNA
gtcaagtgcctctctccatttactggtaagagagagagggtttagaggaactcttgttccggcgctcagctcatgcaaatcaagagaaatgcaaatcccagaatgcattgtagatacgagaattattaccagggttatctgtttgaataataatatttaaactttttttctttgtcaggagattttacccagtgagaacatgtttaggacacttttctacagtggaagaaaagcttctgtctgcaggtccattctcgccatcagttaaaagttaccagtcaatagctgggaagccaggcaaaaggctaacaggcag

SEQ ID NO: 10 - DNA, 107 bp, Mus sp.
  source 1..107: mol_type = genomic DNA
cccaatttcactggtctacaatgaaagcaaaacagttctcttccccgctccccggtgtgtgagaggggctttgatccttctctggtttcctaggaaacgcgtatgtg

SEQ ID NO: 11 - DNA, 106 bp, synthetic construct
  source 1..106: mol_type = other DNA
cccaatttcactggtttcaaaaacagaaaaacagttctcttccccgctccccggtgtgtgagaggggctttgatccttctctggtttcctaggaaacgcgtatgtg

SEQ ID NO: 12 - DNA, 46 bp, synthetic construct
  source 1..46: mol_type = other DNA
cccaatttcactggtttcaaaaacagaaaaacagttctcttccccg

SEQ ID NO: 13 - DNA, 100 bp, synthetic construct
  source 1..100: mol_type = other DNA
atataaagctgttaaaaaatcagattgacttcatttagggtgtttcttacagatatcgtttaagttttcggttctgcttgtaaacgcttcaatcgctcat

SEQ ID NO: 14 - DNA, 100 bp, synthetic construct
  source 1..100: mol_type = other DNA
tattttaagtgttttaaaaacagatgcgattccgttaaatcgcgtgtggagctatgtaaagtgtattatagaacaaatgcgagttacggtttttcagctt

SEQ ID NO: 15 - DNA, 100 bp, synthetic construct
  source 1..100: mol_type = other DNA
attttaagtgtttcaaaaacagatgcgattccgttaaatcgcgtgtggagctatgtaaagtgtattatagaacaaatgcgagttacggtttttcagcttt

SEQ ID NO: 16 - DNA, 100 bp, synthetic construct
  source 1..100: mol_type = other DNA
gaatgttctgttgccaatgatagacgtgtgggtggggtgtttcatgctttgggaggttggggtagctccacaaatgtcacccaggttgtagcggagtggt

SEQ ID NO: 17 - DNA, 32 bp, Homo sapiens
  source 1..32: mol_type = genomic DNA
taggctttctggctttttaccggaaagcccct

SEQ ID NO: 18 - RNA, 32 nt, Homo sapiens
  source 1..32: mol_type = genomic RNA
taggctttctggctttttaccggaaagcccct

SEQ ID NO: 19 - DNA, 31 bp, Mus sp.
  source 1..31: mol_type = genomic DNA
caggttttctgacttcggtcggaaaacccct

SEQ ID NO: 20 - RNA, 31 nt, Mus sp.
  source 1..31: mol_type = genomic RNA
caggttttctgacttcggtcggaaaacccct

SEQ ID NO: 21 - DNA, 11 bp, synthetic construct
  source 1..11: mol_type = other DNA
aatttttggag

SEQ ID NO: 22 - RNA, 11 nt, synthetic construct
  source 1..11: mol_type = other RNA
aatttttggag

SEQ ID NO: 23 - DNA, 149 bp, synthetic construct
  source 1..149: mol_type = other DNA
aatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 24 - DNA, 257 bp, synthetic construct
  source 1..257: mol_type = other DNA
gaccgcaccagcaccgcgacgtggaggacgatgatactcagcaacaggaggagcccactggcggcaagttctgctcagcggagtttctgcccggccaaacagcgtgtggaatttttggagcaggttttctgacttcggtcggaaaacccctcccaatttcactggtttcaaaaacagaaaaacagttctcttccccgctccccggtgtgtgagaggggctttgatccttctctggtttcctaggaaacgcgtatgtg

SEQ ID NO: 25 - DNA, 257 bp, synthetic construct
  source 1..257: mol_type = other DNA
gaccggccacaactccctccttggcctttgaaagtcctttcatgaatacatccacggctaatgaattcctttacaccacactggaaaacataaaatacactttgagtggaatttttggagcaggttttctgacttcggtcggaaaacccctcccaatttcactggtttcaaaaacagaaaaacagttctcttccccgctccccggtgtgtgagaggggctttgatccttctctggtttcctaggaaacgcgtatgtg

SEQ ID NO: 26 - DNA, 1784 bp, synthetic construct
  source 1..1784: mol_type = other DNA
ggatcctatcaagaaccaacctagaacaggataccacagtgaatatcactgttagataaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccgcaccagcaccgcgacgtggaggacgatgatactcagcaacaggaggagcccactggcggcaagttctgctcagcggagtttctgcccggccaaacagcgtgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgactgttaacaagacacagtgtgttgtctattcggttggaatacctaacgccctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcagtactaagattgcggcctggccactgactaagatagaagctgtaacttgcctcagattgtggccgctgatctgcag

SEQ ID NO: 27 - DNA, 2197 bp, synthetic construct
  source 1..2197: mol_type = other DNA
ggatcctaaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccgcaccagcaccgcgacgtggaggacgatgatactcagcaacaggaggagcccactggcggcaagttctgctcagcggagtttctgcccggccaaacagcgtgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgtaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaccgcaccagcaccgcgacgtggaggacgatgatactcagcaacaggaggagcccactggcggcaagttctgctcagcggagtttctgcccggccaaacagcgtgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcagtactaagattgcggcctggccactgactaagatagaagctgtaacttgcctcagattgtggccgctgatctgcag

SEQ ID NO: 28 - DNA, 2197 bp, synthetic construct
  source 1..2197: mol_type = other DNA
ggatcccacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattccacacgctgtttggccgggcagaaactccgctgagcagaacttgccgccagtgggctcctcctgttgctgagtatcatcgtcctccacgtcgcggtgctggtgcggtgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttataaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccgcaccagcaccgcgacgtggaggacgatgatactcagcaacaggaggagcccactggcggcaagttctgctcagcggagtttctgcccggccaaacagcgtgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcagtactaagattgcggcctggccactgactaagatagaagctgtaacttgcctcagattgtggccgctgatctgcag

SEQ ID NO: 29 - DNA, 2197 bp, synthetic construct
  source 1..2197: mol_type = other DNA
ggatcctaaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccgcaccagcaccgcgacgtggaggacgatgatactcagcaacaggaggagcccactggcggcaagttctgctcagcggagtttctgcccggccaaacagcgtgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgcacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattccacacgctgtttggccgggcagaaactccgctgagcagaacttgccgccagtgggctcctcctgttgctgagtatcatcgtcctccacgtcgcggtgctggtgcggtgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttacctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcagtactaagattgcggcctggccactgactaagatagaagctgtaacttgcctcagattgtggccgctgatctgcag

SEQ ID NO: 30 - DNA, 1784 bp, synthetic construct
  source 1..1784: mol_type = other DNA
ggatcctatcaagaaccaacctagaacaggataccacagtgaatatcactgttagataaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccggccacaactccctccttggcctttgaaagtcctttcatgaatacatccacggctaatgaattcctttacaccacactggaaaacataaaatacactttgagtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgactgttaacaagacacagtgtgttgtctattcggttggaatacctaacgccctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcagtactaagattgcggcctggccactgactaagatagaagctgtaacttgcctcagattgtggccgctgatctgcag

SEQ ID NO: 31 - DNA, 2197 bp, synthetic construct
  source 1..2197: mol_type = other DNA
ggatcctaaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccggccacaactccctccttggcctttgaaagtcctttcatgaatacatccacggctaatgaattcctttacaccacactggaaaacataaaatacactttgagtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgtaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaccggccacaactccctccttggcctttgaaagtcctttcatgaatacatccacggctaatgaattcctttacaccacactggaaaacataaaatacactttgagtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcagtactaagattgcggcctggccactgactaagatagaagctgtaacttgcctcagattgtggccgctgatctgcag

SEQ ID NO: 32 - DNA, 2197 bp, synthetic construct
  source 1..2197: mol_type = other DNA
ggatcccacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattccactcaaagtgtattttatgttttccagtgtggtgtaaaggaattcattagccgtggatgtattcatgaaaggactttcaaaggccaaggagggagttgtggccggtgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttataaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccggccacaactccctccttggcctttgaaagtcctttcatgaatacatccacggctaatgaattcctttacaccacactggaaaacataaaatacactttgagtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcagtactaagattgcggcctggccactgactaagatagaagctgtaacttgcctcagattgtggccgctgatctgcag

SEQ ID NO: 33 - DNA, 2197 bp, synthetic construct
  source 1..2197: mol_type = other DNA
ggatcctaaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccggccacaactccctccttggcctttgaaagtcctttcatgaatacatccacggctaatgaattcctttacaccacactggaaaacataaaatacactttgagtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgcacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattccactcaaagtgtattttatgttttccagtgtggtgtaaaggaattcattagccgtggatgtattcatgaaaggactttcaaaggccaaggagggagttgtggccggtgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttacctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcagtactaagattgcggcctggccactgactaagatagaagctgtaacttgcctcagattgtggccgctgatctgcag

SEQ ID NO: 34 - DNA, 100 bp, synthetic construct
  source 1..100: mol_type = other DNA
aatttttgtaatgaaaaaatagacggcaagggttattcttaaaactgcagttttgtagcttgggtggcatgttaagtgttctccttacagtcgcaacgat

SEQ ID NO: 35 - DNA, 100 bp, synthetic construct
  source 1..100: mol_type = other DNA
atcatgttttataaaaaaagacttaaagaggaaaacattatggtgcaactttaggcttaagtgattcattgtcactgtttgtttaaacattgtgtaacag

SEQ ID NO: 36 - RNA, 100 nt, synthetic construct
  source 1..100: mol_type = other RNA
gtgtcctttagcagggccttggagccacctaggactctcctggcccttaccacaggggccattcaccttgtgcctcctgccgtggcactggagctgcggt

SEQ ID NO: 37 - DNA, 100 bp, synthetic construct
  source 1..100: mol_type = other DNA
gtgtcctttagcagggccttggagccacctaggactctcctggcccttaccacaggggccattcaccttgtgcctcctgccgtggcactggagctgcggt

SEQ ID NO: 38 - RNA, 100 nt, synthetic construct
  source 1..100: mol_type = other RNA
gtgtcctttagcagggccttggagagcaacaggactctcctggcccttaccacaggggccattcaacctttgcctcctgtcgtggtactggagttgcggt

SEQ ID NO: 39 - DNA, 100 bp, synthetic construct
  source 1..100: mol_type = other DNA
gtgtcctttagcagggccttggagagcaacaggactctcctggcccttaccacaggggccattcaacctttgcctcctgtcgtggtactggagttgcggt

SEQ ID NO: 40 - RNA, 100 nt, synthetic construct
  source 1..100: mol_type = other RNA
gtgtcctttagcagggccttggagagcaacaggactctcttggctcttatcacaggggccattcaacctttgccttctgtcgtggtactggagttgcggt

SEQ ID NO: 41 - DNA, 100 bp, synthetic construct
  source 1..100: mol_type = other DNA
gtgtcctttagcagggccttggagagcaacaggactctcttggctcttatcacaggggccattcaacctttgccttctgtcgtggtactggagttgcggt

SEQ ID NO: 42 - RNA, 100 nt, synthetic construct
  source 1..100: mol_type = other RNA
gtgtcctttagcagggccttggagagcaacagggctctcttggctcttatcgcaggggccattcaacctttgccttctgtcgtggtactggagttgcggt

SEQ ID NO: 43 - DNA, 100 bp, synthetic construct
  source 1..100: mol_type = other DNA
gtgtcctttagcagggccttggagagcaacagggctctcttggctcttatcgcaggggccattcaacctttgccttctgtcgtggtactggagttgcggt

SEQ ID NO: 44 - RNA, 100 nt, synthetic construct
  source 1..100: mol_type = other RNA
gtgtcctttggcagggtcttggagagcaacagggctctcttggctcttatcgcaggggccattcaacctttgccttctgtcgtggtactggagttgcggt

SEQ ID NO: 45 - DNA, 100 bp, synthetic construct
  source 1..100: mol_type = other DNA
gtgtcctttggcagggtcttggagagcaacagggctctcttggctcttatcgcaggggccattcaacctttgccttctgtcgtggtactggagttgcggt

SEQ ID NO: 46 - RNA, 100 nt, synthetic construct
  source 1..100: mol_type = other RNA
gtgtcctttggcagggtcttggagagcaacagggctctcttggctcttatcgcaggggccattcaacctttgtcttctgtcgtggtactggagttgcggt

SEQ ID NO: 47 - DNA, 100 bp, synthetic construct
  source 1..100: mol_type = other DNA
gtgtcctttggcagggtcttggagagcaacagggctctcttggctcttatcgcaggggccattcaacctttgtcttctgtcgtggtactggagttgcggt

SEQ ID NO: 48 - RNA, 100 nt, synthetic construct
  source 1..100: mol_type = other RNA
gtgtcctttggcagggtcttggagagcaacagggctctcttggctcttatcgcaggggccattcaacctttgtcttctgtcgtggtactggggttgcggt

SEQ ID NO: 49 - DNA, 100 bp, synthetic construct
  source 1..100: mol_type = other DNA
gtgtcctttggcagggtcttggagagcaacagggctctcttggctcttatcgcaggggccattcaacctttgtcttctgtcgtggtactggggttgcggt

SEQ ID NO: 50 - RNA, 100 nt, synthetic construct
  source 1..100: mol_type = other RNA
gtgtcctttggcagggtcttggagagcaacagggctctcttggctcttatcgcaggggccatttaacctttgtcttctgtcgtggtactggggttgcggt

SEQ ID NO: 51 - DNA, 100 bp, synthetic construct
  source 1..100: mol_type = other DNA
gtgtcctttggcagggtcttggagagcaacagggctctcttggctcttatcgcaggggccatttaacctttgtcttctgtcgtggtactggggttgcggt

SEQ ID NO: 52 - RNA, 100 nt, synthetic construct
  source 1..100: mol_type = other RNA
gtgtcctttggcagggtcttggagagcaacagggctctcttggctcttatcgcaggggccatttaacctttgtcttctgtcgtggtactggggttgtggt

SEQ ID NO: 53 - DNA, 100 bp, synthetic construct
  source 1..100: mol_type = other DNA
gtgtcctttggcagggtcttggagagcaacagggctctcttggctcttatcgcaggggccatttaacctttgtcttctgtcgtggtactggggttgtggt

SEQ ID NO: 54 - RNA, 100 nt, synthetic construct
  source 1..100: mol_type = other RNA
gtgtcctttggcagggtcttggagagcaacagggctttcttggctcttatcgcaggggccatttaacctttgtcttctgtcgtggtactggggttgtggt

SEQ ID NO: 55 - DNA, 100 bp, synthetic construct
  source 1..100: mol_type = other DNA
gtgtcctttggcagggtcttggagagcaacagggctttcttggctcttatcgcaggggccatttaacctttgtcttctgtcgtggtactggggttgtggt

SEQ ID NO: 56 - RNA, 100 nt, synthetic construct
  source 1..100: mol_type = other RNA
gtgttctttggcagggtcttggagagcaacagggctttcttggctcttatcgcaggggccatttaacctttgtcttctgtcgtggtactggggttgtggt

SEQ ID NO: 57 - DNA, 100 bp, synthetic construct
  source 1..100: mol_type = other DNA
gtgttctttggcagggtcttggagagcaacagggctttcttggctcttatcgcaggggccatttaacctttgtcttctgtcgtggtactggggttgtggt

SEQ ID NO: 58 - RNA, 100 nt, synthetic construct
  source 1..100: mol_type = other RNA
gtgttctttggcagggtcttggagagcaacagggctttcttggctcttatcgcaggggccatttaacctttgtcttctgtcgtggtattggggttgtggt

SEQ ID NO: 59 - DNA, 100 bp, synthetic construct
  source 1..100: mol_type = other DNA
gtgttctttggcagggtcttggagagcaacagggctttcttggctcttatcgcaggggccatttaacctttgtcttctgtcgtggtattggggttgtggt

SEQ ID NO: 60 - RNA, 100 nt, synthetic construct
  source 1..100: mol_type = other RNA
gtgttctttggcagggtcttggagagcaacagggctttcttggctcttatcgcaggggccatttaacctttgtcttctgttgtggtattggggttgtggt

SEQ ID NO: 61 - DNA, 100 bp, synthetic construct
  source 1..100: mol_type = other DNA
gtgttctttggcagggtcttggagagcaacagggctttcttggctcttatcgcaggggccatttaacctttgtcttctgttgtggtattggggttgtggt

SEQ ID NO: 62 - RNA, 100 nt, synthetic construct
  source 1..100: mol_type = other RNA
gtgttctttggcagggtcttggagagcaacagggctttcttggctcttgtcgcaggggccatttaacctttgtcttctgttgtggtattggggttgtggt

SEQ ID NO: 63 - DNA, 100 bp, synthetic construct
  source 1..100: mol_type = other DNA
gtgttctttggcagggtcttggagagcaacagggctttcttggctcttgtcgcaggggccatttaacctttgtcttctgttgtggtattggggttgtggt

SEQ ID NO: 64 - RNA, 100 nt, synthetic construct
  source 1..100: mol_type = other RNA
gtgttctttggcggggtcttggagagcaacagggctttcttggctcttgtcgcaggggccatttaacctttgtcttctgttgtggtattggggttgtggt

SEQ ID NO: 65 - DNA, 100 bp, synthetic construct
  source 1..100: mol_type = other DNA
gtgttctttggcggggtcttggagagcaacagggctttcttggctcttgtcgcaggggccatttaacctttgtcttctgttgtggtattggggttgtggt

SEQ ID NO: 66 - DNA, 282 bp, synthetic construct
  source 1..282: mol_type = other DNA
tcgccccctaacggtgacataaggcactctgtgaaatgctctgttccggaatcaaaagattgatccgattatttgcatacccataatgcactgctcacagtacaaatttaaaaaggcaaaatcaaacatttttattctaagcatattctgtgaaagttagacttttgtttaaacaatactcttaaaatttttttctaggtatagaaccttggcattcactagtcaccatcactatactaggagtttctgttacccgagaaacgagttatgaaattaacaagc

SEQ ID NO: 67 - DNA, 400 bp, synthetic construct
  source 1..400: mol_type = other DNA
atttaatagcagtctttatttaaaagaaatcaaactcagacgtacaaatacacaaaacagataaaacccgagtctctgaccaggaaagcgttattttccagccagccagtcttcggcttcgccccctaacggtgacataaggcactctgtgaaatgctctgttccggaatcaaaagattgatccgattatttgcatacccataatgcactgctcacagtacaaatttaaaaaggcaaaatcaaacatttttattctaagcatattctgtgaaagttagacttttgtttaaacaatactcttaaaatttttttctaggtatagaaccttggcattcactagtcaccatcactatactaggagtttctgttacccgagaaacgagttatgaaattaacaagc

SEQ ID NO: 68 - DNA, 282 bp, synthetic construct
  source 1..282: mol_type = other DNA
tcgccccctaacggtgacataaggcactctgtgaaatgctctgttccggaatcaaaagattgatccgattatttgcatacccataatgcactgctcacagtacaaatttaaaaaggcaaaatcaaacatttttattctaagcatattctgtgaaagttagacttttgtttaaacaatactcttaaaatttttttctaggtatagaaccttggcattcacaagtcaccatgagtgtaaagggagtttctgttacccgagaaacgagttatgaaattaacaagc

SEQ ID NO: 69 - DNA, 282 bp, synthetic construct
  source 1..282: mol_type = other DNA
tcgccccctaacggtgacataaggcactctgtgaaatgctctgttccggaatcaaaagattgatccgattatttgcatacccataatgcactgctcacagtacaaatttaaaaaggcaaaatcaaacatttttattctaagcatattctgtgaaagttagacttttgtttaaacaatactcttaaaatttttttctaggtatagaaccttggcattcagatctcaccatgagtgtaaagggagtttctgttacccgagaaacgagttatgaaattaacaagc

SEQ ID NO: 70 - DNA, 282 bp, synthetic construct
  source 1..282: mol_type = other DNA
tcgccccctaacggtgacataaggcactctgtgaaatgctctgttccggaatcaaaagattgatccgattatttgcatacccataatgcactgctcacagtacaaatttaaaaaggcaaaatcaaacatttttattctaagcatattctgtgaaagttagacttttgtttaaacaatactcttaaaatttttttctaggtatagaaccttggcattcacaaatcaccataaacgtgaaatgagtttctgttacccgagaaacgagttatgaaattaacaagc

SEQ ID NO: 71 - DNA, 282 bp, synthetic construct
  source 1..282: mol_type = other DNA
tcgccccctaacggtgacataaggcactctgtgaaatgctctgttccggaatcaaaagattgatccgattatttgcatacccataatgcactgctcacagtacaaatttaaaaaggcaaaatcaaacatttttattctaagcatattctgtgaaagttagacttttgtttaaacaatactcttaaaatttttttctaggtatagaaccttggcattcagatctcaccataaacgtgaaatgagtttctgttacccgagaaacgagttatgaaattaacaagc

SEQ ID NO: 72 - DNA, 282 bp, synthetic construct
  source 1..282: mol_type = other DNA
tcgccccctaacggtgacataaggcactctgtgaaatgctctgttccggaatcaaaagattgatccgattatttgcatacccataatgcactgctcacagtacaaatttaaaaaggcaaaatcaaacatttttattctaagcatattctgtgaaagttagacttttgtttaaacaatactcttaaaatttttttctaggtatagaaccttggcattcagatctcaccatcactatactaggagtttctgttacccgagaaacgagttatgaaattaacaagc

SEQ ID NO: 73 - DNA, 282 bp, synthetic construct
  source 1..282: mol_type = other DNA
tcgccccctaacggtgacataaggcactctgtgaaatgctctgttccggaatcaaaagattgatccgattatttgcatacccataatgcactgctcacagtacaaatttaaaaaggcaaaatcaaacatttttattctaagcatattctgtgaaagttagacttttgtttaaacaatactcttaaaatttttttctaggtatagaaccttggcattcacaatttgccatgagtatgttgtgagtttctgttacccgagaaacgagttatgaaattaacaagc

SEQ ID NO: 74 - DNA, 282 bp, synthetic construct
  source 1..282: mol_type = other DNA
tcgccccctaacggtgacataaggcactctgtgaaatgctctgttccggaatcaaaagattgatccgattatttgcatacccataatgcactgctcacagtacaaatttaaaaaggcaaaatcaaacatttttattctaagcatattctgtgaaagttagacttttgtttaaacaatactcttaaaatttttttctaggtatagaaccttggcattcagatcttgccatgagtatgttgtgagtttctgttacccgagaaacgagttatgaaattaacaagc

SEQ ID NO: 75 - DNA, 282 bp, synthetic construct
  source 1..282: mol_type = other DNA
tcgccccctaacggtgacataaggcactctgtgaaatgctctgttccggaatcaaaagattgatccgattatttgcatacccataatgcactgctcacagtacaaatttaaaaaggcaaaatcaaacatttttattctaagcatattctgtgaaagttagacttttgtttaaacaatactcttaaaatttttttctaggtatagaaccttggcattcactagacaccatcagtgtactaggagtttctgttacccgagaaacgagttatgaaattaacaagc

SEQ ID NO: 76 - DNA, 282 bp, synthetic construct
  source 1..282: mol_type = other DNA
tcgccccctaacggtgacataaggcactctgtgaaatgctctgttccggaatcaaaagattgatccgattatttgcatacccataatgcactgctcacagtacaaatttaaaaaggcaaaatcaaacatttttattctaagcatattctgtgaaagttagacttttgtttaaacaatactcttaaaatttttttctaggtatagaaccttggcattcagatcacaccatcagtgtactaggagtttctgttacccgagaaacgagttatgaaattaacaagc

SEQ ID NO: 77 - DNA, 95 bp, synthetic construct
  source 1..95: mol_type = other DNA
tataaagctgttaaaaaatcagattgacttcatttagggtgtttcttacagatatcgtttaagttttcggttctgcttgtaaacgcttcaatcgc

SEQ ID NO: 78 - DNA, 200 bp, synthetic construct
  source 1..200: mol_type = other DNA
aatttttgtaatgaaaaaatagacggcaagggttattcttaaaactgcagttttgtagcttgggtggcatgttaagtgttctccttacagtcgcaacgatgggaaacagaaagtaacgtgttatcctctccgccgccgtgagctcttttaacactagctaagtggccgcagggctcttctctttcctttccacttggggc

SEQ ID NO: 79 - DNA, 200 bp, synthetic construct
  source 1..200: mol_type = other DNA
atcatgttttataaaaaaagacttaaagaggaaaacattatggtgcaactttaggcttaagtgattcattgtcactgtttgtttaaacattgtgtaacagaacttgcaaagacagttaactcttgttttccatgtcaaaggtctgaatacttgcatgataaaagtctgtgtaactttccctggtgacatctgacttgcta

SEQ ID NO: 80 - DNA, 200 bp, synthetic construct
  source 1..200: mol_type = other DNA
cttagtaagtttaaaaacagaaaaaaaaccgtgttgctacagctataaacttcaaacatgcagtttatagcagtgggcaacacgtctcatctcaaaaattcagcactcaaacattacacaggaagaggatgtaattttttaaaatgaaagctctgaagtaatttgagtattctctgtcctttttgtaaaaaaaattacga

SEQ ID NO: 81 - DNA, 199 bp, synthetic construct
  source 1..199: mol_type = other DNA
cccaatttcactggtttcaaaaacagaaaaacagttctcttccccgctccccggtgtgtgagaggggctttgatccttctctggtttcctaggaaacgcgtatgtgctagagccacgctctgagacttccgcctcgtgcggtcccgcttcctttctgcctcctctggcctgcatccgtgggggaggtggctggctgcag

SEQ ID NO: 82 - DNA, 100 bp, synthetic construct
  source 1..100: mol_type = other DNA
cttagtaagtttaaaaacagaaaaaaaaccgtgttgctacagctataaacttcaaacatgcagtttatagcagtgggcaacacgtctcatctcaaaaatt

SEQ ID NO: 83 - DNA, 35 bp, synthetic construct
  source 1..35: mol_type = other DNA
aacagggttttctgacttcggtcggaaaaccccct

SEQ ID NO: 84 - DNA, 31 bp, synthetic construct
  source 1..31: mol_type = other DNA
tagtgggggactgcgttcgcgctttcccctg

SEQ ID NO: 85 - DNA, 200 bp, synthetic construct
  source 1..200: mol_type = other DNA
tataaagctgttaaaaaatcagattgacttcatttagggtgtttcttacagatatcgtttaagttttcggttctgcttgtaaacgcttcaatcgctcatgcgtttaaactttgtgtaagtgcaagcgttctttttctagtgaagcttcttccaacaagttagaagtatttacttcagtgtcattaatgtgaaagattctt

SEQ ID NO: 86 - DNA, 100 bp, synthetic construct
  source 1..100: mol_type = other DNA
gtcactcttgtccaatgagagatcataacttgaagtcggtggtctttattgtataatttatttattataaaaatgcatacaacataaaagcatcttcagc

SEQ ID NO: 87 - DNA, 200 bp, synthetic construct
  source 1..200: mol_type = other DNA
gtcactcttgtccaatgagagatcataacttgaagtcggtggtctttattgtataatttatttattataaaaatgcatacaacataaaagcatcttcagcgttttggagtgtacactgagtggtattacatacattcacattgtcgtgcacccatcacccccatccatctccagagctcatttcatcttgcagaactgaa

SEQ ID NO: 88 - DNA, 100 bp, synthetic construct
  source 1..100: mol_type = other DNA
aatttttgtaatgaaaaaatagactcccctataagggttattcttaaaactgcagttttgtggcttgggtggcatgttaagtgttctccttacagtcgca

SEQ ID NO: 89 - DNA, 200 bp, synthetic construct
  source 1..200: mol_type = other DNA
aatttttgtaatgaaaaaatagactcccctataagggttattcttaaaactgcagttttgtggcttgggtggcatgttaagtgttctccttacagtcgcaatgatgggaaacagaaagtaacgtgttatcctctccgccgccgtgagctcttttaacactaagtggccgcagggctcttctcttccctttctacttgggg

SEQ ID NO: 90 - DNA, 416 bp, synthetic construct
  source 1..416: mol_type = other DNA
caattacttttgcaccgatctaatagctcgcccactgtaagtaaagcaggtaaagtcagcctttttcttctgggaccagactctgctctgccccgcggtggtggcctcaggcgcagcgctaaaacgcatgaaccatttaaggtatttcctgaaactggagcgtgattggtgagactttatgcaaatcaagagaaatgcaaatacccacaatgcattgcgcactaaataatgttcgtctttaaaattatttcccctttttccttcaacatatctttctcggaaccgagattgctgtcccagaattgtctgaagaaaaaggctgaagtcaatagctcttttgggccgaaggaaagtcaccatgagtgtaaagggagtagccgttacctgagaactgtagtgtcgacgactgatgttat

SEQ ID NO: 91 - DNA, 416 bp, synthetic construct
  source 1..416: mol_type = other DNA
gattacaggtgtgagtcactgtgcaccgggcactgctactctgaacacatgtggctacatggaagacaggacttggccatgtgctctccccagctcagtagtcaagtgcctctctccatttactggtaagagagagagggtttagaggaactcttgttccggcgctcagctcatgcaaatcaagagaaatgcaaatcccagaatgcattgtagatacgagaattattaccagggttatctgtttgaataataatatttaaactttttttctttgtcaggagattttacccagtgagaacatgtttaggacacttttctacagtggaagaaaagcttctgtctgcaggtccattctcgccatcagttaaaagttaccagtcaatagctgggaagccaggcaaaaggctaacaggcag

SEQ ID NO: 92 - DNA, 44 bp, synthetic construct
  source 1..44: mol_type = other DNA
aatttttggaacagggttttctgccttcgggcggaaaaccccct

SEQ ID NO: 93 - RNA, 45 nt, synthetic construct
  source 1..45: mol_type = other RNA
gtggaatagtataacaatatgctaaatgttgttatagtatcccac

SEQ ID NO: 94 - DNA, 10 bp, synthetic construct
  source 1..10: mol_type = other DNA
aaaaaggggg

SEQ ID NO: 95 - DNA, 10 bp, synthetic construct
  source 1..10: mol_type = other DNA
aaaaaaaaaa

SEQ ID NO: 96 - DNA, 20 bp, synthetic construct
  source 1..20: mol_type = other DNA
cccccggggggggggccccc

SEQ ID NO: 97 - DNA, 10 bp, synthetic construct
  source 1..10: mol_type = other DNA
gggggggggg

SEQ ID NO: 98 - protein, 10 aa, synthetic construct
FFFFFYYYYY

SEQ ID NO: 99 - protein, 10 aa, synthetic construct
YYYYYYYYYY

SEQ ID NO: 100 - protein, 20 aa, synthetic construct
LLLLLFFFFFYYYYYLLLLL